SEQUENCE LISTING (167 sequences)

SEQ ID NO: 1 - DNA, 1046 bp, Homo sapiens
  source 1..1046: mol_type = genomic DNA
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 2 - DNA, 957 bp, synthetic construct
  source 1..957: mol_type = other DNA; note = Modified hIns - 2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 3 - DNA, 957 bp, synthetic construct
  source 1..957: mol_type = other DNA; note = Modified hIns - 3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgaggctcctgcccctgctggccctgctggccctctggggacctgacccagctgcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 4 - DNA, 957 bp, synthetic construct
  source 1..957: mol_type = other DNA; note = Modified hIns - 4
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgaggctcctgcccctgctggctctgctggccctctggggacctgacccagcagcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 5 - DNA, 957 bp, synthetic construct
  source 1..957: mol_type = other DNA; note = Modified hIns - 5
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 6 - DNA, 953 bp, synthetic construct
  source 1..953: mol_type = other DNA; note = Modified hIns -6
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 7 - DNA, 957 bp, synthetic construct
  source 1..957: mol_type = other DNA; note = Modified hIns - 7
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 8 - DNA, 953 bp, synthetic construct
  source 1..953: mol_type = other DNA; note = Modified hIns - 8
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 9 - DNA, 957 bp, synthetic construct
  source 1..957: mol_type = other DNA; note = Modified hIns - 9
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggccctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggagcagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 10 - DNA, 957 bp, synthetic construct
  source 1..957: mol_type = other DNA; note = Modified hIns - 10
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggagtagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 11 - DNA, 957 bp, synthetic construct
  source 1..957: mol_type = other DNA; note = Modified hIns - 11
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggtgggggccctggtgcaggcagcctgcagcccttggccctggaggggtctagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 12 - DNA, 957 bp, synthetic construct
  source 1..957: mol_type = other DNA; note = Modified hIns - 12
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgctttcgtgaaccagcacctctgcggaagcgatctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 13 - DNA, 957 bp, synthetic construct
  source 1..957: mol_type = other DNA; note = Modified hIns - 13
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgcttttgtgaaccagcacctctgcggatctgatctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 14 - DNA, 957 bp, synthetic construct
  source 1..957: mol_type = other DNA; note = Modified hIns - 14
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcggctcttgccccttctggctttactggcactatggggccctgacccagcggccgcttttgtgaaccagcacctctgcggatctgatctggtggaggccttgtacctggtttgtggggaaagaggttttttctatacccccaggacaaagcgcgaggcagaagaccttcaagtgggccaggtagagctgggagggggcccgggtgcaggatccctgcagcctctggctctggaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 15 - DNA, 957 bp, synthetic construct
  source 1..957: mol_type = other DNA; note = Modified hIns - 15
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcttgccccttctggctttactggcactatggggccctgacccagcagctgcttttgtgaaccagcacctctgtggatctgatctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 16 - DNA, 957 bp, synthetic construct
  source 1..957: mol_type = other DNA; note = Modified hIns - 16
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcttgccccttctggctttactggcactatggggccctgacccagcagctgcttttgtgaaccagcacctctgtggatctgatctggtggaggccttgtacctggtttgtggggaaagaggttttttctatacccccaggacaaagagagaggcagaagaccttcaagtgggccaggtagagctgggagggggcccaggtgcaggatccctgcagcctctggctctggaagggtctagacaaaaaagaggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 17-18 - skipped (2 entries)

SEQ ID NO: 19 - DNA, 2118 bp, Homo sapiens
  source 1..2118: mol_type = genomic DNA
ctagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaacgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 20 - DNA, 2025 bp, synthetic construct
  source 1..2025: mol_type = other DNA; note = Modified hGck - 2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 21 - DNA, 2025 bp, synthetic construct
  source 1..2025: mol_type = other DNA; note = Modified hGck-3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagggatgctatcaaaaggagaggggactttgaaatggatgtggtggcaatggtgaatgacacagtggccacaatgatctcctgctactatgaagaccatcagtgtgaggtgggcatgattgtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacacaggagacaggaagcagatctacaacatcctgagcaccctggggctgaggccctccaccacagactgtgacattgtgagaagagcctgtgagtctgtgtctaccagggctgcccacatgtgctctgctgggctggctggggtcatcaacagaatgagggagagcaggtctgaggatgtaatgagaatcactgtgggagtggatggctctgtgtacaagctgcacccctcattcaaggagagattccatgcctctgtgagaaggctgacccccagctgtgagatcacctttattgagtctgaggagggcagtggcagaggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 22 - DNA, 2025 bp, synthetic construct
  source 1..2025: mol_type = other DNA; note = Modified hGck-4
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagggatgctatcaaaaggagaggggactttgaaatggatgtggtggcaatggtgaatgacactgtggccactatgatctcctgctactatgaagaccatcagtgtgaggtaggcatgattgtgggcactggctgcaatgcctgctacatggaggagatgcagaatgtggagctggtggagggggatgagggcagaatgtgtgtcaatactgagtggggtgcctttggggactcaggggagctggatgagttcctgctggagtatgacagactggtggatgagagctctgcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacactggtgacaggaagcagatctacaacatcctgagcacactggggctgaggccctccaccacagactgtgacattgtgagaagagcttgtgagagtgtgtctaccagggctgctcacatgtgcagtgctgggctggctggggtcatcaacagaatgagggagagcaggtctgaggatgtaatgagaatcactgtgggtgtggatggcagtgtgtacaagctgcacccctcattcaaggagagattccatgccagtgtgagaaggctgacccccagctgtgagatcaccttcattgagtctgaggagggcagtggcaggggtgcagccctggtcagtgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 23 - DNA, 2025 bp, synthetic construct
  source 1..2025: mol_type = other DNA; note = Modified hGck-5
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacatagataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccactatgatctcctgctactatgaagaccatcagtgtgaggtgggcatgatagtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctctgagcagctgagaacaaggggagcctttgagaccaggtttgtgtcccaggtggagtctgacactggggacaggaagcagatctacaacatcctgagcaccctggggctgaggccctccaccactgactgtgacatagtgaggagagcctgtgagtctgtgtctaccagggctgcccacatgtgctctgctgggctggcaggggtcatcaacaggatgagggagagcagatctgaggatgtaatgaggatcactgtgggtgtggatggctctgtgtacaagctgcacccctcattcaaggagaggttccatgcctctgtgaggaggctgacccccagctgtgagatcacctttatagagtctgaggagggcagtggcagaggggcagccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 24 - DNA, 2025 bp, synthetic construct
  source 1..2025: mol_type = other DNA; note = Modified hGck-6
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 25 - DNA, 2025 bp, synthetic construct
  source 1..2025: mol_type = other DNA; note = Modified hGck-7
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccacaatgatctcctgctactatgaagaccatcagtgtgaggtgggcatgatagtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaagaggagcctttgagaccagatttgtgtcacaggtggagtctgacactggagacagaaagcagatctacaacatcctgagcacactggggctgaggccctccaccactgactgtgacatagtgaggagagcttgtgagagtgtgtctaccagagctgcccacatgtgctctgctgggctggctggggtcatcaacaggatgagagagagcaggagtgaggatgtaatgagaatcactgtgggggtggatggctctgtgtacaagctgcacccctcattcaaggagaggttccatgcctctgtgaggaggctgacccccagctgtgagatcacctttattgagtctgaggagggcagtggaaggggagcagccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 26 - DNA, 2025 bp, synthetic construct
  source 1..2025: mol_type = other DNA; note = Modified hGck-8
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 27 - DNA, 2025 bp, synthetic construct
  source 1..2025: mol_type = other DNA; note = Modified hGck-9
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 28 - DNA, 2025 bp, synthetic construct
  source 1..2025: mol_type = other DNA; note = Modified hGck-10
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 29 - DNA, 2025 bp, synthetic construct
  source 1..2025: mol_type = other DNA; note = Modified hGck-11
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccggaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggccgcatgtgtgtcaatactgagtggggcgcctttggggacagtggagagctggatgagttcctgctggagtatgaccgcctggtggatgagagctctgcaaacccaggtcagcagctgtatgagaagctcataggtggcaagtacatgggagagctggtgcggcttgtgctgctcaggctagtggatgaaaacctgctcttccatggggaggcctcagagcagctgcgcacacgcggagcctttgagacgcgctttgtgtctcaagtggagagtgacacaggtgaccgcaagcagatctacaacatcctgagcactctggggctgcggccctcaaccacagactgtgacattgtgcgccgcgcctgtgagtctgtgtctacgcgcgctgcgcacatgtgcagtgcggggctggcgggagtcatcaaccgcatgcgcgagagccgctcagaggatgtaatgcgcatcactgtgggtgtggatggctctgtgtacaagctgcaccccagcttcaaggagcggttccatgccagtgtgcgcaggctgactcccagctgtgagatcacctttatagagtcagaggagggcagtggcagaggcgcggccctggtctccgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 30 - DNA, 2025 bp, synthetic construct
  source 1..2025: mol_type = other DNA; note = Modified hGck-12
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggccgcatgtgtgtcaatactgagtggggcgcctttggggactctggtgagctggatgagttcctgctggagtatgaccgcctggtggatgagagctctgcaaaccctggtcagcagctgtatgagaagctcataggtggcaagtacatgggtgagctggtgcggcttgtgctgctcaggttagtggatgaaaacctgctcttccatggggaggcctctgagcagctgcgcacacgcggagcctttgagacgcgctttgtgtctcaagtggagtctgacactggtgaccgcaagcagatctacaacatcctgagcactctggggctgagaccctctaccactgactgtgacattgtgcgccgcgcctgtgagtctgtgtctacgcgcgctgcgcacatgtgctctgcggggctggcgggtgtcatcaaccgcatgcgcgagagccgctctgaggatgtaatgcgcatcactgtgggtgtggatggctctgtgtacaagctgcacccctcattcaaggagcggttccatgcctctgtgcgcaggctgactcccagctgtgagatcacctttattgagtctgaggagggcagtggcagaggcgcggccctggtctctgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 31 - DNA, 2025 bp, synthetic construct
  source 1..2025: mol_type = other DNA; note = Modified hGck-13
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 32 - DNA, 2025 bp, synthetic construct
  source 1..2025: mol_type = other DNA; note = Modified hGck-14
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 33 - DNA, 2025 bp, synthetic construct
  source 1..2025: mol_type = other DNA; note = Modified hGck-15
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccagaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggcaggatgtgtgtcaatactgagtggggagcctttggggacagtggagagctggatgagttcctgctggagtatgacagactggtggatgagagctctgcaaaccctggtcagcagctgtatgagaagctcataggtggcaagtacatgggtgagctggtgaggcttgtgctgctcaggctagtggatgaaaacctgctcttccatggggaggcctcagagcagctgagaacaaggggagcctttgagacaagatttgtgtctcaggtggagagtgacactggagacaggaagcagatctacaacatcctgagcacactggggctgagaccctcaaccactgactgtgacattgtgaggagagcctgtgagtctgtgtctacaagggctgcacacatgtgcagtgctgggctggcaggtgtcatcaacagaatgagggagagcagatcagaggatgtaatgaggatcactgtgggagtggatggctctgtgtacaagctgcaccccagcttcaaggagagattccatgccagtgtgaggaggctgacccccagctgtgagatcacctttatagagtcagaggagggcagtggcagaggtgctgccctggtctctgcagtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 34 - DNA, 2025 bp, synthetic construct
  source 1..2025: mol_type = other DNA; note = Modified hGck-16
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggcagaatgtgtgtcaatactgagtggggtgcctttggggactctggtgagctggatgagttcctgctggagtatgacagactggtggatgagagctctgcaaaccctggtcagcagctgtatgagaagctcataggtggcaagtacatgggtgagctggtgagacttgtgctgctcaggttagtggatgaaaacctgctcttccatggggaggcctctgagcagctgagaacaagaggagcctttgagactagatttgtgtctcaggtggagtctgacactggtgacagaaagcagatctacaacatcctgagcactctggggctgagaccctctaccactgactgtgacattgtgagaagagcctgtgagtctgtgtctactagagctgctcacatgtgctctgctgggctggctggtgtcatcaacagaatgagagagagcagatctgaggatgtaatgagaatcactgtgggtgtggatggctctgtgtacaagctgcacccctcattcaaggagagattccatgcctctgtgagaaggctgactcccagctgtgagatcaccttcattgagtctgaggagggcagtggcagaggtgctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 35 - DNA, 2025 bp, synthetic construct
  source 1..2025: mol_type = other DNA; note = Modified hGck-17
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggacgtgacccggagccaggctcaaacagcactgactctcgtcgagcagatcttggcggaattccagcttcaagaggaagatctgaagaaagttatgagaaggatgcagaaggagatggaccgcggcttacgactggaaacgcacgaggaggcctccgtgaaaatgctacccacctacgtgcgttctacacctgaaggatcagaggtgggggattttctcagtctggacttgggtggcactaacttccgggtcatgctggtgaaggttggagaaggggaggaaggccagtggtcggtgaaaaccaagcatcagatgtatagcattccagaggatgctatgacaggcaccgccgagatgctttttgactacatatccgaatgcatctctgatttcctcgacaagcaccaaatgaaacataagaaactgccgctgggatttactttctcatttcccgtgagacacgaggatattgacaaggggatcctcttgaattggacgaaaggcttcaaggcaagtggcgccgaaggaaacaatgtagtggggcttctgagggatgctattaagcgccggggtgactttgagatggacgttgtggcaatggtcaacgatacagtggccacaatgatcagctgttattacgaggaccatcagtgcgaagtcggcatgatagtgggaactgggtgtaatgcgtgctatatggaggaaatgcagaacgttgagttagtggaaggcgatgagggtagaatgtgtgtaaataccgagtggggagctttcggggactccggcgaactggatgagttcctactcgaatacgacaggctggtggatgagtctagcgccaaccctggccagcaattgtatgaaaaactgatcggagggaagtacatgggtgagcttgtccgcctcgttctgctgcgattagtggacgagaatctcctgtttcacggcgaagcatccgagcagttgcgtacacggggagccttcgaaaccagatttgtctcacaagtggagagtgatactggggacaggaaacagatttacaacatccttagcacactgggcctacgcccatctacgaccgattgcgacattgtgcggcgagcttgtgagtccgtttcgactagagccgcacacatgtgctcagctggtctcgccggagtaatcaataggatgcgcgaaagccggagtgaggacgtgatgagaataacagtcggcgtggatgggtctgtctataagctgcatccctccttcaaggaaaggtttcacgcgagcgtgcgccgtctgaccccttcatgtgagattactttcatcgaatccgaggagggcagtggacggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 36 - DNA, 2025 bp, synthetic construct
  source 1..2025: mol_type = other DNA; note = Modified hGck-18
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtagagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctagacaagcaccaaatgaaacataagaaactgccgctgggatttactttctcatttcctgtgagacatgaggatattgacaaggggatcctcttgaattggacaaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctattagaatatgacaggctggtggatgagtctagcgccaaccctggccagcaattgtatgaaaaactgattggagggaagtacatgggtgagcttgtccgccttgttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccactgattgtgacattgtgcggcgggcttgtgagtcagtttccactagagccgcacacatgtgctcagctggtttggctggagtaatcaataggatgcgcgaaagcaggagtgaggatgtgatgagaataacagttggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 37 - DNA, 2025 bp, synthetic construct
  source 1..2025: mol_type = other DNA; note = Modified hGck-19
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtggagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctggacaagcaccaaatgaaacataagaaactgccgctgggatttactttctcatttcctgtgagacatgaggatattgacaaggggatcctcttgaattggaccaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctactggaatatgacaggctggtggatgagtctagcgccaaccctggccagcaattgtatgaaaaactgattggagggaagtacatgggtgagcttgtccgcctggttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccacagattgtgacattgtgcggcgggcttgtgagtcagttagcactagagccgcacacatgtgctcagctggtctggctggagtaatcaataggatgcgcgaaagcaggagtgaggatgtgatgagaataacagtgggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 38 - DNA, 2025 bp, synthetic construct
  source 1..2025: mol_type = other DNA; note = Modified hGck-20
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtagagcagatcttggctgaattccagcttcaagaggaagatctgaagaaagttatgagaaggatgcagaaggagatggacaggggcttaagactggaaacccatgaggaggccagtgtgaaaatgctacccacctatgtaaggtctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctagacaagcaccaaatgaaacataagaaactgccactgggatttactttctcatttcctgtgagacatgaggatattgacaaggggatcctcttgaattggaccaaaggcttcaaggcaagtggtgctgaaggaaacaatgtagtggggcttctgagggatgctattaagaggagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctattagaatatgacaggctggtggatgagtcttcagccaaccctggccagcaattgtatgaaaaactgattggagggaagtacatgggtgagcttgtcaggcttgttctgctgagattagtggatgagaatctcctgtttcatggtgaagcatctgagcagttgaggacaagaggagcctttgaaaccagatttgtctcacaagtggagagtgatactggggacaggaaacagatttacaacatccttagcacactgggcctaaggccatctaccactgattgtgacattgtgagaagggcttgtgagagtgtttccactagagcagcacacatgtgctcagctggtttggctggagtaatcaataggatgagagaaagcaggagtgaggatgtgatgagaataacagttggagtggatgggtctgtctataagctgcatccctccttcaaggaaaggtttcatgcctctgtgagaaggctgaccccttcatgtgagattactttcatagaatcagaggagggcagtggaagaggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 39 - DNA, 2025 bp, synthetic construct
  source 1..2025: mol_type = other DNA; note = Modified hGck-21
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtgaccagaagccaggctcaaacagcactgactctggtggagcagatcttggctgaattccagcttcaagaggaagatctgaagaaagttatgagaaggatgcagaaggagatggacagaggcttaagactggaaacccatgaggaggccagtgtgaaaatgctacccacctatgtaagatctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctggacaagcaccaaatgaaacataagaaactgcccctgggatttactttctcatttcctgtgagacatgaggatattgacaaggggatcctcttgaattggaccaaaggcttcaaggcaagtggtgctgaaggaaacaatgtagtggggcttctgagggatgctattaagagaagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctactggaatatgacaggctggtggatgagtcttcagccaaccctggccagcaattgtatgaaaaactgattggagggaagtacatgggtgagcttgtcagactggttctgctgagattagtggatgagaatctcctgtttcatggtgaagcatctgagcagttgagaacaagaggagcctttgaaaccagatttgtctcacaagtggagagtgatactggggacaggaaacagatttacaacatccttagcacactgggcctaagaccatctaccactgattgtgacattgtgagaagagcttgtgagagtgttagcactagagcagcacacatgtgctcagctggtctggctggagtaatcaataggatgagagaaagcagaagtgaggatgtgatgagaataacagtgggagtggatgggtctgtctataagctgcatccctccttcaaggaaaggtttcatgcctctgtgagaagactgaccccttcatgtgagattactttcatagaatcagaggagggcagtggaagaggggcagccttggtttctgctgtggcctgcaaaaaggcatgcatgcttggccaatgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 40 - skipped

SEQ ID NO: 41 - protein, 110 aa, synthetic construct
  source 1..110: note = wild type insulin aa
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 42 - DNA, 329 bp, synthetic construct
  source 1..329: mol_type = other DNA; note = 5' UTR
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccacc

SEQ ID NO: 43 - DNA, 333 bp, synthetic construct
  source 1..333: mol_type = other DNA; note = ORF insulin
atggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactag

SEQ ID NO: 44 - DNA, 333 bp, synthetic construct
  source 1..333: mol_type = other DNA; note = ORF insulin
atggccctgtggatgaggctcctgcccctgctggccctgctggccctctggggacctgacccagctgcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagaggggcattgtggaacaatgctgtaccagcatctgctccctctaccagctggagaactactgcaactag

SEQ ID NO: 45 - DNA, 333 bp, synthetic construct
  source 1..333: mol_type = other DNA; note = ORF insulin
atggccctgtggatgaggctcctgcccctgctggctctgctggccctctggggacctgacccagcagcagcctttgtgaaccaacacctgtgtggctcagatctggtggaagctctctacctagtgtgtggggaaaggggcttcttctacacacccaggaccaagagagaggcagaggacctgcaggtggggcaggtggagctgggagggggccctggtgcaggcagcctgcagcccttggccctggaggggagcaggcagaagagaggcattgtggaacaatgctgtaccagcatctgctccctctaccagctggagaactactgcaactag

SEQ ID NO: 46 - DNA, 333 bp, synthetic construct
  source 1..333: mol_type = other DNA; note = ORF insulin
atggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaa

SEQ ID NO: 47 - DNA, 333 bp, synthetic construct
  source 1..333: mol_type = other DNA; note = ORF insulin
atggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 48 - DNA, 333 bp, synthetic construct
  source 1..333: mol_type = other DNA; note = ORF insulin
atggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagagaggcattgtggaacaatgctgtaccagcatctgctccctctaccagctggagaactactgcaactag

SEQ ID NO: 49 - DNA, 333 bp, synthetic construct
  source 1..333: mol_type = other DNA; note = ORF insulin
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactag

SEQ ID NO: 50 - DNA, 333 bp, synthetic construct
  source 1..333: mol_type = other DNA; note = ORF insulin
atggccctgtggatgagactcctgcccctgctggccctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggagcagacagaagagaggcattgtggaacaatgctgtaccagcatctgctccctctaccagctggagaactactgcaactag

SEQ ID NO: 51 - DNA, 333 bp, synthetic construct
  source 1..333: mol_type = other DNA; note = ORF insulin
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggagtagacagaagaggggcattgtggaacaatgctgtaccagcatctgctccctctaccagctggagaactactgcaactag

SEQ ID NO: 52 - DNA, 333 bp, synthetic construct
  source 1..333: mol_type = other DNA; note = ORF insulin
atggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagagaggcagaggacctgcaggtggggcaggtggagctgggtgggggccctggtgcaggcagcctgcagcccttggccctggaggggtctagacagaagagaggcattgtggaacaatgctgtaccagcatctgctccctctaccagctggagaactactgcaactag

SEQ ID NO: 53 - DNA, 333 bp, synthetic construct
  source 1..333: mol_type = other DNA; note = ORF insulin
atggccctgtggatgcggctcttgccccttctggctttactggcactatggggccctgacccagcggccgctttcgtgaaccagcacctctgcggaagcgatctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactga

SEQ ID NO: 54 - DNA, 333 bp, synthetic construct
  source 1..333: mol_type = other DNA; note = ORF insulin
atggccctgtggatgcggctcttgccccttctggctttactggcactatggggccctgacccagcggccgcttttgtgaaccagcacctctgcggatctgatctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactga

SEQ ID NO: 55 - DNA, 333 bp, synthetic construct
  source 1..333: mol_type = other DNA; note = ORF insulin
atggccctgtggatgcggctcttgccccttctggctttactggcactatggggccctgacccagcggccgcttttgtgaaccagcacctctgcggatctgatctggtggaggccttgtacctggtttgtggggaaagaggttttttctatacccccaggacaaagcgcgaggcagaagaccttcaagtgggccaggtagagctgggagggggcccgggtgcaggatccctgcagcctctggctctggaagggtctagacaaaaaaggggcatagtggagcagtgctgtacttcaatttgcagtttgtaccagcttgagaattattgtaactga

SEQ ID NO: 56 - DNA, 333 bp, synthetic construct
  source 1..333: mol_type = other DNA; note = ORF insulin
atggccctgtggatgagactcttgccccttctggctttactggcactatggggccctgacccagcagctgcttttgtgaaccagcacctctgtggatctgatctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactga

SEQ ID NO: 57 - DNA, 333 bp, synthetic construct
  source 1..333: mol_type = other DNA; note = ORF insulin
atggccctgtggatgagactcttgccccttctggctttactggcactatggggccctgacccagcagctgcttttgtgaaccagcacctctgtggatctgatctggtggaggccttgtacctggtttgtggggaaagaggttttttctatacccccaggacaaagagagaggcagaagaccttcaagtgggccaggtagagctgggagggggcccaggtgcaggatccctgcagcctctggctctggaagggtctagacaaaaaagaggcatagtggagcagtgctgtacttcaatttgcagtttgtaccagcttgagaattattgtaactga

SEQ ID NO: 58-59 - skipped (2 entries)

SEQ ID NO: 60 - DNA, 295 bp, synthetic construct
  source 1..295: mol_type = other DNA; note = 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 61 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtga

SEQ ID NO: 62 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagggatgctatcaaaaggagaggggactttgaaatggatgtggtggcaatggtgaatgacacagtggccacaatgatctcctgctactatgaagaccatcagtgtgaggtgggcatgattgtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacacaggagacaggaagcagatctacaacatcctgagcaccctggggctgaggccctccaccacagactgtgacattgtgagaagagcctgtgagtctgtgtctaccagggctgcccacatgtgctctgctgggctggctggggtcatcaacagaatgagggagagcaggtctgaggatgtaatgagaatcactgtgggagtggatggctctgtgtacaagctgcacccctcattcaaggagagattccatgcctctgtgagaaggctgacccccagctgtgagatcacctttattgagtctgaggagggcagtggcagaggagctgccctggtctctgctgtggcctgtaagaaggcctgtatgctgggccagtga

SEQ ID NO: 63 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagggatgctatcaaaaggagaggggactttgaaatggatgtggtggcaatggtgaatgacactgtggccactatgatctcctgctactatgaagaccatcagtgtgaggtaggcatgattgtgggcactggctgcaatgcctgctacatggaggagatgcagaatgtggagctggtggagggggatgagggcagaatgtgtgtcaatactgagtggggtgcctttggggactcaggggagctggatgagttcctgctggagtatgacagactggtggatgagagctctgcaaaccctggtcagcagctgtatgagaagctcataggtggcaagtacatgggagagctggtgagacttgtgctgctcaggctggtggatgaaaacctgctcttccatggggaggcctctgagcagctgagaacaagaggagcctttgagaccagatttgtgtcccaggtggagtctgacactggtgacaggaagcagatctacaacatcctgagcacactggggctgaggccctccaccacagactgtgacattgtgagaagagcttgtgagagtgtgtctaccagggctgctcacatgtgcagtgctgggctggctggggtcatcaacagaatgagggagagcaggtctgaggatgtaatgagaatcactgtgggtgtggatggcagtgtgtacaagctgcacccctcattcaaggagagattccatgccagtgtgagaaggctgacccccagctgtgagatcaccttcattgagtctgaggagggcagtggcaggggtgcagccctggtcagtgcagtggcctgtaagaaggcctgtatgctgggccagtga

SEQ ID NO: 64 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaagaatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctcagacttcctggacaagcatcagatgaaacacaagaagctgcccctgggcttcaccttctcctttcctgtgaggcatgaagacatagataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccactatgatctcctgctactatgaagaccatcagtgtgaggtgggcatgatagtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctctgagcagctgagaacaaggggagcctttgagaccaggtttgtgtcccaggtggagtctgacactggggacaggaagcagatctacaacatcctgagcaccctggggctgaggccctccaccactgactgtgacatagtgaggagagcctgtgagtctgtgtctaccagggctgcccacatgtgctctgctgggctggcaggggtcatcaacaggatgagggagagcagatctgaggatgtaatgaggatcactgtgggtgtggatggctctgtgtacaagctgcacccctcattcaaggagaggttccatgcctctgtgaggaggctgacccccagctgtgagatcacctttatagagtctgaggagggcagtggcagaggggcagccctggtctctgcagtggcctgtaagaaggcctgtatgctgggccagtga

SEQ ID NO: 65 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtga

SEQ ID NO: 66 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccacaatgatctcctgctactatgaagaccatcagtgtgaggtgggcatgatagtgggcacaggctgcaatgcctgctacatggaggagatgcagaatgtggagctggtggagggggatgagggcagaatgtgtgtcaatacagagtggggagcctttggggactctggggagctggatgagttcctgctggagtatgacagactggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaagaggagcctttgagaccagatttgtgtcacaggtggagtctgacactggagacagaaagcagatctacaacatcctgagcacactggggctgaggccctccaccactgactgtgacatagtgaggagagcttgtgagagtgtgtctaccagagctgcccacatgtgctctgctgggctggctggggtcatcaacaggatgagagagagcaggagtgaggatgtaatgagaatcactgtgggggtggatggctctgtgtacaagctgcacccctcattcaaggagaggttccatgcctctgtgaggaggctgacccccagctgtgagatcacctttattgagtctgaggagggcagtggaaggggagcagccctggtctctgcagtggcctgtaagaaggcctgtatgctgggccagtga

SEQ ID NO: 67 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtga

SEQ ID NO: 68 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtga

SEQ ID NO: 69 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtga

SEQ ID NO: 70 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccggaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggccgcatgtgtgtcaatactgagtggggcgcctttggggacagtggagagctggatgagttcctgctggagtatgaccgcctggtggatgagagctctgcaaacccaggtcagcagctgtatgagaagctcataggtggcaagtacatgggagagctggtgcggcttgtgctgctcaggctagtggatgaaaacctgctcttccatggggaggcctcagagcagctgcgcacacgcggagcctttgagacgcgctttgtgtctcaagtggagagtgacacaggtgaccgcaagcagatctacaacatcctgagcactctggggctgcggccctcaaccacagactgtgacattgtgcgccgcgcctgtgagtctgtgtctacgcgcgctgcgcacatgtgcagtgcggggctggcgggagtcatcaaccgcatgcgcgagagccgctcagaggatgtaatgcgcatcactgtgggtgtggatggctctgtgtacaagctgcaccccagcttcaaggagcggttccatgccagtgtgcgcaggctgactcccagctgtgagatcacctttatagagtcagaggagggcagtggcagaggcgcggccctggtctccgcggtggcctgtaagaaggcctgtatgctgggccagtga

SEQ ID NO: 71 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggccgcatgtgtgtcaatactgagtggggcgcctttggggactctggtgagctggatgagttcctgctggagtatgaccgcctggtggatgagagctctgcaaaccctggtcagcagctgtatgagaagctcataggtggcaagtacatgggtgagctggtgcggcttgtgctgctcaggttagtggatgaaaacctgctcttccatggggaggcctctgagcagctgcgcacacgcggagcctttgagacgcgctttgtgtctcaagtggagtctgacactggtgaccgcaagcagatctacaacatcctgagcactctggggctgagaccctctaccactgactgtgacattgtgcgccgcgcctgtgagtctgtgtctacgcgcgctgcgcacatgtgctctgcggggctggcgggtgtcatcaaccgcatgcgcgagagccgctctgaggatgtaatgcgcatcactgtgggtgtggatggctctgtgtacaagctgcacccctcattcaaggagcggttccatgcctctgtgcgcaggctgactcccagctgtgagatcacctttattgagtctgaggagggcagtggcagaggcgcggccctggtctctgcggtggcctgtaagaaggcctgtatgctgggccagtga

SEQ ID NO: 72 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtga

SEQ ID NO: 73 - DNA, 1401 bp, synthetic construct
  source 1..1401: mol_type = other DNA; note = ORF Gck
atggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtga

SEQ ID NO: 74 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatcccagaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggtaggcatgatagtgggcacaggctgcaatgcctgctacatggaggagatgcagaatgtggagctggtggagggggatgagggcaggatgtgtgtcaatactgagtggggagcctttggggacagtggagagctggatgagttcctgctggagtatgacagactggtggatgagagctctgcaaaccctggtcagcagctgtatgagaagctcataggtggcaagtacatgggtgagctggtgaggcttgtgctgctcaggctagtggatgaaaacctgctcttccatggggaggcctcagagcagctgagaacaaggggagcctttgagacaagatttgtgtctcaggtggagagtgacactggagacaggaagcagatctacaacatcctgagcacactggggctgagaccctcaaccactgactgtgacattgtgaggagagcctgtgagtctgtgtctacaagggctgcacacatgtgcagtgctgggctggcaggtgtcatcaacagaatgagggagagcagatcagaggatgtaatgaggatcactgtgggagtggatggctctgtgtacaagctgcaccccagcttcaaggagagattccatgccagtgtgaggaggctgacccccagctgtgagatcacctttatagagtcagaggagggcagtggcagaggtgctgccctggtctctgcagtggcctgtaagaaggcctgtatgctgggccagtga

SEQ ID NO: 75 - DNA, 1401 bp, synthetic construct
  source 1..1401: mol_type = other DNA; note = ORF Gck
atggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggtctgtgaagaccaaacaccagatgtactccatccctgaggatgccatgactggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacactgtggccactatgatctcctgctactatgaagaccatcagtgtgaggttggcatgattgtgggcactggctgcaatgcctgctacatggaggagatgcagaatgtggagctggtggagggggatgagggcagaatgtgtgtcaatactgagtggggtgcctttggggactctggtgagctggatgagttcctgctggagtatgacagactggtggatgagagctctgcaaaccctggtcagcagctgtatgagaagctcataggtggcaagtacatgggtgagctggtgagacttgtgctgctcaggttagtggatgaaaacctgctcttccatggggaggcctctgagcagctgagaacaagaggagcctttgagactagatttgtgtctcaggtggagtctgacactggtgacagaaagcagatctacaacatcctgagcactctggggctgagaccctctaccactgactgtgacattgtgagaagagcctgtgagtctgtgtctactagagctgctcacatgtgctctgctgggctggctggtgtcatcaacagaatgagagagagcagatctgaggatgtaatgagaatcactgtgggtgtggatggctctgtgtacaagctgcacccctcattcaaggagagattccatgcctctgtgagaaggctgactcccagctgtgagatcaccttcattgagtctgaggagggcagtggcagaggtgctgccctggtctctgctgtggcctgtaagaaggcctgtatgctgggccagtga

SEQ ID NO: 76 - DNA, 1401 bp, synthetic construct
  source 1..1401: mol_type = other DNA; note = ORF Gck
atggccatggacgtgacccggagccaggctcaaacagcactgactctcgtcgagcagatcttggcggaattccagcttcaagaggaagatctgaagaaagttatgagaaggatgcagaaggagatggaccgcggcttacgactggaaacgcacgaggaggcctccgtgaaaatgctacccacctacgtgcgttctacacctgaaggatcagaggtgggggattttctcagtctggacttgggtggcactaacttccgggtcatgctggtgaaggttggagaaggggaggaaggccagtggtcggtgaaaaccaagcatcagatgtatagcattccagaggatgctatgacaggcaccgccgagatgctttttgactacatatccgaatgcatctctgatttcctcgacaagcaccaaatgaaacataagaaactgccgctgggatttactttctcatttcccgtgagacacgaggatattgacaaggggatcctcttgaattggacgaaaggcttcaaggcaagtggcgccgaaggaaacaatgtagtggggcttctgagggatgctattaagcgccggggtgactttgagatggacgttgtggcaatggtcaacgatacagtggccacaatgatcagctgttattacgaggaccatcagtgcgaagtcggcatgatagtgggaactgggtgtaatgcgtgctatatggaggaaatgcagaacgttgagttagtggaaggcgatgagggtagaatgtgtgtaaataccgagtggggagctttcggggactccggcgaactggatgagttcctactcgaatacgacaggctggtggatgagtctagcgccaaccctggccagcaattgtatgaaaaactgatcggagggaagtacatgggtgagcttgtccgcctcgttctgctgcgattagtggacgagaatctcctgtttcacggcgaagcatccgagcagttgcgtacacggggagccttcgaaaccagatttgtctcacaagtggagagtgatactggggacaggaaacagatttacaacatccttagcacactgggcctacgcccatctacgaccgattgcgacattgtgcggcgagcttgtgagtccgtttcgactagagccgcacacatgtgctcagctggtctcgccggagtaatcaataggatgcgcgaaagccggagtgaggacgtgatgagaataacagtcggcgtggatgggtctgtctataagctgcatccctccttcaaggaaaggtttcacgcgagcgtgcgccgtctgaccccttcatgtgagattactttcatcgaatccgaggagggcagtggacggggggcagccttggtttctgctgtggcctgcaaaaaggcatgcatgcttggccaatga

SEQ ID NO: 77 - DNA, 1401 bp, synthetic construct
  source 1..1401: mol_type = other DNA; note = ORF Gck
atggccatggatgtgaccagaagccaggctcaaacagcactgactctggtagagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctagacaagcaccaaatgaaacataagaaactgccgctgggatttactttctcatttcctgtgagacatgaggatattgacaaggggatcctcttgaattggacaaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctattagaatatgacaggctggtggatgagtctagcgccaaccctggccagcaattgtatgaaaaactgattggagggaagtacatgggtgagcttgtccgccttgttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccactgattgtgacattgtgcggcgggcttgtgagtcagtttccactagagccgcacacatgtgctcagctggtttggctggagtaatcaataggatgcgcgaaagcaggagtgaggatgtgatgagaataacagttggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatga

SEQ ID NO: 78 - DNA, 1401 bp, synthetic construct
  source 1..1401: mol_type = other DNA; note = ORF Gck
atggccatggatgtgaccagaagccaggctcaaacagcactgactctggtggagcagatcttggcggaattccagcttcaagaggaagatctgaagaaagttatgagaaggatgcagaaggagatggaccgcggcttaaggctggaaacccatgaggaggccagtgtgaaaatgctacccacctatgtaagatctacacctgaaggatcagaggtgggggattttctcagtctggacttgggtggcactaacttcagggtcatgctggtgaaggttggagaaggggaggaaggccagtggtcagtgaaaaccaagcatcagatgtatagcattccagaggatgctatgacaggtaccgctgagatgctttttgactacatatctgaatgcatctctgatttcctggacaagcaccaaatgaaacataagaaactgccgctgggatttactttctcatttcctgtgagacatgaggatattgacaaggggatcctcttgaattggaccaaaggcttcaaggcaagtggcgcagaaggaaacaatgtagtggggcttctgagggatgctattaagcgcagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggtgatgagggtagaatgtgtgtaaatacagagtggggagcttttggggacagcggagaactggatgagttcctactggaatatgacaggctggtggatgagtctagcgccaaccctggccagcaattgtatgaaaaactgattggagggaagtacatgggtgagcttgtccgcctggttctgctgcggttagtggatgagaatctcctgtttcatggggaagcaagtgagcagttgcggacaaggggagcctttgaaaccagatttgtctcacaagtggagagtgatactggggacaggaaacagatttacaacatccttagcacactgggcctaagaccatctaccacagattgtgacattgtgcggcgggcttgtgagtcagttagcactagagccgcacacatgtgctcagctggtctggctggagtaatcaataggatgcgcgaaagcaggagtgaggatgtgatgagaataacagtgggtgtggatgggtctgtctataagctgcatccctccttcaaggaaaggtttcacgcgtctgtgcgcagactgaccccttcatgtgagattactttcatagaatctgaggagggcagtggaaggggggcagccttggtttctgctgtggcctgcaaaaaggcatgcatgcttggccaatga

SEQ ID NO: 79 - DNA, 1401 bp, synthetic construct
  source 1..1401: mol_type = other DNA; note = ORF Gck
atggccatggatgtgaccagaagccaggctcaaacagcactgactctggtagagcagatcttggctgaattccagcttcaagaggaagatctgaagaaagttatgagaaggatgcagaaggagatggacaggggcttaagactggaaacccatgaggaggccagtgtgaaaatgctacccacctatgtaaggtctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctagacaagcaccaaatgaaacataagaaactgccactgggatttactttctcatttcctgtgagacatgaggatattgacaaggggatcctcttgaattggaccaaaggcttcaaggcaagtggtgctgaaggaaacaatgtagtggggcttctgagggatgctattaagaggagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctattagaatatgacaggctggtggatgagtcttcagccaaccctggccagcaattgtatgaaaaactgattggagggaagtacatgggtgagcttgtcaggcttgttctgctgagattagtggatgagaatctcctgtttcatggtgaagcatctgagcagttgaggacaagaggagcctttgaaaccagatttgtctcacaagtggagagtgatactggggacaggaaacagatttacaacatccttagcacactgggcctaaggccatctaccactgattgtgacattgtgagaagggcttgtgagagtgtttccactagagcagcacacatgtgctcagctggtttggctggagtaatcaataggatgagagaaagcaggagtgaggatgtgatgagaataacagttggagtggatgggtctgtctataagctgcatccctccttcaaggaaaggtttcatgcctctgtgagaaggctgaccccttcatgtgagattactttcatagaatcagaggagggcagtggaagaggggcagccttggtttctgctgtggcctgcaaaaaggcatgcatgcttggccaatga

SEQ ID NO: 80 - DNA, 1401 bp, synthetic construct
  source 1..1401: mol_type = other DNA; note = ORF Gck
atggccatggatgtgaccagaagccaggctcaaacagcactgactctggtggagcagatcttggctgaattccagcttcaagaggaagatctgaagaaagttatgagaaggatgcagaaggagatggacagaggcttaagactggaaacccatgaggaggccagtgtgaaaatgctacccacctatgtaagatctacacctgaaggatcagaggtgggggattttctcagtctggacttgggtggcactaacttcagagtcatgctggtgaaggttggagaaggggaggaaggccagtggtcagtgaaaaccaagcatcagatgtatagcattccagaggatgctatgacaggcacagcagagatgctttttgactacatatctgaatgcatctctgatttcctggacaagcaccaaatgaaacataagaaactgcccctgggatttactttctcatttcctgtgagacatgaggatattgacaaggggatcctcttgaattggaccaaaggcttcaaggcaagtggtgctgaaggaaacaatgtagtggggcttctgagggatgctattaagagaagaggtgactttgagatggatgttgtggcaatggtcaatgatacagtggccacaatgatcagctgttattatgaggaccatcagtgtgaagtgggcatgatagtgggaactgggtgtaatgcctgctatatggaggaaatgcagaatgttgagttagtggaaggagatgagggtagaatgtgtgtaaatacagagtggggagcttttggggacagtggggaactggatgagttcctactggaatatgacaggctggtggatgagtcttcagccaaccctggccagcaattgtatgaaaaactgattggagggaagtacatgggtgagcttgtcagactggttctgctgagattagtggatgagaatctcctgtttcatggtgaagcatctgagcagttgagaacaagaggagcctttgaaaccagatttgtctcacaagtggagagtgatactggggacaggaaacagatttacaacatccttagcacactgggcctaagaccatctaccactgattgtgacattgtgagaagagcttgtgagagtgttagcactagagcagcacacatgtgctcagctggtctggctggagtaatcaataggatgagagaaagcagaagtgaggatgtgatgagaataacagtgggagtggatgggtctgtctataagctgcatccctccttcaaggaaaggtttcatgcctctgtgagaagactgaccccttcatgtgagattactttcatagaatcagaggagggcagtggaagaggggcagccttggtttctgctgtggcctgcaaaaaggcatgcatgcttggccaatga

SEQ ID NO: 81 - skipped

SEQ ID NO: 82 - protein, 465 aa, synthetic construct [sequence omitted]

SEQ ID NO: 83 - DNA, 325 bp, synthetic construct
  source 1..325: mol_type = other DNA; note = 5' UTR
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccacc

SEQ ID NO: 84 - DNA, 951 bp, synthetic construct
  source 1..951: mol_type = other DNA; note = Modified hIns - 17
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggatccaagctt

SEQ ID NO: 85 - DNA, 951 bp, synthetic construct
  source 1..951: mol_type = other DNA; note = Modified hIns18
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggcactgctggccctctggggacctgacccagcagcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtccagacagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggaattcaagctt

SEQ ID NO: 86 - DNA, 951 bp, synthetic construct
  source 1..951: mol_type = other DNA; note = Modified hIns - 19
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactgctccccctcctggctttgctggccctgtggggaccagatcctgcagctgcctttgtgaatcagcacctgtgtggctcagatctggttgaggcactctacctggtgtgtggggagagaggcttcttttacacaccaaggaccaagagggaggcagaggatctgcaggtaggtcaggtggagctgggaggtggccctggagcaggatccctgcagcccttggcactggagggctccagacagaagaggggcattgtggaacagtgttgcactagcatctgcagcctgtaccagctggagaactactgcaactaaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagcatatgaagctt

SEQ ID NO: 87 - DNA, 951 bp, synthetic construct
  source 1..951: mol_type = other DNA; note = Modified hIns - 20
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggatatcaagctt

SEQ ID NO: 88 - DNA, 951 bp, synthetic construct
  source 1..951: mol_type = other DNA; note = Modified hIns - 21
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagactagtaagctt

SEQ ID NO: 89 - DNA, 2019 bp, synthetic construct
  source 1..2019: mol_type = other DNA; note = Modified hGck - 22
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagtctagaaagctt

SEQ ID NO: 90 - DNA, 2019 bp, synthetic construct
  source 1..2019: mol_type = other DNA; note = Modified hGck - 23
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacactgtggccaccatgatctcctgctactatgaagaccatcagtgtgaggtgggcatgattgtgggcacaggctgcaatgcctgctacatggaggagatgcagaatgtggagctggtggagggggatgagggcaggatgtgtgtcaatacagagtggggagcctttggggactctggggagctggatgagttcctgctggagtatgacaggctggtggatgagagctcagcaaaccctggtcagcagctgtatgagaagctcataggtggcaagtacatgggagagctggtgaggcttgtgctgctcaggctggtggatgaaaacctgctcttccatggggaggcctctgagcagctgaggacaaggggagcctttgagaccaggtttgtgtcccaggtggagagtgacacaggagacaggaagcagatctacaacatcctgagcaccctggggctgagaccctccaccacagactgtgacattgtgaggagggcttgtgagagtgtgtctaccagggctgcccacatgtgctctgctgggctggctggggtcatcaacaggatgagggagagcaggagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggctagcaagctt

SEQ ID NO: 91 - DNA, 2019 bp, synthetic construct
  source 1..2019: mol_type = other DNA; note = Modified hGck - 24
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccaccatgatctcctgctactatgaagaccatcagtgtgaggtgggcatgatagtgggcacaggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggggagctggtgaggcttgtgctgctcaggctggtggatgaaaacctgctcttccatggggaggcctcagagcagctgagaacaaggggagcctttgagaccaggtttgtgtcccaggtggagagtgacacaggtgacaggaagcagatctacaacatcctgagcaccctggggctgagaccctccaccacagactgtgacattgtgaggagggcctgtgagagtgtgtctaccagggctgcccacatgtgctctgctgggctggcaggagtcatcaacaggatgagggagagcaggagtgaggatgtaatgaggatcactgtgggagtggatggctctgtgtacaagctgcacccctcattcaaggagaggttccatgccagtgtgagaaggctgacccccagctgtgagatcacctttatagagtctgaggagggcagtggcaggggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagattaataagctt

SEQ ID NO: 92 - DNA, 2019 bp, synthetic construct
  source 1..2019: mol_type = other DNA; note = Modified hGck - 25
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaaggatgcagaaggagatggacaggggcctgaggctggagacccatgaagaggccagtgtgaagatgctgcccacctatgtgaggtccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctctgacttcctggacaagcatcagatgaaacacaagaagctgcccctgggcttcaccttctcatttcctgtgaggcatgaagacattgataagggcatccttctcaactggaccaagggcttcaaggcctcaggagcagaagggaacaatgtggtggggcttctgagagatgctatcaaaaggagaggggactttgaaatggatgtggtggcaatggtgaatgacacagtggccactatgatctcctgctactatgaagaccatcagtgtgaggtgggcatgattgtgggcactggctgcaatgcctgctacatggaggagatgcagaatgtggagctggtggagggggatgagggcaggatgtgtgtcaatactgagtggggagcctttggggactctggggagctggatgagttcctgctggagtatgacaggctggtggatgagagctcagcaaaccctggtcagcagctgtatgagaagctcataggtggcaagtacatgggagagctggtgagacttgtgctgctcaggctggtggatgaaaacctgctcttccatggggaggcctcagagcagctgaggacaaggggagcctttgagaccaggtttgtgtcccaggtggagagtgacactggagacaggaagcagatctacaacatcctgagcaccctggggctgaggccctctaccacagactgtgacatagtgagaagggcctgtgagtctgtgtctaccagggctgcccacatgtgctctgctgggctggctggagtcatcaacagaatgagggagagcagaagtgaggatgtaatgaggatcactgtgggagtggatggctctgtgtacaagctgcacccctcattcaaggagaggttccatgccagtgtgaggaggctgacccccagctgtgagatcacctttattgagtctgaggagggcagtggcaggggagctgccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagatgcataagctt

SEQ ID NO: 93 - DNA, 2019 bp, synthetic construct
  source 1..2019: mol_type = other DNA; note = Modified hGck - 26
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggccgcatgtgtgtcaatactgagtggggcgcctttggggactctggagagctggatgagttcctgctggagtatgaccgcctggtggatgagagctcagcaaacccaggtcagcagctgtatgagaagctcataggtggcaagtacatgggagagctggtgcggcttgtgctgctcaggttggtggatgaaaacctgctcttccatggggaggccagtgagcagctgcgcacacgcggagcctttgagacgcgctttgtgtcacaagtggagtctgacacaggagaccgcaagcagatctacaacatcctgagcacactggggctgcggccctccaccactgactgtgacattgtgcgccgcgcctgtgagagtgtgtctacgcgcgctgcgcacatgtgctcagcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagagtactaagctt

SEQ ID NO: 94 - DNA, 2019 bp, synthetic construct
  source 1..2019: mol_type = other DNA; note = Modified hGck - 27
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggaccgcggcctgaggctggagacccatgaagaggccagtgtgaagatgctgcccacctatgtgcgctccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgcgggatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggccgcatgtgtgtcaatacagagtggggcgcctttggggacagcggagagctggatgagttcctgctggagtatgaccgcctggtggatgagagctcagcaaacccaggtcagcagctgtatgagaagctcataggtggcaagtacatgggagagctggtgcggcttgtgctgctcaggctggtggatgaaaacctgctcttccatggggaggcctctgagcagctgcgcacacgcggagcctttgagacgcgctttgtgagccaggtggagagtgacacaggagaccgcaagcagatctacaacatcctgagcaccctggggctgcggcccagcaccactgactgtgacattgtgcgccgcgcctgtgagtcagtgtctacgcgcgctgcgcacatgtgcagcgcggggctggcgggagtcatcaaccgcatgcgcgagagccgctctgaggatgtaatgcgcatcactgtgggagtggatggcagtgtgtacaagctgcaccccagcttcaaggagcggttccatgcctcagtgcgcaggctgacccccagctgtgagatcacctttattgagtctgaggagggcagtggcagaggcgcggccctggtcagcgcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggtaccaagctt

SEQ ID NO: 95 - DNA, 2019 bp, synthetic construct
  source 1..2019: mol_type = other DNA; note = Modified hGck - 28
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggctatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagttggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgttgtggggcttctgagagatgctatcaaaagaagaggggactttgaaatggatgtggtggcaatggtgaatgacacagtggccactatgatctcctgctactatgaagaccatcagtgtgaggttggcatgattgtgggcacaggctgcaatgcctgctacatggaggagatgcagaatgtggagctggtggagggggatgagggcaggatgtgtgtcaatactgagtggggagcctttggggactctggagagctggatgagttcctgctggagtatgacagactggtggatgagagctcagcaaacccaggtcagcagctgtatgagaagctcataggtggcaagtacatgggagagctggtgaggcttgtgctgctcaggttggtggatgaaaacctgctcttccatggggaggccagtgagcagctgagaacaaggggagcctttgagacaagatttgtgtcacaggtggagtctgacacaggagacaggaagcagatctacaacatcctgagcaccctggggctgagaccctccaccactgactgtgacattgtgaggagagcctgtgagagtgtgtctaccagggctgctcacatgtgctcagcagggctggctggagtcatcaacagaatgagggagagcagatctgaggatgtaatgaggatcactgtgggtgtggatggcagtgtgtacaagctgcaccccagcttcaaggagagattccatgcctcagtgaggaggctgacccccagctgtgagatcaccttcattgagtctgaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagaatattaagctt

SEQ ID NO: 96 - DNA, 2019 bp, synthetic construct
  source 1..2019: mol_type = other DNA; note = Modified hGck - 29
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccatggatgtcacaaggagccaggcccagacagccttgactctggtagagcagatcctggcagagttccagctgcaggaggaggacctgaagaaggtgatgagaagaatgcagaaggagatggacagaggcctgaggctggagacccatgaagaggccagtgtgaagatgctgcccacctatgtgagatccaccccagaaggctcagaagtgggggacttcctctccctggacctgggtggcactaacttcagggtgatgctggtgaaggtgggagaaggtgaggaggggcagtggagtgtgaagaccaaacaccagatgtactccatccctgaggatgccatgacaggcactgctgagatgctctttgactacatctctgagtgcatctcagacttcctggacaagcatcagatgaaacacaagaagctgcccctgggcttcaccttctcctttcctgtgaggcatgaagacattgataagggcatccttctcaactggaccaagggcttcaaggcctcaggagcagaagggaacaatgtggtggggcttctgagagatgctatcaaaagaagaggggactttgaaatggatgtggtggcaatggtgaatgacacagtggccaccatgatctcctgctactatgaagaccatcagtgtgaggtgggcatgattgtgggcactggctgcaatgcctgctacatggaggagatgcagaatgtggagctggtggagggggatgagggcagaatgtgtgtcaatacagagtggggagcctttggggactctggagagctggatgagttcctgctggagtatgacagactggtggatgagagctcagcaaacccaggtcagcagctgtatgagaagctcataggtggcaagtacatgggagagctggtgagacttgtgctgctcaggctggtggatgaaaacctgctcttccatggggaggccagtgagcagctgagaacaagaggagcctttgagaccagatttgtgagccaggtggagtcagacacaggagacagaaagcagatctacaacatcctgagcaccctggggctgagacccagcaccactgactgtgacattgtgagaagagcctgtgagtctgtgtctaccagagctgcccacatgtgcagtgctgggctggctggagtcatcaacagaatgagagagagcagatcagaggatgtaatgagaatcactgtgggtgtggatggctctgtgtacaagctgcaccccagcttcaaggagagattccatgccagtgtgagaaggctgacccccagctgtgagatcacctttattgagtcagaggagggcagtggcagaggagcagccctggtctctgctgtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagttaattaagctt

SEQ ID NO: 97 - DNA, 293 bp, synthetic construct
  source 1..293: mol_type = other DNA; note = hIns 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggatccaagctt

SEQ ID NO: 98 - DNA, 293 bp, synthetic construct
  source 1..293: mol_type = other DNA; note = hIns 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggaattcaagctt

SEQ ID NO: 99 - DNA, 293 bp, synthetic construct
  source 1..293: mol_type = other DNA; note = hIns 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagcatatgaagctt

SEQ ID NO: 100 - DNA, 293 bp, synthetic construct
  source 1..293: mol_type = other DNA; note = hIns 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggatatcaagctt

SEQ ID NO: 101 - DNA, 293 bp, synthetic construct
  source 1..293: mol_type = other DNA; note = hIns 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagactagtaagctt

SEQ ID NO: 102 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagtctagaaagctt

SEQ ID NO: 103 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcaggctagcaagctt

SEQ ID NO: 104 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagattaataagctt

SEQ ID NO: 105 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagatgcataagctt

SEQ ID NO: 106 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagagtactaagctt

SEQ ID NO: 107 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagggtaccaagctt

SEQ ID NO: 108 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagaatattaagctt

SEQ ID NO: 109 - DNA, 293 bp, synthetic construct
  source 1..293: mol_type = other DNA; note = hGck 3' UTR
agccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataagggactagagcagttaattaagctt

SEQ ID NO: 110 - DNA, 333 bp, synthetic construct
  source 1..333: mol_type = other DNA; note = hIns (B10D) ORF - 22
atggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 111 - DNA, 333 bp, synthetic construct
  source 1..333: mol_type = other DNA; note = hIns (B10H) ORF - 2
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 112 - DNA, 333 bp, synthetic construct
  source 1..333: mol_type = other DNA; note = hIns (B10H) ORF - 27
atggccctgtggatgcgcctcctgcccctgctggcgctgctggccctctggggacctgacccagccgcagcctttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 113 - DNA, 333 bp, synthetic construct
  source 1..333: mol_type = other DNA; note = hIns (B10H) ORF -28
atggccctgtggatgagactcctgcccctgctggctctgctggccctctggggacctgacccagctgcagcctttgtgaaccaacacctgtgtggctcacacctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactag

SEQ ID NO: 114 - DNA, 333 bp, synthetic construct
  source 1..333: mol_type = other DNA; note = hIns (B10H) ORF
atggccctgtggatgcggctcttgccccttctggctttactggcactatggggccctgacccagcggccgctttcgtgaaccagcacctctgcggaagccacctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactga

SEQ ID NO: 115 - DNA, 333 bp, synthetic construct
  source 1..333: mol_type = other DNA; note = hIns (B10H) ORF
atggccctgtggatgcggctcttgccccttctggctttactggcactatggggccctgacccagcggccgcttttgtgaaccagcacctctgcggatctcacctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactga

SEQ ID NO: 116 - DNA, 333 bp, synthetic construct
  source 1..333: mol_type = other DNA; note = hIns (B10H) ORF
atggccctgtggatgagactcttgccccttctggctttactggcactatggggccctgacccagcagctgcttttgtgaaccagcacctctgtggatctcacctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactga

SEQ ID NO: 117 - DNA, 348 bp, synthetic construct
  source 1..348: mol_type = other DNA; note = hIns (B10D[IL-6]) ORF - 32
atgaacagcttcagcacaagcgccttcggccccgtggctttctctctgggactgctgctggtgctgcctgccgctttccctgccccttttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 118 - DNA, 348 bp, synthetic construct
  source 1..348: mol_type = other DNA; note = hIns (B10D[IL-6]) ORF - 33
atgaacagcttcagcacaagcgcctttggcccagtggctttctctctgggactgctgctggtgctgcctgccgctttccctgccccttttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 119 - DNA, 348 bp, synthetic construct
  source 1..348: mol_type = other DNA; note = hIns (B10D[IL-6]) ORF - 34
atgaacagcttcagcacaagtgcctttggcccagtggctttctctctgggactgctgctggtgctgcctgcagctttccctgccccttttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactag

SEQ ID NO: 120 - DNA, 348 bp, synthetic construct
  source 1..348: mol_type = other DNA; note = hIns (B10H[IL-6]) ORF -35
atgaacagcttcagcacaagcgccttcggccccgtggctttctctctgggactgctgctggtgctgcctgccgctttccctgccccttttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 121 - DNA, 348 bp, synthetic construct
  source 1..348: mol_type = other DNA; note = hIns (B10H[IL-6]) ORF - 36
atgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactag

SEQ ID NO: 122 - DNA, 348 bp, synthetic construct
  source 1..348: mol_type = other DNA; note = hIns (B10H[IL-6]) ORF - 37
atgaacagcttcagcacaagtgcctttggcccagtggctttctctctgggactgctgctggtgctgcctgcagctttccctgccccttttgtgaaccaacacctgtgtggctcacacctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactag

SEQ ID NO: 123 - DNA, 953 bp, synthetic construct
  source 1..953: mol_type = other DNA; note = Ins B10D: 4 mutations - 22
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 124 - DNA, 1870 bp, synthetic construct
  source 1..1870: mol_type = other DNA; note = Ins B10Dx2: B10D->IRES->RE - 23
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgctttcgtgaaccagcacctctgcggaagcgatctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 125 - DNA, 1870 bp, synthetic construct
  source 1..1870: mol_type = other DNA; note = Ins B10Dx2: 111->IRES->RE-111 - 24
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgcttttgtgaaccagcacctctgcggatctgatctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 126 - DNA, 1870 bp, synthetic construct
  source 1..1870: mol_type = other DNA; note = Ins B10Dx2: 211->IRES->RE-211 - 25
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcagatctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgagactcttgccccttctggctttactggcactatggggccctgacccagcagctgcttttgtgaaccagcacctctgtggatctgatctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 127 - DNA, 953 bp, synthetic construct
  source 1..953: mol_type = other DNA; note = Ins (B10H)_2
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 128 - DNA, 953 bp, synthetic construct
  source 1..953: mol_type = other DNA; note = Ins (B10H) - 27
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 129 - DNA, 953 bp, synthetic construct
  source 1..953: mol_type = other DNA; note = Ins (B10H) - 28
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcacacctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 130 - DNA, 1870 bp, synthetic construct
  source 1..1870: mol_type = other DNA; note = Ins B10Hx2: B10H->IRES->RE - 29
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgctttcgtgaaccagcacctctgcggaagccacctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 131 - DNA, 1870 bp, synthetic construct
  source 1..1870: mol_type = other DNA; note = Ins B10Hx2: 111->IRES->RE-111 - 30
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgcttttgtgaaccagcacctctgcggatctcacctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 132 - DNA, 1870 bp, synthetic construct
  source 1..1870: mol_type = other DNA; note = Ins B10Hx2: 211->IRES->RE-211 -31
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgagactcctgcccctgctggctctgctggccctctggggacctgacccagctgcagcctttgtgaaccaacacctgtgtggctcacacctggtggaagctctctacctagtgtgtggggaaagaggcttcttctacacacccaggaccaagagggaggcagaggacctgcaggtggggcaggtggagctgggagggggccctggtgcaggcagcctgcagcccttggccctggaggggtctagacagaagaggggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgagactcttgccccttctggctttactggcactatggggccctgacccagcagctgcttttgtgaaccagcacctctgtggatctcacctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 133 - DNA, 968 bp, synthetic construct
  source 1..968: mol_type = other DNA; note = Ins (B10D[IL-6]) - 32
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgccttcggccccgtggctttctctctgggactgctgctggtgctgcctgccgctttccctgccccttttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 134 - DNA, 968 bp, synthetic construct
  source 1..968: mol_type = other DNA; note = Ins (B10D[IL-6]) - 33
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcagatctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 135 - DNA, 968 bp, synthetic construct
  source 1..968: mol_type = other DNA; note = Ins (B10D[IL-6]) - 34
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagtgcctttggcccagtggctttctctctgggactgctgctggtgctgcctgcagctttccctgccccttttgtgaaccaacacctgtgtggctcagatctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 136 - DNA, 968 bp, synthetic construct
  source 1..968: mol_type = other DNA; note = Ins (B10H[IL-6]) - 35
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgccttcggccccgtggctttctctctgggactgctgctggtgctgcctgccgctttccctgccccttttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 137 - DNA, 968 bp, synthetic construct
  source 1..968: mol_type = other DNA; note = Ins (B10H[IL-6]) - 36
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 138 - DNA, 968 bp, synthetic construct
  source 1..968: mol_type = other DNA; note = Ins (B10H[IL-6]) - 37
aagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagtgcctttggcccagtggctttctctctgggactgctgctggtgctgcctgcagctttccctgccccttttgtgaaccaacacctgtgtggctcacacctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 139 - DNA, 1885 bp, synthetic construct
  source 1..1885: mol_type = other DNA; note = Ins B10Hx2[IL-6/ISP]: IL-6->B10H->IRES->ISP->RE - 38
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgccttcggccccgtggctttctctctgggactgctgctggtgctgcctgccgctttccctgccccttttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgctttcgtgaaccagcacctctgcggaagccacctggtcgaggccttgtacctggtttgtggggaaagaggttttttctataccccgaggacaaagcgcgaggcagaagaccttcaagtgggccaggtagagctcggagggggccccggtgccggatccctgcagcctctggctctcgaagggtctcgacaaaaacgtggcatcgtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 140 - DNA, 1885 bp, synthetic construct
  source 1..1885: mol_type = other DNA; note = Ins B10Hx2[IL-6/ISP]: IL-6->111->IRES->ISP->RE-111 - 39
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagcgcctttggcccagtggctttctctctgggactgctgctggtgctgcctgccgctttccctgccccttttgtgaaccaacacctgtgcggctcacacctggtggaagctctctacctagtgtgcggggaaagaggcttcttctacacacccaggaccaagcgggaggcagaggacctgcaggtggggcaggtggagctgggcgggggccctggtgcaggcagcctgcagcccttggccctggaggggtcgcgacagaagcggggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgcggctcttgccccttctggctttactggcactatggggccctgacccagcggccgcttttgtgaaccagcacctctgcggatctcacctggttgaggccttgtacctggtttgtggggaaagaggttttttctataccccaaggacaaagcgcgaggcagaagaccttcaagtgggccaggtagagctgggagggggcccgggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 141 - DNA, 1885 bp, synthetic construct
  source 1..1885: mol_type = other DNA; note = Ins B10Hx2[IL-6/ISP]: IL-6->211->IRES->ISP->RE-211 - 40
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatgaacagcttcagcacaagtgcctttggcccagtggctttctctctgggactgctgctggtgctgcctgcagctttccctgccccttttgtgaaccaacacctgtgtggctcacacctggtggaagctctctacctagtgtgtggggaaagaggcttcttctacacacccaggaccaagagagaggcagaggacctgcaggtggggcaggtggagctgggagggggccctggtgcaggcagcctgcagcccttggccctggaggggtccaggcagaagagaggcattgtggaacaatgctgtaccagcatctgctccctctaccagctggagaactactgcaactaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatggccctgtggatgagactcttgccccttctggctttactggcactatggggccctgacccagcagctgcttttgtgaaccagcacctctgtggatctcacctggttgaggccttgtacctggtttgtggggaaagaggttttttctataccccaaggacaaagagggaggcagaagaccttcaagtgggccaggtagagctgggagggggccctggtgcaggatccctgcagcctctggctctagaagggtctagacaaaaaaggggcatagtggagcagtgctgtacttcaatttgcagtttgtaccagcttgagaattattgtaactgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 142 - DNA, 567 bp, synthetic construct
  source 1..567: mol_type = other DNA; note = IRES
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaacacgatgataa

SEQ ID NO: 143 - DNA, 585 bp, synthetic construct
  source 1..585: mol_type = other DNA; note = IRES v2
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaca

SEQ ID NO: 144 - protein, 110 aa, synthetic construct
  source 1..110: note = mutant ins B10D
MALWMRLLPLLALLALWGPDPAAAFVNQHLCGSDLVEALYLVCGERGFFYTPRTKREAEDLQVGQVELGGGPGAGSLQPLALEGSRQKRGIVEQCCTSICSLYQLENYCN

SEQ ID NO: 145 - protein, 110 aa, synthetic construct
  source 1..110: note = mutant ins B10H
MALWMRLLPLLALLALWGPDPAAAFVNQHLCGSHLVEALYLVCGERGFFYTPRTKREAEDLQVGQVELGGGPGAGSLQPLALEGSRQKRGIVEQCCTSICSLYQLENYCN

SEQ ID NO: 146 - DNA, 324 bp, synthetic construct
  source 1..324: mol_type = other DNA; note = 5' UTR
agctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccacc

SEQ ID NO: 147 - DNA, 333 bp, synthetic construct
  source 1..333: mol_type = other DNA; note = wildtype sequence
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 148 - DNA, 350 bp, synthetic construct
  source 1..350: mol_type = other DNA; note = 5 UTR (B10D-Ins unmodified)
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgcc

SEQ ID NO: 149 - DNA, 363 bp, synthetic construct
  source 1..363: mol_type = other DNA; note = 3' UTR (B10D-Ins unmodified)
acgcagctgctagctaatcgataccgtcgacctcgaggaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 150 - DNA, 333 bp, synthetic construct
  source 1..333: mol_type = other DNA; note = Ins1 - furin cleavage sites and histidine substituted by ileucine in B10 position
atggctctgtggatgagactgctgcccctgctggctctgctggcactgtggggacctgatcctgccgccgctttcgtgaaccagcacctgtgtggcagcatcctggtggaagccctgtacctcgtgtgcggcgagagaggcttcttctacacccctaggaccaagagagaggccgaggatctgcaagtgggccaggtggaactgggcggaggacctggcgctggatctctgcagcctctggctctggaaggcagcagacagaaaaggggcatcgtggaacagtgctgcaccagcatctgctccctgtaccagctggaaaactactgcaactga

SEQ ID NO: 151 - DNA, 333 bp, synthetic construct
  source 1..333: mol_type = other DNA; note = Ins2 - furin cleavage sites and histidine substituted by valine in B10 position
atggctctgtggatgagactgctgcccctgctggctctgctggcactgtggggacctgatcctgccgccgctttcgtgaaccagcacctgtgtggaagcgtgctggtggaagccctgtacctcgtgtgtggcgagagaggcttcttctacacccctaggaccaagagagaggccgaggatctgcaagtgggccaggtggaactgggcggaggacctggcgctggatctctgcagcctctggctctggaaggcagcagacagaaaaggggcatcgtggaacagtgctgcaccagcatctgctccctgtaccagctggaaaactactgcaactga

SEQ ID NO: 152 - DNA, 348 bp, synthetic construct
  source 1..348: mol_type = other DNA; note = Ins3 - furin cleavage sites and WT signal peptide substituted by signal peptide of interleukin 6 (IL6)
atgaacagcttcagcacaagcgccttcggccccgtggctttctctctgggactgctgctggtgctgcctgccgctttccctgcccctttcgtgaaccagcacctgtgcggcagccacctggtggaagctctgtacctcgtgtgcggcgagagaggcttcttctacacccctaggaccaagagagaggccgaggatctgcaagtgggccaggtggaactgggcggaggacctggcgctggatctctgcagcctctggctctggaaggcagcagacagaaaaggggcatcgtggaacagtgctgcaccagcatctgctccctgtaccagctggaaaactactgcaactga

SEQ ID NO: 153 - DNA, 339 bp, synthetic construct
  source 1..339: mol_type = other DNA; note = Ins4 - furin cleavage sites and WT signal peptide substituted by signal peptide of fibronectin
atgctgagaggacctggaccaggactgctgctgctggccgtgcagtgtctgggaacagccgtgcctagcacaggcgccttcgtgaaccagcacctgtgtggcagccacctggtggaagccctgtacctcgtgtgtggcgagagaggcttcttctacacccctaggaccaagagagaggccgaggatctgcaagtgggccaggtggaactgggcggaggaccaggcgctggatctctgcagcctctggctctggaaggcagcagacagaaaaggggcatcgtggaacagtgctgcaccagcatctgctccctgtaccagctggaaaactactgcaactga

SEQ ID NO: 154 - DNA, 333 bp, synthetic construct
  source 1..333: mol_type = other DNA; note = Ins5 - furin cleavage sites
atggctctgtggatgagactgctgcccctgctggctctgctggcactgtggggacctgatcctgccgccgctttcgtgaaccagcacctgtgtggcagccacctggtggaagccctgtacctcgtgtgtggcgagagaggcttcttctacacccctaggaccaagagagaggccgaggatctgcaagtgggccaggtggaactgggcggaggacctggcgctggatctctgcagcctctggctctggaaggcagcagacagaaaaggggcatcgtggaacagtgctgcaccagcatctgctccctgtaccagctggaaaactactgcaactga

SEQ ID NO: 155 - DNA, 333 bp, synthetic construct; identical to SEQ ID NO: 154
  source 1..333: mol_type = other DNA; note = Ins6 - furin cleavage sites

SEQ ID NO: 156 - DNA, 348 bp, synthetic construct
  source 1..348: mol_type = other DNA; note = Ins7 - furin cleavage sites, WT signal peptide substituted signal peptide of IL6 and histidine substituted by valine in position B10
atgaacagcttcagcacaagcgctttcggccccgtggctttttctctgggactgctgctggttctgcctgccgcttttcctgctcctttcgtgaaccagcacctgtgcggaagcgtgctggtggaagctctgtatctcgtgtgtggcgagagaggcttcttctacacccctaggaccaagagagaggccgaggatctgcaagtcggccaggttgaacttggcggaggacctggtgctggatctctgcagcctcttgctctggaaggcagcagacagaagaggggcatcgtcgagcagtgctgcaccagcatctgtagcctgtaccagctggaaaactactgcaactga

SEQ ID NO: 157 - DNA, 333 bp, synthetic construct
  source 1..333: mol_type = other DNA; note = Ins8 - furin cleavage sites and proline substituted by aspart in B28 position
atggcactttggatgagactgctgccactgctggccctgcttgctctttggggacctgatcctgctgccgcctttgtgaaccagcacctgtgtggaagccatctggtggaagccctgtacctcgtgtgtggcgagagaggcttcttctacaccgacaggaccaagagagaggccgaggatctgcaagtcggccaggttgaacttggcggaggacctggtgctggatctctgcagcctcttgctctggaaggcagcagacagaagaggggcatcgtcgagcagtgctgcaccagcatctgtagcctgtaccagctggaaaactactgcaactga

SEQ ID NO: 158 - DNA, 333 bp, synthetic construct
  source 1..333: mol_type = other DNA; note = Ins9 - furin cleavage sites
atggccttgtggatgcggcttttgccgctgctcgcgctcttggccctctgggggcccgacccggctgccgctttcgtcaatcagcacctctgcggcagccacctcgtggaagcgctttacttggtctgcggcgagaggggcttcttttatactccacggactaagcgggaggccgaagacttgcaagtcgggcaagtggaactgggaggcgggccgggtgcggggtctctgcaacctcttgcccttgaggggtcacgacaaaagagagggatagttgagcagtgttgtacgtcaatttgttccctgtatcaacttgaaaattattgcaattag

SEQ ID NO: 159 - DNA, 333 bp, synthetic construct
  source 1..333: mol_type = other DNA; note = Ins10 - furin cleavage sites
atggctctgtggatgagacttcttcctcttttggccctgctggccctttggggccctgatcctgccgccgccttcgttaaccaacatctgtgcggttcacacctggttgaagctctttacctcgtttgtggagaaagaggctttttttacacaccacggaccaaaagggaggcagaggacctgcaggtgggacaggttgagctgggcgggggccctggggcaggctcacttcagccactcgccctggagggctctcgccagaagagaggtatagtggagcaatgctgtaccagcatttgttctctctatcagctggagaattactgtaactaa

SEQ ID NO: 160 - DNA, 683 bp, synthetic construct
  source 1..683: mol_type = other DNA; note = Unmodified hIns_2 (B10D)
aagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 161 - DNA, 957 bp, synthetic construct
  source 1..957: mol_type = other DNA; note = Unmodified hIns_3 (B10D)
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 162 - DNA, 1401 bp, synthetic construct
  source 1..1401: mol_type = other DNA; note = Unmodified hGck_2 ORF
atggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtga

SEQ ID NO: 163 - DNA, 2016 bp, synthetic construct
  source 1..2016: mol_type = other DNA; note = Unmodified hGck_2
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcat

SEQ ID NO: 164 - DNA, 2025 bp, synthetic construct
  source 1..2025: mol_type = other DNA; note = Unmodified hGck_3
ctagaagctttattgtggtagtttatcacagttaaattgctaaggcagtcagtgcttctgacacaacagtctccaacttaagctgcagtgactctcttaaggtagccttgcagaagttggtcctgaggcactgggcaggtaagtatcaaggttacaagacaggtttaaggagaccaatagaaactgggcttgtggagacagagaagactcttggctttctgataggcacctattggtcttactgacatccactttgcctttctctccacaggtgtccactcccagttcaattacagctcttaaggctagagtcctgcagaattgccaccatggcgatggatgtcacaaggagccaggcccagacagccttgactctggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgaagccagctgccttccctttactgtgggttaatgcttgcagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcagaagcttttg

SEQ ID NO: 165 - protein, 24 aa, synthetic construct
  source 1..24: note = Wild-type insulin signal sequence
MALWMRLLPLLALLALWGPDPAAA

SEQ ID NO: 166 - protein, 29 aa, synthetic construct
  source 1..29: note = IL-6 signal sequence
MNSFSTSAFGPVAFSLGLLLVLPAAFPAP

SEQ ID NO: 167 - protein, 26 aa, synthetic construct
  source 1..26: note = Fibronectin signal sequence
MLRGPGPGLLLLAVQCLGTAVPSTGA

(19) United States
(12) Patent Application Publication
(10) Pub. No.: US 2026/0139024 A1
(43) Pub. Date: May 21, 2026

(54) MODIFIED GLUCOKINASE NUCLEIC ACIDS FOR TREATING DIABETES

(71) Applicants: KRIYA THERAPEUTICS, INC., Morrisville, NC (US); UNIVERSITAT AUTONOMA DE BARCELONA, Barcelona (ES)

(72) Inventors: Nachiketa GUPTA, Morrisville, NC (US); Weiran SHEN, Morrisville, NC (US); Miquel GARCIA MARTINEZ, Barcelona (ES); Veronica JIMENEZ CENZANO, Barcelona (ES); Maria Fatima BOSCH TUBERT, Barcelona (ES)

(21) Appl. No.: 19/431,912

(22) Filed: Dec. 23, 2025

Related U.S. Application Data
(63) Continuation of application No. 18/003,980.
(60) Provisional application No. 63/047,965, filed on Jul. 3, 2020.
(60) Provisional application No. 63/054,162, filed on Jul. 20, 2020.
(60) Provisional application No. 63/067,264, filed on Aug. 18, 2020.
(60) Provisional application No. 63/141,918, filed on Jan. 26, 2021.
(60) Provisional application No. 63/188,778, filed on May 14, 2021.

(51) Int. Cl. C07K 14/62; A61K 48/00; A61P 3/10; C07K 14/54; C12N 9/12; C12N 15/86

(52) U.S. Cl. CPC C07K 14/62; A61K 48/005; A61P 3/10; C07K 14/5412; C12N 9/1205; C12N 15/86; C07K 2319/02; C07K 2319/50; C12N 2750/14143; C12N 2830/008; C12Y 207/01002

(57) ABSTRACT

The present disclosure relates to modified nucleic acid sequences encoding insulin and glucokinase, expression cassettes and delivery vectors comprising the same, and methods for delivery of the same for treating diabetes.

### CROSS REFERENCE TO RELATED APPLICATIONS

The present application is a continuation of U.S. patent application Ser. No. 18/003,980, filed Dec. 30, 2022, which is a National Stage of International Patent Application No. PCT/US2021/040366, filed Jul. 2, 2021, which claims the priority benefit of U.S. Provisional Application No. 63/047,965 filed Jul. 3, 2020; U.S. Provisional Application No. 63/054,162 filed Jul. 20, 2020; U.S. Provisional Application No. 63/067,264 filed Aug. 18, 2020; U.S. Provisional Application No. 63/141,918 filed Jan. 26, 2021; and U.S. Provisional Application No. 63/188,788 filed May 14, 2021, each of which are hereby incorporated by reference in their entirety.

### REFERENCE TO SEQUENCE LISTING SUBMITTED ELECTRONICALLY

The content of the electronically submitted sequence listing (4525_0160006_SequenceListing_ST26; Size: 331,843 bytes; Date of Creation: Dec. 22, 2025) filed with the application is incorporated herein by reference in its entirety.

### BACKGROUND

The two main forms of diabetes mellitus are type 1 (T1DM) and type 2 (T2DM) (Diabetes care, 1997, 20-1183-1197).

T1DM is characterized by a severe lack of insulin production due to specific destruction of the pancreatic B-cells. B-cell loss in T1DM is the result of an autoimmune mediated process, in which a chronic inflammation called insulitis causes β-cell destruction (Eizirik D. L. et al, 2001, Diabetologia, 44:2115-2133 and Mathis D et al, 2001, Nature, 414:792-798). T1DM is one of the most common endocrine and metabolic conditions of childhood; incidence is rapidly increasing, especially among young children. T1DM is diagnosed when the autoimmune-mediated β-cell destruction is almost complete causing patients to need insulin-replacement therapy to survive. T1DM in an adult may present itself similar to T2DM, with a slow deterioration in metabolic control, and subsequent progression to insulin dependency. This form is called latent autoimmune diabetes mellitus in adults (LADA) (Diabetes Atlas 4th edition, 2009, International Diabetes Federation).

T2DM is the most common form of diabetes mellitus and has been attributed to an interaction between genetic, environmental, and behavioral risk factors. T2DM is characterized by insulin insensitivity, declining insulin production, and eventual pancreatic beta-cell failure (Olokoba, A. et al, 2012, Oman Med. J. 27 (4): 269-273).

Lifelong insulin treatment is often the therapy of choice for both T1DM and T2DM. While lifelong treatment with exogenous insulin has been largely successful in managing diabetes, diabetic complications can still occur due to difficulties with maintaining tight glycemic control. States of prolonged hyperglycemia can lead to severe microvascular or macrovascular complications, most commonly presenting as retinopathies, neuropathies, nephropathies, cerebrovascular accidents, or myocardial infarctions. These devastating complications can be prevented with improvements in glycemic control. Of note, brittle diabetes, which is a particularly labile form, can be very difficult to manage even with lifelong exogenous insulin.

Additionally, in many underdeveloped countries, access to self-care tools and to insulin can be limited, which may lead to severe handicap and early death in diabetic children (Diabetes Atlas 4th edition, 2009, International Diabetes Federation, Beran D. et al 2006, Lancet, 368:1689-1695, and Gale E. A., et al, 2006, Lancet, 368:1626-1628). The most common cause of death in a child with diabetes, from a global perspective, is lack of access to insulin. Thus, the availability of a one-time gene therapy approach could have a profound effect in situations where access to insulin is limited (Greenwood H. L. et al, 2006, PLOS Med 3.e381).

The reduction of hyperglycemia and maintenance of normoglycemia is a goal of any therapeutic approach to TIDM and T2DM. The current therapy for most diabetic patients is based on regular subcutaneous injections of both of short-acting and long-acting insulin preparations.

### FIELD OF DISCLOSURE

The present disclosure pertains to the medical field, including gene therapy compositions comprising modified nucleic acids encoding insulin and/or glucokinase for use in treatment of Diabetes.

### BRIEF SUMMARY

Certain aspects of the disclosure are directed to a polynucleotide encoding a human insulin (Ins) protein (e.g., a preproinsulin or variant thereof) comprising (i) a nucleotide sequence encoding a signal peptide, optionally wherein the signal peptide is not a wild-type preproinsulin signal sequence, and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid position in wild-type proinsulin, and optionally the polynucleotide further comprises a cleavage site. In some aspects, the signal peptide is a wild-type preproinsulin signal sequence, an IL-6 signal sequence, or a fibronectin signal sequence. In some aspects, the cleavage site is a furin cleavage site.

Certain aspects of the disclosure are directed to a polynucleotide comprising a nucleic acid encoding a human insulin (Ins) protein, wherein the nucleic acid comprises an open reading frame (ORF) comprising: (i) a nucleotide sequence encoding a signal peptide and (ii) a nucleotide sequence at least 85%, 86%, 87%, 88%, 89%, 90%, 91%, 92%, 93%, 94%, 95%, 96%, 97%, 98%, 99%, or 100% identical to nucleic acids 73-330 of any of SEQ ID NOs: 43-57, 110-116, 150-151, 154-155, and 157-159, nucleic acids 88-345 of any of SEQ ID NOs: 117-122, 152, and 156, or nucleic acids 79-336 of SEQ ID NO: 153.

In some aspects, the encoded human Ins protein comprises (i) a signal peptide (e.g., a wild-type preproinsulin signal sequence, an IL-6 signal sequence, or a fibronectin signal sequence) and (ii) amino acids 25-110 of SEQ ID NO: 41, amino acids 25-110 of SEQ ID NO: 144, or amino acids 25-110 of SEQ ID NO: 145. In some aspects, the encoded human insulin protein further comprises a cleavage site (e.g., a furin cleavage site).

Certain aspects of the disclosure are directed to a polynucleotide comprising a nucleic acid encoding a human insulin (Ins) protein, wherein the nucleic acid comprises an open reading frame (ORF) comprising: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159. In some aspects, the polynucleotide comprises at least two nucleic acid sequences encoding a human Ins protein. In some aspects, the polynucleotide comprises at least two ORF nucleotide sequences at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159, wherein the two ORF nucleotide sequences can be the same or different. In some aspects, the polynucleotide further comprises an IRES sequences. In some aspects, the at least two ORF nucleotide sequences are separated by an IRES sequences.

In some aspects, the encoded human Ins protein comprises a signal sequence and a proinsulin polypeptide. In some aspects, the encoded human Ins protein comprises the amino acid sequence of any of amino acids 25-110 of SEQ ID NO: 41, amino acids 25-110 of SEQ ID NO: 144, or amino acids 25-110 of SEQ ID NO: 145. In some aspects, the encoded human Ins protein is a preproinsulin. In some aspects, the encoded human Ins protein comprises the amino acid sequence of SEQ ID NO: 41, SEQ ID NO: 144, or SEQ ID NO: 145.

In some aspects, the polynucleotide or nucleic acid sequence further comprises a 5′ UTR and/or a 3′ UTR. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 1-16, 84-88, 123-141, or 160-161.

Certain aspects of the disclosure are directed to a polynucleotide comprising a nucleic acid encoding a human insulin (Ins) protein (e.g., a preproinsulin or variant thereof), wherein the nucleic acid comprises: (i) a nucleotide sequence encoding a signal peptide (e.g., a wild-type preproinsulin signal sequence, an IL-6 signal sequence, or a fibronectin signal sequence) and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid in wild-type proinsulin (or an amino acid modification at a position selected from amino acid H34, P52, K53, R55, L86, or any combination thereof relative to the corresponding amino acid in wild-type preproinsulin).

In some aspects, the signal peptide is not a wild-type preproinsulin signal sequence (e.g., the wild-type preproinsulin sequence is replaced with an IL-6 signal sequence or fibronectin signal sequence). In some aspects, the proinsulin polypeptide comprises the amino acid sequence of any of amino acids 25-110 of SEQ ID NO: 41, amino acids 25-110 of SEQ ID NO: 144, or amino acids 25-110 of SEQ ID NO: 145.

In some aspects, the polynucleotide further comprises a cleavage site (e.g., a furin cleavage site).

In some aspects, the encoded human Ins protein (e.g., a preproinsulin or variant thereof) comprises an amino acid modification selected from (i) H34D, H34I, or H34V (or a histidine (H) to aspartic acid (D), isoleucine (I) or valine (V) at position B10 of the proinsulin B-chain), and/or (ii) one or more amino acid modifications at P52, K53, R55, and/or L86 relative to the wild-type preproinsulin sequence (or positions B28 and/or B29 of the proinsulin B-chain or positions C1 and/or C32 of the proinsulin C-chain). In some aspects, the one or more amino acid modifications at P52, K53, R55, and/or L86 comprise P52D, K53R, R55K, L86R, or any combination thereof (or the one or more modifications in the proinsulin B-chain or C-chain comprise a proline (P) to aspartic acid (D) at position B28 of the proinsulin B-chain, a lysine (K) to arginine (R) at position B29 of the proinsulin B-chain, arginine (R) to lysine (K) at position C1 of the proinsulin C-chain, leucine (L) to arginine (R) at position C32 of the proinsulin C-chain, or any combination thereof).

Certain aspects of the disclosure are directed to a polynucleotide comprising a nucleic acid encoding a human glucokinase (Gck) protein, wherein the nucleic acid comprises an ORF comprising: a nucleotide sequence at least 85%, 86%, 87%, 88%, 89%, 90%, 91%, 92%, 93%, 94%, 95%, 96%, 97%, 98%, 99%, or 100% identical to any of nucleic acids 1-1398 of any of SEQ ID NO: 61-80 or 162; or SEQ ID NO: 61-80 and 162. In some aspects, the encoded human Gck protein comprises the amino acid sequence of SEQ ID NO: 82.

In some aspects, the polynucleotide or nucleic acid sequence encoding a Gck protein further comprises a 5′ UTR and/or a 3′ UTR. In some aspects, the nucleic acid further comprises a 5′ UTR comprising a nucleotide sequence at least 85%, 90%, 95%, 99%, or 100% identical to SEQ ID NO: 42, 5-329 of SEQ ID NO: 42, SEQ ID NO: 83, SEQ ID NO: 146, or SEQ ID NO: 148. In some aspects, the nucleic acid further comprises a 3′ UTR comprising a nucleotide sequence at least 85%, 90%, 95%, 99%, or 100% identical to SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 20-39 and 89-96, and 163-164.

In some aspects, the nucleic acid is operably linked to a promoter (e.g., a eukaryotic promoter). Certain aspects of the disclosure are directed to an expression cassette comprising a polynucleotide of the disclosure and a heterologous expression control sequence operably linked to the nucleic acid sequence. In some aspects, the nucleic acid is operably linked to a polyadenylation (poly A) element.

Certain aspects of the disclosure are directed to a vector (e.g., viral vector, a non-viral vector, a plasmid, a lipid, or a lysosome) comprising a polynucleotide or an expression cassette of the disclosure. In some aspects, the vector is an adeno-associated virus (AAV) vector or a Lentivirus vector. Certain aspects of the disclosure are directed to a recombinant AAV (rAAV) particle, comprising an AAV capsid and a vector genome comprising the polynucleotide or the expression cassette of the disclosure. In some aspects, the AAV serotype is selected from the group consisting of AAV1, AAV2, AAV3, AAV4, AAV5, AAV6, AAV7, AAV8, AAVRH8, AAVrh9, AAV9, AAVrh10, AAV10, AAVRH10, AAV11, and AAV12. Certain aspects of the disclosure are directed to a host cell (e.g., a mammalian cell) comprising a polynucleotide, an expression cassette, a vector, or a rAAV particle of the disclosure.

Certain aspects of the disclosure are directed to a method of producing human Ins protein and/or a human Gck protein in a subject, comprising administering to the subject a polynucleotide, an expression cassette, a vector, or a rAAV particle of the disclosure, thereby producing the human Ins protein and/or human Gck in the subject. Certain aspects of the disclosure are directed to a method of treating or ameliorating the symptoms associated with diabetes in a subject in need thereof, comprising delivering to the subject a therapeutically effective amount of a polynucleotide, an expression cassette, a vector, or a rAAV particle of the disclosure, thereby treating diabetes in the subject. In some aspects, the diabetes is diabetes mellitus type 1 (T1DM) or diabetes mellitus type 2 (T2DM).

In some aspects, the methods disclosed herein comprise administering to the subject a plurality of polynucleotides comprising a first polynucleotide encoding human insulin and a second polynucleotide encoding human Gck; a plurality of expression cassette comprising a first expression cassette comprising a polynucleotide encoding human insulin and a second expression cassette comprising a polynucleotide encoding human Gck; a plurality of vectors comprising a first vector comprising an expression cassette comprising a polynucleotide encoding human insulin and a second vector comprising an expression cassette comprising a polynucleotide encoding human Gck; or a plurality of rAAV particles comprising a first rAAV particle comprising an expression cassette comprising a polynucleotide encoding human insulin and a second rAAV particle comprising an expression cassette comprising a polynucleotide encoding human Gck. In some aspects, the plurality of polynucleotides, expression cassettes, vectors, or rAAV particles are administered simultaneously or sequentially. In some aspects, the delivery and/or administration of a polynucleotide, an expression cassette, a vector, or a rAAV particle of the disclosure is intramuscular. In some aspects, the methods of the disclosure provide (i) reduction and/or regulation of glycated blood hemoglobin (HbA1c) levels in the subject; (ii) reduction in circulating ketones in the subject, (iii) reduction in triglycerides in the subject, or (iv) any combination thereof.

### BRIEF DESCRIPTION OF FIGURES

FIG. 1A shows a listing of nucleic acid sequence constructs including the human insulin (hIns) unmodified nucleic acid sequence (SEQ ID NO: 1, SEQ ID NO: 127, and SEQ ID NO: 160) and modified hIns nucleic acid sequences (SEQ ID NOs: 2-16, 84-88, 123-126, and 128-141). The sequences include 3′ UTR, ORF, and 5′ UTR nucleic acid sequences. Certain sequences also include an IRES sequence. Exemplary pAAV-Ins plasmids transfected into HEK cells are shown in the right-hand column.

FIGS. 1B and 1C are graphs showing insulin secretion from HEK cells transfected with either 0.5 ug/well (FIG. 1B) or 0.1 ug/well (FIG. 1C) pAAV-insulin plasmids. The Insulin expression level of each plasmid was compared with the control plasmid (AAV1-CMV-hInsB10D_2).

FIG. 2A shows a listing of nucleic acid sequence constructs including the human glucokinase (hGcK) wild-type nucleic acid sequence (SEQ ID NO: 19 and SEQ ID NO: 163) and modified hGcK nucleic acid sequences (SEQ ID NOs: 20-39 and 89-96). The sequences include 3′ UTR, ORF, and 5′ UTR nucleic acid sequences. Exemplary pAAV-Gck plasmids transfected into HEK cells are shown in the right-hand column.

FIG. 2B is a graph showing glucokinase expression in HEK cells transfected with 2.5 ug/well pAAV-Gck plasmids. Gck expression level of each plasmid was compared with a control plasmid (AAV1-CMV-hGcKWT_2).

FIGS. 3A-3C are graphs showing intracellular AAV1-hInsulin vector genome (vg) quantities in cell extracts of 2v6.11 cells infected with vectors AAV1-CMV-hInsB10D-9 (SEQ ID NO: 110), AAV1-CMV-Ins5 (SEQ ID NO: 87) and AAV1-CMV-Ins7 (SEQ ID NO: 88) at three different MOIs (1000 vg/cell (1K), 2000 vg/cell (2K) and 4000 vg/cell (4K)) and in three independent studies. FIG. 3A is assay 1, FIG. 3B is assay 2, and FIG. 3C is assay 3.

FIGS. 4A-4C are graphs showing human insulin mRNA expression levels in 2v6.11 cells infected with vectors AAV1-CMV-hInsB10D (SEQ ID NO: 110), AAV1-CMV-Ins5 (SEQ ID NO: 87) and AAV1-CMV-Ins7 (SEQ ID NO: 88) at three different MOIs (1000 vg/cell (1K), 2000 vg/cell (2K) and 4000 vg/cell (4K)) and in three independent studies. FIG. 4A is assay 1, FIG. 4B is assay 2, and FIG. 4C is assay 3.

FIGS. 5A-5C are graphs showing secreted human insulin (mU/L) levels measured after three independent infection studies of 2v6.11 cells with vectors AAV1-CMV-hInsB10D (SEQ ID NO: 110), AAV1-CMV-Ins5 (SEQ ID NO: 87) and AAV1-CMV-Ins7 (SEQ ID NO: 88) at three different MOIs (1000 vg/cell (1K), 2000 vg/cell (2K) and 4000 vg/cell (4K)). FIG. 5A is assay 1, FIG. 5B is assay 2, and FIG. 5C is assay 3.

FIGS. 6A-6C are graphs showing the functionality of secreted human insulin measured after three independent infection studies of 2v6.11 cells with vectors A AAV1-CMV-hInsB10D (SEQ ID NO: 110), AAV1-CMV-Ins5 (SEQ ID NO: 87) and AAV1-CMV-Ins7 (SEQ ID NO: 88) at three different MOIs (1000 vg/cell (1K), 2000 vg/cell (2K) and 4000 vg/cell (4K)). Activity is expressed as ng/ml using recombinant human insulin (Life Technologies) as the standard reference. FIG. 6A is assay 1, FIG. 6B is assay 2, and FIG. 6C is assay 3.

FIGS. 7A-7C are graphs showing intracellular AAV1-hGlucokinase vector genome (vg) quantities in cell extracts of 2v6.11 cells infected with vectors AAV1-CMV-hGckWT (Wild type) (SEQ ID NO: 19), AAV1-CMV-Gck8 (SEQ ID NO: 93) and AAV1-CMV-Gck12 (SEQ ID NO: 95) at three different MOIs (1000 vg/cell (1K), 2000 vg/cell (2K) and 4000 vg/cell (4K)) in three independent assays. FIG. 7A is assay 1, FIG. 7B is assay 2, and FIG. 7C is assay 3.

FIGS. 8A-8C are graphs showing human glucokinase mRNA expression levels in 2v6.11 cells infected with vectors AAV1-CMV-hGckWT (Wild type) (SEQ ID NO: 19), AAV1-CMV-Gck8 (SEQ ID NO: 93) and AAV1-CMV-Gck12 (SEQ ID NO: 95) at three different MOIs (1000 vg/cell (1K), 2000 vg/cell (2K) and 4000 vg/cell (4K)) in three independent assays. FIG. 8A is assay 1, FIG. 8B is assay 2, and FIG. 8C is assay 3.

FIGS. 9A-9C are graphs showing intracellular glucokinase levels (ng/mg) measured after three independent infection studies of 2v6.11 cells with vectors AAV1-CMV-hGckWT (Wild-type) (SEQ ID NO: 19), AAV1-CMV-Gck8 (SEQ ID NO: 93) and AAV1-CMV-Gck12 (SEQ ID NO: 95) at three different MOIs (1000 vg/cell (1K), 2000 vg/cell (2K) and 4000 vg/cell (4K)) in three independent assays. FIG. 9A is assay 1, FIG. 9B is assay 2, and FIG. 9C is assay 3.

FIGS. 10A-10C are graphs showing glucokinase enzymatic activity (mU/mg) measured in cell extracts after three independent infection studies of 2v6.11 cells with vectors AAV1-CMV-hGckWT (Wild type) (SEQ ID NO: 19), AAV1-CMV-Gck8 (SEQ ID NO: 93) and AAV1-CMV-Gck12 (SEQ ID NO: 95) at three different MOIs (1000 vg/cell (1K), 2000 vg/cell (2K) and 4000 vg/cell (4K)) in three independent assays. FIG. 10A is assay 1, FIG. 10B is assay 2, and FIG. 10C is assay 3.

FIGS. 11A-11C are graphs showing glucose levels under fed or fasted conditions for individual C57Blk6 mice injected with an AAV1 containing a human insulin gene (SEQ ID NO: 1) and an AAV1 containing a rat glucokinase gene three weeks (FIG. 11A), four weeks (FIG. 11B), and five weeks (FIG. 11C) after injection. The four (4) week time point in FIG. 11B was collected under fasted conditions.

FIG. 12 is a graph showing mean glucose levels under fed or fasted conditions in C57Blk6 mice injected with an AAV1 containing a human insulin gene (SEQ ID NO: 1) and an AAV1 containing a rat glucokinase gene tested over a five-week period after injection. The timepoints were collected under fed conditions except for the four (4) week time point, which was collected under fasted conditions.

FIGS. 13A-13B are graphs showing intracellular insulin content and secreted insulin levels for HEK293 cells transfected with AAV plasmids (pAAV) comprising modified insulin nucleic acid sequences.

FIGS. 14A-14D are graphs showing expression levels of insulin in HEK293 cells transfected with pAAV comprising modified insulin nucleic acid sequences.

FIG. 15 is a graph showing secreted insulin levels for HEK293 cells transfected with pAAV comprising modified insulin nucleic acid sequences.

FIGS. 16A-16F are graphs showing blood glucose levels following an oral glucose tolerance test of CD1 mice treated with AAV vectors comprising modified insulin nucleic acid sequences.

FIG. 17 is a graph showing the fasting glucose levels of healthy mice treated with AAV vectors comprising modified insulin nucleic acid sequences. The measurements were taken three (3) weeks post-AAV administration.

FIGS. 18A-18C are graphs showing TLR9 stimulation in HEK cells, modified to overexpress human TLR9, subsequently transduced with AAV-ratGck or AAV-hInsB10_2 (FIG. 18A); AAV1-hGckWT, AAV1-hGck8, or AAV1-hGck12 (FIG. 18B); and AAV1-CMV-hInsB10D, AAV1-hIns5, or AAV1-hIns7 (FIG. 18C).

FIG. 19 is a graph showing mean blood glucose levels over time in an STZ-induced mouse model of type 1 diabetes. Filled circles represent non-STZ+PBS vehicle controls; open circles STZ+PBS controls; open triangles AAV1_926+AAV1_927 (high dose); filled triangles with a dashed line B10H AAV1-Gck (high dose); filled diamonds Ins-B10H+IL6 AAV1-Gck (low dose); filled triangles with a solid line B10H AAV1-Gck (mid dose); open diamonds B10H+IL6AAV1-Gck (high dose).

FIGS. 20A and 20B are graphs showing the levels of circulating human insulin (ng/mL) in fasted control or STZ-treated mice 4 weeks after i.m. administration of AAV1_926+AAV1_927 (high dose), B10D AAV1-Gck2 (low dose), B10H-IL6 AAV1-Gck (low dose), B10H-IL6 AAV1-Gck (high dose), or PBS control. Circulating insulin levels were not determined in animals that had expired or were euthanized due to poor health/hypoglycemia. The dashed lines in FIG. 20B represent circulating Insulin levels in non-diabetic C57BL/6 mice under fasted conditions.

FIG. 21 is a graph showing the results of an oral glucose tolerance test in control or STZ-treated mice performed 8 weeks after administration of B10H AAV1-Gck (high dose), Ins-B10H-IL6 AAV1-Gck (low dose), or vehicle controls.

FIG. 22 is a graph showing the area under the curve (AUC) of blood glucose levels calculated from 0 to 120 min post-glucose challenge in control or STZ treated mice performed 8 weeks after administration of B10H AAV1-Gck (high dose), B10H+IL6 AAV1-Gck (low dose), or vehicle controls.

FIGS. 23A and 23B are graphs showing HbA1c levels in control and STZ-treated mice 8 weeks after administration of B10H AAV1-Gck (high dose), Ins-B10D AAV-Gck (low dose), Ins-B10D AAV-Gck (mid dose), Ins-B10H+IL6 AAV1-Gck (low dose), Ins-B10H+IL6 AAV1-Gck (high dose) or vehicle controls (FIG. 23A) or B10H-AAV1-Gck (high dose), Ins-B10H+IL6 AAV1-Gck (low dose), or vehicle controls (FIG. 23B). HbA1C levels were not determined in animals that had expired or were euthanized due to poor health/hypoglycemia.

FIGS. 24A-24B are graphs showing serum triglyceride (FIG. 24A) or ketone (FIG. 24B) levels in control or STZ-treated mice after administration of B10H AAV1-Gck (low dose), B10H AAV1-Gck (high dose), Ins-B10D AAV1-Gck (low dose), Ins-B10D AAV1-Gck (mid dose), Ins-B10H+IL6 AAV1-Gck (low dose), Ins-B10H+IL6 AAV1-Gck (high dose), or vehicle controls.

FIG. 25 is a graph showing hINS expression in the liver of STZ treated mice after administration of AAVImTWhIns+AAVIrGck (KT1+AAV926; high dose), AAVImWTINS+AAVIrGck (INS-17+AAV926; low dose), or AAVImWThINS+AAVIrGck (INS-17+AAV926; high dose).

### DETAILED DESCRIPTION OF THE DISCLOSURE

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with, 37 CFR § 1.822 and established usage.

“Polynucleotide” or “nucleic acid” as used herein means a sequence of nucleotides connected by phosphodiester linkages. Polynucleotides are presented herein in the direction from the 5′ to the 3′ direction. A polynucleotide of the present disclosure can be a deoxyribonucleic acid (DNA) molecule or ribonucleic acid (RNA) molecule. Nucleotide bases are indicated herein by a single letter code: adenine (A), guanine (G), thymine (T), cytosine (C), inosine (I) and uracil (U).

As used herein, the term “polypeptide” encompasses both peptides and proteins, unless indicated otherwise.

The term “coding sequence” or “sequence encoding” is used herein to mean a DNA or RNA region (the transcribed region) which “encodes” a particular protein, e.g., such as an insulin or a glucokinase. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

A gene can comprise several operably linked fragments, such as a promoter, a 5′ leader sequence, an intron, a coding sequence and a 3′-nontranslated sequence, e.g., comprising a polyadenylation site or a signal sequence. As used herein, “expression of a gene” refers to the process wherein a gene is transcribed into an RNA and/or translated into an active protein.

An open reading frame (ORF) as used herein is the part of a reading frame that has the ability to be translated. An ORF is a continuous stretch of codons that begins with a start codon and ends at a stop codon. In some aspects, an ORF sequence can be shown or referenced with or without the start codon sequence and/or the stop codon sequence. A Kozak consensus sequence, Kozak consensus or Kozak sequence, is known as a sequence which occurs on eukaryotic mRNA and has the consensus (gcc) gccRccAUGG, where R is a purine (adenine or guanine) three bases upstream of the start codon (AUG), which is followed by another “G.” In some aspects, the polynucleotide comprises a nucleic acid sequence having at least 95%, at least 99% sequence identity, or more to the Kozak consensus sequence. In some aspects, the polynucleotide comprises a Kozak consensus sequence.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case may be, as determined by the match between strings of such sequences.

In certain aspects, methods to determine identity are designed to give the largest match between the sequences tested. Methods to determine identity and similarity are codified in publicly available computer programs.

“Substantial homology” or “substantial similarity,” means, when referring to a nucleic acid or fragment thereof, indicates that, when optimally aligned with appropriate nucleotide insertions or deletions with another nucleic acid (or its complementary strand), there is nucleotide sequence identity in at least about 95 to 99% of the sequence.

As used herein, and unless otherwise indicated, the term “complementary,” when used to describe a first nucleic acid sequence in relation to a second nucleic acid sequence, refers to the ability of an oligonucleotide or polynucleotide comprising the first nucleic acid sequence to hybridize and form a duplex structure under certain conditions with an oligonucleotide or polynucleotide comprising the second nucleic acid sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can be used. The skilled person will be able to determine the set of conditions most appropriate for a test of complementarity of two sequences in accordance with the ultimate application of the hybridized nucleotides.

The term “promoter” is used herein to mean a nucleic acid sequence or fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “transgene” is used herein to mean a gene or a nucleic acid molecule that is introduced into a cell. An example of a transgene is a nucleic acid encoding a therapeutic polypeptide (e.g., a gene encoding an insulin and/or a gene encoding a glucokinase). In some embodiments, the gene can be present but in some cases normally not expressed or expressed at an insufficient level in the cell. In this context, “insufficient” means that although said gene, e.g., insulin and/or glucokinase, is normally expressed in a cell, a condition and/or disease as disclosed herein (e.g., diabetes) could still be developed. In certain aspects, the transgene allows for the increased expression or over-expression of the gene, e.g., an insulin and/or a glucokinase. The transgene can comprise sequences that are native to the cell, comprise sequences that do not naturally occur in the cell, or it can comprise combinations of both. In certain aspects, the transgene can comprise modified sequences coding for an insulin, a glucokinase, both an insulin and a glucokinase, and/or additional protein(s) that can be operably linked to appropriate regulatory sequences for expression of the sequences coding for an insulin, a glucokinase, or both an insulin and a glucokinase in the cell. In some aspects, the transgene is not integrated into the host cell's genome.

The terms “modified genes”, “modified nucleic acids”, and the like are used interchangeably herein to mean the introduction of one or more modifications or changes relative to the in the natural sequence of the genes or nucleic acid sequence. Such modifications may or may not result in mutations to the encoded protein sequence. In some embodiments, the modified nucleic acid encodes a wild-type or mutant protein sequence or fragment thereof.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., a modified human insulin gene) that is derived from a second nucleic acid sequence (e.g., a wild-type human insulin gene) can include a nucleotide sequence or portion thereof that is identical or substantially similar to the nucleotide sequence of the second nucleic acid sequence. In some aspects, mutants, analogs or derivatives can be derived from a wild-type sequence.

In the case of a polynucleotide, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each.

As used herein, the term “delivery vector” or “vector” includes any genetic element, such as a plasmid, phage, transposon, cosmid, chromosome, artificial chromosome, virus, virion, etc., which is capable of replication when associated with the proper control elements and which can transfer gene or nucleic acid sequences between cells. Thus, the term includes cloning and expression vehicles, as well as viral vectors. In some aspects, useful vectors are contemplated to be those vectors in which the nucleic acid segment to be transcribed is positioned under the transcriptional control of a promoter. In some aspects, the delivery vector is selected from the group consisting of a viral vector, a plasmid, lipid, and a lysosome.

In some aspects, the biological vectors include viruses, particularly attenuated and/or replication-deficient viruses. In some embodiments, chemical vectors include lipid complexes and naked DNA constructs.

As used herein, the term “naked DNA” or “naked nucleic acid” and the like refers to a nucleic acid molecule that is not contained within a viral particle, bacterial cell, or other encapsulating means that facilitates delivery of nucleic acid into the cytoplasm of the target cell. Naked nucleic acid can be associated with means for facilitating delivery of the nucleic acid to the site of the target cell (e.g., to facilitate travel into the target cell of the nucleic acid through the alimentary canal, protect the nucleic acid from stomach acid, and/or serve to penetrate intestinal mucus) and/or to the surface of the target epithelial cell.

A “viral genome” or “vector genome” or “viral vector” refers to a sequence that comprises one or more polynucleotide regions encoding or comprising a molecule of interest, e.g., a protein, a peptide, and a polynucleotide or a plurality thereof. Viral vectors are used to deliver genetic materials into cells. Viral vectors can be modified for specific applications. In some aspects, the delivery vectors comprises a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector which comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably.

As used herein, an “AAV particle” is an AAV virus that comprises an AAV vector having at least one payload region (e.g., a polynucleotide encoding insulin and/or Gck) and at least one inverted terminal repeat (ITR) region. In some aspects, the terms “AAV vectors of the present disclosure” or “AAV vectors disclosed herein” refer to AAV vectors comprising a polynucleotide or nucleic acid disclosed herein encoding an insulin, a Gck, or a combination thereof, e.g., encapsulated in an AAV particle.

“Transduction” of a cell by a virus means that there is transfer of a nucleic acid from the virus particle to the cell. In some aspects, transduction refers to the delivery of a nucleic acid or nucleic acids encoding an insulin and/or a glucokinase into a recipient host cell by a viral vector. For example, transduction of a target cell by a rAAV vector of the disclosure leads to transfer of the rAAV genome (e.g., comprising a polynucleotide of the disclosure) contained in that vector into the transduced cell.

“Transfection” of a cell means that genetic material is introduced into a cell for the purpose of genetically modifying the cell. Transfection can be accomplished by a variety of means known in the art, e.g., transduction or electroporation.

“Vector” as used herein means a recombinant plasmid or virus that comprises a polynucleotide to be delivered into a host cell, either in vitro or in vivo.

The term “host cell” or “target cell” is used herein to mean the cell into which the polynucleotide delivery takes place, either in vitro or in vivo. AAV vectors are able to transduce both dividing and non-dividing cells.

“Recombinant” means distinct from that generally found in nature.

“Serotype” with respect to vector or virus capsid is defined by a distinct immunological profile based on the capsid protein sequences and capsid structure.

“AAV Cap” means AAV Cap proteins, VP1, VP2 and VP3 and analogs thereof.

“AAV Rep” means AAV Rep proteins and analogs thereof.

“Flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., ITRs), indicates that one element is located 5′ to the sequence and the other is located 3′ to the sequence; however, there may be intervening sequences between.

As used herein, the terms “effective amount,” “therapeutically effective amount,” and a “sufficient amount” of, e.g., a gene therapy composition comprising a polynucleotide disclosed herein, refer to a quantity sufficient to, when administered to the subject, including a human, effect beneficial or desired results, including clinical results, and, as such, an “effective amount” or synonym thereto depends on the context in which it is being applied.

The amount of a given therapeutic agent or composition will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., a nucleic acid comprising a promoter operably linked to a polynucleotide encoding a therapeutic molecule as defined herein) into an individual's cells and/or tissues to treat a disease or condition. Gene therapy also includes insertion of a transgene that is inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases can be amenable to gene therapy.

In some aspects, the disclosure provides modified nucleic acids encoding wild-type or mutant insulin and/or wild-type glucokinase or a functional fragment thereof. The disclosure also provides nucleic acid constructs that include as part of their sequence the modified nucleic acid(s) encoding wild-type or mutant insulin and/or wild-type glucokinase or a functional fragment thereof. For example, the disclosure includes expression cassettes, plasmids and/or other vectors that include the modified nucleic acid sequence(s) along with other elements, such as regulatory elements. In some aspects, the disclosure provides a packaged gene delivery vehicle, such as a viral capsid, including the modified nucleic acid sequence(s) encoding wild-type or mutant insulin and/or wild-type glucokinase or a functional fragment thereof. The disclosure also includes methods of expressing wild-type or mutant insulin and/or wild-type glucokinase or a functional fragment thereof by delivering the modified nucleic acid sequence(s) into a cell along with elements required to promote expression in the cell. The disclosure also provides gene therapy methods in which the modified nucleic acid sequence(s) encoding wild-type or mutant insulin and/or wild-type glucokinase or a functional fragment thereof is/are administered to a subject, e.g., as a component of one or more vectors and/or packaged as a component of one or more viral gene delivery vehicles. Treatment can, for example, be effected to treat or reduce the symptoms of diabetes in a subject in need thereof. Each of these aspects of the disclosure is discussed in further detail herein.

### Modified Nucleic Acids

In some aspects, the present disclosure provides polynucleotides that comprise modified (e.g., codon-optimized and/or reduced CpG content) nucleic acids encoding insulin, glucokinase, or a combination thereof. In some aspects, the modified nucleic acid encodes human insulin (e.g., preproinsulin or proinsulin, a mutant, an analogue, or a variant thereof). In some aspects, the modified nucleic acid encodes human glucokinase (e.g., Gck, a mutant, an analogue, or variant thereof). In some aspects, the modifications to the coding sequence preserve the wild-type or mutant amino acid sequence for insulin and/or glucokinase. In some aspects, the encoded human Ins protein comprises a signal sequence and a proinsulin polypeptide. In some aspects, the encoded human Ins protein comprises the amino acid sequence of any of amino acids 25-110 of SEQ ID NO: 41, amino acids 25-110 of SEQ ID NO: 144, or amino acids 25-110 of SEQ ID NO: 145. In some aspects, the modified nucleic acid sequence encodes a human preproinsulin (e.g., SEQ ID NO: 41, SEQ ID NO: 144, or SEQ ID NO: 145). In some aspects, the modified nucleic acid sequence encodes a human Gck (e.g., SEQ ID NO: 82).

In some aspects, the modified nucleic acids are codon optimized. In some aspects, the codon optimization includes modifying codons in the open reading frame of the nucleic acid encoding insulin or glucokinase. In some aspects, the modified nucleic acids comprise reduced CpG content relative to the corresponding wild-type sequence and/or unmodified sequence.

In some aspects, the modified nucleic acid has reduced innate immunogenicity relative to the corresponding wild-type sequence and/or unmodified sequence. In some aspects, the modified nucleic acid has increased expression relative to the corresponding wild-type sequence and/or unmodified sequence. In some aspects, the modified nucleic acid has decreased expression relative to the corresponding wild-type sequence and/or unmodified sequence. In some aspects, the modified sequences are developed through in silico methods followed by manual sequence examination. Nucleic acids of the disclosure can be produced using molecular biology techniques, e.g., modified cDNAs encoding insulin or glucokinase can be obtained by PCR amplification or cDNA cloning techniques.

In some aspects, the nucleic acid sequences are modified to reduce CpG content, e.g., to minimize the inflammatory response through TLR9 dimerization and related pathways. In some aspects, certain CpG motifs are inhibitory or neutralizing for their inflammatory effects. In some embodiments, one or more of these motifs can be preserved. In some aspects, such CpG motifs can be introduced into a nucleic acid sequence for inhibition of the downstream effects of TLR9 dimerization.

In some aspects, the codon modifications can reduce the immunogenicity of the insulin and/or glucokinase encoding polynucleotides relative to a corresponding wild-type polynucleotide and/or unmodified polynucleotide. In some aspects, the codon modifications improve the expression of the insulin or glucokinase encoding polynucleotide relative to a corresponding wild-type and/or unmodified polynucleotide. In some aspects, the codon modifications can reduce the immunogenicity of the glucokinase encoding polynucleotides relative to a corresponding wild-type Gck polynucleotide and/or unmodified Gck polynucleotide.

The modified nucleic acids of the disclosure can be present in whole cells, in a cell lysate, or in a partially purified or substantially pure form. The modified nucleic acids can be isolated. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art, see e.g. F. Ausubel, et al., ed. (1987) Current Protocols in Molecular Biology, Greene Publishing and Wiley Interscience, New York. In some aspects, a modified nucleic acid of the disclosure can be, for example, DNA or RNA and may or may not contain intron sequences. In some aspects, the nucleic acid can be a cDNA molecule.

### Modified Insulin Nucleic Acids

In some aspects, a polynucleotide or nucleic acid sequence disclosed herein is modified relative to a wild-type (SEQ ID NO: 147) and/or unmodified human insulin (Ins) or a human Ins mutant or analogue (e.g., SEQ ID NO: 110 or SEQ ID NO: 111). In some aspects, the polynucleotide or nucleic acid is modified relative to a sequence including a 5′ UTR, an ORF, and/or a 3′ UTR, e.g., corresponding to SEQ ID NO: 1 or SEQ ID NO: 127. In some aspects, the modified nucleic acid encodes wild-type human insulin (SEQ ID NO: 41), variants or mutants thereof (e.g., SEQ ID NO: 144 or SEQ ID NO: 145) or a functional fragment thereof.

Insulin includes two polypeptide chains, the A- and B-chains, linked together by disulfide bonds. It is first synthesized as a single polypeptide called preproinsulin. “Preproinsulin” is the primary translational product of the insulin gene. It is a peptide that is 110 amino acids in length. Preproinsulin includes a proinsulin molecule with a signal peptide attached to its N-terminus. Part of the N-terminus including the signal peptide of the preproinsulin is cleaved off, leaving the remaining amino acids as “proinsulin”. Amino acids 1-30 of the resulting cleaved sequence is the “B chain”, and here “B10” corresponds to position 34 of preproinsulin. Thus, for example, a “B10” proinsulin mutation corresponds to a H34 mutation in preproinsulin. In certain aspects, as referenced herein, “B10H” refers the wild-type histidine amino acid at the B10 position (also referenced as H34 in the wild-type preproinsulin sequence). The preproinsulin and proinsulin also include a C-peptide between the A- and B-chains. In the mature insulin protein, the C-peptide is proteolytically cleaved and the A- and B-chains are linked by disulfide bonds.

In some aspects, the modified coding sequence disclosed herein encodes a preproinsulin mutant comprising one or more mutations at position(s) H34, P52, K53, R55, and/or L86 relative to the corresponding position in wild-type preproinsulin (SEQ ID NO: 41). In some aspects, the modified coding sequence encodes a preproinsulin mutant comprising one or more of the following mutations H34D, H34I, H34V, P52D, K53R, R55K, and/or L86R relative to the corresponding position in SEQ ID NO: 41. In some aspects, the modified coding sequence encodes a preproinsulin mutant comprising mutations H34D, H34I, H34V, P52D, K53R, R55K, and/or L86R relative to the corresponding position in SEQ ID NO: 41. In some aspects, the modified coding sequence encodes a preproinsulin mutant comprising mutations P52D, K53R, R55K, and/or L86R relative to the corresponding position in SEQ ID NO: 41. In some aspects, a modified coding sequence encodes an amino acids sequence at least 90%, 95%, 99% or 100% similar to an amino acid sequence selected from SEQ ID NO: 41, SEQ ID NO: 144, or SEQ ID NO: 145. In some aspects, a modified coding sequence encodes an amino acids sequence at least 90%, 95%, 99% or 100% similar to an amino acid sequence selected from SEQ ID NO: 41, SEQ ID NO: 144, or SEQ ID NO: 145, wherein the amino acid sequence comprises one or more of the following mutations H34D, H34I, H34V, P52D, K53R, R55K, and/or L86R relative to the corresponding position in SEQ ID NO: 41. In some aspects, the modified coding sequence encodes an amino acids sequence that does not include a H34 mutation relative to the corresponding position in SEQ ID NO: 41.

In some aspects, the modified nucleic acid sequence comprises a cleavage site, e.g., a furin endoprotease cleavage site.

In some aspects, the modified nucleic acid sequence comprises a nucleic acid encoding a signal peptide (e.g., a wild-type preproinsulin signal sequence, an IL-6 signal sequence, or a fibronectin signal sequence). In some aspects, the preproinsulin comprises a wild-type insulin signal sequence (e.g., MALWMRLLPLLALLALWGPDPAAA (SEQ ID NO: 165) or amino acids 1-24 of SEQ ID NO: 41). In some aspects, the signal sequence of wild-type preproinsulin is replaced with a non-insulin secretion peptide, e.g., an IL-6 signal sequence (e.g., MNSFSTSAFGPVAFSLGLLLVLPAAFPAP (SEQ ID NO: 166)) or a fibronectin signal sequence (e.g., MLRGPGPGLLLLAVQCLGTAVPSTGA (SEQ ID NO: 167)).

In some aspects, the modified nucleic acid encodes a human insulin comprising an amino acid modification selected from H34D, H34I, or H34V corresponding to wild-type preproinsulin amino acid positions (or a histidine (H) to aspartic acid (D), isoleucine (I) or valine (V) at position B10 of the proinsulin B-chain). In some aspects, the modified nucleic acid encodes a human insulin comprising an amino acid modification H34D corresponding to wild-type preproinsulin amino acid positions (or a histidine (H) to aspartic acid (D) at position B10 of the proinsulin B-chain). In some aspects, the modified nucleic acid encodes a human insulin comprising an amino acid modification selected from H34D, H34I, or H34V corresponding to wild-type preproinsulin amino acid positions (or a histidine (H) to aspartic acid (D), isoleucine (I) or valine (V) at position B10 of the proinsulin B-chain), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin comprising amino acid modifications K53R, R55K, and L86R corresponding to wild-type preproinsulin amino acid positions (or modifications corresponding to the proinsulin lysine (K) to arginine (R) at position B29, arginine (R) to lysine (K) at position C1, and leucine (L) to arginine (R) at position C32). In some aspects, the modified nucleic acid encodes a human insulin comprising amino acid modifications K53R, R55K, and L86R corresponding to wild-type preproinsulin amino acid positions (or modifications corresponding to the proinsulin lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin comprising amino acid modifications H34D, K53R, R55K, and L86R corresponding to wild-type preproinsulin amino acid positions (or modifications corresponding to the proinsulin a histidine (H) to aspartic acid (D) at position B10, lysine (K) to arginine (R) at position B29, arginine (R) to lysine (K) at position C1, and leucine (L) to arginine (R) at position C32). In some aspects, the modified nucleic acid encodes a human insulin comprising amino acid modifications H34D, K53R, R55K, and L86R corresponding to wild-type preproinsulin amino acid positions (or modifications corresponding to the proinsulin a histidine (H) to aspartic acid (D) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin comprising amino acid modifications H34I, K53R, R55K, and L86R corresponding to wild-type preproinsulin amino acid positions (or modifications corresponding to the proinsulin a histidine (H) to isoleucine (I) at position B10, lysine (K) to arginine (R) at position B29, arginine (R) to lysine (K) at position C1, and leucine (L) to arginine (R) at position C32). In some aspects, the modified nucleic acid encodes a human insulin comprising amino acid modifications H34I, K53R, R55K, and L86R corresponding to wild-type preproinsulin amino acid positions (or modifications corresponding to the proinsulin a histidine (H) to isoleucine (I) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin comprising amino acid modifications H34V, K53R, R55K, and L86R corresponding to wild-type preproinsulin amino acid positions (or modifications corresponding to the proinsulin a histidine (H) to valine (V) at position B10, lysine (K) to arginine (R) at position B29, arginine (R) to lysine (K) at position C1, and leucine (L) to arginine (R) at position C32). In some aspects, the modified nucleic acid encodes a human insulin comprising amino acid modifications H34V, K53R, R55K, and L86R corresponding to wild-type preproinsulin amino acid positions (or modifications corresponding to the proinsulin a histidine (H) to valine (V) at position B10, lysine (K) to arginine (R) at position B29, arginine (R) to lysine (K) at position C1, and leucine (L) to arginine (R) at position C32), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin comprising amino acid modifications P49D, K53R, R55K, and L86R corresponding to wild-type preproinsulin amino acid positions (or modifications corresponding to the proinsulin a proline (P) to aspartic acid (D) at position B28, lysine (K) to arginine (R) at position B29, arginine (R) to lysine (K) at position C1, and leucine (L) to arginine (R) at position C32). In some aspects, the modified nucleic acid encodes a human insulin comprising amino acid modifications P49D, K53R, R55K, and L86R (corresponding to wild-type preproinsulin amino acid positions), wherein the human insulin optionally comprises a cleavage site, e.g., a furin cleavage site, and a signal peptide (e.g., a wild-type preproinsulin signal sequence, an IL-6 signal sequence, or a fibronectin signal sequence).

In some aspects, the modified nucleic acid encodes a human insulin (Ins) protein (e.g., a preproinsulin or variant thereof), wherein the nucleic acid comprises: (i) a nucleotide sequence encoding a signal peptide (e.g., a wild-type preproinsulin signal sequence, an IL-6 signal sequence, or a fibronectin signal sequence) and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid in wild-type proinsulin (or an amino acid modification at a position selected from amino acid H34, P52, K53, R55, L86, or any combination thereof relative to the corresponding amino acid in wild-type preproinsulin). In some aspects, the signal peptide is not a wild-type preproinsulin signal sequence, e.g., the wild-type preproinsulin sequence is replaced with an IL-6 signal sequence or fibronectin signal sequence). In some aspects, the polynucleotide further comprises a cleavage site (e.g., a furin cleavage site). In some aspects, the encoded human Ins protein (e.g., a preproinsulin or variant thereof) comprises an amino acid modification selected from (i) H34D, H34I, or H34V (or a histidine (H) to aspartic acid (D), isoleucine (I) or valine (V) at position B10 of the proinsulin B-chain), and/or (ii) one or more amino acid modifications at P52, K53, R55, and/or L86 relative to the wild-type preproinsulin sequence (or positions B28 and/or B29 of the proinsulin B-chain or positions C1 and/or C32 of the proinsulin C-chain). In some aspects, the one or more amino acid modifications at P52, K53, R55, and/or L86 comprise P52D, K53R, R55K, L86R, or any combination thereof (or the one or more modifications in the proinsulin B-chain or C-chain comprise a proline (P) to aspartic acid (D) at position B28 of the proinsulin B-chain, a lysine (K) to arginine (R) at position B29 of the proinsulin B-chain, arginine (R) to lysine (K) at position C1 of the proinsulin C-chain, leucine (L) to arginine (R) at position C32 of the proinsulin C-chain, or any combination thereof).

In some aspects, the modified nucleic acid encodes a variant or mutant human insulin protein or a functional fragment thereof. In some aspects, the human insulin protein comprises an amino acid sequence having least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to amino acids 25-110 of SEQ ID NO: 41, amino acids 25-110 of SEQ ID NO: 144, or amino acids 25-110 of SEQ ID NO: 145. In some aspects, the human insulin protein comprises an amino acid sequence having least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 41, SEQ ID NO: 144, or SEQ ID NO: 145. In some aspects, the human insulin protein comprises an insertion, a deletion, a substitution, or combinations thereof relative to wild-type human insulin. In some aspects, the human insulin protein comprises at least one substitution. In some aspects, the at least one substitution is a conservative substitution. In some aspects, the at least one substitution is a non-conservative substitution.

In some aspects, a polynucleotide of the disclosure comprises an open reading frame (ORF) comprising a nucleic acid sequence having a nucleotide sequence at least 85%, 86%, 87%, 88%, 89%, 90%, 91%, 92%, 93%, 94%, 95%, 96%, 97%, 98%, 99%, or 100% identical to nucleic acids 73-330 of any of SEQ ID NOs: 43-57, 110-116, 150-151, 154-155, and 157-159, nucleic acids 88-345 of any of SEQ ID NOs: 117-122, 152, and 156, or nucleic acids 79-336 of SEQ ID NO: 153. In some aspects, the ORF further comprises a nucleic acid sequence encoding a signal peptide.

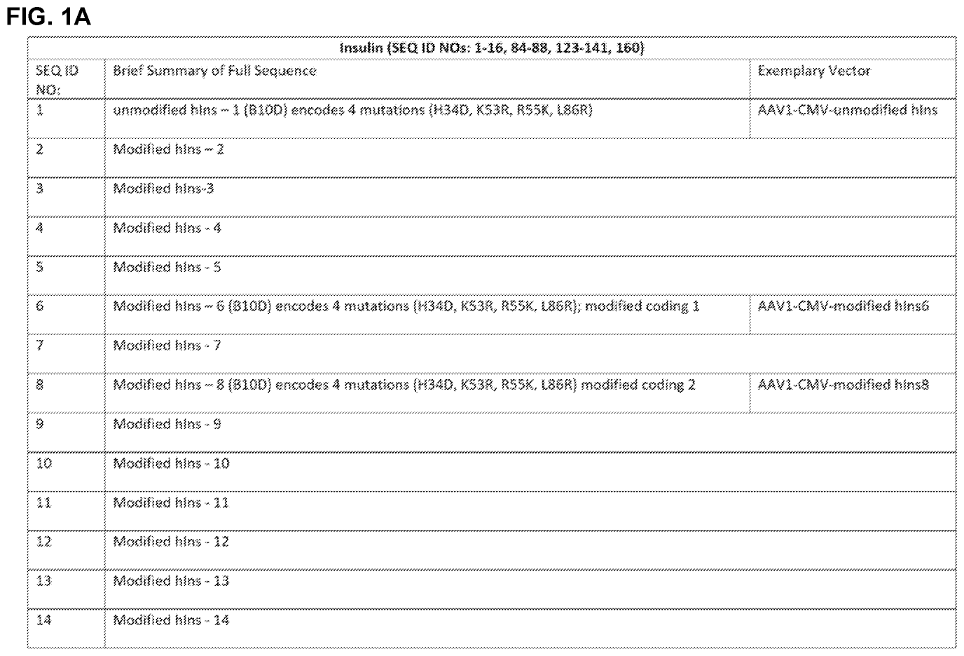

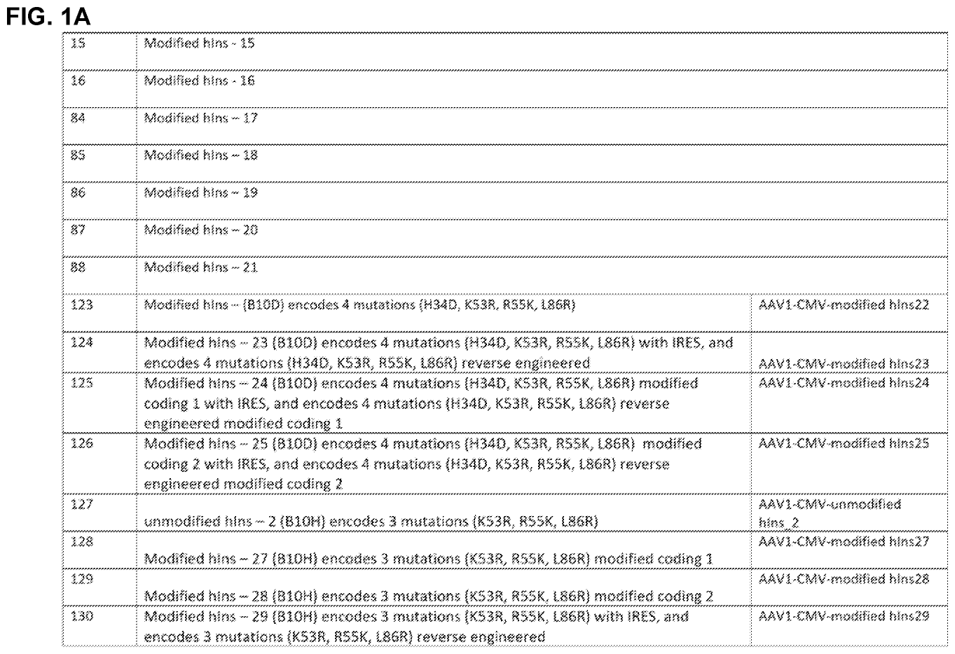

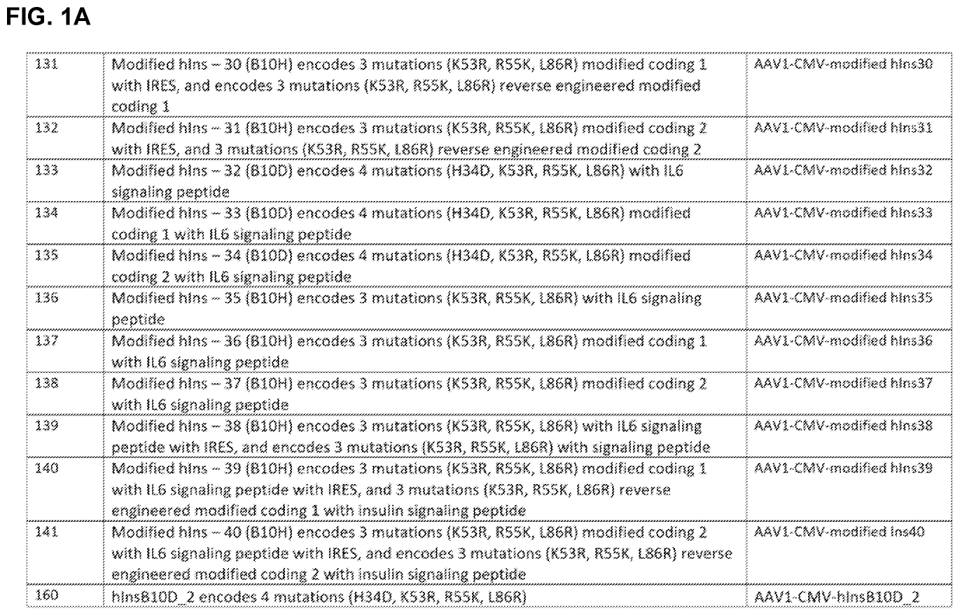

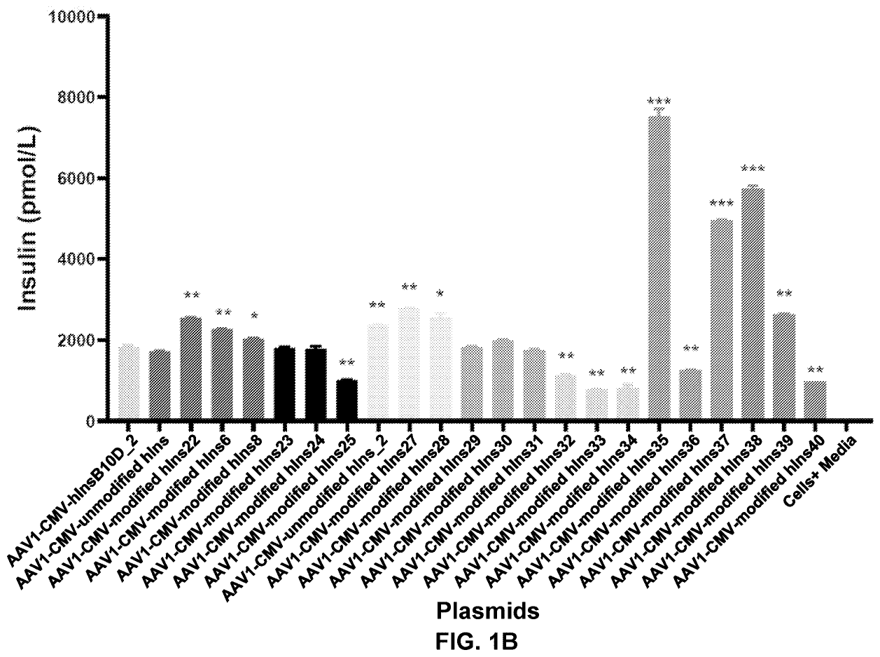

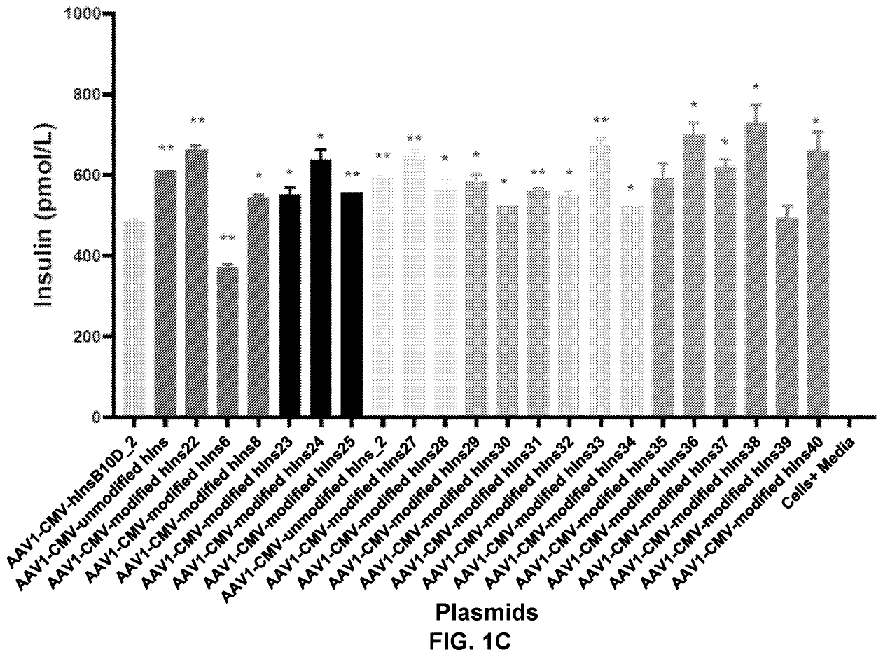

In some aspects, a polynucleotide of the disclosure comprises an open reading frame (ORF) comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the modified nucleic acid sequence encodes a human insulin protein (e.g., SEQ ID NO: 41, SEQ ID NO: 144, or SEQ ID NO: 145) or a functional fragment thereof. In some aspects, a polynucleotide of the disclosure comprises an open reading frame (ORF) comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122. In some aspects, a polynucleotide of the disclosure comprises an open reading frame comprising a nucleic acid having the sequence of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, a polynucleotide of the disclosure comprises an open reading frame comprising a nucleic acid having the sequence of SEQ ID NO: 122. In some aspects, the polynucleotide comprises an ORF sequence present or referenced in Table 1, Table 13, and/or FIG. 1A.

In some aspects, a polynucleotide of the disclosure comprises two or more ORFs selected from the group consisting of a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, and any combinations thereof. In some aspects, one of the two or more ORFs have at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122. In some aspects, the two or more ORFS are selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, and any combinations thereof. In some aspects, one of the two or more ORFs have the nucleic acid sequence of SEQ ID NO: 122.

In some aspects, the two or more ORFs are operably linked. In some aspects, the ORFs are operably linked by an IRES. In some aspects, the IRES comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 142 or SEQ ID NO: 143. In some aspects, the IRES comprises a nucleic acid sequence of SEQ ID NO: 142 or SEQ ID NO: 143.

In some aspects, the two or more ORFs linked by an IRES comprise a nucleic acid sequence having at least at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence comprising SEQ ID NO: 110 and SEQ ID NO: 53, SEQ ID NO: 47 and SEQ ID NO: 54, SEQ ID NO: 49 and SEQ ID NO: 56, SEQ ID NO: 111 and SEQ ID NO: 114, SEQ ID NO: 112 and SEQ ID NO: 115, SEQ ID NO: 113 and SEQ ID NO: 116, SEQ ID NO: 120 and SEQ ID NO: 114, SEQ ID NO: 121 and SEQ ID NO: 115, or SEQ ID NO: 122 and SEQ ID NO: 116. In some aspects, the two or more ORFs linked through an IRES comprise a nucleic acid sequence comprising SEQ ID NO: 110 and SEQ ID NO: 53, SEQ ID NO: 47 and SEQ ID NO: 54, SEQ ID NO: 49 and SEQ ID NO: 56, SEQ ID NO: 111 and SEQ ID NO: 114, SEQ ID NO: 112 and SEQ ID NO: 115, SEQ ID NO: 113 and SEQ ID NO: 116, SEQ ID NO: 120 and SEQ ID NO: 114, SEQ ID NO: 121 and SEQ ID NO: 115, or SEQ ID NO: 122 and SEQ ID NO: 116.

In some aspects, a polynucleotide of the disclosure further comprises a modified 5′ UTR nucleic acid sequence. In some aspects, a polynucleotide of the disclosure further comprises a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, or SEQ ID NO: 83, SEQ ID NO: 146, or SEQ ID NO: 148. In some aspects, the polynucleotide further comprises a Kozak consensus sequence (Kozak consensus or Kozak sequence). In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR nucleic acid sequence present or referenced in Table 1 and/or FIG. 1A.

In some aspects, a polynucleotide of the disclosure further comprises a modified 3′ UTR nucleic acid sequence. In some aspects, a polynucleotide of the disclosure further comprises a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide comprises a 3′ UTR nucleic acid sequence present or referenced in Table 1 and/or FIG. 1A.

In some aspects, a polynucleotide of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161 wherein the nucleic acid sequence encodes a human insulin protein (e.g., SEQ ID NO: 41, SEQ ID NO: 144, or SEQ ID NO: 145) or a functional fragment thereof. In some aspects, a polynucleotide of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138. In some aspects, the polynucleotide of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the polynucleotide of the disclosure comprises a nucleic acid having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161. In some aspects, the polynucleotide of the disclosure comprises a nucleic acid having the sequence of SEQ ID NO: 138. In some aspects, the polynucleotide of the disclosure comprises nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the polynucleotide comprises a modified nucleic acid comprising a 5′ UTR, an ORF, and a 3′ UTR presented in Table 1 and/or FIG. 1A.

In some aspects, the polynucleotide of the disclosure encodes a human insulin comprising a wild-type preproinsulin secretion signal peptide. In some aspects, the polynucleotide of the disclosure does not encode a wild-type preproinsulin secretion signal peptide. In some aspects, the wild-type preproinsulin is replaced by a non-insulin secretion signal. In some aspects, the polynucleotide of the disclosure encodes a human preproinsulin comprising an interleukin 6 (IL-6) secretion signal peptide. In some aspects, the polynucleotide of the disclosure encodes a human preproinsulin comprising a fibronectin secretion signal peptide.

### Modified Glucokinase Nucleic Acids

In some aspects, a polynucleotide or nucleic acid sequence disclosed herein is modified relative to the wild-type and/or unmodified human glucokinase (Gck), e.g., including a 5′ UTR, an ORF, and/or a 3′ UTR, nucleic acid sequence, e.g., corresponding to SEQ ID NO: 19. In some aspects, the modified nucleic acid encodes wild-type human glucokinase (SEQ ID NO: 82) or a functional fragment thereof.

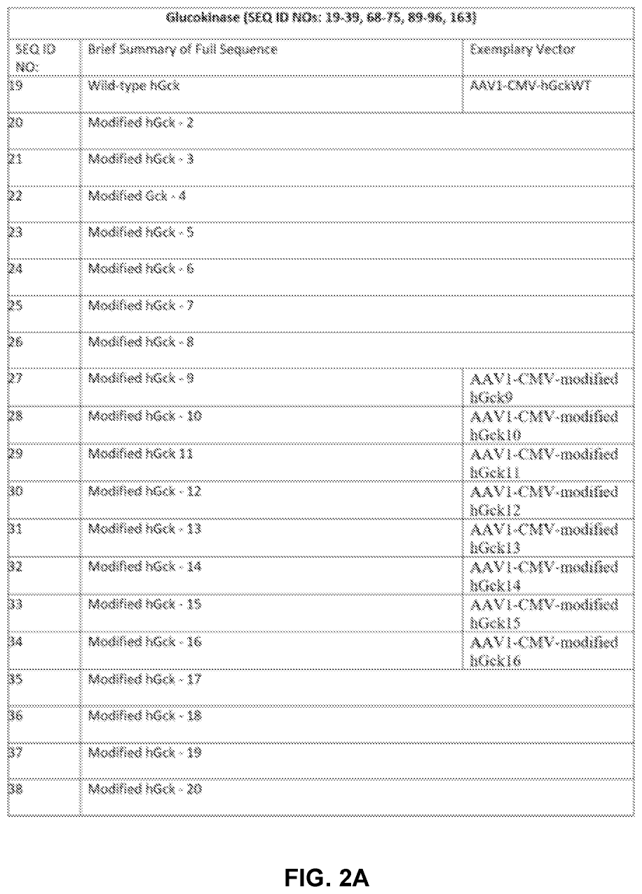

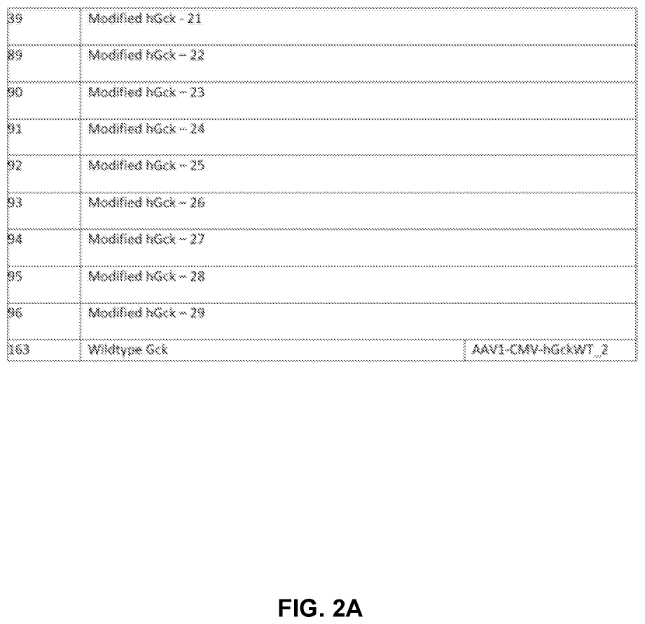

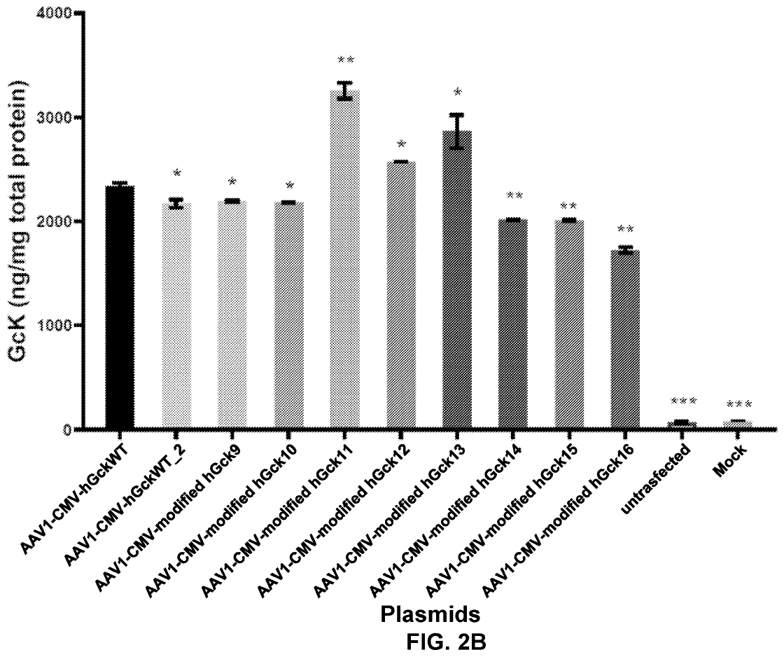

In some aspects, a polynucleotide of the disclosure comprises an ORF comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162 wherein the nucleic acid sequence encodes a human glucokinase protein (SEQ ID NO: 82) or a functional fragment thereof. In some aspects, the polynucleotide of the disclosure comprises an open reading frame comprising a nucleic acid having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the polynucleotide comprises an ORF sequence present or referenced in Table 2 and/or FIG. 2A.

In some aspects, a polynucleotide of the disclosure further comprises a modified 5′ UTR nucleic acid sequence. In some aspects, a polynucleotide of the disclosure further comprises a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the polynucleotide further comprises a Kozak consensus sequence (Kozak consensus or Kozak sequence). In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR sequence present or referenced in Table 2 and/or FIG. 2A.

In some aspects, a polynucleotide of the disclosure further comprises a modified 3′ UTR nucleic acid sequence. In some aspects, a polynucleotide of the disclosure further comprises a 3′ UTR comprising a nucleic acid sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide comprises a 3′ UTR sequence present or referenced in Table 2 and/or FIG. 2A.

In some aspects, the polynucleotide of the disclosure comprises a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164 wherein the nucleic acid sequence encodes a human glucokinase protein (e.g., SEQ ID NO: 82) or functional fragment thereof. In some aspects, the polynucleotide of the disclosure comprises a nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the polynucleotide of the disclosure comprises a nucleic acid having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164. In some aspects, the polynucleotide of the disclosure comprises nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the polynucleotide is a modified nucleic acid present or referenced in Table 2 and/or FIG. 2A.

### Expression Constructs

In some aspects, the present disclosure also provides an expression cassette comprising a nucleic acid sequence, e.g. a modified nucleic acid sequence encoding an insulin, a glucokinase, a combination thereof, or a functional fragment thereof, disclosed herein and a heterologous control sequence operably linked to the nucleic acid sequence. In some aspects, the heterologous control sequence is a promoter.

A nucleic acid construct having a eukaryotic promoter operably linked to a DNA of interest can be used in the disclosure. The constructs containing the DNA sequence (or the corresponding RNA sequence), which can be used in accordance with the disclosure, can be any eukaryotic expression construct containing the DNA or the RNA sequence of interest. For example, a plasmid or viral construct (e.g., an AAV vector) can be cleaved to provide linear DNA having ligatable termini. These termini are bound to exogenous DNA having complementary, like ligatable termini to provide a biologically functional recombinant DNA molecule having an intact replicon and a desired phenotypic property. In some aspects, the construct is capable of replication in both eukaryotic and prokaryotic hosts.

In some aspects, the exogenous DNA used in the disclosure is obtained from suitable cells, and the constructs prepared using techniques known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the DNA construct contains a promoter to facilitate expression of the DNA of interest (e.g., a modified nucleic acid encoding an insulin, a glucokinase, a combination thereof, or a fragment thereof) within a secretory cell. In some aspects, the promoter is a strong, eukaryotic promoter such as a promoter from cytomegalovirus (CMV), mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)). Alternatively, the promoter used can be a tissue-specific promoter.

The constructs of the disclosure can also include other components such as a marker (e.g., an antibiotic resistance gene (such as an ampicillin resistance gene) or β-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both.

For eukaryotic expression, the construct can contain at a minimum a eukaryotic promoter operably linked to a DNA of interest (e.g., a modified nucleic acid encoding an insulin, a glucokinase, a combination thereof, or a fragment thereof), which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. The construct can also include one or more introns, which can increase levels of expression of the DNA of interest, particularly where the DNA of interest is a cDNA (e.g., contains no introns of the naturally-occurring sequence). Any of a variety of introns known in the art can be used (e.g., the human β-globin intron, which is inserted in the construct at a position 5′ to the DNA of interest).

The DNA of interest (e.g., a modified nucleic acid encoding an insulin, a glucokinase, a combination thereof, or a fragment thereof) can be inserted into a construct so that the therapeutic molecule (e.g., a protein) is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

The vectors for delivery of the DNA of interest (e.g., a modified nucleic acid encoding an insulin, a glucokinase, a combination thereof, or a fragment thereof) can be either viral or non-viral, or can be composed of naked DNA admixed with an adjuvant such as viral particles (e.g., AAV particle) or cationic lipids or liposomes. An “adjuvant” is a substance that does not by itself produce the desired effect, but acts to enhance or otherwise improve the action of the active compound. The precise vector and vector formulation used will depend upon several factors such as the cell and/or organ targeted for gene transfer.

Examples of suitable promoters include cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter, RSV promoter, and the herpes simplex virus thymidine kinase promoter. In some aspects, the promoter is a cell-specific and/or a tissue-specific promoter. In some aspects, the promoter is used together with an intronic sequence. In some aspects, the promoter is tissue specific. In some aspects, the promoter is a CMV promoter. In some aspects, the CMV promoter is a mini CMV promoter.

In some aspects, the expression cassette comprises a promoter operably linked to a modified nucleic acid sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the modified nucleic acid sequence encodes a human insulin protein (e.g., SEQ ID NO: 41, SEQ ID NO: 144, or SEQ ID NO: 145) or a functional fragment thereof. In some aspects, the expression cassette comprises a promoter operably linked to a modified nucleic acid sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122. In some aspects, the polynucleotide of the disclosure comprises an open reading frame comprising a nucleic acid having the sequence of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, the polynucleotide of the disclosure comprises an open reading frame comprising a nucleic acid having the sequence of SEQ ID NO: 122. In some aspects, the polynucleotide comprises an ORF sequence present or referenced in Table 1, Table 13, and/or FIG. 1A.

In some aspects, the expression cassette comprises a polynucleotide encoding a human insulin comprising a wild-type preproinsulin secretion signal peptide. In some aspects, the polynucleotide of the disclosure does not encode a wild-type preproinsulin secretion signal peptide. In some aspects, the wild-type preproinsulin is replaced by a non-insulin secretion signal. In some aspects, the expression cassette comprises a polynucleotide encoding a human preproinsulin comprising an interleukin 6 (IL-6) secretion signal peptide. In some aspects, the expression cassette comprises a polynucleotide encoding a human preproinsulin comprising a fibronectin secretion signal peptide.

In some aspects, the expression cassette comprises a modified nucleic acid further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, or SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR sequence present or referenced in Table 1 and/or FIG. 1A.

In some aspects, the expression cassette comprises a modified nucleic acid further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the polynucleotide comprises a 3′ UTR sequence present or referenced in Table 1 and/or FIG. 1A.

In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87 SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161, wherein the modified nucleic acid sequence encodes a human insulin protein (e.g., SEQ ID NO: 41, SEQ ID NO: 144, or SEQ ID NO: 145) or a functional fragment thereof. In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138. In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the expression cassette comprises a modified nucleic acid having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87 SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161. In some aspects, the expression cassette comprises a modified nucleic acid having the sequence of SEQ ID NO: 138. In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the expression cassette comprises a modified nucleic acid comprising a 5′ UTR, an ORF, and a 3′ UTR present or referenced in Table 1 and/or FIG. 1A.

In some aspects, the expression cassette comprises a promoter operably linked to a modified nucleic acid sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the expression cassette comprises a promoter operably linked to a modified nucleic acid having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the polynucleotide comprises an ORF sequence present or referenced in Table 2 and/or FIG. 2A.

In some aspects, the expression cassette comprises a modified nucleic acid further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR sequence present or referenced in Table 2 and/or FIG. 2A.

In some aspects, the expression cassette comprises a modified nucleic acid further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, or any combination thereof. In some aspects, the polynucleotide comprises a 3′ UTR sequence present or referenced in Table 2 and/or FIG. 2A.

In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164 wherein the nucleic acid sequence encodes a human glucokinase protein (e.g., SEQ ID NO: 82) or functional fragment thereof. In some aspects, the expression cassette comprises a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the expression cassette comprises a modified nucleic acid having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164. In some aspects, the expression cassette comprises a modified nucleic acid comprising nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the expression cassette comprises modified nucleic acid comprising a 5′ UTR, an ORF, and a 3′ UTR present or referenced in Table 2 and/or FIG. 2A.

In some aspects, the expression cassette comprises a first modified nucleic acid comprising a first ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159 and a second modified nucleic acid sequence comprising a second ORF having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162.

In some aspects, the expression cassette comprises a promoter operably linked to a modified nucleic acid, wherein each the first and second modified nucleic acids are linked to a first and a second promoter, respectively. In some aspects, the first modified nucleic acid sequence comprising a first ORF is selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, and SEQ ID NO: 159 and the second modified nucleic acid sequence comprising a second ORF is selected from the group consisting of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 162.

In some aspects, the first modified nucleic acid sequence encodes a human insulin comprising a wild-type preproinsulin secretion signal peptide. In some aspects, the first modified nucleic acid sequence does not encode a wild-type preproinsulin secretion signal peptide. In some aspects, the wild-type preproinsulin is replaced by a non-insulin secretion signal. In some aspects, the first modified nucleic acid sequence encodes a human preproinsulin comprising an interleukin 6 (IL-6) secretion signal peptide. In some aspects, the first modified nucleic acid sequence encodes a human preproinsulin comprising a fibronectin secretion signal peptide.

In some aspects, the first and second modified nucleic acid sequences further comprise a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148.

In some aspects, the first and second modified nucleic acid sequences further comprise a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, or any combination thereof. In some aspects, the 3′ UTR comprises a nucleic acid having the sequence of SEQ ID NO: 60, SEQ ID NO: 97, SEQ ID NO: 98, SEQ ID NO 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149.

In some aspects, the expression cassette comprises a first modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161 and a second modified nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164 wherein each the first and second modified nucleic acids are linked to a first and a second promoter, respectively. In some aspects, the first modified nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 and the second modified nucleic acid has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the first modified nucleic acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161 and the second nucleic acid sequence is selected from the group consisting of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164. In some aspects, the first modified nucleic acid comprises nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 and the second modified nucleic acid comprises nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the first modified nucleic acid sequence comprises a 5′ UTR, an ORF, and a 3′ UTR present or referenced in Table 1 and/or FIG. 1A and the second modified nucleic acid sequence comprises a 5′ UTR, an ORF, and a 3′ UTR present or referenced in Table 2 and/or FIG. 2A.

Certain aspects of the disclosure are directed to an expression construct, e.g., a vector. In some aspects, the expression construct comprises an expression cassette. In some aspects, the expression construct further comprises a genome that is able to stabilize and remain episomal in a cell. Within the context of the disclosure, in some aspects, a cell or host cell can encompass a cell used to make the construct or a cell to which the construct is administered. In some aspects, a construct is capable of integrating into a cell's genome, e.g. through homologous recombination or otherwise. In some aspects, the expression construct is one wherein a nucleotide sequence encoding an insulin and/or a glucokinase as disclosed herein, is operably linked to a promoter as provided herein wherein the promoter is capable of directing expression of the nucleotide sequence(s) (i.e. coding sequence(s)) in a cell. In some aspects, an expression cassette as used herein comprises or consists of a nucleotide sequence encoding an insulin and/or a nucleotide sequence encoding a glucokinase, in each case the nucleotide sequence is operably linked to a promoter wherein the promoter is capable of directing expression of said nucleotide sequences. In some aspects, a viral expression construct is an expression construct that is intended to be used in gene therapy. It can be designed to comprise part of a viral genome as disclosed herein.

In some aspects, the expression construct further comprises one or more of: an ITR sequence (e.g., AAV2 ITRs), a polyA sequence (e.g., a SV40 polyadenylation signal, a bGH polyadenylation signal), and an enhancer sequence (e.g., a SV40 enhancer sequence).

In some aspects, expression constructs disclosed herein are prepared using recombinant techniques in which modified nucleic acid sequences encoding an insulin and/or a glucokinase are expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

### Delivery Vectors

The present disclosure also provides vectors comprising any of the modified nucleic acids, polynucleotides, or expression cassettes described herein. In some aspects, the delivery vector is a viral vector, a non-viral vectors, a plasmid, a lipid, or a lysosome. In some aspects, the delivery vector is a viral vector. In some aspects, the viral vector is an adeno-associated virus (AAV) expression vector.

In some aspects, a modified nucleic acid or nucleotide sequence encoding an insulin and/or a glucokinase are used in an expression construct or expression vector. The phrase “expression vector” generally refers to a nucleotide sequence that is capable of effecting expression of a gene in a host compatible with such sequences. These expression vectors can include at least suitable promoter sequences and optionally, transcription termination signals. An additional factor necessary or helpful in effecting expression can also be used as disclosed herein. A modified nucleic acid or DNA or codon-optimized nucleotide sequence encoding an insulin and/or a glucokinase can be incorporated into an expression vector capable of introduction into and expression in an in vitro cell culture. In some aspects, the expression vector is suitable for replication in a prokaryotic host, such as bacteria, e.g., E. coli, or can be introduced into a cultured mammalian, plant, insect, (e.g., Sf9), yeast, fungi or other eukaryotic cell lines. In some aspects, the expression construct is suitable for expression in vivo.

In some aspects, the delivery vector comprises an expression cassette comprises a promoter operably linked to a modified nucleic acid sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, wherein the modified nucleic acid sequence encodes a human insulin protein (e.g., SEQ ID NO: 41, SEQ ID NO: 144, or SEQ ID NO: 145) or a functional fragment thereof. In some aspects, the delivery vector comprises an expression cassette comprises a promoter operably linked to a modified nucleic acid sequence comprising an ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 122. In some aspects, the modified nucleic acid comprises an ORF having the sequence of SEQ ID NOs: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159. In some aspects, the modified nucleic acid comprises an ORF having the sequence of SEQ ID NO: 122. In some aspects, the polynucleotide comprises an ORF sequence present or referenced in Table 1, Table 13, and/or FIG. 1A.

In some aspects, delivery vector comprises an expression cassette comprising a modified nucleic acid sequence encodes a human insulin comprising a wild-type preproinsulin secretion signal peptide. In some aspects, delivery vector comprises an expression cassette comprising a modified nucleic acid sequence does not encode a wild-type preproinsulin secretion signal peptide. In some aspects, the wild-type preproinsulin is replaced by a non-insulin secretion signal. In some aspects, delivery vector comprises an expression cassette comprising a modified nucleic acid sequence encodes a human preproinsulin comprising an interleukin 6 (IL-6) secretion signal peptide. In some aspects, delivery vector comprises an expression cassette comprising a modified nucleic acid sequence encodes a human preproinsulin comprising a fibronectin secretion signal peptide.

In some aspects, the delivery vector comprises an expression cassette comprising a modified nucleic acid further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR sequence present or referenced in Table 1 and/or FIG. 1A.

In some aspects, the delivery vector comprises an expression cassette comprising a modified nucleic acid further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the polynucleotide comprises a 3′ UTR sequence present or referenced in Table 1 and/or FIG. 1A.

In some aspects, the delivery vector comprises an expression cassette comprising a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161, wherein the modified nucleic acid sequence encodes a human insulin protein (e.g., SEQ ID NO: 41, SEQ ID NO: 144, or SEQ ID NO: 145) or a functional fragment thereof. In some aspects, the delivery vector comprises an expression cassette comprising a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 138. In some aspects, the delivery vector comprises an expression cassette comprising a modified nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the delivery vector comprises an expression cassette comprising a modified nucleic acid having the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161. In some aspects, the delivery vector comprises an expression cassette comprising a modified nucleic acid having the sequence of SEQ ID NO: 138. In some aspects, the delivery vector comprises an expression cassette comprising a modified nucleic acid comprising nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some aspects, the delivery vector comprises an expression cassette comprising a modified nucleic acid comprising a 5′ UTR, an ORF, and a 3′ UTR present or referenced in Table 1 and/or FIG. 1A.

In some aspects, the delivery vector comprises an expression cassette comprising a promoter operably linked to a modified nucleic acid comprising an ORF sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the delivery vector comprises an expression cassette comprising a promoter operably linked to a modified nucleic acid having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the polynucleotide comprises an ORF sequence present or referenced in Table 2 and/or FIG. 2A.

In some aspects, the delivery vector comprises an expression cassette comprising a modified nucleic acid further comprising a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148. In some aspects, the polynucleotide comprises a 5′ UTR sequence present or referenced in Table 2 and/or FIG. 2A.

In some aspects, the delivery vector comprises an expression cassette comprising a modified nucleic acid further comprising a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the 3′ UTR comprises a nucleic acid having the sequence of SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the polynucleotide comprises a 3′ UTR sequence present or referenced in Table 2 and/or FIG. 2A.

In some aspects, the delivery vector comprises an expression cassette comprising a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164, wherein the nucleic acid sequence encodes a human glucokinase protein (e.g., SEQ ID NO: 82) or functional fragment thereof. In some aspects, the delivery vector comprises an expression cassette comprising a modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the delivery vector comprises an expression cassette comprising a modified nucleic acid having the sequence of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164. In some aspects, the delivery vector comprises an expression cassette comprising a modified nucleic acid comprising nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the delivery vector comprises an expression cassette comprising modified nucleic acid comprising a 5′ UTR, an ORF, and a 3′ UTR present or referenced in Table 2 and/or FIG. 2A.

In some aspects, the delivery vector comprises an expression cassette comprising a first modified nucleic acid comprising a first ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, or SEQ ID NO: 159, and a second modified nucleic acid sequence comprising a second ORF having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162. In some aspects, the delivery vector comprises an expression cassette comprising a promoter operably linked to a modified nucleic acid having the sequence of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or SEQ ID NO: 162, wherein each the first and second modified nucleic acids are linked to a first and a second promoter, respectively. In some aspects, the first modified nucleic acid sequence comprising a first ORF is selected from the group consisting of SEQ ID NOs: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, and SEQ ID NO: 159, and the second modified nucleic acid sequence comprising a second ORF is selected from the group consisting of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, and SEQ ID NO: 80.

In some aspects, the first modified nucleic acid sequence encodes a human insulin comprising a wild-type preproinsulin secretion signal peptide. In some aspects, the first modified nucleic acid sequence does not encode a wild-type preproinsulin secretion signal peptide. In some aspects, the wild-type preproinsulin is replaced by a non-insulin secretion signal. In some aspects, the first modified nucleic acid sequence encodes a human preproinsulin comprising an interleukin 6 (IL-6) secretion signal peptide. In some aspects, the first modified nucleic acid sequence encodes a human preproinsulin comprising a fibronectin secretion signal peptide.

In some aspects, the first and second modified nucleic acid sequences further comprise a 5′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, 100% sequence identity to SEQ ID NO: 42, nucleic acids 5-329 of SEQ ID NO: 42, SEQ ID NO: 83, SEQ ID NO: 146, or SEQ ID NO: 148. In some aspects, the 5′ UTR comprises a nucleic acid having the sequence of SEQ ID NO: 42, nucleic acids 5-329 of SEQ ID NO: 42, SEQ ID NO: 83, SEQ ID NO: 146, or SEQ ID NO: 148.

In some aspects, the first and second modified nucleic acid sequences further comprise a 3′ UTR comprising a nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149. In some aspects, the 3′ UTR comprises a restriction site selected from the group consisting of BamHI, EcoRI, NdeI, EcoRV, SpeI, XbaI, NheI, VspI, NsiI, ScaI, KpnI, SspI, and PacI, and any combination thereof. In some aspects, the 3′ UTR comprises a nucleic acid having the sequence of SEQ ID NO: 60, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149.

In some aspects, the delivery vector comprises an expression cassette comprising a first modified nucleic acid having at least 85%, at least 86%, at least 87%, at least 88%, at least 89%, at least 90%, at least 91%, at least 92%, at least 93%, at least 94%, at least 95%, at least 96%, at least 97%, at least 98%, least 99%, or 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161 and a second modified nucleic acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164, wherein each the first and second modified nucleic acids are linked to a first and a second promoter, respectively. In some aspects, the first modified nucleic acid has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-957 of a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 and the second modified nucleic acid sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleic acids 5-2025 of a sequence selected from SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the first modified nucleic acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 84, SEQ ID NO: 85, SEQ ID NO: 86, SEQ ID NO: 87, SEQ ID NO: 88, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO 137, SEQ ID NO: 138, SEQ ID NO: 139, SEQ ID NO: 140, SEQ ID NO: 141, SEQ ID NO: 160, or SEQ ID NO: 161 and the second nucleic acid sequence is selected from the group consisting of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, SEQ ID NO: 39, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 163, or SEQ ID NO: 164. In some aspects, the first modified nucleic acid sequence comprising nucleic acids 5-957 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16 and the second modified nucleic acid sequence comprising nucleic acids 5-2025 of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 35, SEQ ID NO: 36, SEQ ID NO: 37, SEQ ID NO: 38, or SEQ ID NO: 39. In some aspects, the first modified nucleic acid sequence comprises a 5′ UTR, an ORF, and a 3′ UTR present or referenced in Table 1 and/or FIG. 1A and the second modified nucleic acid sequence comprises a 5′ UTR, an ORF, and a 3′ UTR present or referenced in Table 2 and/or FIG. 2A.

In some aspects, the delivery vectors can comprise sequences encoding a protein (e.g., insulin and/or Gck) operably linked with control or regulatory sequences, selectable markers, any fusion partners, and/or additional elements. In certain aspects, the modified nucleic acid is placed into a functional relationship with another nucleic acid sequence. The term “regulatory sequence” includes promoters, enhancers and other expression control elements (e.g., polyadenylation signals) that control the transcription or translation of the protein. Such regulatory sequences are described, for example, in Goeddel (Gene Expression Technology, Methods in Enzymology 185, Academic Press, San Diego, CA (1990)). In some aspects, the expression vectors include transcriptional and translational regulatory nucleic acid operably linked to the nucleic acid encoding the protein, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors can contain a selection gene or marker to allow the selection of transformed host cells containing the expression vector. Selection genes are known in the art and will vary with the host cell used. For example, typically the selectable marker gene confers resistance to drugs, such as G418, hygromycin or methotrexate, on a host cell into which the vector has been introduced. In some aspects, selectable marker genes include the dihydrofolate reductase (DHFR) gene (for use in dhfr-host cells with methotrexate selection/amplification) and the neo gene (for G418 selection).

In some aspects, the delivery vector is a viral vector or a gene therapy vector comprising a viral expression construct. In certain aspects, the viral vector or a gene therapy vector is a vector that is suitable for gene therapy.

In some aspects, the gene therapy vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81:2573-2604; Goncalves, 2005, Virol J. 2 (1): 43) as indicated above. AAV vectors can result in very stable long term expression of transgene expression (up to 9 years in dog (Niemeyer et al, Blood. 2009 Jan. 22; 113 (4): 797-806) and up to 2 years in human (Nathwani et al, N Engl J Med. 2011 Dec. 22; 365 (25): 2357-65, Simonelli et al, Mol Ther. 2010 March; 18 (3): 643-50. Epub 2009 Dec. 1.)). In some aspects, adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6 (5): 482-90, and Martin et al., 2004, Eye 18 (11): 1049-55, Nathwani et al, N Engl J Med. 2011 Dec. 22; 365 (25): 2357-65, Apparailly et al, Hum Gene Ther. 2005 April; 16 (4): 426-34.

In some aspects, the gene therapy vector includes a retroviral vector. In some aspects, the retroviral vector is a lentiviral based expression construct. Lentiviral vectors have the ability to infect and to stably integrate into the genome of dividing and non-dividing cells (Amado and Chen, 1999 Science 285:674-6). Methods for the construction and use of lentiviral based expression constructs are described in U.S. Pat. Nos. 6,165,782, 6,207,455, 6,218, 181, 6,277,633 and 6,323,031 and in Federico (1999, Curr Opin Biotechnol 10:448-53) and Vigna et al. (2000, J Gene Med 2000; 2:308-16).

In some aspects, the gene therapy vector is a herpes virus vector, a polyoma virus vector or a vaccinia virus vector.

In some aspects, the gene therapy vector comprises a modified nucleotide sequence encoding an insulin and/or a glucokinase, whereby each of said modified nucleotide sequence is operably linked to the appropriate regulatory sequences. Such regulatory sequence can at least comprise a promoter sequence. Suitable promoters for expression of a nucleotide sequence encoding an insulin and/or a glucokinase from gene therapy vectors can include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter.

In some aspects, the gene therapy vector includes a further nucleotide sequence coding for a further polypeptide. A further polypeptide can be a (selectable) marker polypeptide that allows for the identification, selection and/or screening for cells containing the expression construct. In some aspects, suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

### Non-Viral Vectors

In some aspects, a modified nucleic acid, polynucleotide, or expression construct of the disclosure can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, a modified nucleic acid, polynucleotide, or expression construct of the disclosure can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

In some aspects, a modified nucleic acid, polynucleotide, or expression construct of the disclosure can also be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof. Exemplary protein carrier molecules include antibodies specific to the target cells, i.e., molecules capable of interacting with receptors associated with a cell targeted for delivery.

### Adeno Associated Virus Vector (AAV Vector)

In some aspects, the modified nucleic acids, polynucleotides, or expression constructs of disclosed herein can be administered as a component of a packaged viral vector. In general, packaged viral vectors include a viral vector packaged in a capsid.

In some aspects, the viral vector is an AAV vector. In some aspects, an AAV vector as used herein can comprise a recombinant AAV vector (rAAV). A “rAAV vector” as used herein refers to a recombinant vector comprising part of an AAV genome encapsidated in a protein shell of capsid protein derived from an AAV serotype as disclosed herein. Part of an AAV genome can contain the inverted terminal repeats (ITR) derived from an adeno-associated virus serotype, such as AAV1, AAV2, AAV3, AAV4, AAV5, AAV6, AAV7, AAV8, AAVrh8, AAV9, AAV10, AAVRH10, AAV11, AAV12, and others. In some aspects, the ITR is derived from AAV2.

Typically, a vector genome requires the use of flanking 5′ and a 3′ ITR sequences to allow for efficient packaging of the vector genome into the rAAV capsid. In some aspects, the rAAV genome present in a rAAV vector comprises at least the nucleotide sequences of the inverted terminal repeat regions (ITR) of one of the AAV serotypes (e.g., of serotype AAV2 as disclosed earlier herein), or nucleotide sequences substantially identical thereto, and a modified nucleic acid sequence encoding an insulin and/or a glucokinase under control of a suitable regulatory element (e.g., a promoter), wherein the regulatory element and modified nucleic acid sequence(s) are inserted between the two ITRs.

The complete genome of several AAV serotypes and corresponding ITR has been sequenced (Chiorini et al. 1999, J. of Virology Vol. 73, No. 2, p 1309-1319). They can be either cloned or made by chemical synthesis as known in the art, using for example an oligonucleotide synthesizer as supplied e.g. by Applied Biosystems Inc. (Fosters, Calif., USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the wild type AAV sequence between the ITRs can be replaced with the desired nucleotide sequence.

The viral capsid component of the packaged viral vectors can be a parvovirus capsid, e.g., AAV Cap and/or chimeric capsids. Examples of suitable parvovirus viral capsid components are capsid components from the family Parvoviridae, such as an autonomous parvovirus or a Dependovirus. For example, the viral capsid may be an AAV capsid (e.g., AAV1, AAV2, AAV3, AAV4, AAV5, AAV6, AAV7, AAV8, AAVRH8 AAV 9, AAV10, AAVRH10, AAV11 or AAV12 capsid; one skilled in the art would know there are likely other variants not yet identified that perform the same or similar function), or may include components from two or more AAV capsids. A full complement of AAV Cap proteins includes VP1, VP2, and VP3. The ORF comprising nucleotide sequences encoding AAV VP capsid proteins can comprise less than a full complement AAV Cap proteins or the full complement of AAV Cap proteins can be provided.

One or more of the AAV Cap proteins can be a chimeric protein, including amino acid sequences AAV Caps from two or more viruses, preferably two or more AAVs. For example, the chimeric virus capsid can include an AAV1 Cap protein or subunit and at least one AAV2 Cap or subunit. In some aspects, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

In some aspects, the rAAV genome as present in said rAAV vector further comprises a promoter sequence operably linked to the nucleotide sequence encoding an insulin and/or a glucokinase. In some aspects, the promoter sequences are promoters which confer expression in muscle cells and/or muscle tissues. Examples of such promoters include a CMV and a RSV promoters as disclosed herein.

In some aspects, suitable 3′ untranslated sequence can also be operably linked to the modified nucleic acid sequences encoding an insulin and/or a glucokinase. Suitable 3′ untranslated regions can be those naturally associated with the nucleotide sequence or can be derived from different genes, such as for example the bovine growth hormone 3′ untranslated region (e.g., bGH polyadenylation signal, SV40 polyadenylation signal, SV40 polyadenylation signal and enhancer sequence).

In some aspects, additional nucleotide sequences can be operably linked to the modified nucleic acid sequence(s) encoding an insulin and/or a glucokinase, such as nucleotide sequences encoding signal sequences, nuclear localization signals, expression enhancers, and the like.

Except as otherwise indicated, methods known to those skilled in the art may be used for the construction of recombinant parvovirus and AAV (rAAV) constructs, packaging vectors expressing the parvovirus Rep and/or Cap sequences, and transiently and stably transacted packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); AUSUBEL el al., CURRENT PROTOCOLS IN MOLECULAR BIOLOGY (Green Publishing Associates, Inc. and John Wiley Sons, Inc., New York).

### Lentiviral Expression Constructs

Lentiviruses are complex retroviruses that in addition to the common retroviral genes gag, pol and env, contain other genes with regulatory or structural function. The higher complexity enables the lentivirus to modulate the life cycle thereof, as in the course of latent infection.

A typical lentivirus is the human immunodeficiency virus (HIV), the etiologic agent of AIDS. In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or γ irradiation.

Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The κ′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

In some aspects, the recombinant lentivirus is capable of infecting a non-dividing cell by transfecting a suitable host cell with two or more vectors carrying the packaging functions, namely gag, pol and env, as well as rev and tat. In some examples, vectors lacking a functional tat gene are desirable. Thus, for example, a first vector can provide a nucleic acid encoding a viral gag and a viral pol and another vector can provide a nucleic acid encoding a viral env to produce a packaging cell. Introducing a vector providing a heterologous gene, identified as a transfer vector, into that packaging cell yields a producer cell which releases infectious viral particles carrying the foreign gene of interest.

The gag, pol and env genes of the vectors of interest also are known in the art. Thus, the relevant genes are cloned into the selected vector and then used to transform the target cell of interest.

According to the above-indicated configuration of vectors and foreign genes, the second vector can provide a nucleic acid encoding a viral envelope (env) gene. The env gene can be derived from any virus, including retroviruses. The env preferably is an amphotropic envelope protein which allows transduction of cells of human and other species.

It may be desirable to target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific. Retroviral vectors can be made target-specific by inserting, for example, a glycolipid or a protein. Targeting often is accomplished by using an antigen-binding portion of an antibody or a recombinant antibody-type molecule, such as a single chain antibody, to target the retroviral vector. Those of skill in the art will know of, or can readily ascertain without undue experimentation, specific methods to achieve delivery of a retroviral vector to a specific target.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV G), that of hepatitis viruses and of influenza also can be used.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

In some aspects, the lentiviral genome as present in said lentiviral vector further comprises a promoter sequence operably linked to the nucleotide sequence encoding an insulin and/or a glucokinase. In some aspects, the promoter sequences are promoters which confer expression in muscle cells and/or muscle tissues. Examples of such promoters include a CMV and a RSV promoters as disclosed herein.

In some aspects, suitable 3′ untranslated sequence can also be operably linked to the modified nucleic acid sequences encoding an insulin and/or a glucokinase. Suitable 3′ untranslated regions can be those naturally associated with the nucleotide sequence or can be derived from different genes, such as for example the bovine growth hormone 3′ untranslated region (e.g., bGH polyadenylation signal, SV40 polyadenylation signal, SV40 polyadenylation signal and enhancer sequence).

In some aspects, additional nucleotide sequences can be operably linked to the modified nucleic acid sequence(s) encoding an insulin and/or a glucokinase, such as nucleotide sequences encoding signal sequences, nuclear localization signals, expression enhancers, and the like.

Except as otherwise indicated, methods known to those skilled in the art may be used for the construction of lentiviral constructs, vectors, and transiently and stably transacted packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); AUSUBEL el al., CURRENT PROTOCOLS IN MOLECULAR BIOLOGY (Green Publishing Associates, Inc. and John Wiley Sons, Inc., New York).

### Host Cells

In some aspects, the present disclosure also provides host cells comprising the modified nucleic acid sequences, polynucleotides, expression cassettes, vectors, or expression constructs disclosed herein. In some aspects, the host cell is a mammalian cell.

A construct prepared for introduction into a particular host can include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment can be employed. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334:31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

The methods of introducing exogenous nucleic acid into host cells are well known in the art, and will vary with the host cell used. Techniques include but are not limited to dextran-mediated transfection, calcium phosphate precipitation, calcium chloride treatment, polyethylenimine mediated transfection, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

Host cells may be yeast, e.g. S. cerevisiae, e.g., insect cells, e.g., Sf9 cells, mammalian cells, e.g., CHO cells, and bacterial cells, e.g., E. coli. A cell may thus be a prokaryotic or eukaryotic host cell. A cell may be a cell that is suitable for culture in liquid or on solid media. Alternatively, a host cell is a cell that is part of a multicellular organism such as a transgenic plant or animal. In some aspects, the host cell is a mammalian cell.

In some aspects, methods of introducing the viral vectors comprising the modified nucleic acids disclosed herein into a cellular host for replication and packaging can be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

In some aspects, packaging functions can include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions can be supplied together or separately to the packaging cell using a genetic construct such as a plasmid or an amplicon. The packaging functions can exist extrachromosomally within the packaging cell, or can be integrated into the cell's chromosomal DNA. Examples include genes encoding AAV Rep and Cap proteins.

In some aspects, helper functions can include helper virus elements needed for establishing active infection of the packaging cell, which is required to initiate packaging of the viral vector. Examples include functions derived from adenovirus, baculovirus and/or herpes virus sufficient to result in packaging of the viral vector. For example, adenovirus helper functions will typically include adenovirus components E1a, E1b, E2a, E4, and VA RNA. The packaging functions can be supplied by infection of the packaging cell with the required virus. The packaging functions can be supplied together or separately to the packaging cell using a genetic construct such as a plasmid or an amplicon. The packaging functions can exist extrachromosomally within the packaging cell, or can be integrated into the cell's chromosomal DNA.

Any suitable helper virus functions may be employed. For example, where the packaging cells are insect cells, baculovirus can serve as a helper virus. Herpes virus can also be used as a helper virus in AAV packaging methods.

Any method of introducing the nucleotide sequence carrying the helper functions into a cellular host for replication and packaging can be employed, including but not limited to, electroporation, calcium phosphate precipitation, microinjection, cationic or anionic liposomes, and liposomes in combination with a nuclear localization signal. In embodiments wherein the helper functions are provided by transfection using a virus vector or infection using a helper virus; standard methods for producing viral infection may be used.

Any suitable permissive or packaging cell known in the art can be employed in the production of the packaged viral vector. Mammalian cells or insect cells are preferred. Examples of cells useful for the production of packaging cells in the practice of the invention include, for example, human cell lines or primate cells, such as VERO, WI38, MRC5, A549, 293 cells, B-50 or any other Hela cells, HepG2, Saos-2, HuH7, and HT1080 cell lines.

In some aspects, the cell lines for use as packaging cells are insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, NJ (1995); O'Reilly et al., Baculovirus Expression Vectors: A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059.

During production, the packaging cells can include one or more viral vector functions along with helper functions and packaging functions sufficient to result in replication and packaging of the viral vector. These various functions can be supplied together or separately to the packaging cell using a genetic construct such as a plasmid or an amplicon, and they can exist extrachromosomally within the cell line or integrated into the cell's chromosomes.

The cells can be supplied with any one or more of the functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA.

### Pharmaceutical Compositions

In some aspects, the present disclosure also provides pharmaceutical compositions comprising the modified nucleic acid sequences, polynucleotides, expression cassettes, vectors, or expression constructs disclosed herein. In some aspects there is provided a composition comprising an expression construct or a delivery vector (e.g., a viral vector packaged in an AAV capsid) comprising a modified nucleic acid sequence encoding an insulin and/or glucokinase as disclosed herein. In some aspects, a composition is a gene therapy composition. In some aspects, the composition is a pharmaceutical composition said pharmaceutical composition comprising a pharmaceutically acceptable carrier, adjuvant, diluents, solubilizer, filler, preservative and/or excipient.

Such pharmaceutically acceptable carrier, filler, preservative, solubilizer, diluent and/or excipient may for instance be found in Remington: The Science and Practice of Pharmacy, 20th Edition. Baltimore, Md.: Lippincott Williams & Wilkins, 2000.

In some aspects, the composition is for use as a medicament. In some aspects, the medicament is used for preventing, reducing or ameliorating the symptoms of, delaying, curing, reverting and/or treating a diabetes. In some aspects, diabetes can be Diabetes Type 1, Diabetes Type 2 or Monogenic Diabetes. In some aspects, the subject treated is a mammal, e.g. cats, rodent, (mice, rats, gerbils, guinea pigs, mice or rats), dogs, or human beings.

In some aspects, the modified nucleic acid, expression construct, delivery vector and/or composition is used for preventing, reducing or ameliorating the symptoms of, delaying, reverting, curing and/or treating a diabetes, when said the modified nucleic acid, expression construct, delivery vector and/or composition is able to exhibit an anti-diabetes effect. An anti-diabetes effect can be reached when glucose disposal in blood is increased and/or when glucose tolerance is improved. This can be assessed using techniques known to the skilled person. In this context, “increase” (respectively “improvement”) means at least a detectable increase (respectively a detectable improvement) using an assay known to the skilled person or using assays as carried out in the experimental part.

An anti-diabetes effect can also be observed when the progression of a typical symptom (i.e. insulitis, beta cell loss) has been slowed down as assessed by a physician. A decrease of a typical symptom associated with diabetes can mean a slowdown in progression of symptom development or a complete disappearance of symptoms. Symptoms, and also a decrease in symptoms, can be assessed using a variety of methods, to a large extent the same methods as used in diagnosis of diabetes, including clinical examination and routine laboratory tests. Such methods include both macroscopic and microscopic methods, as well as molecular methods, biochemical, immunohistochemical and others.

A medicament as defined herein (modified nucleic acid, expression construct, delivery vector, composition, etc.) is preferably able to alleviate one symptom or one characteristic of a patient or of a cell, tissue or organ of said diabetes patient if after at least one week, one month, six month, one year or more of treatment using the modified nucleic acid, viral expression construct, viral vector, or composition disclosed herein, said symptom or characteristic is decreased or no longer detectable.

A modified nucleic acid, expression construct, delivery vector, or composition as disclosed herein for use in preventing, reducing or ameliorating the symptoms of, delaying, reverting, curing and/or treating a diabetes can be suitable for administration to a cell, tissue and/or an organ in vivo of individuals affected by or at risk of developing a diabetes, and may be administered in vivo, ex vivo or in vitro. Said combination and/or composition can be directly or indirectly administrated to a cell, tissue and/or an organ in vivo of an individual affected by or at risk of developing a diabetes, and may be administered directly or indirectly in vivo, ex vivo or in vitro. In some aspects, the administration mode is intramuscular.

In some aspects, the modified nucleic acid, expression construct, delivery vector, or composition as disclosed herein can be directly or indirectly administered using suitable means known in the art. In some aspects, the modified nucleic acid, expression construct, delivery vector, or composition as disclosed herein can be delivered as is to an individual, a cell, tissue or organ of said individual. Depending on the disease or condition, a cell, tissue or organ of said individual may be as earlier defined herein. In some aspects, the modified nucleic acid, expression construct, delivery vector, or composition as disclosed herein is dissolved in a solution that is compatible with the delivery method. For intravenous, subcutaneous, intramuscular, intrathecal, intraarticular and/or intraventricular administration the solution may be a physiological salt solution. In some aspects, administration is intramuscular administration. In some aspects, intramuscular administration is carried out using a multineedle. In some aspects, a therapeutically effective dose of the modified nucleic acid, expression construct, the vector, or the composition as described herein is administered in a single and unique dose hence avoiding repeated periodical administration. In some aspects, the single dose is administered to muscle tissue. In some aspects, the single dose is administered to skeletal muscle tissue. In some aspects, the single dose comprise multiple injections (e.g., two, three, four, or five) to one or more muscles (e.g., multiple muscle groups).

In some aspects, a compound can be present in a composition of the invention. Said compound can help in delivery of the modified nucleic acid or composition comprising the same. In some aspects, the compound is a compound capable of forming complexes, nanoparticles, micelles, liposomes that deliver each constituent as defined herein, complexed or trapped in a vesicle or liposome through a cell membrane, or combinations thereof. Many of these compounds are known in the art. In some aspects, the further compound is polyethylenimine (PEI), or similar cationic polymers, including polypropyleneimine or polyethylenimine copolymers (PECs) and derivatives, synthetic amphiphiles (SAINT-18), Lipofectin™, DOTAP, or combinations thereof.

### Methods of Use

The present disclosure also provides a method for preventing, reducing or ameliorating the symptoms of, delaying, reverting, curing and/or treating diabetes comprising administering to a subject in need thereof any of a modified nucleic acid, a polynucleotide, an expression cassette, a delivery vectors, or expression construct disclosed herein. In some aspects, the diabetes can be T1DM. In some aspects, the diabetes can be T2DM. In some aspects, the method is a gene therapy. In certain aspects, the methods of the disclosure comprise administration (e.g., intramuscular administration) of any of a modified nucleic acid, a polynucleotide, an expression cassette, a delivery vectors, or expression construct disclosed herein to a cell, tissue, or subject in need thereof. In certain aspects, the methods comprise administration of (i) a modified (or wild-type or unmodified) nucleic acid, a polynucleotide, an expression cassette, a delivery vectors, or expression construct encoding a human insulin (Ins) protein (e.g., a preproinsulin or variant thereof) and/or (ii) a modified (or wild-type or unmodified) nucleic acid, a polynucleotide, an expression cassette, a delivery vectors, or expression construct comprising a nucleic acid encoding a human glucokinase (Gck) protein. In some aspects, the hIns nucleic acid sequence is a modified hIns sequence disclosed herein and the hGck nucleic acid sequence is a modified hGck sequence disclosed herein. In some aspects, the hIns nucleic acid sequence is a wild-type or an unmodified hIns sequence disclosed herein and the hGck sequences is a modified hGck sequence disclosed herein. In certain aspects, the administration of (i) and (ii) is simultaneous or sequential.

Certain aspects of the disclosure are directed methods of use comprising administering a polynucleotide encoding a human insulin (Ins) protein (e.g., a preproinsulin or variant thereof) comprising (i) a nucleotide sequence encoding a signal peptide, optionally wherein the signal peptide is not a wild-type preproinsulin signal sequence, and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid position in wild-type proinsulin, and optionally the polynucleotide further comprises a cleavage site. In some aspects, the signal peptide is a wild-type preproinsulin signal sequence, an IL-6 signal sequence, or a fibronectin signal sequence. In some aspects, the cleavage site is a furin cleavage site.

Certain aspects of the disclosure are directed to methods of use comprising administering a polynucleotide comprising a nucleic acid encoding a human insulin (Ins) protein, wherein the nucleic acid comprises an open reading frame (ORF) comprising: (i) a nucleotide sequence encoding a signal peptide and (ii) a nucleotide sequence at least 85%, 86%, 87%, 88%, 89%, 90%, 91%, 92%, 93%, 94%, 95%, 96%, 97%, 98%, 99%, or 100% identical to nucleic acids 73-330 of any of SEQ ID NOs: 43-57, 110-116, 150-151, 154-155, and 157-159, nucleic acids 88-345 of any of SEQ ID NOs: 117-122, 152, and 156, or nucleic acids 79-336 of SEQ ID NO: 153. In some aspects, the encoded human Ins protein comprises (i) a signal peptide (e.g., a wild-type preproinsulin signal sequence, an IL-6 signal sequence, or a fibronectin signal sequence) and (ii) amino acids 25-110 of SEQ ID NO: 41, amino acids 25-110 of SEQ ID NO: 144, or amino acids 25-110 of SEQ ID NO: 145. In some aspects, the encoded human insulin protein further comprises a cleavage site (e.g., a furin cleavage site).

Certain aspects of the disclosure are directed to a method of use comprising administering a polynucleotide comprising a nucleic acid encoding a human insulin (Ins) protein, wherein the nucleic acid comprises an open reading frame (ORF) comprising: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159. In some aspects, the polynucleotide comprises at least two nucleic acid sequences encoding a human Ins protein. In some aspects, the polynucleotide comprises at least two ORF nucleotide sequences at least 85%, 86%, 87%, 88%, 89%, 90%, 91%, 92%, 93%, 94%, 95%, 96%, 97%, 98%, 99%, or 100% identical to any of SEQ ID NOs: 43-57, 110-122, or 150-159, wherein the two ORF nucleotide sequences can be the same or different. In some aspects, the polynucleotide further comprises an IRES sequences. In some aspects, the at least two ORF nucleotide sequences are separated by an IRES sequences. In some aspects, the encoded human Ins protein comprises a signal sequence and a proinsulin polypeptide. In some aspects, the encoded human Ins protein comprises the amino acid sequence of any of amino acids 25-110 of SEQ ID NO: 41, amino acids 25-110 of SEQ ID NO: 144, or amino acids 25-110 of SEQ ID NO: 145. In some aspects, the encoded human Ins protein is a preproinsulin. In some aspects, the encoded human Ins protein comprises the amino acid sequence of SEQ ID NO: 41, SEQ ID NO: 144, or SEQ ID NO: 145. In some aspects, the polynucleotide or nucleic acid sequence further comprises a 5′ UTR and/or a 3′ UTR. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 1-16, 84-88, 123-141, or 160-161.

Certain aspects of the disclosure are directed to a method of use comprising administering a polynucleotide comprising a nucleic acid encoding a human insulin (Ins) protein (e.g., a preproinsulin or variant thereof), wherein the nucleic acid comprises: (i) a nucleotide sequence encoding a signal peptide (e.g., a wild-type preproinsulin signal sequence, an IL-6 signal sequence, or a fibronectin signal sequence) and (ii) a nucleotide sequence encoding a proinsulin polypeptide comprising an amino acid modification at a position selected from amino acid B10, B28, and/or B29 of the human insulin B-chain, C1 and/or C32 of the human insulin C-chain, or any combination thereof relative to the corresponding amino acid in wild-type proinsulin (or an amino acid modification at a position selected from amino acid H34, P52, K53, R55, L86, or any combination thereof relative to the corresponding amino acid in wild-type preproinsulin). In some aspects, the signal peptide is not a wild-type preproinsulin signal sequence (e.g., the wild-type preproinsulin sequence is replaced with an IL-6 signal sequence or fibronectin signal sequence). In some aspects, the proinsulin polypeptide comprises the amino acid sequence of any of amino acids 25-110 of SEQ ID NO: 41, amino acids 25-110 of SEQ ID NO: 144, or amino acids 25-110 of SEQ ID NO: 145. In some aspects, the polynucleotide further comprises a cleavage site (e.g., a furin cleavage site).

Certain aspects of the disclosure are directed to a method of use comprising administering a polynucleotide comprising a nucleic acid encoding a human glucokinase (Gck) protein, wherein the nucleic acid comprises an ORF comprising: a nucleotide sequence at least 85%, 86%, 87%, 88%, 89%, 90%, 91%, 92%, 93%, 94%, 95%, 96%, 97%, 98%, 99%, or 100% identical to any of nucleic acids 1-1398 of any of SEQ ID NO: 61-80 or 162; or SEQ ID NO: 61-80 and 162. In some aspects, the encoded human Gck protein comprises the amino acid sequence of SEQ ID NO: 82. In some aspects, the polynucleotide or nucleic acid sequence encoding a Gck protein further comprises a 5′ UTR and/or a 3′ UTR. In some aspects, the nucleic acid further comprises a 5′ UTR comprising a nucleotide sequence at least 85%, 90%, 95%, 99%, or 100% identical to SEQ ID NO: 42, 5-329 of SEQ ID NO: 42, SEQ ID NO: 83, SEQ ID NO: 146, or SEQ ID NO: 148. In some aspects, the nucleic acid further comprises a 3′ UTR comprising a nucleotide sequence at least 85%, 90%, 95%, 99%, or 100% identical to SEQ ID NO: 60, SEQ ID NO: 97, SEQ ID NO: 98, SEQ ID NO: 99, SEQ ID NO: 100, SEQ ID NO: 101, or SEQ ID NO: 149. In some aspects, the polynucleotide or nucleic acid comprises: a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 20-39 and 89-96, and 163-164. In some aspects, the nucleic acid is operably linked to a promoter (e.g., a eukaryotic promoter). Certain aspects of the disclosure are directed to an expression cassette comprising a polynucleotide of the disclosure and a heterologous expression control sequence operably linked to the nucleic acid sequence. In some aspects, the nucleic acid is operably linked to a polyadenylation (polyA) element.

Certain aspects of the disclosure are directed to methods of use comprising administering a vector (e.g., viral vector, a non-viral vector, a plasmid, a lipid, or a lysosome) comprising a polynucleotide or an expression cassette of the disclosure. In some aspects, the vector is an adeno-associated virus (AAV) vector or a Lentivirus vector. Certain aspects of the disclosure are directed to a method of administering a recombinant AAV (rAAV) particle, comprising an AAV capsid and a vector genome comprising the polynucleotide or the expression cassette of the disclosure. In some aspects, the AAV serotype is selected from the group consisting of AAV1, AAV2, AAV3, AAV4, AAV5, AAV6, AAV7, AAV8, AAVRH8, AAVrh9, AAV9, AAVrh10, AAV10, AAVRH10, AAV11, and AAV12.

Certain advantages for the gene therapy methods disclosed herein include the potential for administration of a modified nucleic acid, a polynucleotide, an expression cassette, a delivery vectors, or expression construct disclosed herein that provides the therapeutic gene expression through the lifetime of the diabetic subject. WO 2012/007458 discloses the generation of two viral vectors, one expressing the insulin gene and one expressing the glucokinase gene as a treatment of diabetes. Furthermore, WO 2016/110518 discloses single-vector gene constructs comprising insulin and glucokinase genes. In certain aspects, the present disclosure provides improved nucleic acid sequences, expression constructs, and/or delivery vectors for diabetes treatment or prevention having increased expression of insulin and/or glucokinase, decreased adverse immune reaction, and/or allowing for administration of a lower dose of viral vector.

In some aspects, the methods of the disclosure alleviates or reduces one or more symptom(s) of diabetes in an individual, in a cell, tissue or organ of said individual or alleviates or reduces one or more characteristic(s) or symptom(s) of a cell, tissue or organ of said individual, the method comprising administering to said individual one or more of the modified nucleic acids, polynucleotides, expression cassettes, vectors, or expression constructs disclosed herein.

Treatment recommendations for adults with diabetes generally target a HbA1c<7.0% without significant hypoglycemia. In some aspects, the ‘normal range’ for HbA1c is <7.0%, e.g., <6.5%, <6.0%, <5.7%, e.g., between about 5.0% and about 6.5%. Most marketed products lower HbA1c between 0.5% and 1.50%. In some aspects, the methods of the disclosure normalize HbA1c levels in a treated diabetic subject to HbA1c levels in a non-diabetic subject, e.g., within 8 weeks. In some aspects, the methods of the disclosure allow for reduction and/or regulation of glycated blood hemoglobin (HbA1c) levels in the subject. In some aspects, the HbA1c levels in a subject after treatment are <7.0% (e.g., <6.5%, <6.0%, <5.7%, e.g., between 5.0% and 6.5%), e.g., within 8 weeks after treatment.

Insulin plays a central role in the regulation of lipid metabolism in liver, adipose, and gut (Vergès B. Insulin sensitivity and lipids. Diabetes Metab. 2001 April; 27 (2 Pt 2): 223-7. PMID: 11452214.). In uncontrolled Type 1 diabetes, patients are unable to utilize glucose, requiring an alternative fuel source. In adipose tissue, insulin inhibits hormone-sensitive lipase normally promoting storage of triglycerides in the adipocytes and reducing release of free fatty acids from adipose tissue in the circulation. When circulating insulin levels are low, there is a profound reduction of lipoprotein catabolism (Taskinen M R. Lipoprotein lipase in diabetes. Diabetes Metab Rev. 3:551-570. 1987 doi: 10.1002/dmr.5610030208. 1987). Lipolysis, resulting in an increased level of circulating triglyceride-rich lipoproteins (chylomicrons, VLDLs), occurs leading to hypertriglyceridemia. In some aspects, the methods of the disclosure reduce the level of a triglyceride-rich lipoprotein (e.g., chylomicrons or VLDLs) in a subject (e.g., a subject suffering from diabetes), in a cell, tissue or organ of said subject, the method comprising administering to the subject one or more of the modified nucleic acids, polynucleotides, expression cassettes, vectors, or expression constructs disclosed herein.

In the liver, ketone bodies (β-hydroxybutyrate (β-HB) and acetoacetate (AcAc)) are produced by the β-oxidation of fatty acids. During fasting or dietary carbohydrate restriction, ketones serve as a source of alternative energy in glucose limiting conditions and can provide up to 80% of the brain's energy requirements. While useful in the short term, a chronic elevation of circulating ketones can produce unwanted effects in the brain, kidney, liver, and microvasculature (Kanikarla-Marie P, Jain S K. Hyperketonemia and ketosis increase the risk of complications in type 1 diabetes. Free Radic Biol Med. 95:268-277, 2016. doi: 10.1016/j.freeradbiomed.2016.03.020) and a resulting ketoacidosis which can be fatal. In some aspects, the methods of the disclosure reduce the level of a ketones in a subject (e.g., a subject suffering from diabetes), in a cell, tissue or organ of said subject, the method comprising administering to the subject one or more of the modified nucleic acids, polynucleotides, expression cassettes, vectors, or expression constructs disclosed herein

In some aspects, the methods of the disclosure provide (i) reduction and/or regulation of glycated blood hemoglobin (HbA1c) levels in the subject; (ii) reduction in circulating ketones in the subject, (iii) reduction in triglycerides in the subject, or (iv) any combination thereof.

In some aspects, the method or use is performed in vitro, for instance using a cell culture. In some aspects, the method or use is performed in vivo. In some aspects, a modified nucleic acid, a polynucleotide, an expression cassette, a delivery vectors, or expression construct disclosed herein is combined with an additional compound known to be used for treating diabetes in an individual. In some aspects, the method further comprises administering recombinant insulin, e.g., via regular injections.

In some aspects, the method disclosed herein is not repeated. In some aspects, the method disclosed herein is repeated each year or each 2, 3, 4, 5, 6, 7, 8, 9, or 10 years.

In some aspects, the method comprises administering a therapeutically effective dose of the modified nucleic acid, expression construct, the vector, or the composition as described herein, wherein the administration is a single, e.g., avoiding repeated periodical administration. In some aspects, the single dose is administered to muscle tissue. In some aspects, the single dose is administered to skeletal muscle tissue. In some aspects, the single dose comprise multiple injections (e.g., two, three, four, or five) to one or more muscles (e.g., multiple muscle groups).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The breadth and scope of the present invention should not be limited by any of the above-described exemplary embodiments, but should be defined in accordance with the following claims and their equivalents.

Having described the present invention, the same will be explained in greater detail in the following examples, which are included herein for illustration purposes only, and which are not intended to be limiting to the invention.

### EXAMPLES

### Example 1: Modified Insulin Nucleic Acids

The following modified human insulin nucleic acid sequences (shown in Table 1) corresponding to SEQ ID Nos: 1-16, 84-88, 123-141, and 160-161 were designed in silico. 5′UTR sequences (SEQ ID NO: 42, SEQ ID NO: 83, SEQ ID NO: 146, or SEQ ID NO: 148) are in BOLD, ORF sequences are underlined (SEQ ID NOs: 43-57, 110-122), and 3′ UTR sequences are italicized (SEQ ID NO: 60, SEQ ID NO: 97, SEQ ID NO: 98, SEQ ID NO: 99, SEQ ID NO: 100, SEQ ID NO: 101, or SEQ ID NO: 149). The 5′UTRs having the sequence of SEQ ID NO: 42 was further modified to remove the CTAG at positions 1-4. Accordingly, in certain constructs the 5′UTR included nucleic acids 5-329 of SEQ ID NO: 42. In some constructs, an IRES sequence was added between two insulin ORF sequences, the IRES sequences are shown in BOLD and italicized (SEQ ID NO: 143).

TABLE 1
Modified Nucleic Acid Sequences Encoding Human Insulin

| SEQ ID | | |
|---|---|---|
| NOs | Name | Sequence |
| Full: 1 | Unmodified | AAGCTTTATTGCGGTAGTTTATCACAGTTAAATTGCTAACGCAGTCAG |
| 5′ UTR: 148 | hIns- | TGCTTCTGACACAACAGTCTCGAACTTAAGCTGCAGTGACTCTCTTAA |
| ORF: 110 | 1 (B10D) | GGTAGCCTTGCAGAAGTTGGTCGTGAGGCACTGGGCAGGTAAGTATC |
| 3′ UTR: 149 | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTACTTAATACGACTCACTATAGGCTA |
| | | GCCTCGAGAATTCTGCCATGGCCCTGTGGATGCGCCTCCTGCCCCTGCT |
| | | GGCGCTGCTGGCCCTCTGGGGACCTGACCCAGCCGCAGCCTTTGTGAACC |
| | | AACACCTGTGCGGCTCAGATCTGGTGGAAGCTCTCTACCTAGTGTGCGGG |
| | | GAACGAGGCTTCTTCTACACACCCAGGACCAAGCGGGAGGCAGAGGACC |
| | | TGCAGGTGGGGCAGGTGGAGCTGGGCGGGGGCCCTGGTGCAGGCAGCCT |
| | | GCAGCCCTTGGCCCTGGAGGGGTCGCGACAGAAGCGTGGCATTGTGGAA |
| | | CAATGCTGTACCAGCATCTGCTCCCTCTACCAGCTGGAGAACTACTGCAA |
| | | CTAGACGCAGCTGCTAGCTAATCGATACCGTCGACCTCGAGGAATTCACGCGT |
| | | GGTACCTCTAGAGTCGACCCGGGCGGCCGCTTCCCTTTAGTGAGGGTTAATG |
| | | CTTCGAGCAGACATGATAAGATACATTGATGAGTTTGGACAAACCACAACTAGA |
| | | ATGCAGTGAAAAAAATGCTTTATTTGTGAAATTTGTGATGCTATTGCTTTATTTGT |
| | | AACCATTATAAGCTGCAATAAACAAGTTAACAACAACAATTGCATTCATTTTATG |
| | | TTTCAGGTTCAGGGGGAGATGTGGGAGGTTTTTTAAAGCAAGTAAAACCTCTAC |
| | | AAATGTGGTAAAATCCGATAAGGGACTAGAGCAGAAGCTTTTG |
| Full: 2 | Modified | [CTAG]AAGCTTTATTGTGGTAGTTTATCACAGTTAAATTGCTAAGGCA |
| 5′UTR: 42 | hIns-2 | GTCAGTGCTTCTGACACAACAGTCTCCAACTTAAGCTGCAGTGACTC |
| ORF: 43 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCACTGCTGGCCCTCTGG |
| | | GGACCTGACCCAGCAGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGTCCAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 3 | Modified | [CTAG]AAGCTTTATTGTGGTAGTTTATCACAGTTAAATTGCTAAGGCA |
| 5′UTR: 42 | hIns-3 | GTCAGTGCTTCTGACACAACAGTCTCCAACTTAAGCTGCAGTGACTC |
| ORF: 44 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGGCTCCTGCCCCTGCTGGCCCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGGGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGGCAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 4 | Modified | [CTAG]AAGCTTTATTGTGGTAGTTTATCACAGTTAAATTGCTAAGGCA |
| 5′UTR: 42 | hIns-4 | GTCAGTGCTTCTGACACAACAGTCTCCAACTTAAGCTGCAGTGACTC |
| ORF: 45 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGGCTCCTGCCCCTGCTGGCTCTGCTGGCCCTCTGG |
| | | GGACCTGACCCAGCAGCAGCCTTTGTGAACCAACACCTGTGTGGCTCAGA |
| | | TCTGGTGGAAGCTCTCTACCTAGTGTGTGGGGAAAGGGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGGCAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 5 | Modified | [CTAG]AAGCTTTATTGTGGTAGTTTATCACAGTTAAATTGCTAAGGCA |
| 5′UTR: 42 | hIns-5 | GTCAGTGCTTCTGACACAACAGTCTCCAACTTAAGCTGCAGTGACTC |
| ORF: 46 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTGCTCCCCCTCCTGGCTTTGCTGGCCCTGTGGG |
| | | GACCAGATCCTGCAGCTGCCTTTGTGAATCAGCACCTGTGTGGCTCAGAT |
| | | CTGGTTGAGGCACTCTACCTGGTGTGTGGGGAGAGAGGCTTCTTTTACAC |
| | | ACCAAGGACCAAGAGGGAGGCAGAGGATCTGCAGGTAGGTCAGGTGGA |
| | | GCTGGGAGGTGGCCCTGGAGCAGGATCCCTGCAGCCCTTGGCACTGGAG |
| | | GGCTCCAGACAGAAGAGGGGCATTGTGGAACAGTGTTGCACTAGCATCT |
| | | GCAGCCTGTACCAGCTGGAGAACTACTGCAACTAAAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 6 | Modified | [CTAG]AAGCTTTATTGTGGTAGTTTATCACAGTTAAATTGCTAAGGCA |
| 5′UTR: 42 | hIns-6 | GTCAGTGCTTCTGACACAACAGTCTCCAACTTAAGCTGCAGTGACTC |
| ORF: 47 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAAAGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGCGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 7 | Modified | [CTAG]AAGCTTTATTGTGGTAGTTTATCACAGTTAAATTGCTAAGGCA |
| 5′UTR: 42 | hIns-7 | GTCAGTGCTTCTGACACAACAGTCTCCAACTTAAGCTGCAGTGACTC |
| ORF: 48 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAAAGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 8 | Modified | [CTAG]AAGCTTTATTGTGGTAGTTTATCACAGTTAAATTGCTAAGGCA |
| 5′UTR: 42 | hIns-8 | GTCAGTGCTTCTGACACAACAGTCTCCAACTTAAGCTGCAGTGACTC |
| ORF: 49 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGTCTAGACAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 9 | Modified | [CTAG]AAGCTTTATTGTGGTAGTTTATCACAGTTAAATTGCTAAGGCA |
| 5′UTR: 42 | hIns-9 | GTCAGTGCTTCTGACACAACAGTCTCCAACTTAAGCTGCAGTGACTC |
| ORF: 50 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCCCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGAGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGCAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 10 | Modified | [CTAG]AAGCTTTATTGTGGTAGTTTATCACAGTTAAATTGCTAAGGCA |
| 5′UTR: 42 | hIns-10 | GTCAGTGCTTCTGACACAACAGTCTCCAACTTAAGCTGCAGTGACTC |
| ORF: 51 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGGGAGGCAGAGGACCTGCAGGTGGGGCAGGTGG |
| | | AGCTGGGAGGGGGCCCTGGTGCAGGCAGCCTGCAGCCCTTGGCCCTGGA |
| | | GGGGAGTAGACAGAAGAGGGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 11 | Modified | [CTAG]AAGCTTTATTGTGGTAGTTTATCACAGTTAAATTGCTAAGGCA |
| 5′UTR: 42 | hIns-11 | GTCAGTGCTTCTGACACAACAGTCTCCAACTTAAGCTGCAGTGACTC |
| ORF: 52 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCCTGCCCCTGCTGGCTCTGCTGGCCCTCTGG |
| | | GGACCTGACCCAGCTGCAGCCTTTGTGAACCAACACCTGTGTGGCTCAGA |
| | | TCTGGTGGAAGCTCTCTACCTAGTGTGTGGGGAAAGAGGCTTCTTCTACA |
| | | CACCCAGGACCAAGAGAGAGGCAGAGGACCTGCAGGTGGGGCAGGTGG |
| | | AGCTGGGTGGGGGCCCTGGTGCAGGCAGCCTGCAGCCCTTGGCCCTGGA |
| | | GGGGTCTAGACAGAAGAGAGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |
| Full: 12 | Modified | [CTAG]AAGCTTTATTGTGGTAGTTTATCACAGTTAAATTGCTAAGGCA |
| 5′UTR: 42 | hIns -12 | GTCAGTGCTTCTGACACAACAGTCTCCAACTTAAGCTGCAGTGACTC |
| ORF: 53 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTCGTGAACCAGCACCTCTGCGGAAGCGAT |
| | | CTGGTCGAGGCCTTGTACCTGGTTTGTGGGGAAAGAGGTTTTTTCTATACC |
| | | CCGAGGACAAAGCGCGAGGCAGAAGACCTTCAAGTGGGCCAGGTAGAGC |
| | | TCGGAGGGGGCCCCGGTGCCGGATCCCTGCAGCCTCTGGCTCTCGAAGGG |
| | | TCTCGACAAAAACGTGGCATCGTGGAGCAGTGCTGTACTTCAATTTGCAG |
| | | TTTGTACCAGCTTGAGAATTATTGTAACTGAAGCCAGCTGCCTTCCCTTTAC |
| | | TGTGGGTTAATGCTTGCAGCAGACATGATAAGATACATTGATGAGTTTGGACAA |
| | | ACCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCAA |
| | | GTAAAACCTCTACAAATGTGGTAAAATCCGATAAGGGACTAGAGCAGAAGCTTT |
| | | TG |
| Full: 13 | Modified | [CTAG]AAGCTTTATTGTGGTAGTTTATCACAGTTAAATTGCTAAGGCA |
| 5′UTR: 42 | hIns-13 | GTCAGTGCTTCTGACACAACAGTCTCCAACTTAAGCTGCAGTGACTC |
| ORF: 54 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTTGTGAACCAGCACCTCTGCGGATCTGAT |
| | | CTGGTTGAGGCCTTGTACCTGGTTTGTGGGGAAAGAGGTTTTTTCTATACC |
| | | CCAAGGACAAAGCGCGAGGCAGAAGACCTTCAAGTGGGCCAGGTAGAGC |
| | | TGGGAGGGGGCCCGGGTGCAGGATCCCTGCAGCCTCTGGCTCTAGAAGG |
| | | GTCTAGACAAAAAAGGGGCATAGTGGAGCAGTGCTGTACTTCAATTTGCA |
| | | GTTTGTACCAGCTTGAGAATTATTGTAACTGAAGCCAGCTGCCTTCCCTTTA |
| | | CTGTGGGTTAATGCTTGCAGCAGACATGATAAGATACATTGATGAGTTTGGACA |
| | | AACCACAACTAGAATGCAGTGAAAAAAATGCTTTATTTGTGAAATTTGTGATGCT |
| | | ATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 14 | Modified | [CTAG]AAGCTTTATTGTGGTAGTTTATCACAGTTAAATTGCTAAGGCA |
| 5′UTR: 42 | hIns-14 | GTCAGTGCTTCTGACACAACAGTCTCCAACTTAAGCTGCAGTGACTC |
| ORF: 55 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGGCTCTTGCCCCTTCTGGCTTTACTGGCACTATGGG |
| | | GCCCTGACCCAGCGGCCGCTTTTGTGAACCAGCACCTCTGCGGATCTGAT |
| | | CTGGTGGAGGCCTTGTACCTGGTTTGTGGGGAAAGAGGTTTTTTCTATAC |
| | | CCCCAGGACAAAGCGCGAGGCAGAAGACCTTCAAGTGGGCCAGGTAGAG |
| | | CTGGGAGGGGGCCCGGGTGCAGGATCCCTGCAGCCTCTGGCTCTGGAAG |
| | | GGTCTAGACAAAAAAGGGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 15 | Modified | [CTAG]AAGCTTTATTGTGGTAGTTTATCACAGTTAAATTGCTAAGGCA |
| 5′UTR: 42 | hIns-15 | GTCAGTGCTTCTGACACAACAGTCTCCAACTTAAGCTGCAGTGACTC |
| ORF: 56 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCTTGCCCCTTCTGGCTTTACTGGCACTATGGG |
| | | GCCCTGACCCAGCAGCTGCTTTTGTGAACCAGCACCTCTGTGGATCTGAT |
| | | CTGGTTGAGGCCTTGTACCTGGTTTGTGGGGAAAGAGGTTTTTTCTATACC |
| | | CCAAGGACAAAGAGGGAGGCAGAAGACCTTCAAGTGGGCCAGGTAGAG |
| | | CTGGGAGGGGGCCCTGGTGCAGGATCCCTGCAGCCTCTGGCTCTAGAAGG |
| | | GTCTAGACAAAAAAGGGGCATAGTGGAGCAGTGCTGTACTTCAATTTGCA |
| | | GTTTGTACCAGCTTGAGAATTATTGTAACTGAAGCCAGCTGCCTTCCCTTTA |
| | | CTGTGGGTTAATGCTTGCAGCAGACATGATAAGATACATTGATGAGTTTGGACA |
| | | AACCACAACTAGAATGCAGTGAAAAAAATGCTTTATTTGTGAAATTTGTGATGCT |
| | | ATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 16 | Modified | [CTAG]AAGCTTTATTGTGGTAGTTTATCACAGTTAAATTGCTAAGGCA |
| 5′UTR: 42 | hIns-16 | GTCAGTGCTTCTGACACAACAGTCTCCAACTTAAGCTGCAGTGACTC |
| ORF: 57 | | TCTTAAGGTAGCCTTGCAGAAGTTGGTCCTGAGGCACTGGGCAGGTA |
| 3′UTR: 60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGAGACTCTTGCCCCTTCTGGCTTTACTGGCACTATGGG |
| | | GCCCTGACCCAGCAGCTGCTTTTGTGAACCAGCACCTCTGTGGATCTGAT |
| | | CTGGTGGAGGCCTTGTACCTGGTTTGTGGGGAAAGAGGTTTTTTCTATAC |
| | | CCCCAGGACAAAGAGAGAGGCAGAAGACCTTCAAGTGGGCCAGGTAGAG |
| | | CTGGGAGGGGGCCCAGGTGCAGGATCCCTGCAGCCTCTGGCTCTGGAAG |
| | | GGTCTAGACAAAAAAGAGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 84 | Modified | AAGCTTTATTGCGGTAGTTTATCACAGTTAAATTGCTAACGCAGTCAG |
| 5′UTR: 83 | hIns-17 | TGCTTCTGACACAACAGTCTCGAACTTAAGCTGCAGTGACTCTCTTAA |
| ORF: 43 | | GGTAGCCTTGCAGAAGTTGGTCGTGAGGCACTGGGCAGGTAAGTATC |
| 3′UTR: 97 | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGAGACTCCTGCCCCTGCTGGCACTGCTGGCCCTCTGGGGACCTG |
| | | ACCCAGCAGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCC |
| | | AGACAGAAGAGAGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGGATCCAAGCTT |
| Full: 85 | Modified | AAGCTTTATTGTGGTAGTTTATCACAGTTAAATTGCTAAGGCAGTCAG |
| 5′UTR: | hIns-18 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 43 | | CTGACATCCACTTTGCCTTTCTCTCCACAGGTGTCCACTCCCAGTTCA |
| 3′UTR: 98 | | ATTACAGCTCTTAAGGCTAGAGTCCTGCAGAATTGCCACCATGGCCCT |
| | | GTGGATGAGACTCCTGCCCCTGCTGGCACTGCTGGCCCTCTGGGGACCTG |
| | | ACCCAGCAGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCC |
| | | AGACAGAAGAGAGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGAATTCAAGCTT |
| Full: 86 | Modified | AAGCTTTATTGTGGTAGTTTATCACAGTTAAATTGCTAAGGCAGTCAG |
| 5′UTR: | hIns-19 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 46 | | CTGACATCCACTTTGCCTTTCTCTCCACAGGTGTCCACTCCCAGTTCA |
| 3′UTR: 99 | | ATTACAGCTCTTAAGGCTAGAGTCCTGCAGAATTGCCACCATGGCCCT |
| | | GTGGATGAGACTGCTCCCCCTCCTGGCTTTGCTGGCCCTGTGGGGACCAG |
| | | ATCCTGCAGCTGCCTTTGTGAATCAGCACCTGTGTGGCTCAGATCTGGTTG |
| | | AGGCACTCTACCTGGTGTGTGGGGAGAGAGGCTTCTTTTACACACCAAGG |
| | | ACCAAGAGGGAGGCAGAGGATCTGCAGGTAGGTCAGGTGGAGCTGGGAG |
| | | GTGGCCCTGGAGCAGGATCCCTGCAGCCCTTGGCACTGGAGGGCTCCAGA |
| | | CAGAAGAGGGGCATTGTGGAACAGTGTTGCACTAGCATCTGCAGCCTGTA |
| | | CCAGCTGGAGAACTACTGCAACTAAAGCCAGCTGCCTTCCCTTTACTGTGG |
| | | GTTAATGCTTGCAGCAGACATGATAAGATACATTGATGAGTTTGGACAAACCAC |
| | | AACTAGAATGCAGTGAAAAAAATGCTTTATTTGTGAAATTTGTGATGCTATTGCT |
| | | TTATTTGTAACCATTATAAGCTGCAATAAACAAGTTAACAACAACAATTGCATTC |
| | | ATTTTATGTTTCAGGTTCAGGGGGAGATGTGGGAGGTTTTTTAAAGCAAGTAAA |
| | | ACCTCTACAAATGTGGTAAAATAAGGGACTAGAGCAGCATATGAAGCTT |
| Full: 87 | Modified | AAGCTTTATTGTGGTAGTTTATCACAGTTAAATTGCTAAGGCAGTCAG |
| 5′UTR: | hIns-20 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 47 | | CTGACATCCACTTTGCCTTTCTCTCCACAGGTGTCCACTCCCAGTTCA |
| 3′UTR: 100 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGGATATCAAGCTT |
| Full: 88 | Modified | AAGCTTTATTGTGGTAGTTTATCACAGTTAAATTGCTAAGGCAGTCAG |
| 5′UTR: | hIns-21 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 49 | | CTGACATCCACTTTGCCTTTCTCTCCACAGGTGTCCACTCCCAGTTCA |
| 3′UTR: 101 | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATAAGGGACTAGAGCAGACTAGTAAGCTT |
| Full: 123 | Modified | AAGCTTTATTGTGGTAGTTTATCACAGTTAAATTGCTAAGGCAGTCAG |
| 5′ UTR: | hIns-22 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 110 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 124 | Modified | AGCTTTATTGTGGTAGTTTATCACAGTTAAATTGCTAAGGCAGTCAGT |
| 5′ UTR: 146 | hIns-23 | GCTTCTGACACAACAGTCTCCAACTTAAGCTGCAGTGACTCTCTTAA |
| ORF 1: 110 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 53 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGCGGCTCTTGCCCCTTCTGGCTTTACTGGCACTATG |
| | | GGGCCCTGACCCAGCGGCCGCTTTCGTGAACCAGCACCTCTGCGGAAGCG |
| | | ATCTGGTCGAGGCCTTGTACCTGGTTTGTGGGGAAAGAGGTTTTTTCTATA |
| | | CCCCGAGGACAAAGCGCGAGGCAGAAGACCTTCAAGTGGGCCAGGTAGA |
| | | GCTCGGAGGGGGCCCCGGTGCCGGATCCCTGCAGCCTCTGGCTCTCGAAG |
| | | GGTCTCGACAAAAACGTGGCATCGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 125 | Modified | AGCTTTATTGTGGTAGTTTATCACAGTTAAATTGCTAAGGCAGTCAGT |
| 5′ UTR: 146 | hIns-24 | GCTTCTGACACAACAGTCTCCAACTTAAGCTGCAGTGACTCTCTTAA |
| ORF 1: 47 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 54 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCAGATCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGCGGCTCTTGCCCCTTCTGGCTTTACTGGCACTATG |
| | | GGGCCCTGACCCAGCGGCCGCTTTTGTGAACCAGCACCTCTGCGGATCTG |
| | | ATCTGGTTGAGGCCTTGTACCTGGTTTGTGGGGAAAGAGGTTTTTTCTATA |
| | | CCCCAAGGACAAAGCGCGAGGCAGAAGACCTTCAAGTGGGCCAGGTAGA |
| | | GCTGGGAGGGGGCCCGGGTGCAGGATCCCTGCAGCCTCTGGCTCTAGAA |
| | | GGGTCTAGACAAAAAAGGGGCATAGTGGAGCAGTGCTGTACTTCAATTT |
| | | GCAGTTTGTACCAGCTTGAGAATTATTGTAACTGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATCCGATAAGGGACTAGAGCAGAA |
| | | GCTTTTG |
| Full: 126 | Modified | AGCTTTATTGTGGTAGTTTATCACAGTTAAATTGCTAAGGCAGTCAGT |
| 5′ UTR: 146 | hIns-25 | GCTTCTGACACAACAGTCTCCAACTTAAGCTGCAGTGACTCTCTTAA |
| ORF 1: 49 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 56 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCAGATCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGAGACTCTTGCCCCTTCTGGCTTTACTGGCACTATG |
| | | GGGCCCTGACCCAGCAGCTGCTTTTGTGAACCAGCACCTCTGTGGATCTG |
| | | ATCTGGTTGAGGCCTTGTACCTGGTTTGTGGGGAAAGAGGTTTTTTCTATA |
| | | CCCCAAGGACAAAGAGGGAGGCAGAAGACCTTCAAGTGGGCCAGGTAGA |
| | | GCTGGGAGGGGGCCCTGGTGCAGGATCCCTGCAGCCTCTGGCTCTAGAAG |
| | | GGTCTAGACAAAAAAGGGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 127 | Unmodified | AAGCTTTATTGTGGTAGTTTATCACAGTTAAATTGCTAAGGCAGTCAG |
| 5′ UTR: | hIns-2 | TGCTTCTGACACAACAGTCTCCAACTTAAGCTGCAGTGACTCTCTTAA |
| nucleic | (B10H) | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 111 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 128 | Modified | AAGCTTTATTGTGGTAGTTTATCACAGTTAAATTGCTAAGGCAGTCAG |
| 5′ UTR: | hIns-27 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 112 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 129 | Modified | AAGCTTTATTGTGGTAGTTTATCACAGTTAAATTGCTAAGGCAGTCAG |
| 5′ UTR: | hIns-28 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 113 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCACACCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGAGCCAGCTGCCTTCCCTTTACTG |
| | | TGGGTTAATGCTTGCAGCAGACATGATAAGATACATTGATGAGTTTGGACAAAC |
| | | CACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCA |
| | | TTCATTTTATGTTTCAGGTTCAGGGGGAGATGTGGGAGGTTTTTTAAAGCAAGT |
| | | AAAACCTCTACAAATGTGGTAAAATCCGATAAGGGACTAGAGCAGAAGCTTTTG |
| Full: 130 | Modified | AGCTTTATTGTGGTAGTTTATCACAGTTAAATTGCTAAGGCAGTCAGT |
| 5′ UTR: 146 | hIns-29 | GCTTCTGACACAACAGTCTCCAACTTAAGCTGCAGTGACTCTCTTAA |
| ORF 1: 111 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 114 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAACGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGTGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGCGGCTCTTGCCCCTTCTGGCTTTACTGGCACTATG |
| | | GGGCCCTGACCCAGCGGCCGCTTTCGTGAACCAGCACCTCTGCGGAAGCC |
| | | ACCTGGTCGAGGCCTTGTACCTGGTTTGTGGGGAAAGAGGTTTTTTCTAT |
| | | ACCCCGAGGACAAAGCGCGAGGCAGAAGACCTTCAAGTGGGCCAGGTAG |
| | | AGCTCGGAGGGGGCCCCGGTGCCGGATCCCTGCAGCCTCTGGCTCTCGAA |
| | | GGGTCTCGACAAAAACGTGGCATCGTGGAGCAGTGCTGTACTTCAATTTG |
| | | CAGTTTGTACCAGCTTGAGAATTATTGTAACTGAAGCCAGCTGCCTTCCCT |
| | | TTACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGAT |
| | | GCTATTGCTTTATTTGTAACCATTATAAGCTGCAATAAACAAGTTAACAACAACA |
| | | ATTGCATTCATTTTATGTTTCAGGTTCAGGGGGAGATGTGGGAGGTTTTTTAAA |
| | | GCAAGTAAAACCTCTACAAATGTGGTAAAATCCGATAAGGGACTAGAGCAGAA |
| | | GCTTTTG |
| Full: 131 | Modified | AGCTTTATTGTGGTAGTTTATCACAGTTAAATTGCTAAGGCAGTCAGT |
| 5′ UTR: 146 | hIns-30 | GCTTCTGACACAACAGTCTCCAACTTAAGCTGCAGTGACTCTCTTAA |
| ORF 1: 112 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 115 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGCGCCTCCTGCCCCTGCTGGCGCTGCTGGCCCTCTGGGGACCTG |
| | | ACCCAGCCGCAGCCTTTGTGAACCAACACCTGTGCGGCTCACACCTGGTG |
| | | GAAGCTCTCTACCTAGTGTGCGGGGAAAGAGGCTTCTTCTACACACCCAG |
| | | GACCAAGCGGGAGGCAGAGGACCTGCAGGTGGGGCAGGTGGAGCTGGG |
| | | CGGGGGCCCTGGTGCAGGCAGCCTGCAGCCCTTGGCCCTGGAGGGGTCG |
| | | CGACAGAAGCGGGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGCGGCTCTTGCCCCTTCTGGCTTTACTGGCACTATG |
| | | GGGCCCTGACCCAGCGGCCGCTTTTGTGAACCAGCACCTCTGCGGATCTC |
| | | ACCTGGTTGAGGCCTTGTACCTGGTTTGTGGGGAAAGAGGTTTTTTCTATA |
| | | CCCCAAGGACAAAGCGCGAGGCAGAAGACCTTCAAGTGGGCCAGGTAGA |
| | | GCTGGGAGGGGGCCCGGGTGCAGGATCCCTGCAGCCTCTGGCTCTAGAA |
| | | GGGTCTAGACAAAAAAGGGGCATAGTGGAGCAGTGCTGTACTTCAATTT |
| | | GCAGTTTGTACCAGCTTGAGAATTATTGTAACTGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATCCGATAAGGGACTAGAGCAGAA |
| | | GCTTTTG |
| Full: 132 | Modified | AGCTTTATTGTGGTAGTTTATCACAGTTAAATTGCTAAGGCAGTCAGT |
| 5′ UTR: 146 | hIns-31 | GCTTCTGACACAACAGTCTCCAACTTAAGCTGCAGTGACTCTCTTAA |
| ORF 1: 113 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 116 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGGCCCT |
| | | GTGGATGAGACTCCTGCCCCTGCTGGCTCTGCTGGCCCTCTGGGGACCTG |
| | | ACCCAGCTGCAGCCTTTGTGAACCAACACCTGTGTGGCTCACACCTGGTG |
| | | GAAGCTCTCTACCTAGTGTGTGGGGAAAGAGGCTTCTTCTACACACCCAG |
| | | GACCAAGAGGGAGGCAGAGGACCTGCAGGTGGGGCAGGTGGAGCTGGG |
| | | AGGGGGCCCTGGTGCAGGCAGCCTGCAGCCCTTGGCCCTGGAGGGGTCT |
| | | AGACAGAAGAGGGGCATTGTGGAACAATGCTGTACCAGCATCTGCTCCCT |
| | | CTACCAGCTGGAGAACTACTGCAACTAGGCCCCTCTCCCTCCCCCCCCCC |
| | | TAACGTTACTGGCCGAAGCCGCTTGGAATAAGGCCGGTGTGCGTTTGTCTA |
| | | TATGTTATTTTCCACCATATTGCCGTCTTTTGGCAATGTGAGGGCCCGGAAA |
| | | CCTGGCCCTGTCTTCTTGACGAGCATTCCTAGGGGTCTTTCCCCTCTCGCC |
| | | AAAGGAATGCAAGGTCTGTTGAATGTCGTGAAGGAAGCAGTTCCTCTGGAA |
| | | GCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGAT |
| | | ACACCTGCAAAGGCGGCACAACCCCAGTGCCACGTTGTGAGTTGGATAGTT |
| | | GTGGAAAGAGTCAAATGGCTCTCCTCAAGCGTATTCAACAAGGGGCTGAAG |
| | | GATGCCCAGAAGGTACCCCATTGTATGGGATCTGATCTGGGGCCTCGGTAC |
| | | ACATGCTTTACATGTGTTTAGTCGAGGTTAAAAAAACGTCTAGGCCCCCCG |
| | | AACCACGGGGACGTGGTTTTCCTTTGAAAAACACGATGATAATATGGCCAC |
| | | AATGGCCCTGTGGATGAGACTCTTGCCCCTTCTGGCTTTACTGGCACTATG |
| | | GGGCCCTGACCCAGCAGCTGCTTTTGTGAACCAGCACCTCTGTGGATCTC |
| | | ACCTGGTTGAGGCCTTGTACCTGGTTTGTGGGGAAAGAGGTTTTTTCTATA |
| | | CCCCAAGGACAAAGAGGGAGGCAGAAGACCTTCAAGTGGGCCAGGTAGA |
| | | GCTGGGAGGGGGCCCTGGTGCAGGATCCCTGCAGCCTCTGGCTCTAGAAG |
| | | GGTCTAGACAAAAAAGGGGCATAGTGGAGCAGTGCTGTACTTCAATTTGC |
| | | AGTTTGTACCAGCTTGAGAATTATTGTAACTGAAGCCAGCTGCCTTCCCTTT |
| | | ACTGTGGGTTAATGCTTGCAGCAGACATGATAAGATACATTGATGAGTTTGGAC |
| | | AAACCACAACTAGAATGCAGTGAAAAAAATGCTTTATTTGTGAAATTTGTGATGC |
| | | TATTGCTTTATTTGTAACCATTATAAGCTGCAATAAACAAGTTAACAACAACAATT |
| | | GCATTCATTTTATGTTTCAGGTTCAGGGGGAGATGTGGGAGGTTTTTTAAAGCA |
| | | AGTAAAACCTCTACAAATGTGGTAAAATCCGATAAGGGACTAGAGCAGAAGCTT |
| | | TTG |
| Full: 133 | Modified | AAGCTTTATTGTGGTAGTTTATCACAGTTAAATTGCTAAGGCAGTCAG |
| 5′ UTR: | hIns-32 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 117 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTCGGCCCCGTGGCTTTCTCTCTGGGACTGCTGC |
| | | TGGTGCTGCCTGCCGCTTTCCCTGCCCCTTTTGTGAACCAACACCTGTGCG |
| | | GCTCAGATCTGGTGGAAGCTCTCTACCTAGTGTGCGGGGAACGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGTGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 134 | Modified | AAGCTTTATTGTGGTAGTTTATCACAGTTAAATTGCTAAGGCAGTCAG |
| 5′ UTR: | hIns-33 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 118 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCAGATCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 135 | Modified | AAGCTTTATTGTGGTAGTTTATCACAGTTAAATTGCTAAGGCAGTCAG |
| 5′ UTR: | hIns-34 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 119 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGTGCCTTTGGCCCAGTGGCTTTCTCTCTGGGACTGCTGC |
| | | TGGTGCTGCCTGCAGCTTTCCCTGCCCCTTTTGTGAACCAACACCTGTGTG |
| | | GCTCAGATCTGGTGGAAGCTCTCTACCTAGTGTGTGGGGAAAGAGGCTTC |
| | | TTCTACACACCCAGGACCAAGAGAGAGGCAGAGGACCTGCAGGTGGGGC |
| | | AGGTGGAGCTGGGAGGGGGCCCTGGTGCAGGCAGCCTGCAGCCCTTGGC |
| | | CCTGGAGGGGTCCAGGCAGAAGAGAGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 136 | Modified | AAGCTTTATTGTGGTAGTTTATCACAGTTAAATTGCTAAGGCAGTCAG |
| 5′ UTR: | hIns-35 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 120 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTCGGCCCCGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAACGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGTGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 137 | Modified | AAGCTTTATTGTGGTAGTTTATCACAGTTAAATTGCTAAGGCAGTCAG |
| 5′ UTR: | hIns-36 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 121 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 138 | Modified | AAGCTTTATTGTGGTAGTTTATCACAGTTAAATTGCTAAGGCAGTCAG |
| 5′ UTR: | hIns-37 | TGCTTCTGACACAACAGTCTCCAACTTAAGCTGCAGTGACTCTCTTAA |
| nucleic | | GGTAGCCTTGCAGAAGTTGGTCCTGAGGCACTGGGCAGGTAAGTATC |
| acids 5-329 | | AAGGTTACAAGACAGGTTTAAGGAGACCAATAGAAACTGGGCTTGTG |
| of 42 | | GAGACAGAGAAGACTCTTGGCTTTCTGATAGGCACCTATTGGTCTTA |
| ORF: 122 | | CTGACATCCACTTTGCCTTTCTCTCCACAGGTGTCCACTCCCAGTTCA |
| 3′ UTR: 60 | | ATTACAGCTCTTAAGGCTAGAGTCCTGCAGAATTGCCACCATGAACA |
| | | GCTTCAGCACAAGTGCCTTTGGCCCAGTGGCTTTCTCTCTGGGACTGCTGC |
| | | TGGTGCTGCCTGCAGCTTTCCCTGCCCCTTTTGTGAACCAACACCTGTGTG |
| | | GCTCACACCTGGTGGAAGCTCTCTACCTAGTGTGTGGGGAAAGAGGCTTC |
| | | TTCTACACACCCAGGACCAAGAGAGAGGCAGAGGACCTGCAGGTGGGGC |
| | | AGGTGGAGCTGGGAGGGGGCCCTGGTGCAGGCAGCCTGCAGCCCTTGGC |
| | | CCTGGAGGGGTCCAGGCAGAAGAGAGGCATTGTGGAACAATGCTGTACC |
| | | AGCATCTGCTCCCTCTACCAGCTGGAGAACTACTGCAACTAGAGCCAGCT |
| | | GCCTTCCCTTTACTGTGGGTTAATGCTTGCAGCAGACATGATAAGATACATTGA |
| | | TGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTA |
| | | ACAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTAG |
| | | AGCAGAAGCTTTTG |
| Full: 139 | Modified | AGCTTTATTGTGGTAGTTTATCACAGTTAAATTGCTAAGGCAGTCAGT |
| 5′ UTR: 146 | hIns-38 | GCTTCTGACACAACAGTCTCCAACTTAAGCTGCAGTGACTCTCTTAA |
| ORF 1: 120 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 114 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGAACA |
| | | GCTTCAGCACAAGCGCCTTCGGCCCCGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAACGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGTGGCATTGTGGAACAATGCTGTACC |
| | | AGCATCTGCTCCCTCTACCAGCTGGAGAACTACTGCAACTAGGCCCCTCT |
| | | CCCTCCCCCCCCCCTAACGTTACTGGCCGAAGCCGCTTGGAATAAGGCCGG |
| | | TGTGCGTTTGTCTATATGTTATTTTCCACCATATTGCCGTCTTTTGGCAATG |
| | | TGAGGGCCCGGAAACCTGGCCCTGTCTTCTTGACGAGCATTCCTAGGGGTC |
| | | TTTCCCCTCTCGCCAAAGGAATGCAAGGTCTGTTGAATGTCGTGAAGGAAG |
| | | CAGTTCCTCTGGAAGCTTCTTGAAGACAAACAACGTCTGTAGCGACCCTTT |
| | | GCAGGCAGCGGAACCCCCCACCTGGCGACAGGTGCCTCTGCGGCCAAAAG |
| | | CCACGTGTATAAGATACACCTGCAAAGGCGGCACAACCCCAGTGCCACGTT |
| | | GTGAGTTGGATAGTTGTGGAAAGAGTCAAATGGCTCTCCTCAAGCGTATTC |
| | | AACAAGGGGCTGAAGGATGCCCAGAAGGTACCCCATTGTATGGGATCTGAT |
| | | CTGGGGCCTCGGTACACATGCTTTACATGTGTTTAGTCGAGGTTAAAAAAA |
| | | CGTCTAGGCCCCCCGAACCACGGGGACGTGGTTTTCCTTTGAAAAACACGA |
| | | TGATAATATGGCCACAATGGCCCTGTGGATGCGGCTCTTGCCCCTTCTGGC |
| | | TTTACTGGCACTATGGGGCCCTGACCCAGCGGCCGCTTTCGTGAACCAGC |
| | | ACCTCTGCGGAAGCCACCTGGTCGAGGCCTTGTACCTGGTTTGTGGGGAA |
| | | AGAGGTTTTTTCTATACCCCGAGGACAAAGCGCGAGGCAGAAGACCTTCA |
| | | AGTGGGCCAGGTAGAGCTCGGAGGGGGCCCCGGTGCCGGATCCCTGCAG |
| | | CCTCTGGCTCTCGAAGGGTCTCGACAAAAACGTGGCATCGTGGAGCAGTG |
| | | CTGTACTTCAATTTGCAGTTTGTACCAGCTTGAGAATTATTGTAACTGAAG |
| | | CCAGCTGCCTTCCCTTTACTGTGGGTTAATGCTTGCAGCAGACATGATAAGATA |
| | | CATTGATGAGTTTGGACAAACCACAACTAGAATGCAGTGAAAAAAATGCTTTATT |
| | | TGTGAAATTTGTGATGCTATTGCTTTATTTGTAACCATTATAAGCTGCAATAAAC |
| | | AAGTTAACAACAACAATTGCATTCATTTTATGTTTCAGGTTCAGGGGGAGATGT |
| | | GGGAGGTTTTTTAAAGCAAGTAAAACCTCTACAAATGTGGTAAAATCCGATAAG |
| | | GGACTAGAGCAGAAGCTTTTG |
| Full: 140 | Modified | AGCTTTATTGTGGTAGTTTATCACAGTTAAATTGCTAAGGCAGTCAGT |
| 5′ UTR: 146 | hIns-39 | GCTTCTGACACAACAGTCTCCAACTTAAGCTGCAGTGACTCTCTTAA |
| ORF 1: 121 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 115 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGAACA |
| | | GCTTCAGCACAAGCGCCTTTGGCCCAGTGGCTTTCTCTCTGGGACTGCTGC |
| | | TGGTGCTGCCTGCCGCTTTCCCTGCCCCTTTTGTGAACCAACACCTGTGCG |
| | | GCTCACACCTGGTGGAAGCTCTCTACCTAGTGTGCGGGGAAAGAGGCTTC |
| | | TTCTACACACCCAGGACCAAGCGGGAGGCAGAGGACCTGCAGGTGGGGC |
| | | AGGTGGAGCTGGGCGGGGGCCCTGGTGCAGGCAGCCTGCAGCCCTTGGC |
| | | CCTGGAGGGGTCGCGACAGAAGCGGGGCATTGTGGAACAATGCTGTACC |
| | | AGCATCTGCTCCCTCTACCAGCTGGAGAACTACTGCAACTAGGCCCCTCT |
| | | CCCTCCCCCCCCCCTAACGTTACTGGCCGAAGCCGCTTGGAATAAGGCCGG |
| | | TGTGCGTTTGTCTATATGTTATTTTCCACCATATTGCCGTCTTTTGGCAATG |
| | | TGAGGGCCCGGAAACCTGGCCCTGTCTTCTTGACGAGCATTCCTAGGGGTC |
| | | TTTCCCCTCTCGCCAAAGGAATGCAAGGTCTGTTGAATGTCGTGAAGGAAG |
| | | CAGTTCCTCTGGAAGCTTCTTGAAGACAAACAACGTCTGTAGCGACCCTTT |
| | | GCAGGCAGCGGAACCCCCCACCTGGCGACAGGTGCCTCTGCGGCCAAAAG |
| | | CCACGTGTATAAGATACACCTGCAAAGGCGGCACAACCCCAGTGCCACGTT |
| | | GTGAGTTGGATAGTTGTGGAAAGAGTCAAATGGCTCTCCTCAAGCGTATTC |
| | | AACAAGGGGCTGAAGGATGCCCAGAAGGTACCCCATTGTATGGGATCTGAT |
| | | CTGGGGCCTCGGTACACATGCTTTACATGTGTTTAGTCGAGGTTAAAAAAA |
| | | CGTCTAGGCCCCCCGAACCACGGGGACGTGGTTTTCCTTTGAAAAACACGA |
| | | TGATAATATGGCCACAATGGCCCTGTGGATGCGGCTCTTGCCCCTTCTGGC |
| | | TTTACTGGCACTATGGGGCCCTGACCCAGCGGCCGCTTTTGTGAACCAGC |
| | | ACCTCTGCGGATCTCACCTGGTTGAGGCCTTGTACCTGGTTTGTGGGGAA |
| | | AGAGGTTTTTTCTATACCCCAAGGACAAAGCGCGAGGCAGAAGACCTTCA |
| | | AGTGGGCCAGGTAGAGCTGGGAGGGGGCCCGGGTGCAGGATCCCTGCAG |
| | | CCTCTGGCTCTAGAAGGGTCTAGACAAAAAAGGGGCATAGTGGAGCAGT |
| | | GCTGTACTTCAATTTGCAGTTTGTACCAGCTTGAGAATTATTGTAACTGAA |
| | | GCCAGCTGCCTTCCCTTTACTGTGGGTTAATGCTTGCAGCAGACATGATAAGAT |
| | | ACATTGATGAGTTTGGACAAACCACAACTAGAATGCAGTGAAAAAAATGCTTTA |
| | | TTTGTGAAATTTGTGATGCTATTGCTTTATTTGTAACCATTATAAGCTGCAATAAA |
| | | CAAGTTAACAACAACAATTGCATTCATTTTATGTTTCAGGTTCAGGGGGAGATG |
| | | TGGGAGGTTTTTTAAAGCAAGTAAAACCTCTACAAATGTGGTAAAATCCGATAA |
| | | GGGACTAGAGCAGAAGCTTTTG |
| Full: 141 | Modified | AGCTTTATTGTGGTAGTTTATCACAGTTAAATTGCTAAGGCAGTCAGT |
| 5′ UTR: 146 | hIns-40 | GCTTCTGACACAACAGTCTCCAACTTAAGCTGCAGTGACTCTCTTAA |
| ORF 1: 122 | | GGTAGCCTTGCAGAAGTTGGTCCTGAGGCACTGGGCAGGTAAGTATC |
| IRES: 143 | | AAGGTTACAAGACAGGTTTAAGGAGACCAATAGAAACTGGGCTTGTG |
| ORF 2: 116 | | GAGACAGAGAAGACTCTTGGCTTTCTGATAGGCACCTATTGGTCTTA |
| 3′ UTR: 60 | | CTGACATCCACTTTGCCTTTCTCTCCACAGGTGTCCACTCCCAGTTCA |
| | | ATTACAGCTCTTAAGGCTAGAGTCCTGCAGAATTGCCACCATGAACA |
| | | GCTTCAGCACAAGTGCCTTTGGCCCAGTGGCTTTCTCTCTGGGACTGCTGC |
| | | TGGTGCTGCCTGCAGCTTTCCCTGCCCCTTTTGTGAACCAACACCTGTGTG |
| | | GCTCACACCTGGTGGAAGCTCTCTACCTAGTGTGTGGGGAAAGAGGCTTC |
| | | TTCTACACACCCAGGACCAAGAGAGAGGCAGAGGACCTGCAGGTGGGGC |
| | | AGGTGGAGCTGGGAGGGGGCCCTGGTGCAGGCAGCCTGCAGCCCTTGGC |
| | | CCTGGAGGGGTCCAGGCAGAAGAGAGGCATTGTGGAACAATGCTGTACC |
| | | AGCATCTGCTCCCTCTACCAGCTGGAGAACTACTGCAACTAGGCCCCTCT |
| | | CCCTCCCCCCCCCCTAACGTTACTGGCCGAAGCCGCTTGGAATAAGGCCGG |
| | | TGTGCGTTTGTCTATATGTTATTTTCCACCATATTGCCGTCTTTTGGCAATG |
| | | TGAGGGCCCGGAAACCTGGCCCTGTCTTCTTGACGAGCATTCCTAGGGGTC |
| | | TTTCCCCTCTCGCCAAAGGAATGCAAGGTCTGTTGAATGTCGTGAAGGAAG |
| | | CAGTTCCTCTGGAAGCTTCTTGAAGACAAACAACGTCTGTAGCGACCCTTT |
| | | GCAGGCAGCGGAACCCCCCACCTGGCGACAGGTGCCTCTGCGGCCAAAAG |
| | | CCACGTGTATAAGATACACCTGCAAAGGCGGCACAACCCCAGTGCCACGTT |
| | | GTGAGTTGGATAGTTGTGGAAAGAGTCAAATGGCTCTCCTCAAGCGTATTC |
| | | AACAAGGGGCTGAAGGATGCCCAGAAGGTACCCCATTGTATGGGATCTGAT |
| | | CTGGGGCCTCGGTACACATGCTTTACATGTGTTTAGTCGAGGTTAAAAAAA |
| | | CGTCTAGGCCCCCCGAACCACGGGGACGTGGTTTTCCTTTGAAAAACACGA |
| | | TGATAATATGGCCACAATGGCCCTGTGGATGAGACTCTTGCCCCTTCTGGC |
| | | TTTACTGGCACTATGGGGCCCTGACCCAGCAGCTGCTTTTGTGAACCAGC |
| | | ACCTCTGTGGATCTCACCTGGTTGAGGCCTTGTACCTGGTTTGTGGGGAA |
| | | AGAGGTTTTTTCTATACCCCAAGGACAAAGAGGGAGGCAGAAGACCTTC |
| | | AAGTGGGCCAGGTAGAGCTGGGAGGGGGCCCTGGTGCAGGATCCCTGCA |
| | | GCCTCTGGCTCTAGAAGGGTCTAGACAAAAAAGGGGCATAGTGGAGCAG |
| | | TGCTGTACTTCAATTTGCAGTTTGTACCAGCTTGAGAATTATTGTAACTGA |
| | | AGCCAGCTGCCTTCCCTTTACTGTGGGTTAATGCTTGCAGCAGACATGATAAGA |
| | | TACATTGATGAGTTTGGACAAACCACAACTAGAATGCAGTGAAAAAAATGCTTT |
| | | ATTTGTGAAATTTGTGATGCTATTGCTTTATTTGTAACCATTATAAGCTGCAATAA |
| | | ACAAGTTAACAACAACAATTGCATTCATTTTATGTTTCAGGTTCAGGGGGAGAT |
| | | GTGGGAGGTTTTTTAAAGCAAGTAAAACCTCTACAAATGTGGTAAAATCCGATA |
| | | AGGGACTAGAGCAGAAGCTTTTG |
| Full: 160 | Unmodified | AAGCTTTATTGCGGTAGTTTATCACAGTTAAATTGCTAACGCAGTCAG |
| 5′ UTR: 148 | hIns_2 | TGCTTCTGACACAACAGTCTCGAACTTAAGCTGCAGTGACTCTCTTAA |
| ORF: 110 | (B10D) | GGTAGCCTTGCAGAAGTTGGTCGTGAGGCACTGGGCAGGTAAGTATC |
| | | AAGGTTACAAGACAGGTTTAAGGAGACCAATAGAAACTGGGCTTGTC |
| | | GAGACAGAGAAGACTCTTGCGTTTCTGATAGGCACCTATTGGTCTTA |
| | | CTGACATCCACTTTGCCTTTCTCTCCACAGGTGTCCACTCCCAGTTCA |
| | | ATTACAGCTCTTAAGGCTAGAGTACTTAATACGACTCACTATAGGCTA |
| | | GCCTCGAGAATTCTGCCATGGCCCTGTGGATGCGCCTCCTGCCCCTGCT |
| | | GGCGCTGCTGGCCCTCTGGGGACCTGACCCAGCCGCAGCCTTTGTGAACC |
| | | AACACCTGTGCGGCTCAGATCTGGTGGAAGCTCTCTACCTAGTGTGCGGG |
| | | GAACGAGGCTTCTTCTACACACCCAGGACCAAGCGGGAGGCAGAGGACC |
| | | TGCAGGTGGGGCAGGTGGAGCTGGGCGGGGGCCCTGGTGCAGGCAGCCT |
| | | GCAGCCCTTGGCCCTGGAGGGGTCGCGACAGAAGCGTGGCATTGTGGAA |
| | | CAATGCTGTACCAGCATCTGCTCCCTCTACCAGCTGGAGAACTACTGCAA |
| | | CTAG |
| Full: 161 | Unmodified | [CTAG]AAGCTTTATTGTGGTAGTTTATCACAGTTAAATTGCTAAGGCA |
| 5′ UTR: 42 | hIns_3 | GTCAGTGCTTCTGACACAACAGTCTCCAACTTAAGCTGCAGTGACTC |
| ORF-110 | (B10D) | TCTTAAGGTAGCCTTGCAGAAGTTGGTCCTGAGGCACTGGGCAGGTA |
| 3′ UTR-60 | | AGTATCAAGGTTACAAGACAGGTTTAAGGAGACCAATAGAAACTGGG |
| | | CTTGTGGAGACAGAGAAGACTCTTGGCTTTCTGATAGGCACCTATTG |
| | | GTCTTACTGACATCCACTTTGCCTTTCTCTCCACAGGTGTCCACTCCC |
| | | AGTTCAATTACAGCTCTTAAGGCTAGAGTCCTGCAGAATTGCCACCA |
| | | TGGCCCTGTGGATGCGCCTCCTGCCCCTGCTGGCGCTGCTGGCCCTCTGG |
| | | GGACCTGACCCAGCCGCAGCCTTTGTGAACCAACACCTGTGCGGCTCAGA |
| | | TCTGGTGGAAGCTCTCTACCTAGTGTGCGGGGAACGAGGCTTCTTCTACA |
| | | CACCCAGGACCAAGCGGGAGGCAGAGGACCTGCAGGTGGGGCAGGTGG |
| | | AGCTGGGCGGGGGCCCTGGTGCAGGCAGCCTGCAGCCCTTGGCCCTGGA |
| | | GGGGTCGCGACAGAAGCGTGGCATTGTGGAACAATGCTGTACCAGCATC |
| | | TGCTCCCTCTACCAGCTGGAGAACTACTGCAACTAGAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCAG |
| | | AAGCTTTTG |

In some aspects, the nucleic acids include codon optimized and CpG reduced sequences relative to wild-type and/or unmodified human insulin nucleic acid sequences (e.g., SEQ ID NO: 1). The modified nucleic acids were chemically synthesized, prepared in expression cassettes including a CMV promoter, and cloned into an expression plasmid. The modified sequences were confirmed by Sanger sequencing.

Secretion of insulin from HEK cells transfected with pAAV-insulin plasmids was tested. The pAAV-insulin plasmids AAV1-CMV-hInsB10D_2 (SEQ ID NO: 160), AAV1-CMV-unmodified hIns (SEQ ID NO: 1), AAV1-CMV-modified hIns22 (SEQ ID NO: 123), AAV1-CMV-modified hIns6 (SEQ ID NO: 6), AAV1-CMV-modified hIns8 (SEQ ID NO: 8), AAV1-CMV-modified hIns23 (SEQ ID NO: 124), AAV1-CMV-modified hIns24 (SEQ ID NO: 125), AAV1-CMV-modified hIns25 (SEQ ID NO: 126), AAV1-CMV-modified hIns_2 (SEQ ID NO: 127), AAV1-CMV-modified hIns27 (SEQ ID NO: 128), AAV1-CMV-modified hIns28 (SEQ ID NO: 129), AAV1-CMV-modified hIns29 (SEQ ID NO: 130), AAV1-CMV-modified hIns30 (SEQ ID NO: 131), AAV1-CMV-modified hIns31 (SEQ ID NO: 132), AAV1-CMV-modified hIns32 (SEQ ID NO: 133), AAV1-CMV-modified hIns33 (SEQ ID NO: 134), AAV1-CMV-modified hIns34 (SEQ ID NO: 135), AAV1-CMV-modified hIns35 (SEQ ID NO: 136), AAV1-CMV-modified hIns36 (SEQ ID NO: 137), AAV1-CMV-modified hIns37 (SEQ ID NO: 138), AAV1-CMV-modified hIns38 (SEQ ID NO: 139), AAV1-CMV-modified hIns39 (SEQ ID NO: 140), or AAV1-CMV-modified Ins40 (SEQ ID NO: 141) were transfected into HEK293 cells in a 24-well plate at 0.5 ug (FIG. 1B) or 0.1 ug (FIG. 1C). Extracellular insulin levels were determined by ELISA assay. pAAV-Insulin plasmids exhibit insulin expression after transfection.

### Example 2: Modified Gck Nucleic Acids

The following modified human glucokinase (Gck) nucleic acid sequences (shown in Table 2) corresponding to SEQ ID Nos: 20-39 89-96, and 163-164 were designed in silico. 5′UTR sequences (SEQ ID NO: 42 or SEQ ID NO: 83) are in BOLD, ORF sequences are underlined (SEQ ID NOs: 61-80 and 162), and 3′ UTR are italicized (SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109). The 5′UTRs having the sequence of SEQ ID NO: 42 was further modified to remove the CTAG at positions 1-4. Accordingly, in certain constructs the 5′UTR included nucleic acids 5-329 of SEQ ID NO: 42.

TABLE 2
Modified Nucleic Acid Sequences Encoding Glucokinase

| SEQ ID | | |
|---|---|---|
| NOs | Name | Sequence |
| Full: 19 | Wildtype | AAGCTTTATTGCGGTAGTTTATCACAGTTAAATTGCTAACGCAGTCA |
| 5′ UTR: 148 | hGck | GTGCTTCTGACACAACAGTCTCGAACTTAAGCTGCAGTGACTCTCTT |
| ORF: 162 | | AAGGTAGCCTTGCAGAAGTTGGTCGTGAGGCACTGGGCAGGTAAGT |
| 3′ UTR: 149 | | ATCAAGGTTACAAGACAGGTTTAAGGAGACCAATAGAAACTGGGCT |
| | | TGTCGAGACAGAGAAGACTCTTGCGTTTCTGATAGGCACCTATTGGT |
| | | CTTACTGACATCCACTTTGCCTTTCTCTCCACAGGTGTCCACTCCCA |
| | | GTTCAATTACAGCTCTTAAGGCTAGAGTACTTAATACGACTCACTAT |
| | | AGGCTAGCCTCGAGAATTCTGCCATGGCGATGGATGTCACAAGGAGC |
| | | CAGGCCCAGACAGCCTTGACTCTGGTAGAGCAGATCCTGGCAGAGTTCC |
| | | AGCTGCAGGAGGAGGACCTGAAGAAGGTGATGAGACGGATGCAGAAGG |
| | | AGATGGACCGCGGCCTGAGGCTGGAGACCCATGAAGAGGCCAGTGTGA |
| | | AGATGCTGCCCACCTACGTGCGCTCCACCCCAGAAGGCTCAGAAGTCGG |
| | | GGACTTCCTCTCCCTGGACCTGGGTGGCACTAACTTCAGGGTGATGCTGG |
| | | TGAAGGTGGGAGAAGGTGAGGAGGGGCAGTGGAGCGTGAAGACCAAAC |
| | | ACCAGATGTACTCCATCCCCGAGGACGCCATGACCGGCACTGCTGAGAT |
| | | GCTCTTCGACTACATCTCTGAGTGCATCTCCGACTTCCTGGACAAGCATC |
| | | AGATGAAACACAAGAAGCTGCCCCTGGGCTTCACCTTCTCCTTTCCTGTG |
| | | AGGCACGAAGACATCGATAAGGGCATCCTTCTCAACTGGACCAAGGGCT |
| | | TCAAGGCCTCAGGAGCAGAAGGGAACAATGTCGTGGGGCTTCTGCGAG |
| | | ACGCTATCAAACGGAGAGGGGACTTTGAAATGGATGTGGTGGCAATGGT |
| | | GAATGACACGGTGGCCACGATGATCTCCTGCTACTACGAAGACCATCAG |
| | | TGCGAGGTCGGCATGATCGTGGGCACGGGCTGCAATGCCTGCTACATGG |
| | | AGGAGATGCAGAATGTGGAGCTGGTGGAGGGGGACGAGGGCCGCATGT |
| | | GCGTCAATACCGAGTGGGGCGCCTTCGGGGACTCCGGCGAGCTGGACGA |
| | | GTTCCTGCTGGAGTATGACCGCCTGGTGGACGAGAGCTCTGCAAACCCC |
| | | GGTCAGCAGCTGTATGAGAAGCTCATAGGTGGCAAGTACATGGGCGAGC |
| | | TGGTGCGGCTTGTGCTGCTCAGGCTCGTGGACGAAAACCTGCTCTTCCAC |
| | | GGGGAGGCCTCCGAGCAGCTGCGCACACGCGGAGCCTTCGAGACGCGCT |
| | | TCGTGTCGCAGGTGGAGAGCGACACGGGCGACCGCAAGCAGATCTACA |
| | | ACATCCTGAGCACGCTGGGGCTGCGACCCTCGACCACCGACTGCGACAT |
| | | CGTGCGCCGCGCCTGCGAGAGCGTGTCTACGCGCGCTGCGCACATGTGC |
| | | TCGGCGGGGCTGGCGGGCGTCATCAACCGCATGCGCGAGAGCCGCAGC |
| | | GAGGACGTAATGCGCATCACTGTGGGCGTGGATGGCTCCGTGTACAAGC |
| | | TGCACCCCAGCTTCAAGGAGCGGTTCCATGCCAGCGTGCGCAGGCTGAC |
| | | GCCCAGCTGCGAGATCACCTTCATCGAGTCGGAGGAGGGCAGTGGCCGG |
| | | GGCGCGGCCCTGGTCTCGGCGGTGGCCTGTAAGAAGGCCTGTATGCTGG |
| | | GCCAGTGAACGCAGCTGCTAGCTAATCGATACCGTCGACCTCGAGGAATTCA |
| | | CGCGTGGTACCTCTAGAGTCGACCCGGGCGGCCGCTTCCCTTTAGTGAGGG |
| | | TTAATGCTTCGAGCAGACATGATAAGATACATTGATGAGTTTGGACAAACCAC |
| | | AACTAGAATGCAGTGAAAAAAATGCTTTATTTGTGAAATTTGTGATGCTATTGC |
| | | TTTATTTGTAACCATTATAAGCTGCAATAAACAAGTTAACAACAACAATTGCATT |
| | | CATTTTATGTTTCAGGTTCAGGGGGAGATGTGGGAGGTTTTTTAAAGCAAGTA |
| | | AAACCTCTACAAATGTGGTAAAATCCGATAAGGGACTAGAGCAGAAGCTTTTG |
| Full: 20 | Modified | [CTAG]AAGCTTTATTGTGGTAGTTTATCACAGTTAAATTGCTAAGGC |
| 5′ UTR: 42 | hGck-2 | AGTCAGTGCTTCTGACACAACAGTCTCCAACTTAAGCTGCAGTGACT |
| ORF: 61 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACTGTGGCCACCA |
| | | TGATCTCCTGCTACTATGAAGACCATCAGTGTGAGGTGGGCATGATTGT |
| | | GGGCACAGGCTGCAATGCCTGCTACATGGAGGAGATGCAGAATGTGGA |
| | | GCTGGTGGAGGGGGATGAGGGCAGGATGTGTGTCAATACAGAGTGGGG |
| | | AGCCTTTGGGGACTCTGGGGAGCTGGATGAGTTCCTGCTGGAGTATGAC |
| | | AGGCTGGTGGATGAGAGCTCAGCAAACCCTGGTCAGCAGCTGTATGAGA |
| | | AGCTCATAGGTGGCAAGTACATGGGAGAGCTGGTGAGGCTTGTGCTGCT |
| | | CAGGCTGGTGGATGAAAACCTGCTCTTCCATGGGGAGGCCTCTGAGCAG |
| | | CTGAGGACAAGGGGAGCCTTTGAGACCAGGTTTGTGTCCCAGGTGGAGA |
| | | GTGACACAGGAGACAGGAAGCAGATCTACAACATCCTGAGCACCCTGG |
| | | GGCTGAGACCCTCCACCACAGACTGTGACATTGTGAGGAGGGCTTGTGA |
| | | GAGTGTGTCTACCAGGGCTGCCCACATGTGCTCTGCTGGGCTGGCTGGG |
| | | GTCATCAACAGGATGAGGGAGAGCAGGAGTGAGGATGTAATGAGGATC |
| | | ACTGTGGGAGTGGATGGCTCTGTGTACAAGCTGCACCCCTCATTCAAGG |
| | | AGAGGTTCCATGCCAGTGTGAGGAGGCTGACCCCCAGCTGTGAGATCAC |
| | | CTTTATTGAGTCTGAGGAGGGCAGTGGCAGGGGAGCAGCCCTGGTCTCT |
| | | GCTGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 21 | Modified | [CTAG]AAGCTTTATTGTGGTAGTTTATCACAGTTAAATTGCTAAGGC |
| 5′ UTR: 42 | hGck-3 | AGTCAGTGCTTCTGACACAACAGTCTCCAACTTAAGCTGCAGTGACT |
| ORF: 62 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGGGATGCTATCAAAAGGAGAGGG |
| | | GACTTTGAAATGGATGTGGTGGCAATGGTGAATGACACAGTGGCCACAA |
| | | TGATCTCCTGCTACTATGAAGACCATCAGTGTGAGGTGGGCATGATTGT |
| | | GGGCACAGGCTGCAATGCCTGCTACATGGAGGAGATGCAGAATGTGGA |
| | | GCTGGTGGAGGGGGATGAGGGCAGAATGTGTGTCAATACAGAGTGGGG |
| | | AGCCTTTGGGGACTCTGGGGAGCTGGATGAGTTCCTGCTGGAGTATGAC |
| | | AGACTGGTGGATGAGAGCTCAGCAAACCCTGGTCAGCAGCTGTATGAGA |
| | | AGCTCATAGGTGGCAAGTACATGGGAGAGCTGGTGAGACTTGTGCTGCT |
| | | CAGGCTGGTGGATGAAAACCTGCTCTTCCATGGGGAGGCCTCTGAGCAG |
| | | CTGAGAACAAGAGGAGCCTTTGAGACCAGATTTGTGTCCCAGGTGGAGT |
| | | CTGACACAGGAGACAGGAAGCAGATCTACAACATCCTGAGCACCCTGG |
| | | GGCTGAGGCCCTCCACCACAGACTGTGACATTGTGAGAAGAGCCTGTGA |
| | | GTCTGTGTCTACCAGGGCTGCCCACATGTGCTCTGCTGGGCTGGCTGGGG |
| | | TCATCAACAGAATGAGGGAGAGCAGGTCTGAGGATGTAATGAGAATCA |
| | | CTGTGGGAGTGGATGGCTCTGTGTACAAGCTGCACCCCTCATTCAAGGA |
| | | GAGATTCCATGCCTCTGTGAGAAGGCTGACCCCCAGCTGTGAGATCACC |
| | | TTTATTGAGTCTGAGGAGGGCAGTGGCAGAGGAGCTGCCCTGGTCTCTG |
| | | CTGTGGCCTGTAAGAAGGCCTGTATGCTGGGCCAGTGAAGCCAGCTGCCT |
| | | TCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 22 | Modified | [CTAG]AAGCTTTATTGTGGTAGTTTATCACAGTTAAATTGCTAAGGC |
| 5′ UTR: 42 | hGck-4 | AGTCAGTGCTTCTGACACAACAGTCTCCAACTTAAGCTGCAGTGACT |
| ORF: 63 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACTGGCACTGCTGAGATGCTCTTTGACTACATCTCTGAG |
| | | TGCATCTCAGACTTCCTGGACAAGCATCAGATGAAACACAAGAAGCTGC |
| | | CCCTGGGCTTCACCTTCTCCTTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGGGATGCTATCAAAAGGAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTAGGCATGATTGTG |
| | | GGCACTGGCTGCAATGCCTGCTACATGGAGGAGATGCAGAATGTGGAGC |
| | | TGGTGGAGGGGGATGAGGGCAGAATGTGTGTCAATACTGAGTGGGGTG |
| | | CCTTTGGGGACTCAGGGGAGCTGGATGAGTTCCTGCTGGAGTATGACAG |
| | | ACTGGTGGATGAGAGCTCTGCAAACCCTGGTCAGCAGCTGTATGAGAAG |
| | | CTCATAGGTGGCAAGTACATGGGAGAGCTGGTGAGACTTGTGCTGCTCA |
| | | GGCTGGTGGATGAAAACCTGCTCTTCCATGGGGAGGCCTCTGAGCAGCT |
| | | GAGAACAAGAGGAGCCTTTGAGACCAGATTTGTGTCCCAGGTGGAGTCT |
| | | GACACTGGTGACAGGAAGCAGATCTACAACATCCTGAGCACACTGGGGC |
| | | TGAGGCCCTCCACCACAGACTGTGACATTGTGAGAAGAGCTTGTGAGAG |
| | | TGTGTCTACCAGGGCTGCTCACATGTGCAGTGCTGGGCTGGCTGGGGTC |
| | | ATCAACAGAATGAGGGAGAGCAGGTCTGAGGATGTAATGAGAATCACT |
| | | GTGGGTGTGGATGGCAGTGTGTACAAGCTGCACCCCTCATTCAAGGAGA |
| | | GATTCCATGCCAGTGTGAGAAGGCTGACCCCCAGCTGTGAGATCACCTT |
| | | CATTGAGTCTGAGGAGGGCAGTGGCAGGGGTGCAGCCCTGGTCAGTGCA |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 23 | Modified | [CTAG]AAGCTTTATTGTGGTAGTTTATCACAGTTAAATTGCTAAGGC |
| 5′ UTR: 42 | hGck-5 | AGTCAGTGCTTCTGACACAACAGTCTCCAACTTAAGCTGCAGTGACT |
| ORF: 64 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCAGACTTCCTGGACAAGCATCAGATGAAACACAAGAAGCTGC |
| | | CCCTGGGCTTCACCTTCTCCTTTCCTGTGAGGCATGAAGACATAGATAAG |
| | | GGCATCCTTCTCAACTGGACCAAGGGCTTCAAGGCCTCAGGAGCAGAAG |
| | | GGAACAATGTGGTGGGGCTTCTGAGAGATGCTATCAAAAGGAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTGGGCATGATAGTG |
| | | GGCACTGGCTGCAATGCCTGCTACATGGAGGAGATGCAGAATGTGGAGC |
| | | TGGTGGAGGGGGATGAGGGCAGGATGTGTGTCAATACTGAGTGGGGAG |
| | | CCTTTGGGGACTCTGGGGAGCTGGATGAGTTCCTGCTGGAGTATGACAG |
| | | GCTGGTGGATGAGAGCTCAGCAAACCCTGGTCAGCAGCTGTATGAGAAG |
| | | CTCATAGGTGGCAAGTACATGGGGGAGCTGGTGAGGCTTGTGCTGCTCA |
| | | GGCTGGTGGATGAAAACCTGCTCTTCCATGGGGAGGCCTCTGAGCAGCT |
| | | GAGAACAAGGGGAGCCTTTGAGACCAGGTTTGTGTCCCAGGTGGAGTCT |
| | | GACACTGGGGACAGGAAGCAGATCTACAACATCCTGAGCACCCTGGGG |
| | | CTGAGGCCCTCCACCACTGACTGTGACATAGTGAGGAGAGCCTGTGAGT |
| | | CTGTGTCTACCAGGGCTGCCCACATGTGCTCTGCTGGGCTGGCAGGGGT |
| | | CATCAACAGGATGAGGGAGAGCAGATCTGAGGATGTAATGAGGATCAC |
| | | TGTGGGTGTGGATGGCTCTGTGTACAAGCTGCACCCCTCATTCAAGGAG |
| | | AGGTTCCATGCCTCTGTGAGGAGGCTGACCCCCAGCTGTGAGATCACCT |
| | | TTATAGAGTCTGAGGAGGGCAGTGGCAGAGGGGCAGCCCTGGTCTCTGC |
| | | AGTGGCCTGTAAGAAGGCCTGTATGCTGGGCCAGTGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 24 | Modified | [CTAG]AAGCTTTATTGTGGTAGTTTATCACAGTTAAATTGCTAAGGC |
| 5′ UTR: 42 | hGck-6 | AGTCAGTGCTTCTGACACAACAGTCTCCAACTTAAGCTGCAGTGACT |
| ORF: 65 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATAGT |
| | | GGGCACAGGCTGCAATGCCTGCTACATGGAGGAGATGCAGAATGTGGA |
| | | GCTGGTGGAGGGGGATGAGGGCAGGATGTGTGTCAATACTGAGTGGGG |
| | | AGCCTTTGGGGACTCTGGGGAGCTGGATGAGTTCCTGCTGGAGTATGAC |
| | | AGGCTGGTGGATGAGAGCTCAGCAAACCCTGGTCAGCAGCTGTATGAGA |
| | | AGCTCATAGGTGGCAAGTACATGGGGGAGCTGGTGAGGCTTGTGCTGCT |
| | | CAGGCTGGTGGATGAAAACCTGCTCTTCCATGGGGAGGCCTCAGAGCAG |
| | | CTGAGAACAAGGGGAGCCTTTGAGACCAGGTTTGTGTCCCAGGTGGAGA |
| | | GTGACACAGGTGACAGGAAGCAGATCTACAACATCCTGAGCACCCTGGG |
| | | GCTGAGACCCTCCACCACAGACTGTGACATTGTGAGGAGGGCCTGTGAG |
| | | AGTGTGTCTACCAGGGCTGCCCACATGTGCTCTGCTGGGCTGGCAGGAG |
| | | TCATCAACAGGATGAGGGAGAGCAGGAGTGAGGATGTAATGAGGATCA |
| | | CTGTGGGAGTGGATGGCTCTGTGTACAAGCTGCACCCCTCATTCAAGGA |
| | | GAGGTTCCATGCCAGTGTGAGAAGGCTGACCCCCAGCTGTGAGATCACC |
| | | TTTATAGAGTCTGAGGAGGGCAGTGGCAGGGGAGCAGCCCTGGTCTCTG |
| | | CTGTGGCCTGTAAGAAGGCCTGTATGCTGGGCCAGTGAAGCCAGCTGCCT |
| | | TCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 25 | Modified | [CTAG]AAGCTTTATTGTGGTAGTTTATCACAGTTAAATTGCTAAGGC |
| 5′ UTR: 42 | hGck-7 | AGTCAGTGCTTCTGACACAACAGTCTCCAACTTAAGCTGCAGTGACT |
| ORF: 66 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACTGTGGCCACAA |
| | | TGATCTCCTGCTACTATGAAGACCATCAGTGTGAGGTGGGCATGATAGT |
| | | GGGCACAGGCTGCAATGCCTGCTACATGGAGGAGATGCAGAATGTGGA |
| | | GCTGGTGGAGGGGGATGAGGGCAGAATGTGTGTCAATACAGAGTGGGG |
| | | AGCCTTTGGGGACTCTGGGGAGCTGGATGAGTTCCTGCTGGAGTATGAC |
| | | AGACTGGTGGATGAGAGCTCAGCAAACCCTGGTCAGCAGCTGTATGAGA |
| | | AGCTCATAGGTGGCAAGTACATGGGAGAGCTGGTGAGGCTTGTGCTGCT |
| | | CAGGCTGGTGGATGAAAACCTGCTCTTCCATGGGGAGGCCTCTGAGCAG |
| | | CTGAGGACAAGAGGAGCCTTTGAGACCAGATTTGTGTCACAGGTGGAGT |
| | | CTGACACTGGAGACAGAAAGCAGATCTACAACATCCTGAGCACACTGGG |
| | | GCTGAGGCCCTCCACCACTGACTGTGACATAGTGAGGAGAGCTTGTGAG |
| | | AGTGTGTCTACCAGAGCTGCCCACATGTGCTCTGCTGGGCTGGCTGGGG |
| | | TCATCAACAGGATGAGAGAGAGCAGGAGTGAGGATGTAATGAGAATCA |
| | | CTGTGGGGGTGGATGGCTCTGTGTACAAGCTGCACCCCTCATTCAAGGA |
| | | GAGGTTCCATGCCTCTGTGAGGAGGCTGACCCCCAGCTGTGAGATCACC |
| | | TTTATTGAGTCTGAGGAGGGCAGTGGAAGGGGAGCAGCCCTGGTCTCTG |
| | | CAGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |
| Full: 26 | Modified | [CTAG]AAGCTTTATTGTGGTAGTTTATCACAGTTAAATTGCTAAGGC |
| 5′ UTR: 42 | hGck-8 | AGTCAGTGCTTCTGACACAACAGTCTCCAACTTAAGCTGCAGTGACT |
| ORF: 67 | | CTCTTAAGGTAGCCTTGCAGAAGTTGGTCCTGAGGCACTGGGCAGG |
| 3′ UTR: 59 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGGATGCAGAAGGAGATGGACAGGGGCCTGAGGC |
| | | TGGAGACCCATGAAGAGGCCAGTGTGAAGATGCTGCCCACCTATGTGAG |
| | | GTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCTGACTTCCTGGACAAGCATCAGATGAAACACAAGAAGCTG |
| | | CCCCTGGGCTTCACCTTCTCATTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGGAGAGGG |
| | | GACTTTGAAATGGATGTGGTGGCAATGGTGAATGACACAGTGGCCACTA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCAGGATGTGTGTCAATACTGAGTGGGGA |
| | | GCCTTTGGGGACTCTGGGGAGCTGGATGAGTTCCTGCTGGAGTATGACA |
| | | GGCTGGTGGATGAGAGCTCAGCAAACCCTGGTCAGCAGCTGTATGAGAA |
| | | GCTCATAGGTGGCAAGTACATGGGAGAGCTGGTGAGACTTGTGCTGCTC |
| | | AGGCTGGTGGATGAAAACCTGCTCTTCCATGGGGAGGCCTCAGAGCAGC |
| | | TGAGGACAAGGGGAGCCTTTGAGACCAGGTTTGTGTCCCAGGTGGAGAG |
| | | TGACACTGGAGACAGGAAGCAGATCTACAACATCCTGAGCACCCTGGGG |
| | | CTGAGGCCCTCTACCACAGACTGTGACATAGTGAGAAGGGCCTGTGAGT |
| | | CTGTGTCTACCAGGGCTGCCCACATGTGCTCTGCTGGGCTGGCTGGAGTC |
| | | ATCAACAGAATGAGGGAGAGCAGAAGTGAGGATGTAATGAGGATCACT |
| | | GTGGGAGTGGATGGCTCTGTGTACAAGCTGCACCCCTCATTCAAGGAGA |
| | | GGTTCCATGCCAGTGTGAGGAGGCTGACCCCCAGCTGTGAGATCACCTT |
| | | TATTGAGTCTGAGGAGGGCAGTGGCAGGGGAGCTGCCCTGGTCTCTGCT |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 27 | Modified | [CTAG]AAGCTTTATTGTGGTAGTTTATCACAGTTAAATTGCTAAGGC |
| 5′ UTR: 42 | hGck-9 | AGTCAGTGCTTCTGACACAACAGTCTCCAACTTAAGCTGCAGTGACT |
| ORF: 68 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTTGTGGGGCTTCTGAGAGATGCTATCAAAAGAAGAGGG |
| | | GACTTTGAAATGGATGTGGTGGCAATGGTGAATGACACAGTGGCCACTA |
| | | TGATCTCCTGCTACTATGAAGACCATCAGTGTGAGGTTGGCATGATTGTG |
| | | GGCACAGGCTGCAATGCCTGCTACATGGAGGAGATGCAGAATGTGGAG |
| | | CTGGTGGAGGGGGATGAGGGCCGCATGTGTGTCAATACTGAGTGGGGCG |
| | | CCTTTGGGGACTCTGGAGAGCTGGATGAGTTCCTGCTGGAGTATGACCG |
| | | CCTGGTGGATGAGAGCTCAGCAAACCCAGGTCAGCAGCTGTATGAGAAG |
| | | CTCATAGGTGGCAAGTACATGGGAGAGCTGGTGCGGCTTGTGCTGCTCA |
| | | GGTTGGTGGATGAAAACCTGCTCTTCCATGGGGAGGCCAGTGAGCAGCT |
| | | GCGCACACGCGGAGCCTTTGAGACGCGCTTTGTGTCACAAGTGGAGTCT |
| | | GACACAGGAGACCGCAAGCAGATCTACAACATCCTGAGCACACTGGGG |
| | | CTGCGGCCCTCCACCACTGACTGTGACATTGTGCGCCGCGCCTGTGAGA |
| | | GTGTGTCTACGCGCGCTGCGCACATGTGCTCAGCGGGGCTGGCGGGAGT |
| | | CATCAACCGCATGCGCGAGAGCCGCTCTGAGGATGTAATGCGCATCACT |
| | | GTGGGAGTGGATGGCAGTGTGTACAAGCTGCACCCCAGCTTCAAGGAGC |
| | | GGTTCCATGCCTCAGTGCGCAGGCTGACCCCCAGCTGTGAGATCACCTTT |
| | | ATTGAGTCTGAGGAGGGCAGTGGCAGAGGCGCGGCCCTGGTCAGCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 28 | Modified | [CTAG]AAGCTTTATTGTGGTAGTTTATCACAGTTAAATTGCTAAGGC |
| 5′ UTR: 42 | hGck-10 | AGTCAGTGCTTCTGACACAACAGTCTCCAACTTAAGCTGCAGTGACT |
| ORF: 69 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGCGGGATGCTATCAAAAGAAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCCGCATGTGTGTCAATACAGAGTGGGGC |
| | | GCCTTTGGGGACAGCGGAGAGCTGGATGAGTTCCTGCTGGAGTATGACC |
| | | GCCTGGTGGATGAGAGCTCAGCAAACCCAGGTCAGCAGCTGTATGAGAA |
| | | GCTCATAGGTGGCAAGTACATGGGAGAGCTGGTGCGGCTTGTGCTGCTC |
| | | AGGCTGGTGGATGAAAACCTGCTCTTCCATGGGGAGGCCTCTGAGCAGC |
| | | TGCGCACACGCGGAGCCTTTGAGACGCGCTTTGTGAGCCAGGTGGAGAG |
| | | TGACACAGGAGACCGCAAGCAGATCTACAACATCCTGAGCACCCTGGGG |
| | | CTGCGGCCCAGCACCACTGACTGTGACATTGTGCGCCGCGCCTGTGAGT |
| | | CAGTGTCTACGCGCGCTGCGCACATGTGCAGCGCGGGGCTGGCGGGAGT |
| | | CATCAACCGCATGCGCGAGAGCCGCTCTGAGGATGTAATGCGCATCACT |
| | | GTGGGAGTGGATGGCAGTGTGTACAAGCTGCACCCCAGCTTCAAGGAGC |
| | | GGTTCCATGCCTCAGTGCGCAGGCTGACCCCCAGCTGTGAGATCACCTTT |
| | | ATTGAGTCTGAGGAGGGCAGTGGCAGAGGCGCGGCCCTGGTCAGCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 29 | Modified | [CTAG]AAGCTTTATTGTGGTAGTTTATCACAGTTAAATTGCTAAGGC |
| 5′ UTR: 42 | hGck-11 | AGTCAGTGCTTCTGACACAACAGTCTCCAACTTAAGCTGCAGTGACT |
| ORF: 70 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCG |
| | | GAGGATGCCATGACAGGCACTGCTGAGATGCTCTTTGACTACATCTCTG |
| | | AGTGCATCTCAGACTTCCTGGACAAGCATCAGATGAAACACAAGAAGCT |
| | | GCCCCTGGGCTTCACCTTCTCCTTTCCTGTGAGGCATGAAGACATTGATA |
| | | AGGGCATCCTTCTCAACTGGACCAAGGGCTTCAAGGCCTCAGGAGCAGA |
| | | AGGGAACAATGTTGTGGGGCTTCTGAGAGATGCTATCAAAAGAAGAGG |
| | | GGACTTTGAAATGGATGTGGTGGCAATGGTGAATGACACAGTGGCCACT |
| | | ATGATCTCCTGCTACTATGAAGACCATCAGTGTGAGGTAGGCATGATAG |
| | | TGGGCACAGGCTGCAATGCCTGCTACATGGAGGAGATGCAGAATGTGGA |
| | | GCTGGTGGAGGGGGATGAGGGCCGCATGTGTGTCAATACTGAGTGGGGC |
| | | GCCTTTGGGGACAGTGGAGAGCTGGATGAGTTCCTGCTGGAGTATGACC |
| | | GCCTGGTGGATGAGAGCTCTGCAAACCCAGGTCAGCAGCTGTATGAGAA |
| | | GCTCATAGGTGGCAAGTACATGGGAGAGCTGGTGCGGCTTGTGCTGCTC |
| | | AGGCTAGTGGATGAAAACCTGCTCTTCCATGGGGAGGCCTCAGAGCAGC |
| | | TGCGCACACGCGGAGCCTTTGAGACGCGCTTTGTGTCTCAAGTGGAGAG |
| | | TGACACAGGTGACCGCAAGCAGATCTACAACATCCTGAGCACTCTGGGG |
| | | CTGCGGCCCTCAACCACAGACTGTGACATTGTGCGCCGCGCCTGTGAGT |
| | | CTGTGTCTACGCGCGCTGCGCACATGTGCAGTGCGGGGCTGGCGGGAGT |
| | | CATCAACCGCATGCGCGAGAGCCGCTCAGAGGATGTAATGCGCATCACT |
| | | GTGGGTGTGGATGGCTCTGTGTACAAGCTGCACCCCAGCTTCAAGGAGC |
| | | GGTTCCATGCCAGTGTGCGCAGGCTGACTCCCAGCTGTGAGATCACCTTT |
| | | ATAGAGTCAGAGGAGGGCAGTGGCAGAGGCGCGGCCCTGGTCTCCGCG |
| | | GTGGCCTGTAAGAAGGCCTGTATGCTGGGCCAGTGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 30 | Modified | [CTAG]AAGCTTTATTGTGGTAGTTTATCACAGTTAAATTGCTAAGGC |
| 5′ UTR: 42 | hGck-12 | AGTCAGTGCTTCTGACACAACAGTCTCCAACTTAAGCTGCAGTGACT |
| ORF: 71 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACCGCGGCCTGAGGC |
| | | TGGAGACCCATGAAGAGGCCAGTGTGAAGATGCTGCCCACCTATGTGCG |
| | | CTCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCTGACTTCCTGGACAAGCATCAGATGAAACACAAGAAGCTGC |
| | | CCCTGGGCTTCACCTTCTCATTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGAGATGCTATCAAAAGAAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTTGGCATGATTGTG |
| | | GGCACTGGCTGCAATGCCTGCTACATGGAGGAGATGCAGAATGTGGAGC |
| | | TGGTGGAGGGGGATGAGGGCCGCATGTGTGTCAATACTGAGTGGGGCGC |
| | | CTTTGGGGACTCTGGTGAGCTGGATGAGTTCCTGCTGGAGTATGACCGC |
| | | CTGGTGGATGAGAGCTCTGCAAACCCTGGTCAGCAGCTGTATGAGAAGC |
| | | TCATAGGTGGCAAGTACATGGGTGAGCTGGTGCGGCTTGTGCTGCTCAG |
| | | GTTAGTGGATGAAAACCTGCTCTTCCATGGGGAGGCCTCTGAGCAGCTG |
| | | CGCACACGCGGAGCCTTTGAGACGCGCTTTGTGTCTCAAGTGGAGTCTG |
| | | ACACTGGTGACCGCAAGCAGATCTACAACATCCTGAGCACTCTGGGGCT |
| | | GAGACCCTCTACCACTGACTGTGACATTGTGCGCCGCGCCTGTGAGTCTG |
| | | TGTCTACGCGCGCTGCGCACATGTGCTCTGCGGGGCTGGCGGGTGTCAT |
| | | CAACCGCATGCGCGAGAGCCGCTCTGAGGATGTAATGCGCATCACTGTG |
| | | GGTGTGGATGGCTCTGTGTACAAGCTGCACCCCTCATTCAAGGAGCGGT |
| | | TCCATGCCTCTGTGCGCAGGCTGACTCCCAGCTGTGAGATCACCTTTATT |
| | | GAGTCTGAGGAGGGCAGTGGCAGAGGCGCGGCCCTGGTCTCTGCGGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATCCGATAAGGGACTAGAGCAGA |
| | | AGCTTTTG |
| Full: 31 | Modified | [CTAG]AAGCTTTATTGTGGTAGTTTATCACAGTTAAATTGCTAAGGC |
| 5′ UTR: 42 | hGck-13 | AGTCAGTGCTTCTGACACAACAGTCTCCAACTTAAGCTGCAGTGACT |
| ORF: 72 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTTGTGGGGCTTCTGAGAGATGCTATCAAAAGAAGAGGG |
| | | GACTTTGAAATGGATGTGGTGGCAATGGTGAATGACACAGTGGCCACTA |
| | | TGATCTCCTGCTACTATGAAGACCATCAGTGTGAGGTTGGCATGATTGTG |
| | | GGCACAGGCTGCAATGCCTGCTACATGGAGGAGATGCAGAATGTGGAG |
| | | CTGGTGGAGGGGGATGAGGGCAGGATGTGTGTCAATACTGAGTGGGGA |
| | | GCCTTTGGGGACTCTGGAGAGCTGGATGAGTTCCTGCTGGAGTATGACA |
| | | GACTGGTGGATGAGAGCTCAGCAAACCCAGGTCAGCAGCTGTATGAGA |
| | | AGCTCATAGGTGGCAAGTACATGGGAGAGCTGGTGAGGCTTGTGCTGCT |
| | | CAGGTTGGTGGATGAAAACCTGCTCTTCCATGGGGAGGCCAGTGAGCAG |
| | | CTGAGAACAAGGGGAGCCTTTGAGACAAGATTTGTGTCACAGGTGGAGT |
| | | CTGACACAGGAGACAGGAAGCAGATCTACAACATCCTGAGCACCCTGG |
| | | GGCTGAGACCCTCCACCACTGACTGTGACATTGTGAGGAGAGCCTGTGA |
| | | GAGTGTGTCTACCAGGGCTGCTCACATGTGCTCAGCAGGGCTGGCTGGA |
| | | GTCATCAACAGAATGAGGGAGAGCAGATCTGAGGATGTAATGAGGATC |
| | | ACTGTGGGTGTGGATGGCAGTGTGTACAAGCTGCACCCCAGCTTCAAGG |
| | | AGAGATTCCATGCCTCAGTGAGGAGGCTGACCCCCAGCTGTGAGATCAC |
| | | CTTCATTGAGTCTGAGGAGGGCAGTGGCAGAGGAGCAGCCCTGGTCTCT |
| | | GCTGTGGCCTGTAAGAAGGCCTGTATGCTGGGCCAGTGAAGCCAGCGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 32 | Modified | [CTAG]AAGCTTTATTGTGGTAGTTTATCACAGTTAAATTGCTAAGGC |
| 5′ UTR: 42 | hGck-14 | AGTCAGTGCTTCTGACACAACAGTCTCCAACTTAAGCTGCAGTGACT |
| ORF: 73 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTGGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCTG |
| | | AGGATGCCATGACAGGCACTGCTGAGATGCTCTTTGACTACATCTCTGA |
| | | GTGCATCTCAGACTTCCTGGACAAGCATCAGATGAAACACAAGAAGCTG |
| | | CCCCTGGGCTTCACCTTCTCCTTTCCTGTGAGGCATGAAGACATTGATAA |
| | | GGGCATCCTTCTCAACTGGACCAAGGGCTTCAAGGCCTCAGGAGCAGAA |
| | | GGGAACAATGTGGTGGGGCTTCTGAGAGATGCTATCAAAAGAAGAGGG |
| | | GACTTTGAAATGGATGTGGTGGCAATGGTGAATGACACAGTGGCCACCA |
| | | TGATCTCCTGCTACTATGAAGACCATCAGTGTGAGGTGGGCATGATTGT |
| | | GGGCACTGGCTGCAATGCCTGCTACATGGAGGAGATGCAGAATGTGGAG |
| | | CTGGTGGAGGGGGATGAGGGCAGAATGTGTGTCAATACAGAGTGGGGA |
| | | GCCTTTGGGGACTCTGGAGAGCTGGATGAGTTCCTGCTGGAGTATGACA |
| | | GACTGGTGGATGAGAGCTCAGCAAACCCAGGTCAGCAGCTGTATGAGA |
| | | AGCTCATAGGTGGCAAGTACATGGGAGAGCTGGTGAGACTTGTGCTGCT |
| | | CAGGCTGGTGGATGAAAACCTGCTCTTCCATGGGGAGGCCAGTGAGCAG |
| | | CTGAGAACAAGAGGAGCCTTTGAGACCAGATTTGTGAGCCAGGTGGAGT |
| | | CAGACACAGGAGACAGAAAGCAGATCTACAACATCCTGAGCACCCTGG |
| | | GGCTGAGACCCAGCACCACTGACTGTGACATTGTGAGAAGAGCCTGTGA |
| | | GTCTGTGTCTACCAGAGCTGCCCACATGTGCAGTGCTGGGCTGGCTGGA |
| | | GTCATCAACAGAATGAGAGAGAGCAGATCAGAGGATGTAATGAGAATC |
| | | ACTGTGGGTGTGGATGGCTCTGTGTACAAGCTGCACCCCAGCTTCAAGG |
| | | AGAGATTCCATGCCAGTGTGAGAAGGCTGACCCCCAGCTGTGAGATCAC |
| | | CTTTATTGAGTCAGAGGAGGGCAGTGGCAGAGGAGCAGCCCTGGTCTCT |
| | | GCTGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 33 | Modified | [CTAG]AAGCTTTATTGTGGTAGTTTATCACAGTTAAATTGCTAAGGC |
| 5′ UTR: 42 | hGck-15 | AGTCAGTGCTTCTGACACAACAGTCTCCAACTTAAGCTGCAGTGACT |
| ORF: 74 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGTGTGAAGACCAAACACCAGATGTACTCCATCCCA |
| | | GAGGATGCCATGACAGGCACTGCTGAGATGCTCTTTGACTACATCTCTG |
| | | AGTGCATCTCAGACTTCCTGGACAAGCATCAGATGAAACACAAGAAGCT |
| | | GCCCCTGGGCTTCACCTTCTCCTTTCCTGTGAGGCATGAAGACATTGATA |
| | | AGGGCATCCTTCTCAACTGGACCAAGGGCTTCAAGGCCTCAGGAGCAGA |
| | | AGGGAACAATGTTGTGGGGCTTCTGAGAGATGCTATCAAAAGAAGAGG |
| | | GGACTTTGAAATGGATGTGGTGGCAATGGTGAATGACACAGTGGCCACT |
| | | ATGATCTCCTGCTACTATGAAGACCATCAGTGTGAGGTAGGCATGATAG |
| | | TGGGCACAGGCTGCAATGCCTGCTACATGGAGGAGATGCAGAATGTGGA |
| | | GCTGGTGGAGGGGGATGAGGGCAGGATGTGTGTCAATACTGAGTGGGG |
| | | AGCCTTTGGGGACAGTGGAGAGCTGGATGAGTTCCTGCTGGAGTATGAC |
| | | AGACTGGTGGATGAGAGCTCTGCAAACCCTGGTCAGCAGCTGTATGAGA |
| | | AGCTCATAGGTGGCAAGTACATGGGTGAGCTGGTGAGGCTTGTGCTGCT |
| | | CAGGCTAGTGGATGAAAACCTGCTCTTCCATGGGGAGGCCTCAGAGCAG |
| | | CTGAGAACAAGGGGAGCCTTTGAGACAAGATTTGTGTCTCAGGTGGAGA |
| | | GTGACACTGGAGACAGGAAGCAGATCTACAACATCCTGAGCACACTGG |
| | | GGCTGAGACCCTCAACCACTGACTGTGACATTGTGAGGAGAGCCTGTGA |
| | | GTCTGTGTCTACAAGGGCTGCACACATGTGCAGTGCTGGGCTGGCAGGT |
| | | GTCATCAACAGAATGAGGGAGAGCAGATCAGAGGATGTAATGAGGATC |
| | | ACTGTGGGAGTGGATGGCTCTGTGTACAAGCTGCACCCCAGCTTCAAGG |
| | | AGAGATTCCATGCCAGTGTGAGGAGGCTGACCCCCAGCTGTGAGATCAC |
| | | CTTTATAGAGTCAGAGGAGGGCAGTGGCAGAGGTGCTGCCCTGGTCTCT |
| | | GCAGTGGCCTGTAAGAAGGCCTGTATGCTGGGCCAGTGAAGCCAGCTGC |
| | | CTTCCCTTTACTGTGGGTTAATGCTTGCAGCAGACATGATAAGATACATTGATG |
| | | AGTTTGGACAAACCACAACTAGAATGCAGTGAAAAAAATGCTTTATTTGTGAAA |
| | | TTTGTGATGCTATTGCTTTATTTGTAACCATTATAAGCTGCAATAAACAAGTTAA |
| | | CAACAACAATTGCATTCATTTTATGTTTCAGGTTCAGGGGGAGATGTGGGAGG |
| | | TTTTTTAAAGCAAGTAAAACCTCTACAAATGTGGTAAAATCCGATAAGGGACTA |
| | | GAGCAGAAGCTTTTG |
| Full: 34 | Modified | [CTAG]AAGCTTTATTGTGGTAGTTTATCACAGTTAAATTGCTAAGGC |
| 5′ UTR: 42 | hGck-16 | AGTCAGTGCTTCTGACACAACAGTCTCCAACTTAAGCTGCAGTGACT |
| ORF: 75 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCTATGGATGTCACAAGGAGCCAGGCCCAGACAGCCTTGACT |
| | | CTGGTAGAGCAGATCCTGGCAGAGTTCCAGCTGCAGGAGGAGGACCTGA |
| | | AGAAGGTGATGAGAAGAATGCAGAAGGAGATGGACAGAGGCCTGAGGC |
| | | TGGAGACCCATGAAGAGGCCAGTGTGAAGATGCTGCCCACCTATGTGAG |
| | | ATCCACCCCAGAAGGCTCAGAAGTTGGGGACTTCCTCTCCCTGGACCTG |
| | | GGTGGCACTAACTTCAGGGTGATGCTGGTGAAGGTGGGAGAAGGTGAG |
| | | GAGGGGCAGTGGTCTGTGAAGACCAAACACCAGATGTACTCCATCCCTG |
| | | AGGATGCCATGACTGGCACTGCTGAGATGCTCTTTGACTACATCTCTGAG |
| | | TGCATCTCTGACTTCCTGGACAAGCATCAGATGAAACACAAGAAGCTGC |
| | | CCCTGGGCTTCACCTTCTCATTTCCTGTGAGGCATGAAGACATTGATAAG |
| | | GGCATCCTTCTCAACTGGACCAAGGGCTTCAAGGCCTCAGGAGCAGAAG |
| | | GGAACAATGTTGTGGGGCTTCTGAGAGATGCTATCAAAAGAAGAGGGG |
| | | ACTTTGAAATGGATGTGGTGGCAATGGTGAATGACACTGTGGCCACTAT |
| | | GATCTCCTGCTACTATGAAGACCATCAGTGTGAGGTTGGCATGATTGTG |
| | | GGCACTGGCTGCAATGCCTGCTACATGGAGGAGATGCAGAATGTGGAGC |
| | | TGGTGGAGGGGGATGAGGGCAGAATGTGTGTCAATACTGAGTGGGGTG |
| | | CCTTTGGGGACTCTGGTGAGCTGGATGAGTTCCTGCTGGAGTATGACAG |
| | | ACTGGTGGATGAGAGCTCTGCAAACCCTGGTCAGCAGCTGTATGAGAAG |
| | | CTCATAGGTGGCAAGTACATGGGTGAGCTGGTGAGACTTGTGCTGCTCA |
| | | GGTTAGTGGATGAAAACCTGCTCTTCCATGGGGAGGCCTCTGAGCAGCT |
| | | GAGAACAAGAGGAGCCTTTGAGACTAGATTTGTGTCTCAGGTGGAGTCT |
| | | GACACTGGTGACAGAAAGCAGATCTACAACATCCTGAGCACTCTGGGGC |
| | | TGAGACCCTCTACCACTGACTGTGACATTGTGAGAAGAGCCTGTGAGTC |
| | | TGTGTCTACTAGAGCTGCTCACATGTGCTCTGCTGGGCTGGCTGGTGTCA |
| | | TCAACAGAATGAGAGAGAGCAGATCTGAGGATGTAATGAGAATCACTG |
| | | TGGGTGTGGATGGCTCTGTGTACAAGCTGCACCCCTCATTCAAGGAGAG |
| | | ATTCCATGCCTCTGTGAGAAGGCTGACTCCCAGCTGTGAGATCACCTTCA |
| | | TTGAGTCTGAGGAGGGCAGTGGCAGAGGTGCTGCCCTGGTCTCTGCTGT |
| | | GGCCTGTAAGAAGGCCTGTATGCTGGGCCAGTGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATCCGATAAGGGACTAGAGCAG |
| | | AAGCTTTTG |
| Full: 35 | Modified | [CTAG]AAGCTTTATTGTGGTAGTTTATCACAGTTAAATTGCTAAGGC |
| 5′ UTR: 42 | hGck-17 | AGTCAGTGCTTCTGACACAACAGTCTCCAACTTAAGCTGCAGTGACT |
| ORF: 76 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGACGTGACCCGGAGCCAGGCTCAAACAGCACTGAC |
| | | TCTCGTCGAGCAGATCTTGGCGGAATTCCAGCTTCAAGAGGAAGATCTG |
| | | AAGAAAGTTATGAGAAGGATGCAGAAGGAGATGGACCGCGGCTTACGA |
| | | CTGGAAACGCACGAGGAGGCCTCCGTGAAAATGCTACCCACCTACGTGC |
| | | GTTCTACACCTGAAGGATCAGAGGTGGGGGATTTTCTCAGTCTGGACTT |
| | | GGGTGGCACTAACTTCCGGGTCATGCTGGTGAAGGTTGGAGAAGGGGAG |
| | | GAAGGCCAGTGGTCGGTGAAAACCAAGCATCAGATGTATAGCATTCCAG |
| | | AGGATGCTATGACAGGCACCGCCGAGATGCTTTTTGACTACATATCCGA |
| | | ATGCATCTCTGATTTCCTCGACAAGCACCAAATGAAACATAAGAAACTG |
| | | CCGCTGGGATTTACTTTCTCATTTCCCGTGAGACACGAGGATATTGACAA |
| | | GGGGATCCTCTTGAATTGGACGAAAGGCTTCAAGGCAAGTGGCGCCGAA |
| | | GGAAACAATGTAGTGGGGCTTCTGAGGGATGCTATTAAGCGCCGGGGTG |
| | | ACTTTGAGATGGACGTTGTGGCAATGGTCAACGATACAGTGGCCACAAT |
| | | GATCAGCTGTTATTACGAGGACCATCAGTGCGAAGTCGGCATGATAGTG |
| | | GGAACTGGGTGTAATGCGTGCTATATGGAGGAAATGCAGAACGTTGAGT |
| | | TAGTGGAAGGCGATGAGGGTAGAATGTGTGTAAATACCGAGTGGGGAG |
| | | CTTTCGGGGACTCCGGCGAACTGGATGAGTTCCTACTCGAATACGACAG |
| | | GCTGGTGGATGAGTCTAGCGCCAACCCTGGCCAGCAATTGTATGAAAAA |
| | | CTGATCGGAGGGAAGTACATGGGTGAGCTTGTCCGCCTCGTTCTGCTGC |
| | | GATTAGTGGACGAGAATCTCCTGTTTCACGGCGAAGCATCCGAGCAGTT |
| | | GCGTACACGGGGAGCCTTCGAAACCAGATTTGTCTCACAAGTGGAGAGT |
| | | GATACTGGGGACAGGAAACAGATTTACAACATCCTTAGCACACTGGGCC |
| | | TACGCCCATCTACGACCGATTGCGACATTGTGCGGCGAGCTTGTGAGTC |
| | | CGTTTCGACTAGAGCCGCACACATGTGCTCAGCTGGTCTCGCCGGAGTA |
| | | ATCAATAGGATGCGCGAAAGCCGGAGTGAGGACGTGATGAGAATAACA |
| | | GTCGGCGTGGATGGGTCTGTCTATAAGCTGCATCCCTCCTTCAAGGAAA |
| | | GGTTTCACGCGAGCGTGCGCCGTCTGACCCCTTCATGTGAGATTACTTTC |
| | | ATCGAATCCGAGGAGGGCAGTGGACGGGGGGCAGCCTTGGTTTCTGCTG |
| | | TGGCCTGCAAAAAGGCATGCATGCTTGGCCAATGAAGCCAGCTGCCTTCC |
| | | CTTTACTGTGGGTTAATGCTTGCAGCAGACATGATAAGATACATTGATGAGTTT |
| | | GGACAAACCACAACTAGAATGCAGTGAAAAAAATGCTTTATTTGTGAAATTTGT |
| | | GATGCTATTGCTTTATTTGTAACCATTATAAGCTGCAATAAACAAGTTAACAACA |
| | | ACAATTGCATTCATTTTATGTTTCAGGTTCAGGGGGAGATGTGGGAGGTTTTTT |
| | | AAAGCAAGTAAAACCTCTACAAATGTGGTAAAATCCGATAAGGGACTAGAGCA |
| | | GAAGCTTTTG |
| Full: 36 | Modified | [CTAG]AAGCTTTATTGTGGTAGTTTATCACAGTTAAATTGCTAAGGC |
| 5′ UTR: 42 | hGck-18 | AGTCAGTGCTTCTGACACAACAGTCTCCAACTTAAGCTGCAGTGACT |
| ORF: 77 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTAGAGCAGATCTTGGCGGAATTCCAGCTTCAAGAGGAAGATCTG |
| | | AAGAAAGTTATGAGAAGGATGCAGAAGGAGATGGACCGCGGCTTAAGG |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGGGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGTACCGCTGAGATGCTTTTTGACTACATATCTG |
| | | AATGCATCTCTGATTTCCTAGACAAGCACCAAATGAAACATAAGAAACT |
| | | GCCGCTGGGATTTACTTTCTCATTTCCTGTGAGACATGAGGATATTGACA |
| | | AGGGGATCCTCTTGAATTGGACAAAAGGCTTCAAGGCAAGTGGCGCAGA |
| | | AGGAAACAATGTAGTGGGGCTTCTGAGGGATGCTATTAAGCGCAGAGGT |
| | | GACTTTGAGATGGATGTTGTGGCAATGGTCAATGATACAGTGGCCACAA |
| | | TGATCAGCTGTTATTATGAGGACCATCAGTGTGAAGTGGGCATGATAGT |
| | | GGGAACTGGGTGTAATGCCTGCTATATGGAGGAAATGCAGAATGTTGAG |
| | | TTAGTGGAAGGTGATGAGGGTAGAATGTGTGTAAATACAGAGTGGGGA |
| | | GCTTTTGGGGACAGCGGAGAACTGGATGAGTTCCTATTAGAATATGACA |
| | | GGCTGGTGGATGAGTCTAGCGCCAACCCTGGCCAGCAATTGTATGAAAA |
| | | ACTGATTGGAGGGAAGTACATGGGTGAGCTTGTCCGCCTTGTTCTGCTGC |
| | | GGTTAGTGGATGAGAATCTCCTGTTTCATGGGGAAGCAAGTGAGCAGTT |
| | | GCGGACAAGGGGAGCCTTTGAAACCAGATTTGTCTCACAAGTGGAGAGT |
| | | GATACTGGGGACAGGAAACAGATTTACAACATCCTTAGCACACTGGGCC |
| | | TAAGACCATCTACCACTGATTGTGACATTGTGCGGCGGGCTTGTGAGTC |
| | | AGTTTCCACTAGAGCCGCACACATGTGCTCAGCTGGTTTGGCTGGAGTA |
| | | ATCAATAGGATGCGCGAAAGCAGGAGTGAGGATGTGATGAGAATAACA |
| | | GTTGGTGTGGATGGGTCTGTCTATAAGCTGCATCCCTCCTTCAAGGAAAG |
| | | GTTTCACGCGTCTGTGCGCAGACTGACCCCTTCATGTGAGATTACTTTCA |
| | | TAGAATCTGAGGAGGGCAGTGGAAGGGGGGCAGCCTTGGTTTCTGCTGT |
| | | GGCCTGCAAAAAGGCATGCATGCTTGGCCAATGAAGCCAGCTGCCTTCCC |
| | | TTTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATCCGATAAGGGACTAGAGCAG |
| | | AAGCTTTTG |
| Full: 37 | Modified | [CTAG]AAGCTTTATTGTGGTAGTTTATCACAGTTAAATTGCTAAGGC |
| 5′ UTR: 42 | hGck-19 | AGTCAGTGCTTCTGACACAACAGTCTCCAACTTAAGCTGCAGTGACT |
| ORF: 78 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTGGAGCAGATCTTGGCGGAATTCCAGCTTCAAGAGGAAGATCTG |
| | | AAGAAAGTTATGAGAAGGATGCAGAAGGAGATGGACCGCGGCTTAAGG |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGGGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGTACCGCTGAGATGCTTTTTGACTACATATCTG |
| | | AATGCATCTCTGATTTCCTGGACAAGCACCAAATGAAACATAAGAAACT |
| | | GCCGCTGGGATTTACTTTCTCATTTCCTGTGAGACATGAGGATATTGACA |
| | | AGGGGATCCTCTTGAATTGGACCAAAGGCTTCAAGGCAAGTGGCGCAGA |
| | | AGGAAACAATGTAGTGGGGCTTCTGAGGGATGCTATTAAGCGCAGAGGT |
| | | GACTTTGAGATGGATGTTGTGGCAATGGTCAATGATACAGTGGCCACAA |
| | | TGATCAGCTGTTATTATGAGGACCATCAGTGTGAAGTGGGCATGATAGT |
| | | GGGAACTGGGTGTAATGCCTGCTATATGGAGGAAATGCAGAATGTTGAG |
| | | TTAGTGGAAGGTGATGAGGGTAGAATGTGTGTAAATACAGAGTGGGGA |
| | | GCTTTTGGGGACAGCGGAGAACTGGATGAGTTCCTACTGGAATATGACA |
| | | GGCTGGTGGATGAGTCTAGCGCCAACCCTGGCCAGCAATTGTATGAAAA |
| | | ACTGATTGGAGGGAAGTACATGGGTGAGCTTGTCCGCCTGGTTCTGCTG |
| | | CGGTTAGTGGATGAGAATCTCCTGTTTCATGGGGAAGCAAGTGAGCAGT |
| | | TGCGGACAAGGGGAGCCTTTGAAACCAGATTTGTCTCACAAGTGGAGAG |
| | | TGATACTGGGGACAGGAAACAGATTTACAACATCCTTAGCACACTGGGC |
| | | CTAAGACCATCTACCACAGATTGTGACATTGTGCGGCGGGCTTGTGAGT |
| | | CAGTTAGCACTAGAGCCGCACACATGTGCTCAGCTGGTCTGGCTGGAGT |
| | | AATCAATAGGATGCGCGAAAGCAGGAGTGAGGATGTGATGAGAATAAC |
| | | AGTGGGTGTGGATGGGTCTGTCTATAAGCTGCATCCCTCCTTCAAGGAA |
| | | AGGTTTCACGCGTCTGTGCGCAGACTGACCCCTTCATGTGAGATTACTTT |
| | | CATAGAATCTGAGGAGGGCAGTGGAAGGGGGGCAGCCTTGGTTTCTGCT |
| | | GTGGCCTGCAAAAAGGCATGCATGCTTGGCCAATGAAGCCAGCTGCCTTC |
| | | CCTTTACTGTGGGTTAATGCTTGCAGCAGACATGATAAGATACATTGATGAGTT |
| | | TGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAAC |
| | | AACAATTGCATTCATTTTATGTTTCAGGTTCAGGGGGAGATGTGGGAGGTTTTT |
| | | TAAAGCAAGTAAAACCTCTACAAATGTGGTAAAATCCGATAAGGGACTAGAGC |
| | | AGAAGCTTTTG |
| Full: 38 | Modified | [CTAG]AAGCTTTATTGTGGTAGTTTATCACAGTTAAATTGCTAAGGC |
| 5′ UTR: 42 | hGck-20 | AGTCAGTGCTTCTGACACAACAGTCTCCAACTTAAGCTGCAGTGACT |
| ORF: 79 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTAGAGCAGATCTTGGCTGAATTCCAGCTTCAAGAGGAAGATCTG |
| | | AAGAAAGTTATGAGAAGGATGCAGAAGGAGATGGACAGGGGCTTAAGA |
| | | CTGGAAACCCATGAGGAGGCCAGTGTGAAAATGCTACCCACCTATGTAA |
| | | GGTCTACACCTGAAGGATCAGAGGTGGGGGATTTTCTCAGTCTGGACTT |
| | | GGGTGGCACTAACTTCAGAGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGCACAGCAGAGATGCTTTTTGACTACATATCTG |
| | | AATGCATCTCTGATTTCCTAGACAAGCACCAAATGAAACATAAGAAACT |
| | | GCCACTGGGATTTACTTTCTCATTTCCTGTGAGACATGAGGATATTGACA |
| | | AGGGGATCCTCTTGAATTGGACCAAAGGCTTCAAGGCAAGTGGTGCTGA |
| | | AGGAAACAATGTAGTGGGGCTTCTGAGGGATGCTATTAAGAGGAGAGG |
| | | TGACTTTGAGATGGATGTTGTGGCAATGGTCAATGATACAGTGGCCACA |
| | | ATGATCAGCTGTTATTATGAGGACCATCAGTGTGAAGTGGGCATGATAG |
| | | TGGGAACTGGGTGTAATGCCTGCTATATGGAGGAAATGCAGAATGTTGA |
| | | GTTAGTGGAAGGAGATGAGGGTAGAATGTGTGTAAATACAGAGTGGGG |
| | | AGCTTTTGGGGACAGTGGGGAACTGGATGAGTTCCTATTAGAATATGAC |
| | | AGGCTGGTGGATGAGTCTTCAGCCAACCCTGGCCAGCAATTGTATGAAA |
| | | AACTGATTGGAGGGAAGTACATGGGTGAGCTTGTCAGGCTTGTTCTGCT |
| | | GAGATTAGTGGATGAGAATCTCCTGTTTCATGGTGAAGCATCTGAGCAG |
| | | TTGAGGACAAGAGGAGCCTTTGAAACCAGATTTGTCTCACAAGTGGAGA |
| | | GTGATACTGGGGACAGGAAACAGATTTACAACATCCTTAGCACACTGGG |
| | | CCTAAGGCCATCTACCACTGATTGTGACATTGTGAGAAGGGCTTGTGAG |
| | | AGTGTTTCCACTAGAGCAGCACACATGTGCTCAGCTGGTTTGGCTGGAG |
| | | TAATCAATAGGATGAGAGAAAGCAGGAGTGAGGATGTGATGAGAATAA |
| | | CAGTTGGAGTGGATGGGTCTGTCTATAAGCTGCATCCCTCCTTCAAGGA |
| | | AAGGTTTCATGCCTCTGTGAGAAGGCTGACCCCTTCATGTGAGATTACTT |
| | | TCATAGAATCAGAGGAGGGCAGTGGAAGAGGGGCAGCCTTGGTTTCTGC |
| | | TGTGGCCTGCAAAAAGGCATGCATGCTTGGCCAATGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 39 | Modified | [CTAG]AAGCTTTATTGTGGTAGTTTATCACAGTTAAATTGCTAAGGC |
| 5′ UTR: 42 | hGck-21 | AGTCAGTGCTTCTGACACAACAGTCTCCAACTTAAGCTGCAGTGACT |
| ORF: 80 | | CTCTTAAGGTAGCCTTGCAGAAGTTGGTCCTGAGGCACTGGGCAGG |
| 3′ UTR: 60 | | TAAGTATCAAGGTTACAAGACAGGTTTAAGGAGACCAATAGAAACT |
| | | GGGCTTGTGGAGACAGAGAAGACTCTTGGCTTTCTGATAGGCACCT |
| | | ATTGGTCTTACTGACATCCACTTTGCCTTTCTCTCCACAGGTGTCCA |
| | | CTCCCAGTTCAATTACAGCTCTTAAGGCTAGAGTCCTGCAGAATTGC |
| | | CACCATGGCCATGGATGTGACCAGAAGCCAGGCTCAAACAGCACTGAC |
| | | TCTGGTGGAGCAGATCTTGGCTGAATTCCAGCTTCAAGAGGAAGATCTG |
| | | AAGAAAGTTATGAGAAGGATGCAGAAGGAGATGGACAGAGGCTTAAGA |
| | | CTGGAAACCCATGAGGAGGCCAGTGTGAAAATGCTACCCACCTATGTAA |
| | | GATCTACACCTGAAGGATCAGAGGTGGGGGATTTTCTCAGTCTGGACTT |
| | | GGGTGGCACTAACTTCAGAGTCATGCTGGTGAAGGTTGGAGAAGGGGA |
| | | GGAAGGCCAGTGGTCAGTGAAAACCAAGCATCAGATGTATAGCATTCCA |
| | | GAGGATGCTATGACAGGCACAGCAGAGATGCTTTTTGACTACATATCTG |
| | | AATGCATCTCTGATTTCCTGGACAAGCACCAAATGAAACATAAGAAACT |
| | | GCCCCTGGGATTTACTTTCTCATTTCCTGTGAGACATGAGGATATTGACA |
| | | AGGGGATCCTCTTGAATTGGACCAAAGGCTTCAAGGCAAGTGGTGCTGA |
| | | AGGAAACAATGTAGTGGGGCTTCTGAGGGATGCTATTAAGAGAAGAGG |
| | | TGACTTTGAGATGGATGTTGTGGCAATGGTCAATGATACAGTGGCCACA |
| | | ATGATCAGCTGTTATTATGAGGACCATCAGTGTGAAGTGGGCATGATAG |
| | | TGGGAACTGGGTGTAATGCCTGCTATATGGAGGAAATGCAGAATGTTGA |
| | | GTTAGTGGAAGGAGATGAGGGTAGAATGTGTGTAAATACAGAGTGGGG |
| | | AGCTTTTGGGGACAGTGGGGAACTGGATGAGTTCCTACTGGAATATGAC |
| | | AGGCTGGTGGATGAGTCTTCAGCCAACCCTGGCCAGCAATTGTATGAAA |
| | | AACTGATTGGAGGGAAGTACATGGGTGAGCTTGTCAGACTGGTTCTGCT |
| | | GAGATTAGTGGATGAGAATCTCCTGTTTCATGGTGAAGCATCTGAGCAG |
| | | TTGAGAACAAGAGGAGCCTTTGAAACCAGATTTGTCTCACAAGTGGAGA |
| | | GTGATACTGGGGACAGGAAACAGATTTACAACATCCTTAGCACACTGGG |
| | | CCTAAGACCATCTACCACTGATTGTGACATTGTGAGAAGAGCTTGTGAG |
| | | AGTGTTAGCACTAGAGCAGCACACATGTGCTCAGCTGGTCTGGCTGGAG |
| | | TAATCAATAGGATGAGAGAAAGCAGAAGTGAGGATGTGATGAGAATAA |
| | | CAGTGGGAGTGGATGGGTCTGTCTATAAGCTGCATCCCTCCTTCAAGGA |
| | | AAGGTTTCATGCCTCTGTGAGAAGACTGACCCCTTCATGTGAGATTACTT |
| | | TCATAGAATCAGAGGAGGGCAGTGGAAGAGGGGCAGCCTTGGTTTCTGC |
| | | TGTGGCCTGCAAAAAGGCATGCATGCTTGGCCAATGAAGCCAGCTGCCTT |
| | | CCCTTTACTGTGGGTTAATGCTTGCAGCAGACATGATAAGATACATTGATGAG |
| | | TTTGGACAAACCACAACTAGAATGCAGTGAAAAAAATGCTTTATTTGTGAAATT |
| | | TGTGATGCTATTGCTTTATTTGTAACCATTATAAGCTGCAATAAACAAGTTAACA |
| | | ACAACAATTGCATTCATTTTATGTTTCAGGTTCAGGGGGAGATGTGGGAGGTT |
| | | TTTTAAAGCAAGTAAAACCTCTACAAATGTGGTAAAATCCGATAAGGGACTAGA |
| | | GCAGAAGCTTTTG |
| Full: 89 | Modified | AAGCTTTATTGCGGTAGTTTATCACAGTTAAATTGCTAACGCAGTCA |
| 5′ UTR: 83 | hGck-22 | GTGCTTCTGACACAACAGTCTCGAACTTAAGCTGCAGTGACTCTCTT |
| ORF: 61 | | AAGGTAGCCTTGCAGAAGTTGGTCGTGAGGCACTGGGCAGGTAAGT |
| 3′ UTR: 102 | | ATCAAGGTTACAAGACAGGTTTAAGGAGACCAATAGAAACTGGGCT |
| | | TGTCGAGACAGAGAAGACTCTTGCGTTTCTGATAGGCACCTATTGGT |
| | | CTTACTGACATCCACTTTGCCTTTCTCTCCACAGGTGTCCACTCCCA |
| | | GTTCAATTACAGCTCTTAAGGCTAGAGTCCTGCAGAATTGCCACCAT |
| | | GGCCATGGATGTCACAAGGAGCCAGGCCCAGACAGCCTTGACTCTGGTA |
| | | GAGCAGATCCTGGCAGAGTTCCAGCTGCAGGAGGAGGACCTGAAGAAG |
| | | GTGATGAGAAGGATGCAGAAGGAGATGGACAGGGGCCTGAGGCTGGAG |
| | | ACCCATGAAGAGGCCAGTGTGAAGATGCTGCCCACCTATGTGAGGTCCA |
| | | CCCCAGAAGGCTCAGAAGTGGGGGACTTCCTCTCCCTGGACCTGGGTGG |
| | | CACTAACTTCAGGGTGATGCTGGTGAAGGTGGGAGAAGGTGAGGAGGG |
| | | GCAGTGGAGTGTGAAGACCAAACACCAGATGTACTCCATCCCTGAGGAT |
| | | GCCATGACAGGCACTGCTGAGATGCTCTTTGACTACATCTCTGAGTGCAT |
| | | CTCTGACTTCCTGGACAAGCATCAGATGAAACACAAGAAGCTGCCCCTG |
| | | GGCTTCACCTTCTCATTTCCTGTGAGGCATGAAGACATTGATAAGGGCAT |
| | | CCTTCTCAACTGGACCAAGGGCTTCAAGGCCTCAGGAGCAGAAGGGAAC |
| | | AATGTGGTGGGGCTTCTGAGAGATGCTATCAAAAGGAGAGGGGACTTTG |
| | | AAATGGATGTGGTGGCAATGGTGAATGACACTGTGGCCACCATGATCTC |
| | | CTGCTACTATGAAGACCATCAGTGTGAGGTGGGCATGATTGTGGGCACA |
| | | GGCTGCAATGCCTGCTACATGGAGGAGATGCAGAATGTGGAGCTGGTGG |
| | | AGGGGGATGAGGGCAGGATGTGTGTCAATACAGAGTGGGGAGCCTTTG |
| | | GGGACTCTGGGGAGCTGGATGAGTTCCTGCTGGAGTATGACAGGCTGGT |
| | | GGATGAGAGCTCAGCAAACCCTGGTCAGCAGCTGTATGAGAAGCTCATA |
| | | GGTGGCAAGTACATGGGAGAGCTGGTGAGGCTTGTGCTGCTCAGGCTGG |
| | | TGGATGAAAACCTGCTCTTCCATGGGGAGGCCTCTGAGCAGCTGAGGAC |
| | | AAGGGGAGCCTTTGAGACCAGGTTTGTGTCCCAGGTGGAGAGTGACACA |
| | | GGAGACAGGAAGCAGATCTACAACATCCTGAGCACCCTGGGGCTGAGA |
| | | CCCTCCACCACAGACTGTGACATTGTGAGGAGGGCTTGTGAGAGTGTGT |
| | | CTACCAGGGCTGCCCACATGTGCTCTGCTGGGCTGGCTGGGGTCATCAA |
| | | CAGGATGAGGGAGAGCAGGAGTGAGGATGTAATGAGGATCACTGTGGG |
| | | AGTGGATGGCTCTGTGTACAAGCTGCACCCCTCATTCAAGGAGAGGTTC |
| | | CATGCCAGTGTGAGGAGGCTGACCCCCAGCTGTGAGATCACCTTTATTG |
| | | AGTCTGAGGAGGGCAGTGGCAGGGGAGCAGCCCTGGTCTCTGCTGTGGC |
| | | CTGTAAGAAGGCCTGTATGCTGGGCCAGTGAAGCCAGCTGCCTTCCCTTT |
| | | ACTGTGGGTTAATGCTTGCAGCAGACATGATAAGATACATTGATGAGTTTGGA |
| | | CAAACCACAACTAGAATGCAGTGAAAAAAATGCTTTATTTGTGAAATTTGTGAT |
| | | GCTATTGCTTTATTTGTAACCATTATAAGCTGCAATAAACAAGTTAACAACAACA |
| | | ATTGCATTCATTTTATGTTTCAGGTTCAGGGGGAGATGTGGGAGGTTTTTTAAA |
| | | GCAAGTAAAACCTCTACAAATGTGGTAAAATAAGGGACTAGAGCAGTCTAGAA |
| | | AGCTT |
| Full: 90 | Modified | AAGCTTTATTGTGGTAGTTTATCACAGTTAAATTGCTAAGGCAGTCA |
| 5′ UTR: | hGck-23 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 61 | | TCTTACTGACATCCACTTTGCCTTTCTCTCCACAGGTGTCCACTCCC |
| 3′ UTR: 103 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACTGTGGCCACCATGATC |
| | | TCCTGCTACTATGAAGACCATCAGTGTGAGGTGGGCATGATTGTGGGCA |
| | | CAGGCTGCAATGCCTGCTACATGGAGGAGATGCAGAATGTGGAGCTGGT |
| | | GGAGGGGGATGAGGGCAGGATGTGTGTCAATACAGAGTGGGGAGCCTT |
| | | TGGGGACTCTGGGGAGCTGGATGAGTTCCTGCTGGAGTATGACAGGCTG |
| | | GTGGATGAGAGCTCAGCAAACCCTGGTCAGCAGCTGTATGAGAAGCTCA |
| | | TAGGTGGCAAGTACATGGGAGAGCTGGTGAGGCTTGTGCTGCTCAGGCT |
| | | GGTGGATGAAAACCTGCTCTTCCATGGGGAGGCCTCTGAGCAGCTGAGG |
| | | ACAAGGGGAGCCTTTGAGACCAGGTTTGTGTCCCAGGTGGAGAGTGACA |
| | | CAGGAGACAGGAAGCAGATCTACAACATCCTGAGCACCCTGGGGCTGA |
| | | GACCCTCCACCACAGACTGTGACATTGTGAGGAGGGCTTGTGAGAGTGT |
| | | GTCTACCAGGGCTGCCCACATGTGCTCTGCTGGGCTGGCTGGGGTCATC |
| | | AACAGGATGAGGGAGAGCAGGAGTGAGGATGTAATGAGGATCACTGTG |
| | | GGAGTGGATGGCTCTGTGTACAAGCTGCACCCCTCATTCAAGGAGAGGT |
| | | TCCATGCCAGTGTGAGGAGGCTGACCCCCAGCTGTGAGATCACCTTTATT |
| | | GAGTCTGAGGAGGGCAGTGGCAGGGGAGCAGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGGCTAGC |
| | | AAGCTT |
| Full: 91 | Modified | AAGCTTTATTGTGGTAGTTTATCACAGTTAAATTGCTAAGGCAGTCA |
| 5′ UTR: | hGck-24 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 65 | | TCTTACTGACATCCACTTTGCCTTTCTCTCCACAGGTGTCCACTCCC |
| 3′ UTR: 104 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACAGTGGCCACCATGATC |
| | | TCCTGCTACTATGAAGACCATCAGTGTGAGGTGGGCATGATAGTGGGCA |
| | | CAGGCTGCAATGCCTGCTACATGGAGGAGATGCAGAATGTGGAGCTGGT |
| | | GGAGGGGGATGAGGGCAGGATGTGTGTCAATACTGAGTGGGGAGCCTTT |
| | | GGGGACTCTGGGGAGCTGGATGAGTTCCTGCTGGAGTATGACAGGCTGG |
| | | TGGATGAGAGCTCAGCAAACCCTGGTCAGCAGCTGTATGAGAAGCTCAT |
| | | AGGTGGCAAGTACATGGGGGAGCTGGTGAGGCTTGTGCTGCTCAGGCTG |
| | | GTGGATGAAAACCTGCTCTTCCATGGGGAGGCCTCAGAGCAGCTGAGAA |
| | | CAAGGGGAGCCTTTGAGACCAGGTTTGTGTCCCAGGTGGAGAGTGACAC |
| | | AGGTGACAGGAAGCAGATCTACAACATCCTGAGCACCCTGGGGCTGAG |
| | | ACCCTCCACCACAGACTGTGACATTGTGAGGAGGGCCTGTGAGAGTGTG |
| | | TCTACCAGGGCTGCCCACATGTGCTCTGCTGGGCTGGCAGGAGTCATCA |
| | | ACAGGATGAGGGAGAGCAGGAGTGAGGATGTAATGAGGATCACTGTGG |
| | | GAGTGGATGGCTCTGTGTACAAGCTGCACCCCTCATTCAAGGAGAGGTT |
| | | CCATGCCAGTGTGAGAAGGCTGACCCCCAGCTGTGAGATCACCTTTATA |
| | | GAGTCTGAGGAGGGCAGTGGCAGGGGAGCAGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGATTAAT |
| | | AAGCTT |
| Full: 92 | Modified | AAGCTTTATTGTGGTAGTTTATCACAGTTAAATTGCTAAGGCAGTCA |
| 5′ UTR: | hGck-25 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 67 | | TCTTACTGACATCCACTTTGCCTTTCTCTCCACAGGTGTCCACTCCC |
| 3′ UTR: 105 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGGATGCAGAAGGAGATGGACAGGGGCCTGAGGCTGGA |
| | | GACCCATGAAGAGGCCAGTGTGAAGATGCTGCCCACCTATGTGAGGTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCTGACTTCCTGGACAAGCATCAGATGAAACACAAGAAGCTGCCCCT |
| | | GGGCTTCACCTTCTCATTTCCTGTGAGGCATGAAGACATTGATAAGGGC |
| | | ATCCTTCTCAACTGGACCAAGGGCTTCAAGGCCTCAGGAGCAGAAGGGA |
| | | ACAATGTGGTGGGGCTTCTGAGAGATGCTATCAAAAGGAGAGGGGACTT |
| | | TGAAATGGATGTGGTGGCAATGGTGAATGACACAGTGGCCACTATGATC |
| | | TCCTGCTACTATGAAGACCATCAGTGTGAGGTGGGCATGATTGTGGGCA |
| | | CTGGCTGCAATGCCTGCTACATGGAGGAGATGCAGAATGTGGAGCTGGT |
| | | GGAGGGGGATGAGGGCAGGATGTGTGTCAATACTGAGTGGGGAGCCTTT |
| | | GGGGACTCTGGGGAGCTGGATGAGTTCCTGCTGGAGTATGACAGGCTGG |
| | | TGGATGAGAGCTCAGCAAACCCTGGTCAGCAGCTGTATGAGAAGCTCAT |
| | | AGGTGGCAAGTACATGGGAGAGCTGGTGAGACTTGTGCTGCTCAGGCTG |
| | | GTGGATGAAAACCTGCTCTTCCATGGGGAGGCCTCAGAGCAGCTGAGGA |
| | | CAAGGGGAGCCTTTGAGACCAGGTTTGTGTCCCAGGTGGAGAGTGACAC |
| | | TGGAGACAGGAAGCAGATCTACAACATCCTGAGCACCCTGGGGCTGAG |
| | | GCCCTCTACCACAGACTGTGACATAGTGAGAAGGGCCTGTGAGTCTGTG |
| | | TCTACCAGGGCTGCCCACATGTGCTCTGCTGGGCTGGCTGGAGTCATCA |
| | | ACAGAATGAGGGAGAGCAGAAGTGAGGATGTAATGAGGATCACTGTGG |
| | | GAGTGGATGGCTCTGTGTACAAGCTGCACCCCTCATTCAAGGAGAGGTT |
| | | CCATGCCAGTGTGAGGAGGCTGACCCCCAGCTGTGAGATCACCTTTATT |
| | | GAGTCTGAGGAGGGCAGTGGCAGGGGAGCTGCCCTGGTCTCTGCTGTGG |
| | | CCTGTAAGAAGGCCTGTATGCTGGGCCAGTGAAGCCAGCTGCCTTCCCTT |
| | | TACTGTGGGTTAATGCTTGCAGCAGACATGATAAGATACATTGATGAGTTTGG |
| | | ACAAACCACAACTAGAATGCAGTGAAAAAAATGCTTTATTTGTGAAATTTGTGA |
| | | TGCTATTGCTTTATTTGTAACCATTATAAGCTGCAATAAACAAGTTAACAACAAC |
| | | AATTGCATTCATTTTATGTTTCAGGTTCAGGGGGAGATGTGGGAGGTTTTTTAA |
| | | AGCAAGTAAAACCTCTACAAATGTGGTAAAATAAGGGACTAGAGCAGATGCAT |
| | | AAGCTT |
| Full: 93 | Modified | AAGCTTTATTGTGGTAGTTTATCACAGTTAAATTGCTAAGGCAGTCA |
| 5′ UTR: | hGck-26 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 68 | | TCTTACTGACATCCACTTTGCCTTTCTCTCCACAGGTGTCCACTCCC |
| 3′ UTR: 106 | | AGTTCAATTACAGCTCTTAAGGCTAGAGTCCTGCAGAATTGCCACCA |
| | | TGGCTATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACCGCGGCCTGAGGCTGGA |
| | | GACCCATGAAGAGGCCAGTGTGAAGATGCTGCCCACCTATGTGCGCTCC |
| | | ACCCCAGAAGGCTCAGAAGTTGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTTGTGGGGCTTCTGAGAGATGCTATCAAAAGAAGAGGGGACTTT |
| | | GAAATGGATGTGGTGGCAATGGTGAATGACACAGTGGCCACTATGATCT |
| | | CCTGCTACTATGAAGACCATCAGTGTGAGGTTGGCATGATTGTGGGCAC |
| | | AGGCTGCAATGCCTGCTACATGGAGGAGATGCAGAATGTGGAGCTGGTG |
| | | GAGGGGGATGAGGGCCGCATGTGTGTCAATACTGAGTGGGGCGCCTTTG |
| | | GGGACTCTGGAGAGCTGGATGAGTTCCTGCTGGAGTATGACCGCCTGGT |
| | | GGATGAGAGCTCAGCAAACCCAGGTCAGCAGCTGTATGAGAAGCTCATA |
| | | GGTGGCAAGTACATGGGAGAGCTGGTGCGGCTTGTGCTGCTCAGGTTGG |
| | | TGGATGAAAACCTGCTCTTCCATGGGGAGGCCAGTGAGCAGCTGCGCAC |
| | | ACGCGGAGCCTTTGAGACGCGCTTTGTGTCACAAGTGGAGTCTGACACA |
| | | GGAGACCGCAAGCAGATCTACAACATCCTGAGCACACTGGGGCTGCGGC |
| | | CCTCCACCACTGACTGTGACATTGTGCGCCGCGCCTGTGAGAGTGTGTCT |
| | | ACGCGCGCTGCGCACATGTGCTCAGCGGGGCTGGCGGGAGTCATCAACC |
| | | GCATGCGCGAGAGCCGCTCTGAGGATGTAATGCGCATCACTGTGGGAGT |
| | | GGATGGCAGTGTGTACAAGCTGCACCCCAGCTTCAAGGAGCGGTTCCAT |
| | | GCCTCAGTGCGCAGGCTGACCCCCAGCTGTGAGATCACCTTTATTGAGTC |
| | | TGAGGAGGGCAGTGGCAGAGGCGCGGCCCTGGTCAGCGCGGTGGCCTG |
| | | TAAGAAGGCCTGTATGCTGGGCCAGTGAAGCCAGCTGCCTTCCCTTTACTG |
| | | TGGGTTAATGCTTGCAGCAGACATGATAAGATACATTGATGAGTTTGGACAAA |
| | | CCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCA |
| | | AGTAAAACCTCTACAAATGTGGTAAAATAAGGGACTAGAGCAGAGTACTAAGC |
| | | TT |
| Full: 94 | Modified | AAGCTTTATTGTGGTAGTTTATCACAGTTAAATTGCTAAGGCAGTCA |
| 5′ UTR: | hGck-27 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 69 | | TCTTACTGACATCCACTTTGCCTTTCTCTCCACAGGTGTCCACTCCC |
| 3′ UTR: 107 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACCGCGGCCTGAGGCTGGA |
| | | GACCCATGAAGAGGCCAGTGTGAAGATGCTGCCCACCTATGTGCGCTCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTGGTGGGGCTTCTGCGGGATGCTATCAAAAGAAGAGGGGACTTT |
| | | GAAATGGATGTGGTGGCAATGGTGAATGACACAGTGGCCACCATGATCT |
| | | CCTGCTACTATGAAGACCATCAGTGTGAGGTGGGCATGATTGTGGGCAC |
| | | TGGCTGCAATGCCTGCTACATGGAGGAGATGCAGAATGTGGAGCTGGTG |
| | | GAGGGGGATGAGGGCCGCATGTGTGTCAATACAGAGTGGGGCGCCTTTG |
| | | GGGACAGCGGAGAGCTGGATGAGTTCCTGCTGGAGTATGACCGCCTGGT |
| | | GGATGAGAGCTCAGCAAACCCAGGTCAGCAGCTGTATGAGAAGCTCATA |
| | | GGTGGCAAGTACATGGGAGAGCTGGTGCGGCTTGTGCTGCTCAGGCTGG |
| | | TGGATGAAAACCTGCTCTTCCATGGGGAGGCCTCTGAGCAGCTGCGCAC |
| | | ACGCGGAGCCTTTGAGACGCGCTTTGTGAGCCAGGTGGAGAGTGACACA |
| | | GGAGACCGCAAGCAGATCTACAACATCCTGAGCACCCTGGGGCTGCGGC |
| | | CCAGCACCACTGACTGTGACATTGTGCGCCGCGCCTGTGAGTCAGTGTCT |
| | | ACGCGCGCTGCGCACATGTGCAGCGCGGGGCTGGCGGGAGTCATCAACC |
| | | GCATGCGCGAGAGCCGCTCTGAGGATGTAATGCGCATCACTGTGGGAGT |
| | | GGATGGCAGTGTGTACAAGCTGCACCCCAGCTTCAAGGAGCGGTTCCAT |
| | | GCCTCAGTGCGCAGGCTGACCCCCAGCTGTGAGATCACCTTTATTGAGTC |
| | | TGAGGAGGGCAGTGGCAGAGGCGCGGCCCTGGTCAGCGCGGTGGCCTG |
| | | TAAGAAGGCCTGTATGCTGGGCCAGTGAAGCCAGCTGCCTTCCCTTTACTG |
| | | TGGGTTAATGCTTGCAGCAGACATGATAAGATACATTGATGAGTTTGGACAAA |
| | | CCACAACTAGAATGCAGTGAAAAAAATGCTTTATTTGTGAAATTTGTGATGCTA |
| | | TTGCTTTATTTGTAACCATTATAAGCTGCAATAAACAAGTTAACAACAACAATTG |
| | | CATTCATTTTATGTTTCAGGTTCAGGGGGAGATGTGGGAGGTTTTTTAAAGCA |
| | | AGTAAAACCTCTACAAATGTGGTAAAATAAGGGACTAGAGCAGGGTACCAAGC |
| | | TT |
| Full: 95 | Modified | AAGCTTTATTGTGGTAGTTTATCACAGTTAAATTGCTAAGGCAGTCA |
| 5′ UTR: | hGck-28 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 72 | | TCTTACTGACATCCACTTTGCCTTTCTCTCCACAGGTGTCCACTCCC |
| 3′ UTR: 108 | | AGTTCAATTACAGCTCTTAAGGCTAGAGTCCTGCAGAATTGCCACCA |
| | | TGGCTATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACAGAGGCCTGAGGCTGGA |
| | | GACCCATGAAGAGGCCAGTGTGAAGATGCTGCCCACCTATGTGAGATCC |
| | | ACCCCAGAAGGCTCAGAAGTTGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTTGTGGGGCTTCTGAGAGATGCTATCAAAAGAAGAGGGGACTTT |
| | | GAAATGGATGTGGTGGCAATGGTGAATGACACAGTGGCCACTATGATCT |
| | | CCTGCTACTATGAAGACCATCAGTGTGAGGTTGGCATGATTGTGGGCAC |
| | | AGGCTGCAATGCCTGCTACATGGAGGAGATGCAGAATGTGGAGCTGGTG |
| | | GAGGGGGATGAGGGCAGGATGTGTGTCAATACTGAGTGGGGAGCCTTTG |
| | | GGGACTCTGGAGAGCTGGATGAGTTCCTGCTGGAGTATGACAGACTGGT |
| | | GGATGAGAGCTCAGCAAACCCAGGTCAGCAGCTGTATGAGAAGCTCATA |
| | | GGTGGCAAGTACATGGGAGAGCTGGTGAGGCTTGTGCTGCTCAGGTTGG |
| | | TGGATGAAAACCTGCTCTTCCATGGGGAGGCCAGTGAGCAGCTGAGAAC |
| | | AAGGGGAGCCTTTGAGACAAGATTTGTGTCACAGGTGGAGTCTGACACA |
| | | GGAGACAGGAAGCAGATCTACAACATCCTGAGCACCCTGGGGCTGAGA |
| | | CCCTCCACCACTGACTGTGACATTGTGAGGAGAGCCTGTGAGAGTGTGT |
| | | CTACCAGGGCTGCTCACATGTGCTCAGCAGGGCTGGCTGGAGTCATCAA |
| | | CAGAATGAGGGAGAGCAGATCTGAGGATGTAATGAGGATCACTGTGGG |
| | | TGTGGATGGCAGTGTGTACAAGCTGCACCCCAGCTTCAAGGAGAGATTC |
| | | CATGCCTCAGTGAGGAGGCTGACCCCCAGCTGTGAGATCACCTTCATTG |
| | | AGTCTGAGGAGGGCAGTGGCAGAGGAGCAGCCCTGGTCTCTGCTGTGGC |
| | | CTGTAAGAAGGCCTGTATGCTGGGCCAGTGAAGCCAGCTGCCTTCCCTTT |
| | | ACTGTGGGTTAATGCTTGCAGCAGACATGATAAGATACATTGATGAGTTTGGA |
| | | CAAACCACAACTAGAATGCAGTGAAAAAAATGCTTTATTTGTGAAATTTGTGAT |
| | | GCTATTGCTTTATTTGTAACCATTATAAGCTGCAATAAACAAGTTAACAACAACA |
| | | ATTGCATTCATTTTATGTTTCAGGTTCAGGGGGAGATGTGGGAGGTTTTTTAAA |
| | | GCAAGTAAAACCTCTACAAATGTGGTAAAATAAGGGACTAGAGCAGAATATTA |
| | | AGCTT |
| Full: 96 | Modified | AAGCTTTATTGTGGTAGTTTATCACAGTTAAATTGCTAAGGCAGTCA |
| 5′ UTR: | hGck-29 | GTGCTTCTGACACAACAGTCTCCAACTTAAGCTGCAGTGACTCTCTT |
| nucleic | | AAGGTAGCCTTGCAGAAGTTGGTCCTGAGGCACTGGGCAGGTAAGT |
| acids 5-329 | | ATCAAGGTTACAAGACAGGTTTAAGGAGACCAATAGAAACTGGGCT |
| of 42 | | TGTGGAGACAGAGAAGACTCTTGGCTTTCTGATAGGCACCTATTGG |
| ORF: 73 | | TCTTACTGACATCCACTTTGCCTTTCTCTCCACAGGTGTCCACTCCC |
| 3′ UTR: 109 | | AGTTCAATTACAGCTCTTAAGGCTAGAGTCCTGCAGAATTGCCACCA |
| | | TGGCCATGGATGTCACAAGGAGCCAGGCCCAGACAGCCTTGACTCTGGT |
| | | AGAGCAGATCCTGGCAGAGTTCCAGCTGCAGGAGGAGGACCTGAAGAA |
| | | GGTGATGAGAAGAATGCAGAAGGAGATGGACAGAGGCCTGAGGCTGGA |
| | | GACCCATGAAGAGGCCAGTGTGAAGATGCTGCCCACCTATGTGAGATCC |
| | | ACCCCAGAAGGCTCAGAAGTGGGGGACTTCCTCTCCCTGGACCTGGGTG |
| | | GCACTAACTTCAGGGTGATGCTGGTGAAGGTGGGAGAAGGTGAGGAGG |
| | | GGCAGTGGAGTGTGAAGACCAAACACCAGATGTACTCCATCCCTGAGGA |
| | | TGCCATGACAGGCACTGCTGAGATGCTCTTTGACTACATCTCTGAGTGCA |
| | | TCTCAGACTTCCTGGACAAGCATCAGATGAAACACAAGAAGCTGCCCCT |
| | | GGGCTTCACCTTCTCCTTTCCTGTGAGGCATGAAGACATTGATAAGGGCA |
| | | TCCTTCTCAACTGGACCAAGGGCTTCAAGGCCTCAGGAGCAGAAGGGAA |
| | | CAATGTGGTGGGGCTTCTGAGAGATGCTATCAAAAGAAGAGGGGACTTT |
| | | GAAATGGATGTGGTGGCAATGGTGAATGACACAGTGGCCACCATGATCT |
| | | CCTGCTACTATGAAGACCATCAGTGTGAGGTGGGCATGATTGTGGGCAC |
| | | TGGCTGCAATGCCTGCTACATGGAGGAGATGCAGAATGTGGAGCTGGTG |
| | | GAGGGGGATGAGGGCAGAATGTGTGTCAATACAGAGTGGGGAGCCTTT |
| | | GGGGACTCTGGAGAGCTGGATGAGTTCCTGCTGGAGTATGACAGACTGG |
| | | TGGATGAGAGCTCAGCAAACCCAGGTCAGCAGCTGTATGAGAAGCTCAT |
| | | AGGTGGCAAGTACATGGGAGAGCTGGTGAGACTTGTGCTGCTCAGGCTG |
| | | GTGGATGAAAACCTGCTCTTCCATGGGGAGGCCAGTGAGCAGCTGAGAA |
| | | CAAGAGGAGCCTTTGAGACCAGATTTGTGAGCCAGGTGGAGTCAGACAC |
| | | AGGAGACAGAAAGCAGATCTACAACATCCTGAGCACCCTGGGGCTGAG |
| | | ACCCAGCACCACTGACTGTGACATTGTGAGAAGAGCCTGTGAGTCTGTG |
| | | TCTACCAGAGCTGCCCACATGTGCAGTGCTGGGCTGGCTGGAGTCATCA |
| | | ACAGAATGAGAGAGAGCAGATCAGAGGATGTAATGAGAATCACTGTGG |
| | | GTGTGGATGGCTCTGTGTACAAGCTGCACCCCAGCTTCAAGGAGAGATT |
| | | CCATGCCAGTGTGAGAAGGCTGACCCCCAGCTGTGAGATCACCTTTATT |
| | | GAGTCAGAGGAGGGCAGTGGCAGAGGAGCAGCCCTGGTCTCTGCTGTG |
| | | GCCTGTAAGAAGGCCTGTATGCTGGGCCAGTGAAGCCAGCTGCCTTCCCT |
| | | TTACTGTGGGTTAATGCTTGCAGCAGACATGATAAGATACATTGATGAGTTTG |
| | | GACAAACCACAACTAGAATGCAGTGAAAAAAATGCTTTATTTGTGAAATTTGTG |
| | | ATGCTATTGCTTTATTTGTAACCATTATAAGCTGCAATAAACAAGTTAACAACAA |
| | | CAATTGCATTCATTTTATGTTTCAGGTTCAGGGGGAGATGTGGGAGGTTTTTTA |
| | | AAGCAAGTAAAACCTCTACAAATGTGGTAAAATAAGGGACTAGAGCAGTTAAT |
| | | TAAGCTT |
| Full: 163 | Unmodified | CTAGAAGCTTTATTGTGGTAGTTTATCACAGTTAAATTGCTAAGGCA |
| 5′ UTR: 42 | hGck_2 | GTCAGTGCTTCTGACACAACAGTCTCCAACTTAAGCTGCAGTGACTC |
| ORF: 162 | | TCTTAAGGTAGCCTTGCAGAAGTTGGTCCTGAGGCACTGGGCAGGT |
| 3′ UTR: | | AAGTATCAAGGTTACAAGACAGGTTTAAGGAGACCAATAGAAACTG |
| nucleotides | | GGCTTGTGGAGACAGAGAAGACTCTTGGCTTTCTGATAGGCACCTA |
| 1-285 of | | TTGGTCTTACTGACATCCACTTTGCCTTTCTCTCCACAGGTGTCCAC |
| SEQ ID | | TCCCAGTTCAATTACAGCTCTTAAGGCTAGAGTCCTGCAGAATTGCC |
| NO: 60 | | ACCATGGCGATGGATGTCACAAGGAGCCAGGCCCAGACAGCCTTGACTC |
| | | TGGTAGAGCAGATCCTGGCAGAGTTCCAGCTGCAGGAGGAGGACCTGA |
| | | AGAAGGTGATGAGACGGATGCAGAAGGAGATGGACCGCGGCCTGAGGC |
| | | TGGAGACCCATGAAGAGGCCAGTGTGAAGATGCTGCCCACCTACGTGCG |
| | | CTCCACCCCAGAAGGCTCAGAAGTCGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGCGTGAAGACCAAACACCAGATGTACTCCATCCCC |
| | | GAGGACGCCATGACCGGCACTGCTGAGATGCTCTTCGACTACATCTCTG |
| | | AGTGCATCTCCGACTTCCTGGACAAGCATCAGATGAAACACAAGAAGCT |
| | | GCCCCTGGGCTTCACCTTCTCCTTTCCTGTGAGGCACGAAGACATCGATA |
| | | AGGGCATCCTTCTCAACTGGACCAAGGGCTTCAAGGCCTCAGGAGCAGA |
| | | AGGGAACAATGTCGTGGGGCTTCTGCGAGACGCTATCAAACGGAGAGG |
| | | GGACTTTGAAATGGATGTGGTGGCAATGGTGAATGACACGGTGGCCACG |
| | | ATGATCTCCTGCTACTACGAAGACCATCAGTGCGAGGTCGGCATGATCG |
| | | TGGGCACGGGCTGCAATGCCTGCTACATGGAGGAGATGCAGAATGTGGA |
| | | GCTGGTGGAGGGGGACGAGGGCCGCATGTGCGTCAATACCGAGTGGGG |
| | | CGCCTTCGGGGACTCCGGCGAGCTGGACGAGTTCCTGCTGGAGTATGAC |
| | | CGCCTGGTGGACGAGAGCTCTGCAAACCCCGGTCAGCAGCTGTATGAGA |
| | | AGCTCATAGGTGGCAAGTACATGGGCGAGCTGGTGCGGCTTGTGCTGCT |
| | | CAGGCTCGTGGACGAAAACCTGCTCTTCCACGGGGAGGCCTCCGAGCAG |
| | | CTGCGCACACGCGGAGCCTTCGAGACGCGCTTCGTGTCGCAGGTGGAGA |
| | | GCGACACGGGCGACCGCAAGCAGATCTACAACATCCTGAGCACGCTGG |
| | | GGCTGCGACCCTCGACCACCGACTGCGACATCGTGCGCCGCGCCTGCGA |
| | | GAGCGTGTCTACGCGCGCTGCGCACATGTGCTCGGCGGGGCTGGCGGGC |
| | | GTCATCAACCGCATGCGCGAGAGCCGCAGCGAGGACGTAATGCGCATCA |
| | | CTGTGGGCGTGGATGGCTCCGTGTACAAGCTGCACCCCAGCTTCAAGGA |
| | | GCGGTTCCATGCCAGCGTGCGCAGGCTGACGCCCAGCTGCGAGATCACC |
| | | TTCATCGAGTCGGAGGAGGGCAGTGGCCGGGGCGCGGCCCTGGTCTCGG |
| | | CGGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAT |
| Full: 164 | Unmodified | CTAGAAGCTTTATTGTGGTAGTTTATCACAGTTAAATTGCTAAGGCA |
| 5′ UTR: 42 | hGck_3 | GTCAGTGCTTCTGACACAACAGTCTCCAACTTAAGCTGCAGTGACTC |
| ORF: 162 | | TCTTAAGGTAGCCTTGCAGAAGTTGGTCCTGAGGCACTGGGCAGGT |
| 3′ UTR: 60 | | AAGTATCAAGGTTACAAGACAGGTTTAAGGAGACCAATAGAAACTG |
| | | GGCTTGTGGAGACAGAGAAGACTCTTGGCTTTCTGATAGGCACCTA |
| | | TTGGTCTTACTGACATCCACTTTGCCTTTCTCTCCACAGGTGTCCAC |
| | | TCCCAGTTCAATTACAGCTCTTAAGGCTAGAGTCCTGCAGAATTGCC |
| | | ACCATGGCGATGGATGTCACAAGGAGCCAGGCCCAGACAGCCTTGACTC |
| | | TGGTAGAGCAGATCCTGGCAGAGTTCCAGCTGCAGGAGGAGGACCTGA |
| | | AGAAGGTGATGAGACGGATGCAGAAGGAGATGGACCGCGGCCTGAGGC |
| | | TGGAGACCCATGAAGAGGCCAGTGTGAAGATGCTGCCCACCTACGTGCG |
| | | CTCCACCCCAGAAGGCTCAGAAGTCGGGGACTTCCTCTCCCTGGACCTG |
| | | GGTGGCACTAACTTCAGGGTGATGCTGGTGAAGGTGGGAGAAGGTGAG |
| | | GAGGGGCAGTGGAGCGTGAAGACCAAACACCAGATGTACTCCATCCCC |
| | | GAGGACGCCATGACCGGCACTGCTGAGATGCTCTTCGACTACATCTCTG |
| | | AGTGCATCTCCGACTTCCTGGACAAGCATCAGATGAAACACAAGAAGCT |
| | | GCCCCTGGGCTTCACCTTCTCCTTTCCTGTGAGGCACGAAGACATCGATA |
| | | AGGGCATCCTTCTCAACTGGACCAAGGGCTTCAAGGCCTCAGGAGCAGA |
| | | AGGGAACAATGTCGTGGGGCTTCTGCGAGACGCTATCAAACGGAGAGG |
| | | GGACTTTGAAATGGATGTGGTGGCAATGGTGAATGACACGGTGGCCACG |
| | | ATGATCTCCTGCTACTACGAAGACCATCAGTGCGAGGTCGGCATGATCG |
| | | TGGGCACGGGCTGCAATGCCTGCTACATGGAGGAGATGCAGAATGTGGA |
| | | GCTGGTGGAGGGGGACGAGGGCCGCATGTGCGTCAATACCGAGTGGGG |
| | | CGCCTTCGGGGACTCCGGCGAGCTGGACGAGTTCCTGCTGGAGTATGAC |
| | | CGCCTGGTGGACGAGAGCTCTGCAAACCCCGGTCAGCAGCTGTATGAGA |
| | | AGCTCATAGGTGGCAAGTACATGGGCGAGCTGGTGCGGCTTGTGCTGCT |
| | | CAGGCTCGTGGACGAAAACCTGCTCTTCCACGGGGAGGCCTCCGAGCAG |
| | | CTGCGCACACGCGGAGCCTTCGAGACGCGCTTCGTGTCGCAGGTGGAGA |
| | | GCGACACGGGCGACCGCAAGCAGATCTACAACATCCTGAGCACGCTGG |
| | | GGCTGCGACCCTCGACCACCGACTGCGACATCGTGCGCCGCGCCTGCGA |
| | | GAGCGTGTCTACGCGCGCTGCGCACATGTGCTCGGCGGGGCTGGCGGGC |
| | | GTCATCAACCGCATGCGCGAGAGCCGCAGCGAGGACGTAATGCGCATCA |
| | | CTGTGGGCGTGGATGGCTCCGTGTACAAGCTGCACCCCAGCTTCAAGGA |
| | | GCGGTTCCATGCCAGCGTGCGCAGGCTGACGCCCAGCTGCGAGATCACC |
| | | TTCATCGAGTCGGAGGAGGGCAGTGGCCGGGGCGCGGCCCTGGTCTCGG |
| | | CGGTGGCCTGTAAGAAGGCCTGTATGCTGGGCCAGTGAAGCCAGCTGCC |
| | | TTCCCTTTACTGTGGGTTAATGCTTGCAGCAGACATGATAAGATACATTGATGA |
| | | GTTTGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGTTAAC |
| | | AACAACAATTGCATTCATTTTATGTTTCAGGTTCAGGGGGAGATGTGGGAGGT |
| | | TTTTTAAAGCAAGTAAAACCTCTACAAATGTGGTAAAATCCGATAAGGGACTAG |
| | | AGCAGAAGCTTTTG |

In some aspects, the nucleic acids include codon optimized and CpG reduced sequences relative to wild-type and/or unmodified human Gck nucleic acid sequences (e.g., SEQ ID NO: 19). The modified nucleic acids were chemically synthesized, prepared in expression cassettes including a CMV promoter, and cloned into an expression plasmid. The modified sequences were confirmed by Sanger sequencing.

GcK expression in HEK cells transfected with pAAV-Gck plasmids was tested. pAAV-Gck plasmids AAV1-CMV-hGckWT (SEQ ID NO: 19), AAV1-CMV-hGckWT_2 (SEQ ID NO: 163), AAV1-CMV-modified hGck9 (SEQ ID NO: 68), AAV1-CMV-modified hGck10 (SEQ ID NO: 69), AAV1-CMV-modified hGck11 (SEQ ID NO: 70), AAV1-CMV-modified hGck12 (SEQ ID NO: 71), AAV1-CMV-modified hGck13 (SEQ ID NO: 72), AAV1-CMV-modified hGck14 (SEQ ID NO: 73), AAV1-CMV-modified hGck15 (SEQ ID NO: 74), and AAV1-CMV-modified hGck16 (SEQ ID NO: 75) were transfected into HEK293 cells in a 6-well plate at 2.5 ug per well. The cell pellet was collected 48 hr after transfection and intracellular insulin level was determined by ELISA assay. pAAV-GcK plasmids exhibited Gck expression after transfection.

### Example 3: In Vitro Comparative Analysis of hInsulin Vectors

AAV1-CMV-hInsulin vectors bearing insulin variants (SEQ ID NO: 1, SEQ ID NO: 87 and SEQ ID NO: 88) were studied for infectivity and potency (insulin mRNA expression, insulin secretion into cell media and biological activity of the secreted insulin). 2v6.11 cells were infected with AAV1-CMV-hInsWT (SEQ ID NO: 110), AAV1-CMV-Ins5 (SEQ ID NO: 87) and AAV1-CMV-Ins7 (SEQ ID NO: 88) vectors at different MOIs. For the infectivity assay, the intracellular content of vector genomes was quantified. For the potency assay, human insulin mRNA expression and Insulin secreted into the cell media were measured. Secreted Insulin was also assessed for functionality.

### Methods

3.1 Infection of 2v6.11 Cells with AAV1-CMV-hInsulin Vectors

The day before infection, 2v6.11 cells were seeded in 24-well plates at a density of 2E+05 cells/well. Cells were grown at 37° C. and 8.5% CO₂ in growth media (DMEM+10% FBS) supplemented with antibiotics (Penicillin=10,000 U/ml, Streptomycin=10,000 μg/ml) and 1 μg/ml Ponasterone A.

Prior to infection, cells were assessed for correct cell confluence (70-80%) at a bright field microscope. To assess cell count, cells of 4 wells were trypsinized and quantified with a Scepter 2.0 Handheld Automated Cell Counter (Merck-Millipore). Cells were infected with AAV1-CMV-hInsulin vectors AAV1-CMV-hInsB10D (wild-type) (SEQ ID NO: 110), AAV1-CMV-Ins5 (SEQ ID NO: 87) and AAV1-CMV-Ins7 (SEQ ID NO: 88) (Table 3) at MOI=1000, 2000 and 4000 vg/cell. Cells were infected in quadruplicate both for the infectivity test and for the potency test. Non-infected 2v6.11 cells (NI) were used as a negative control. The infection was repeated three times on different days using cells at different cell passage.

TABLE 3
AAV1-CMV-hInsulin vectors

| AAV Vector | SEQ ID NO |
|---|---|
| AAV1-CMV-hInsB10D | SEQ ID NO: 1 |
| AAV1-CMV-Ins5 | SEQ ID NO: 87 |
| AAV1-CMV-Ins7 | SEQ ID NO: 88 |

### 3.2 Infectivity Test

Twenty-four hours post-infection, cells were observed under a bright field microscope for viability. Next, cell media was aspirated, cells were washed twice with 500 μl 1×PBS and collected in 200 μl 1×PBS with a cell scraper. Intracellular vector genomes were extracted with the DNeasy Blood & Tissue Kits (Qiagen) and amplified by Taqman qPCR with an oligo set targeting the ITR2 sequence. Vector genomes were quantified by interpolation from a standard curve generated with the serial dilution of a standard DNA.

### 3.3 Sample Collection for Potency Test

Forty-eight hours post-infection and after assessing cell viability, cell media was aspirated, replaced with 400 μl of DMEM+1% BSA warmed to 37° C. and plates were returned to the incubator. After a 5-hour incubation and for protein readout, 350 μl of cell media were collected into a 1.5 ml microtube, centrifuged at 600×g for 10 min at 4° C. and 300 μl supernatant were transferred to new tubes. For mRNA expression, the remaining media was aspirated from wells, cells were gently washed with 500 μl 1×PBS and collected in 350 μl RLT+β-mercaptoethanol (10 μl/ml) (RNeasy Mini Kit, Qiagen). Samples were stored at −80° C. until processing.

3.4 hInsulin mRNA Expression

RNA was extracted with the RNeasy Mini Kit (Qiagen) and RNase-free DNase I (Qiagen) following the protocol provided by the manufacturer except the extension of the on-column DNAse I digestion from the standard 15 minutes to 30 minutes, to ensure appropriate degradation of the infected AAV vector genome. One ug of each RNA sample was retrotranscribed using the Transcriptor FirstStrand cDNA Synthesis kit (Roche). qPCR was performed in triplicate using Taqman Probes Master (Roche) and 2 μl of sample (diluted 1/10). To quantify expression, a primer-probe mix targeting the SV40 poly A signal (sequence common to all the hInsulin plasmids) was used (Forward primer: AGC AAT AGC ATC ACA AAT TTC ACA A; Reverse primer: CAG ACA TGA TAA GAT ACA TTG ATG AGT T; Probe: /56-FAM/AGC ATT TTT TT/ZEN/CAC TGC ATT CTA GTT GTG GTT TGT C/3IABKFQ/). A primer-probe mix for housekeeping gene hRplp0 was used to normalize (forward primer: CAG ACA GAC ACT GGC AAC AT; Reverse primer: GCA GCA TCT ACA ACC CTG AA; Probe: /5HEX/AA CTC TGC A/ZEN/TT CTC GCT TCC TGG A/3IABKFQ).

3.5 Quantitative Analysis of Secreted hInsulin

Secreted insulin was measured in duplicate in media samples diluted 1/10 in milliQ water using the Insulin ELISA Kit (Crystal Chem).

3.6 Functional Analysis of Secreted hInsulin

The biological activity of insulin produced and secreted into cell media by the infected cells was measured with the iLite Insulin Assay Ready Cells (Svar Life Science). Briefly, 40 μl of standard or cell media from infected cells and 40 μl of iLite Insulin Assay Ready Cells, previously thawed and resuspended in RPMI supplemented with 9% FBS (heat inactivated) and antibiotics (Penicillin=10,000 U/ml, Streptomycin=10,000 μg/ml), were added to a 96-well white plate. After a 5-hour incubation at 37° C. and 5% CO₂, the plate was equilibrated to room temperature and 80 μl of ONE-Glo Luciferase Assay reagent were added into wells. Cells were allowed to lyse for 10 minutes and luminescence was measured in a plate reader. Recombinant human insulin (Life Technologies) was used for the standard curve.

### 3.7 Statistical Analysis

Each MOI was analyzed independently for the 3 studies performed using Anova and Tukey's multiple comparison test.

### Results

2v6.11 cells were infected with AAV1-hInsulin vectors bearing the hInsulin variants Ins5 and Ins7 and the WT hInsulin to assess vector infectivity and potency in three independent studies.

### 3.8 Infectivity Assay

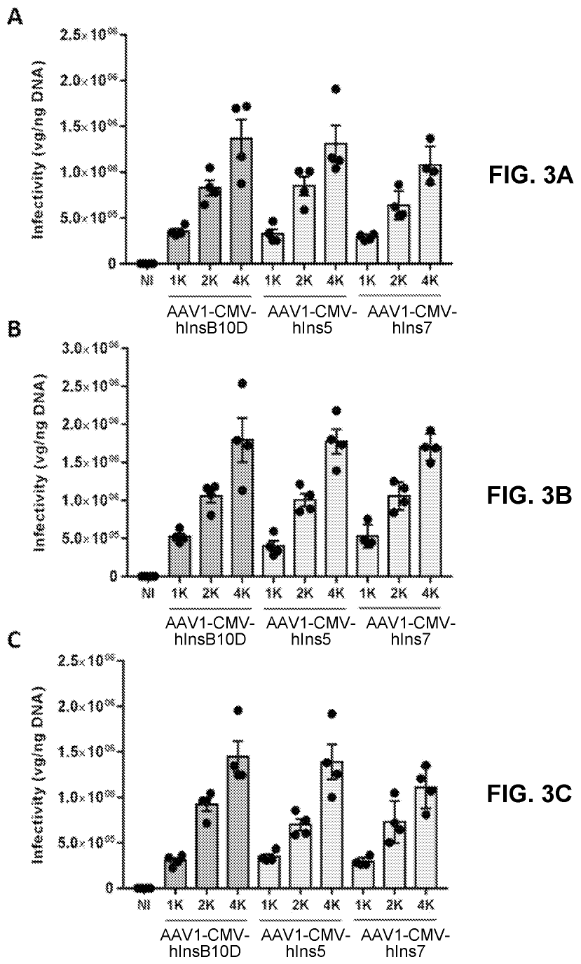

The three independent infection studies performed in 2v6.11 cells with AAV1-hInsulin vectors AAV1-CMV-hInsB10D (SEQ ID NO: 1), AAV1-CMV-Ins5 (SEQ ID NO: 87) and AAV1-CMV-Ins7 (SEQ ID NO: 88) showed no significant differences in the ability of the different vectors to infect cells at the 3 MOIs tested (1000 vg/cell (1K), 2000 vg/cell (2K) and 4000 vg/cell (4K)) as indicated by the intracellular vector genome content (vg/ng DNA) (FIGS. 3A-3C and Table 4).

TABLE 4
Statistical significance (adjusted P value) of the infectivity
assays for the different AAV1-hInsulin vectors.

| | | Adjusted P value | | |
|---|---|---|---|---|
| MOI | Vector comparison | Assay 1 | Assay 2 | Assay 3 |
| 1K | AAV1-CMV-hInsB10D | 0.8265 | 0.3987 | 0.5149 |
| | vs. AAV1-CMV-Ins5 | | | |
| | AAV1-CMV-hInsB10D | 0.369 | 0.9981 | 0.964 |
| | vs. AAV1-CMV-Ins7 | | | |
| | AAV1-CMV-Ins5 vs. | 0.6941 | 0.3711 | 0.3815 |
| | AAV1-CMV-Ins7 | | | |
| 2K | AAV1-CMV-hInsB10D | 0.9836 | 0.9121 | 0.2189 |
| | vs. AAV1-CMV-Ins5 | | | |
| | AAV1-CMV-hInsB10D | 0.3277 | >0.9999 | 0.3067 |
| | vs. AAV1-CMV-Ins7 | | | |
| | AAV1-CMV-hIns5 vs. | 0.2599 | 0.9071 | 0.9672 |
| | AAV1-CMV-Ins7 | | | |
| 4K | AAV1-CMV-hInsB10D | 0.9722 | 0.9972 | 0.9642 |
| | vs. AAV1-CMV-Ins5 | | | |
| | AAV1-CMV-hInsB10D | 0.5074 | 0.9389 | 0.3504 |
| | vs. AAV1-CMV-Ins7 | | | |
| | AAV1-CMV-hIns5 vs. | 0.6376 | 0.9614 | 0.4775 |
| | AAV1-CMV-Ins7 | | | |

Infectivity data corresponding to each MOI and each assay was analyzed with Anova and Tukey's multiple comparison test.

### 3.9 Potency Assay

3.9.1 Human Insulin mRNA Expression

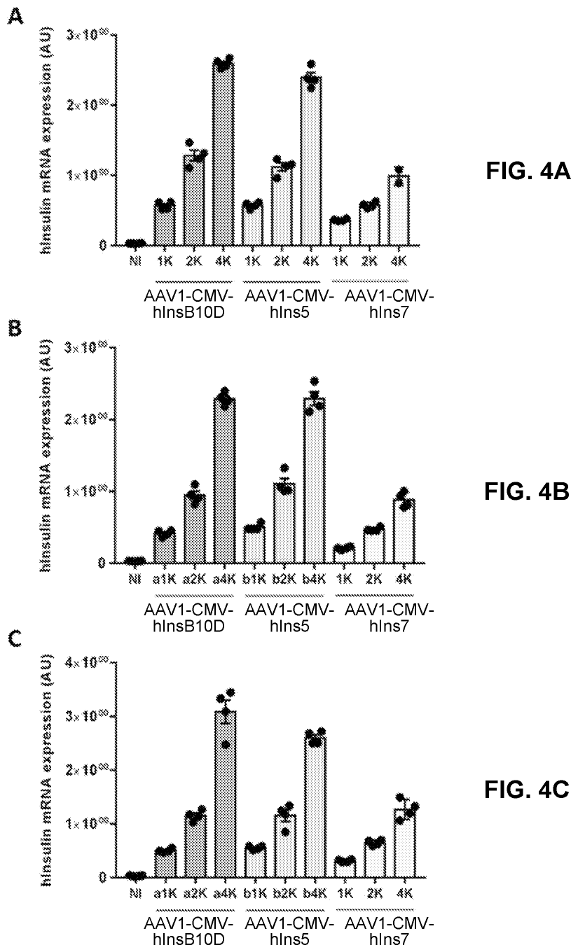

Quantification of hInsulin expression levels revealed no significant differences between vectors AAV1-CMV-hInsB10D (SEQ ID NO: 1) and AAV1-CMV-Ins5 (SEQ ID NO: 87) in the three studies performed (FIGS. 4A-4C and Table 5). On the other hand, the mRNA expression levels mediated by the infection with vector AAV1-CMV-Ins7 (SEQ ID NO: 88) were significantly lower than those mediated by AAV1-CMV-hInsB10D (SEQ ID NO: 110) and AAV1-CMV-Ins5 (SEQ ID NO: 87) (FIGS. 4A-4C and Table 5).

TABLE 5
Statistical significance (adjusted P value) of hInsulin mRNA
expression for the different AAV1-hInsulin vectors.

| | | Adjusted P value | | |
|---|---|---|---|---|
| MOI | Vector comparison | Assay 1 | Assay 2 | Assay 3 |
| 1K | AAV1-CMV-hIns_B10D | 0.9829 | 0.0315 | 0.0929 |
| | vs. AAV1-CMV-Ins5 | | | |
| | AAV1-CMV-hIns_B10D | 0.0002 | 0.0001 | <0.0001 |
| | vs. AAV1-CMV-Ins7 | | | |
| | AAV1-CMV-Ins5 vs. | 0.0001 | <0.0001 | <0.0001 |
| | AAV1-CMV-Ins7 | | | |
| 2K | AAV1-CMV-hIns_B10D | 0.1602 | 0.1626 | 0.9995 |
| | vs. AAV1-CMV-Ins5 | | | |
| | AAV1-CMV-hInsB10D | <0.0001 | 0.0005 | 0.0015 |
| | vs. AAV1-CMV-Ins7 | | | |
| | AAV1-CMV-Ins5 vs. | 0.0002 | <0.0001 | 0.0016 |
| | AAV1-CMV-Ins7 | | | |
| 4K | AAV1-CMV-hIns_B10D | 0.0918 | 0.9985 | 0.0856 |
| | vs. AAV1-CMV-Ins5 | | | |
| | AAV1-CMV-hIns_B10D | <0.0001 | <0.0001 | <0.0001 |
| | vs. AAV1-CMV-Ins7 | | | |
| | AAV1-CMV-Ins5 vs. | <0.0001 | <0.0001 | 0.0002 |
| | AAV1-CMV-Ins7 | | | |

hInsulin mRNA expression data corresponding to each MOI and each assay was analyzed with Anova and Tukey's multiple comparison test. Bold and italic indicates statistical significance.

3.9.2 Quantitative Analysis of Secreted hInsulin

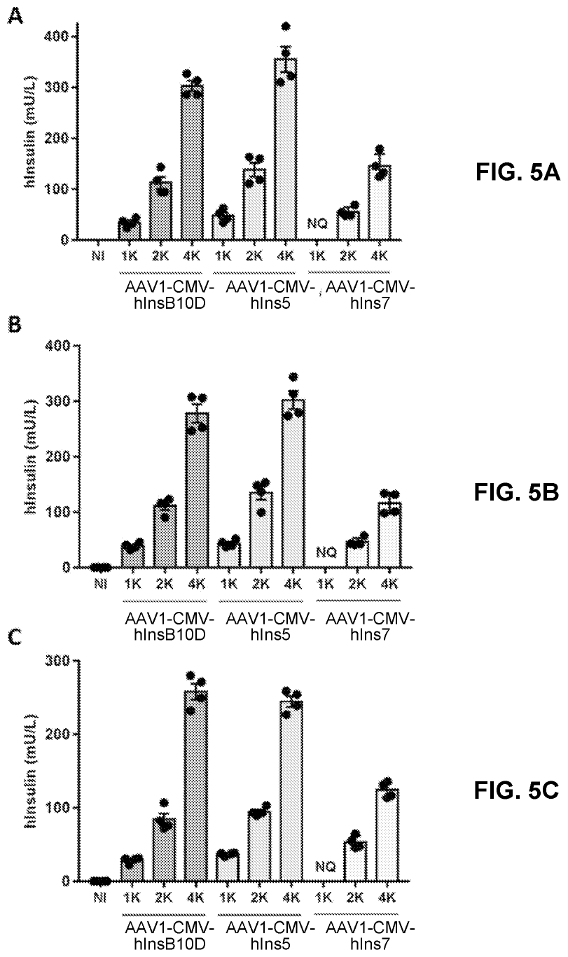

When the insulin secreted into the cell media by 2v6.11 cells infected with the different AAV1-hInsulin vectors was quantified using an Insulin ELISA kit (Crystal Chem), no significant differences were observed between vectors AAV1-CMV-hInsB10D (SEQ ID NO: 110) and AAV1-CMV-Ins5 (SEQ ID NO: 87) (FIGS. 5A-5C and Table 6). As observed for the mRNA expression readout (FIGS. 4A-4C and Table 5), the levels of secreted insulin mediated by vector AAV1-CMV-Ins7 (SEQ ID NO: 88) were significantly lower than those provided by AAV1-CMV-hInsB10D (SEQ ID NO: 1) and AAV1-CMV-Ins5 (SEQ ID NO: 87) vectors (FIGS. 5A-5C and Table 6). Statistical analysis was performed with data from infection at 2K and 4K since data from 1K was below the lowest standard and could not be quantified.

TABLE 6
Statistical significance (adjusted P value) of secreted
hInsulin for the different AAV1-hInsulin vectors.

| | | Adjusted P value | | |
|---|---|---|---|---|
| MOI | Vector comparison | Assay 1 | Assay 2 | Assay 3 |
| 1K | AAV1-CMV-hIns_B10D vs. | 0.2555 | 0.1797 | 0.4003 |
| | AAV1-CMV-Ins5 | | | |
| | AAV1-CMV-hIns_B10D | 0.0107 | 0.001 | 0.0069 |
| | vs. AAV1-CMV-Ins7 | | | |
| | AAV1-CMV-Ins5 vs. | 0.001 | 0.0001 | 0.0011 |
| | AAV1-CMV-Ins7 | | | |
| 2K | AAV1-CMV-hIns_B10D | 0.1317 | 0.4953 | 0.5003 |
| | vs. AAV1-CMV-Ins5 | | | |
| | AAV1-CMV-hIns_B10D | 0.0003 | <0.0001 | <0.0001 |
| | vs. AAV1-CMV-Ins7 | | | |
| | AAV1-CMV-Ins5 vs. | <0.0001 | <0.0001 | <0.0001 |
| | AAV1-CMV-Ins7 | | | |

Data of secreted hInsulin corresponding to each MOI and each assay was analyzed with Anova and Tukey's multiple comparison test. Bold and italic indicates statistical significance.

3.9.3 Functional Analysis of Secreted hInsulin

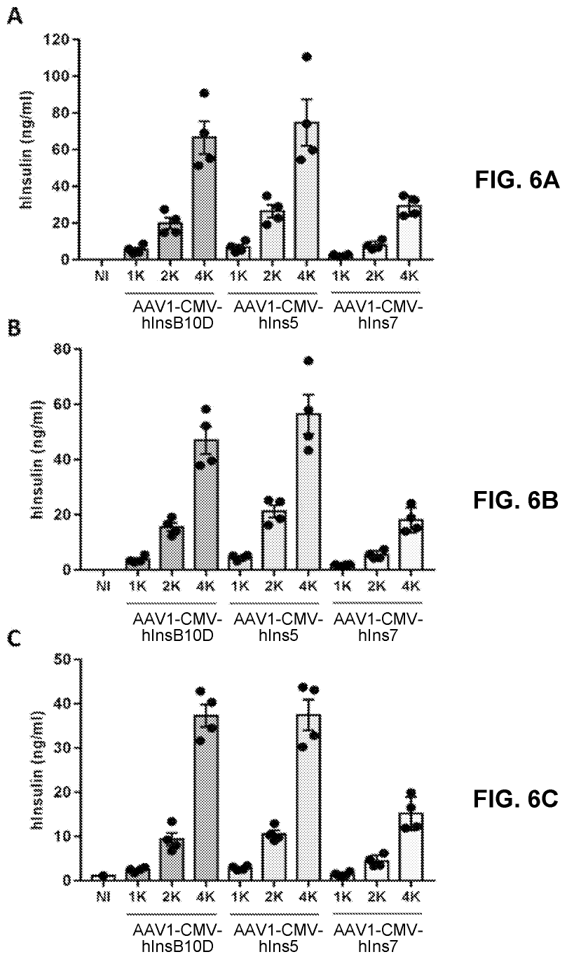

The activity of insulin produced and secreted into cell media by the infected cells was measured with the iLite Insulin Assay Ready Cells (Svar Life Science). Accordingly with the results obtained for mRNA expression and hInsulin protein readouts (FIGS. 4A-4C and 5A-5C and Tables 5 and 6), the insulin activity observed for vectors AAV1-CMV-hIns_B10D (SEQ ID NO: 1) and AAV1-CMV-Ins5 (SEQ ID NO: 87) was not significantly different, while AAV1-CMV-Ins7 (SEQ ID NO: 88) showed again a markedly lower insulin activity (FIGS. 6A-6C and Table 7).

TABLE 7
Statistical significance (adjusted P value) of hInsulin activity for the
different AAV1-hInsulin vectors.

| | | Adjusted P value | | |
|---|---|---|---|---|
| MOI | Vector comparison | Assay 1 | Assay 2 | Assay 3 |
| 1K | AAV1-CMV-hInsB10D vs. AAV1-CMV-Ins5 | 0.6547 | 0.4786 | 0.5085 |
| | AAV1-CMV-hInsB10D vs. AAV1-CMV-Ins7 | 0.173 | 0.0359 | 0.0398 |
| | AAV1-CMV-Ins5 vs. AAV1-CMV-Ins7 | 0.0437 | 0.0058 | 0.0069 |
| 2K | AAV1-CMV-hInsB10D vs. AAV1-CMV-Ins5 | 0.2592 | 0.079 | 0.7067 |
| | AAV1-CMV-hInsB10D vs. AAV1-CMV-Ins7 | 0.0322 | 0.0043 | 0.0217 |
| | AAV1-CMV-Ins5 vs. AAV1-CMV-Ins7 | 0.0026 | 0.0002 | 0.0064 |
| 4K | AAV1-CMV-hInsB10D vs. AAV1-CMV-Ins5 | 0.8049 | 0.4365 | 0.9991 |
| | AAV1-CMV-hInsB10D vs. AAV1-CMV-Ins7 | 0.041 | 0.0084 | 0.0007 |
| | AAV1-CMV-Ins5 vs. AAV1-CMV-Ins7 | 0.0154 | 0.0014 | 0.0007 |

Data of hInsulin activity corresponding to each MOI and each assay was analyzed with Anova and Tukey's multiple comparison test. Bold and italic indicates statistical significance.

### Example 4: In Vitro Comparative Analysis of hGlucokinase Vectors

AAV1-CMV-hGlucokinase vectors bearing the wild-type (SEQ ID NO: 19) and the Gck8 (SEQ ID NO: 93) and Gck12 (SEQ ID NO: 95) hGlucokinase variants were studied for infectivity and potency (mRNA expression, protein content and biological activity). To this end, 2v6.11 cells were infected with AAV1-CMV-hGckWT (SEQ ID NO: 19), AAV1-CMV-Gck8 (SEQ ID NO: 93) and AAV1-CMV-Gck12 (SEQ ID NO: 95) at different MOIs. For the infectivity assay, the intracellular content of vector genomes was quantified. For the potency assay, human glucokinase mRNA expression, glucokinase intracellular content and the glucokinase activity were measured.

### Methods

4.1 Infection of 2v6.11 Cells with AAV1-CMV-hGlucokinase Vectors

The day before infection, 2v6.11 cells were seeded in 24-well plates at a density of 2E+05 cells/well. Cells were grown at 37° C. and 8.5% CO₂ in growth media (DMEM+10% FBS) supplemented with antibiotics (Penicillin=10,000 U/ml, Streptomycin=10,000 μg/ml) and 1 μg/ml Ponasterone A.

Prior to infection, cells were assessed for correct cell confluence (70-80%) at a bright field microscope. To assess cell count, cells of 4 wells were trypsinized and quantified with a Scepter 2.0 Handheld Automated Cell Counter (Merck-Millipore). Cells were infected with AAV1-CMV-hGlucokinase vectors AAV1-CMV-hGckWT, AAV1-CMV-Gck8 and AAV1-CMV-Gck12 (Table 8) at MOI=1000, 2000 and 4000 vg/cell. Cells were infected in quadruplicate for each specific analysis: infectivity, hGlucokinase mRNA expression, intracellular hGlucokinase and glucokinase activity. Non-infected 2v6.11 cells (NI) were used as a negative control. The infection was repeated three times on different days using cells at different cell passage.

TABLE 8
AAV1-CMV-hGck vectors

| AAV Vector | SEQ ID NO |
|---|---|
| AAV1-CMV-hGckWT | SEQ ID NO: 19 |
| AAV1-CMV-Gck8 | SEQ ID NO: 93 |
| AAV1-CMV-Gck12 | SEQ ID NO: 95 |

### 4.2 Infectivity Assay

Twenty-four hours post-infection, cells were observed under a bright field microscope for viability. Next, cell media was aspirated, cells were washed twice with 500 μl 1×PBS and collected in 200 μl 1×PBS with a cell scraper. Intracellular vector genomes were extracted with the DNeasy Blood & Tissue Kits (Qiagen) and amplified by Taqman qPCR with an oligo set targeting the ITR2 sequence. Vector genomes were quantified by interpolation from a standard curve generated with the serial dilution of a standard DNA.

4.3 Human Glucokinase mRNA Expression

Forty-eight hours post-infection and after assessing cell viability, cells were gently washed with 500 μl 1×PBS and collected in 350 μl RLT+β-mercaptoethanol (10 μl/ml) (RNeasy Mini Kit, Qiagen). RNA was extracted with the RNeasy Mini Kit (Qiagen) and RNase-free DNase I (Qiagen) following the protocol provided by the manufacturer except the extension of the on-column DNAse I digestion from the standard 15 minutes to 30 minutes, to ensure appropriate degradation of the infected AAV vector genome. One μg of each RNA sample was retrotranscribed using the Transcriptor FirstStrand cDNA Synthesis kit (Roche). qPCR was performed in triplicate using Taqman Probes Master (Roche) and 2 μl of sample (diluted 1/10). To quantify expression, a primer-probe mix targeting the SV40 poly A signal (sequence common to all the hGlucokinase variants) was used (Forward primer: AGC AAT AGC ATC ACA AAT TTC ACA A; Reverse primer: CAG ACA TGA TAA GAT ACA TTG ATG AGT T; Probe: /56-FAM/AGC ATT TTT TT/ZEN/CAC TGC ATT CTA GTT GTG GTT TGT C/3IABKFQ/). A primer-probe mix for housekeeping gene hRplp0 was used to normalize (forward primer: CAG ACA GAC ACT GGC AAC AT; Reverse primer: GCA GCA TCT ACA ACC CTG AA; Probe: /5HEX/AA CTC TGC A/ZEN/TT CTC GCT TCC TGG A/3IABKFQ).

### 4.4 Quantitative Analysis of Intracellular Glucokinase Content

Forty-eight hours post-infection, cells were gently washed with 500 μl 1×PBS/well. Then, cells were scrapped in 200 μl of ice-cold 1×PBS and collected in a microtube. To obtain cell extracts, cells were frozen (liquid nitrogen) and thawed (37° C. water bath) 3 times, centrifuged at 5000×g for 10 min at 4° C. and the supernatant was saved and stored at −80° C. hGlucokinase was measured in duplicate (Standard and samples) using the Human glucokinase ELISA Kit (Abcam). Samples were diluted 1/20 using 1× diluent N provided by the ELISA kit. Glucokinase content was normalized by total protein content, which was quantified in duplicate in cell extracts by the BCA method using a 1/10 dilution of samples in milliQ water.

### 4.5 Glucokinase Activity Assay

Forty-eight hours post-infection, cells were gently washed with 500 μl 1×PBS/well. Then, 250 μl of trypsin were gently added to the wells, swirled and excess tryspin was removed by aspiration. After a 2-minute incubation at room temperature, cells were collected in 750 μl DMEM+10% FBS by pipetting up and down and transferred into a 1.5 ml microtube. Cells were pelleted at 600×g for 10 min at 4° C., the supernatant was aspirated and cell pellets were stored at −80° C. until processing.

The glucokinase activity was measured with the Glucokinase Activity Assay Kit (AssayGenie). The protocol provided by the manufacturer was followed with the exception that cell pellets were sonicated in 250 μl of Gck assay buffer containing 2.5 mM DTT. Ten μl of a 10-fold dilution of samples were used in the assay.

### 4.6 Statistical Analysis

Each MOI was analyzed independently for the 3 studies performed using Anova and Tukey's multiple comparison test.

### Results

2v6.11 cells were infected with AAV1-hGlucokinase vectors bearing the hGlucokinase variants Gck8 and Gck12 and the WT hGck to assess vector infectivity and potency in three independent studies.

### 4.7 Infectivity Assay

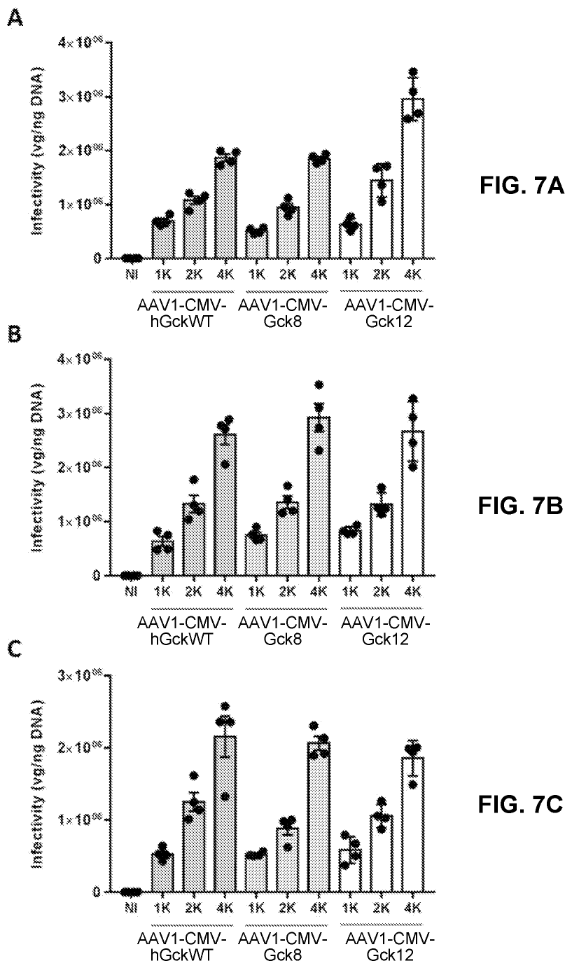

The three AAV1-hGlucokinase vectors AAV1-CMV-hGckWT (SEQ ID NO: 19), AAV1-CMV-Gck8 (SEQ ID NO: 93) and AAV1-CMV-Gck12 (SEQ ID NO: 95) showed no consistent significant differences in their ability to infect 2v6.11 cells at the 3 MOIs tested (1000 vg/cell (1K), 2000 vg/cell (2K) and 4000 vg/cell (4K)) as indicated by the intracellular vector genome content (vg/ng DNA) in three different assays (FIGS. 7A-7C and Table 9).

TABLE 9
Statistical significance (adjusted P value) of the infectivity assays for the
different AAV1-hGlucokinase vectors.

| | | Adjusted P value | | |
|---|---|---|---|---|
| MOI | Vector comparison | Assay 1 | Assay 2 | Assay 3 |
| 1K | AAV1-CMV-hGckWT vs. AAV1-CMV-Gck8 | 0.037 | 0.4745 | 0.9976 |
| | AAV1-CMV-hGckWT v. AAV1-CMV-Gck12 | 0.559 | 0.1414 | 0.7822 |
| | AAV1-CMV-Gck8 v. AAV1-CMV-Gck12 | 0.188 | 0.653 | 0.7453 |
| 2K | AAV1-CMV-hGckWT vs. AAV1-CMV-Gck8 | 0.6554 | 0.9868 | 0.0697 |
| | AAV1-CMV-hGckWT v. AAV1-CMV-Gck12 | 0.0847 | 0.9988 | 0.3986 |
| | AAV1-CMV-Gck8 v. AAV1-CMV-Gck12 | 0.021 | 0.9779 | 0.4742 |
| 4K | AAV1-CMV-hGckWT vs. AAV1-CMV-Gck8 | 0.9945 | 0.6495 | 0.9361 |
| | AAV1-CMV-hGckWT v. AAV1-CMV-Gck12 | 0.0004 | 0.9843 | 0.5164 |
| | AAV1-CMV-Gck8 v. AAV1-CMV-Gck12 | 0.0004 | 0.7482 | 0.7179 |

Infectivity data corresponding to each MOI and each assay was analyzed with Anova and Tukey's multiple comparison test. Bold and italic indicates statistical significance.

### 4.8 Potency Assay

4.8.1 Human Glucokinase mRNA Expression

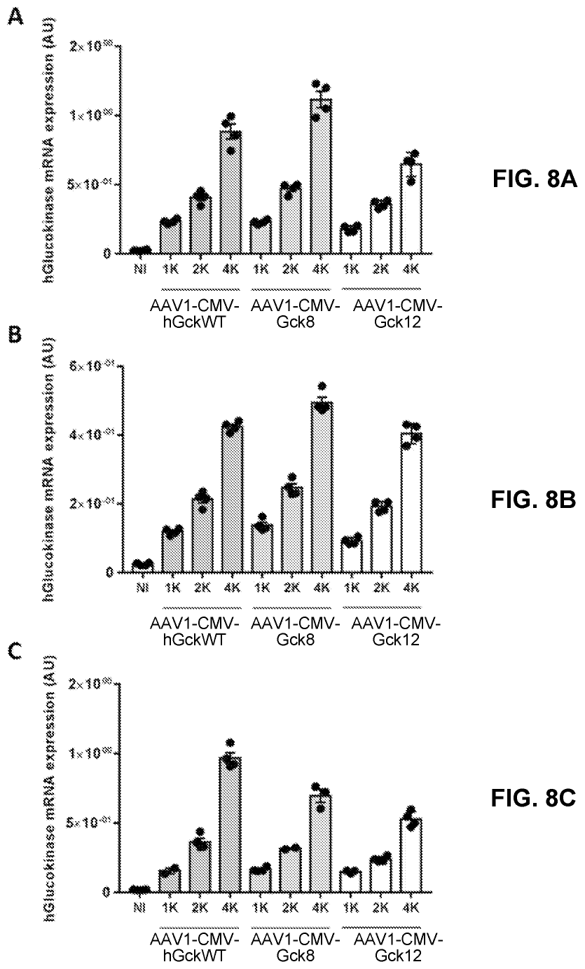

Quantification of hGlucokinase expression levels revealed no consistent significant differences between vectors AAV1-CMV-hGckWT (SEQ ID NO: 19) and AAV1-CMV-Gck8 (SEQ ID NO: 93), bearing the hGckWT and Glucokinase variant Gck8, respectively (FIGS. 8A-8C and Table 10). AAV1-CMV-Gck12 (SEQ ID NO: 95), containing Glucokinase variant Gck12, showed a tendency to mediate a lower mRNA expression when compared to AAV1-CMV-hGckWT (SEQ ID NO: 19) and specially AAV1-CMV-Gck8 (SEQ ID NO: 93), although not consistently among assays and MOIs (FIGS. 8A-8C and Table 10).

TABLE 10
Statistical significance (adjusted P value) of hGlucokinase mRNA
expression for the different AAV1-hGlucokinase vectors.

| | | Adjusted P value | | |
|---|---|---|---|---|
| MOI | Vector comparison | Assay 1 | Assay 2 | Assay 3 |
| 1K | AAV1-CMV-hGckWT vs. AAV1-CMV-Gck8 | 0.9521 | 0.1412 | 0.8088 |
| | AAV1-CMV-hGckWT v. AAV1-CMV-Gck12 | 0.0076 | 0.0333 | 0.9245 |
| | AAV1-CMV-Gck8 v. AAV1-CMV-Gck12 | 0.0119 | 0.0015 | 0.5107 |
| 2K | AAV1-CMV-hGckWT vs. AAV1-CMV-Gck8 | 0.1029 | 0.1016 | 0.3463 |
| | AAV1-CMV-hGckWT v. AAV1-CMV-Gck12 | 0.2076 | 0.328 | 0.0044 |
| | AAV1-CMV-Gck8 v. AAV1-CMV-Gck12 | 0.006 | 0.0098 | 0.0938 |
| 4K | AAV1-CMV-hGckWT vs. AAV1-CMV-Gck8 | 0.0302 | 0.0116 | 0.0026 |
| | AAV1-CMV-hGckWT v. AAV1-CMV-Gck12 | 0.0262 | 0.5546 | <0.0001 |
| | AAV1-CMV-Gck8 v. AAV1-CMV-Gck12 | 0.0004 | 0.0025 | 0.0364 |

hGlucokinase mRNA expression data corresponding to each MOI and each assay was analyzed with Anova and Tukey's multiple comparison test. Bold and italic indicates statistical significance.

### 4.8.2 Quantitative Analysis of Intracellular Glucokinase Content

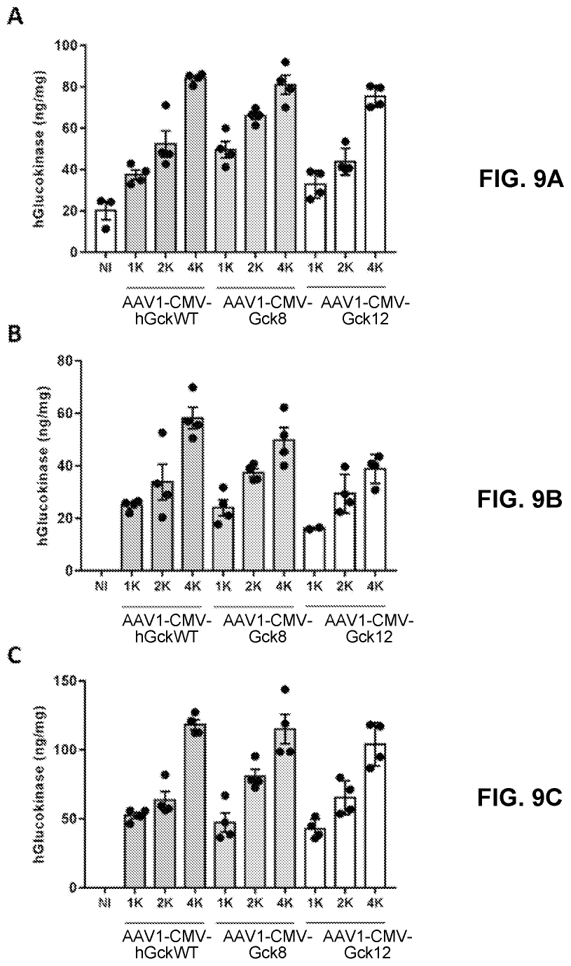

When the intracellular glucokinase content of 2v6.11 cells infected with the different AAV1-hGlucokinase vectors was quantified using a Glucokinase ELISA kit (Abcam), no significant differences that were consistent throughout MOIs and assays were observed among vectors (FIGS. 9A-9C and Table 11).

TABLE 11
Statistical significance (adjusted P value) of intracellular glucokinase
content for the different AAV1-hGlucokinase vectors.

| | | Adjusted P value | | |
|---|---|---|---|---|
| MOI | Vector comparison | Assay 1 | Assay 2 | Assay 3 |
| 1K | AAV1-CMV-hGckWT vs. AAV1-CMV-Gck8 | 0.0613 | 0.9348 | 0.7228 |
| | AAV1-CMV-hGckWT v. AAV1-CMV-Gck12 | 0.595 | 0.1011 | 0.3476 |
| | AAV1-CMV-Gck8 v. AAV1-CMV-Gck12 | 0.013 | 0.1498 | 0.7744 |
| 2K | AAV1-CMV-hGckWT vs. AAV1-CMV-Gck8 | 0.1119 | 0.8494 | 0.1448 |
| | AAV1-CMV-hGckWT v. AAV1-CMV-Gck12 | 0.3677 | 0.7777 | 0.9784 |
| | AAV1-CMV-Gck8 v. AAV1-CMV-Gck12 | 0.0123 | 0.4639 | 0.1938 |
| 4K | AAV1-CMV-hGckWT vs. AAV1-CMV-Gck8 | 0.761 | 0.3349 | 0.9555 |
| | AAV1-CMV-hGckWT v. AAV1-CMV-Gck12 | 0.173 | 0.018 | 0.4525 |
| | AAV1-CMV-Gck8 v. AAV1-CMV-Gck12 | 0.4484 | 0.1813 | 0.6137 |

Data of intracellular glucokinase content corresponding to each MOI and each assay was analyzed with Anova and Tukey's multiple comparison test. Bold and italic indicates statistical significance.

### 4.8.3 Glucokinase Activity Assay

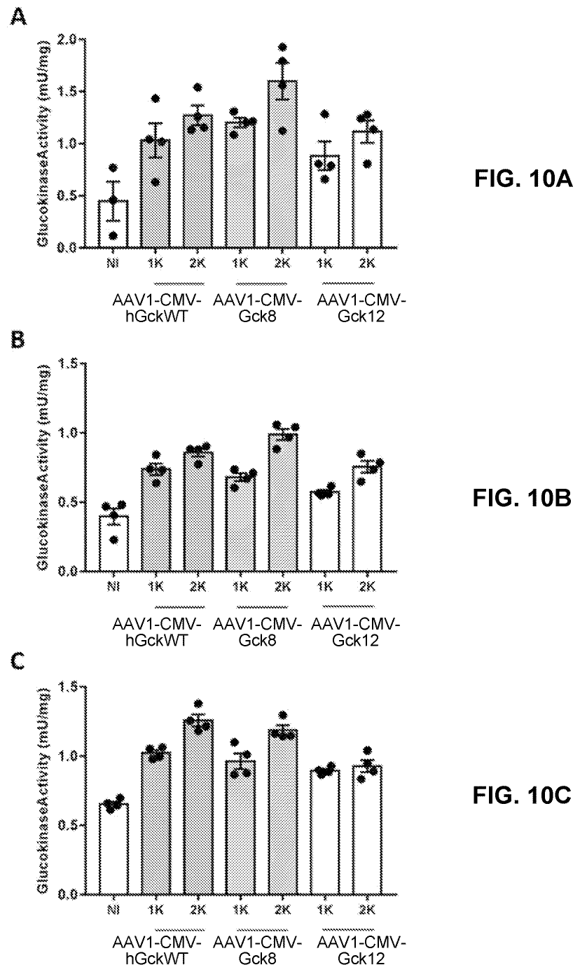

The glucokinase activity in cell extracts of infected cells was measured with the Glucokinase Activity Assay Kit (AssayGenie). Concordant with the results observed for mRNA expression and protein readouts (FIGS. 8A-8C and 9A-9C and Tables 10 and 11), the glucokinase activity observed for vectors AAV1-CMV-hGckWT (SEQ ID NO: 19), AAV1-CMV-Gck8 (SEQ ID NO: 93) and AAV1-CMV-Gck12 (SEQ ID NO: 95) was not significantly different among vectors throughout MOIs and assays (FIGS. 10A-10C and Table 12).

TABLE 12
Statistical significance (adjusted P value) of glucokinase activity for the
different AAV1-hGlucokinase vectors.

| | | Adjusted P value | | |
|---|---|---|---|---|
| MOI | Vector comparison | Assay 1 | Assay 2 | Assay 3 |
| 1K | AAV1-CMV-hGckWT vs. AAV1-CMV-Gck8 | 0.6181 | 0.4162 | 0.4917 |
| | AAV1-CMV-hGckWT v. AAV1-CMV-Gck12 | 0.6977 | 0.0104 | 0.0716 |
| | AAV1-CMV-Gck8 v. AAV1-CMV-Gck12 | 0.2283 | 0.0807 | 0.3913 |
| 2K | AAV1-CMV-hGckWT vs. AAV1-CMV-Gck8 | 0.2323 | 0.0878 | 0.4791 |
| | AAV1-CMV-hGckWT v. AAV1-CMV-Gck12 | 0.6905 | 0.1818 | 0.0008 |
| | AAV1-CMV-Gck8 v. AAV1-CMV-Gck12 | 0.0663 | 0.0046 | 0.0042 |

Data of glucokinase activity corresponding to each MOI and each assay was analyzed with Anova and Tukey's multiple comparison test. Bold and italic indicates statistical significance.

### Example 5: Reversal of Type 1 Diabetic Mice Via Expression of Insulin and Glucokinase in Skeletal Muscle

To assess the effectiveness of a one time administration of AAV vector insulin and glucokinase constructs to remove glucose from blood, skeletal muscles in both hind limbs (quadricep, gastrocnemius, and tibialis cranialis) of C57Blk6 mice were injected with an AAV1 containing a human insulin gene (SEQ ID NO: 1) and an AAV1 containing a rat glucokinase gene.

Mice were first treated with 40 mg of Streptozotocin (STZ) for five consecutive days to deplete beta cells in the pancreas, thereby eliminating production of native mouse insulin and resulting in blood glucose levels reaching ˜600 mg/dL. Animals were then given an equal mixture of AAV1-insulin (SEQ ID NO: 1) and AAV1-rat glucokinase in three identified muscles in both hindlimbs. In a separate group, animals were administered the same total dose of AAV1-insulin (SEQ ID NO: 1) and AAV1-rat glucokinase to two hind limb muscles (quadricep and gastrocnemius) in a lower total volume. These animals were compared to STZ-vehicle-treated and non-diabetic vehicle-treated animals (n=10-11/group). STZ-treated diabetic mice that received AAV1-insulin (SEQ ID NO: 1) and AAV1-rat glucokinase restored and maintained normoglycemia and HbA1C levels in fed and fasted conditions, as observed in earlier studies.

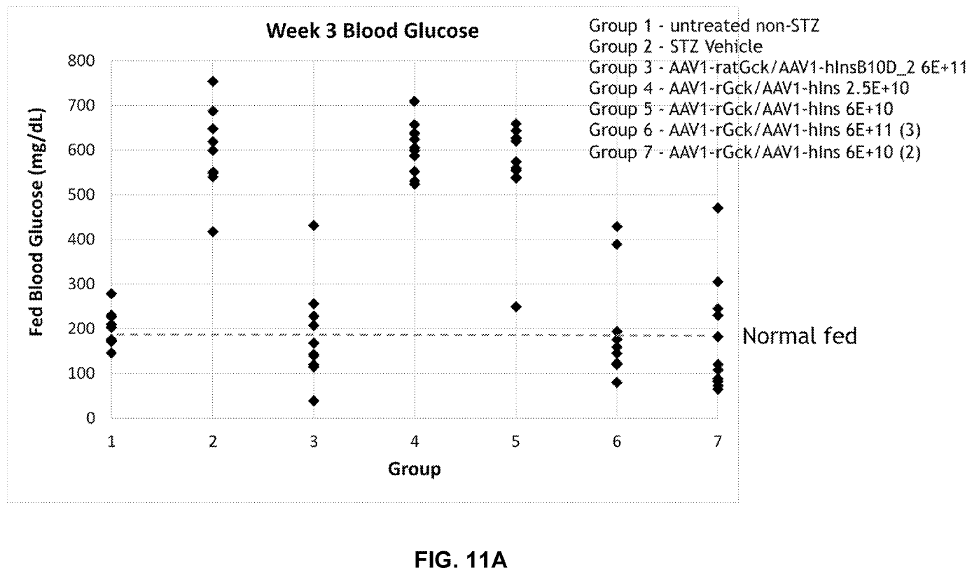

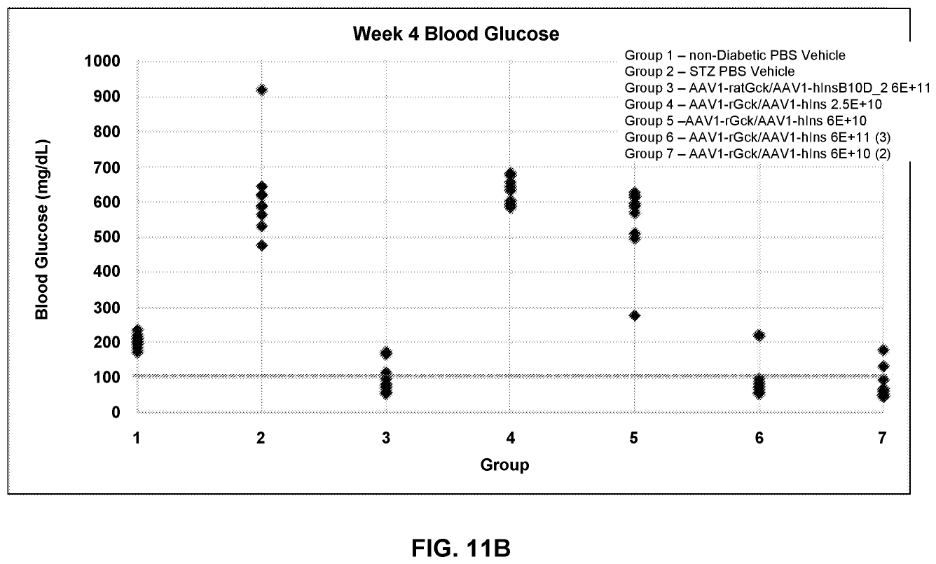

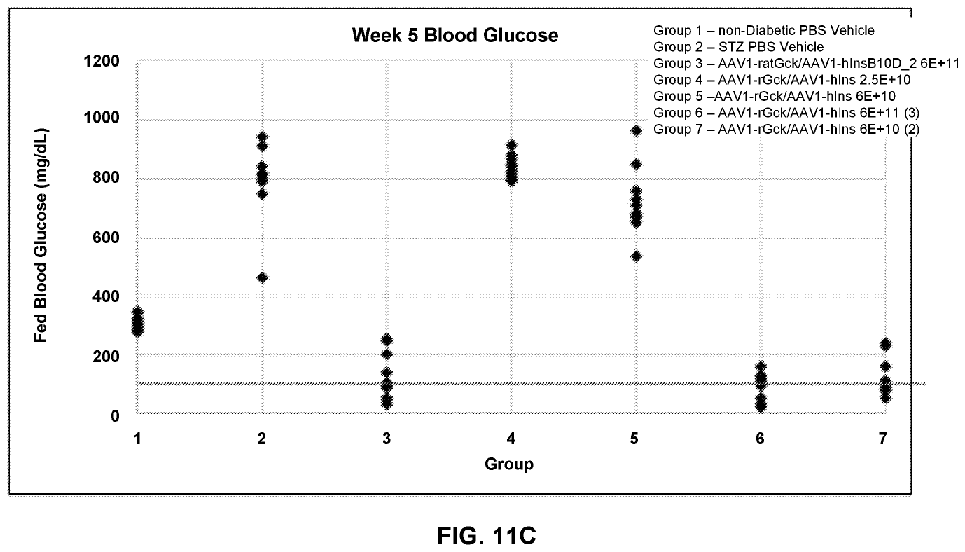

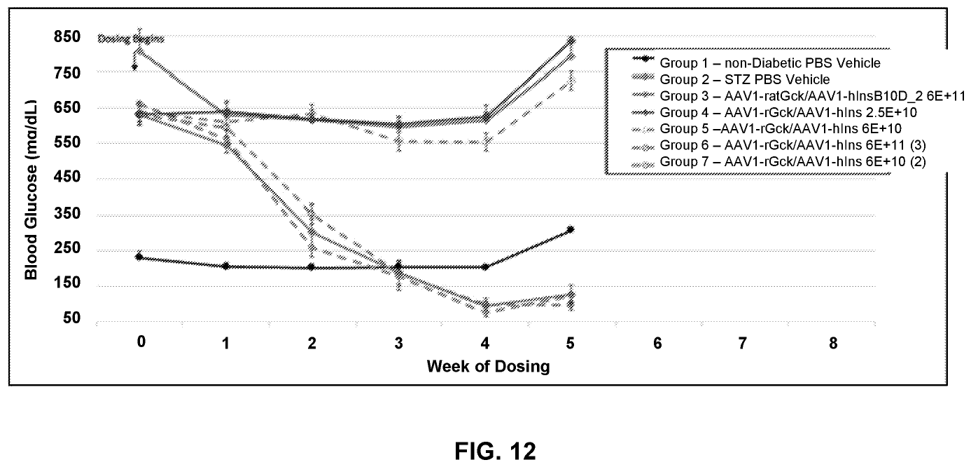

The administration of the gene therapy to two muscles was equally effective as it was when administered to three muscles. Streptozotocin-treated diabetic mice that received AAV1-Insulin (SEQ ID NO: 1) and AAV1-rat Glucokinase restored and maintained normoglycemia in fed (FIG. 11A-11C and FIG. 12) and fasted conditions (week 4 data shown) up to five weeks after injection. In addition, mice treated in only two larger muscle groups (quadricep and gastrocnemius) with higher concentrations of vector appeared to perform equivalent or better than mice treated in three muscle groups (FIGS. 11A-11C and FIG. 12). These results indicate that muscle anatomy and blood flow may be a consideration in allometric translation. Therapeutic effects were observed as early as one week following dosing, with significant reductions in hyperglycemia observed by two weeks in the range of untreated control animals. The joint action of AAV-mediated basal insulin production and glucokinase activity may generate a “glucose sensor” in skeletal muscle that allows proper regulation of glucose in diabetic animals, driving the complete reversal of diabetes in treated animals.

### Example 6: Development of Additional Proinsulin Variants

Ten additional modified nucleic acid human insulin ORF sequences encoding preproinsulin variants were designed (shown in Table 13), corresponding to SEQ ID NOs: 150-159. The ORFs encode preproinsulin variants comprising amino acid mutations in the B chain and/or C chain, substitutions in the signal sequence, addition of furin endoprotease cleavage sites, or combinations thereof.

TABLE 13
Modified Insulin ORF Sequences

| SEQ ID NO. | Nucleic Acid Coding Sequence |
|---|---|
| ORF: 150 | ATGGCTCTGTGGATGAGACTGCTGCCCCTGCTGGCTCTGCTGGCACTGTGGGGACCT |
| | GATCCTGCCGCCGCTTTCGTGAACCAGCACCTGTGTGGCAGCATCCTGGTGGAAGCC |
| | CTGTACCTCGTGTGCGGCGAGAGAGGCTTCTTCTACACCCCTAGGACCAAGAGAGA |
| | GGCCGAGGATCTGCAAGTGGGCCAGGTGGAACTGGGCGGAGGACCTGGCGCTGGAT |
| | CTCTGCAGCCTCTGGCTCTGGAAGGCAGCAGACAGAAAAGGGGCATCGTGGAACAG |
| | TGCTGCACCAGCATCTGCTCCCTGTACCAGCTGGAAAACTACTGCAACTGA |
| ORF: 151 | ATGGCTCTGTGGATGAGACTGCTGCCCCTGCTGGCTCTGCTGGCACTGTGGGGACCT |
| | GATCCTGCCGCCGCTTTCGTGAACCAGCACCTGTGTGGAAGCGTGCTGGTGGAAGCC |
| | CTGTACCTCGTGTGTGGCGAGAGAGGCTTCTTCTACACCCCTAGGACCAAGAGAGA |
| | GGCCGAGGATCTGCAAGTGGGCCAGGTGGAACTGGGCGGAGGACCTGGCGCTGGAT |
| | CTCTGCAGCCTCTGGCTCTGGAAGGCAGCAGACAGAAAAGGGGCATCGTGGAACAG |
| | TGCTGCACCAGCATCTGCTCCCTGTACCAGCTGGAAAACTACTGCAACTGA |
| ORF: 152 | ATGAACAGCTTCAGCACAAGCGCCTTCGGCCCCGTGGCTTTCTCTCTGGGACTGCTG |
| | CTGGTGCTGCCTGCCGCTTTCCCTGCCCCTTTCGTGAACCAGCACCTGTGCGGCAGC |
| | CACCTGGTGGAAGCTCTGTACCTCGTGTGCGGCGAGAGAGGCTTCTTCTACACCCCT |
| | AGGACCAAGAGAGAGGCCGAGGATCTGCAAGTGGGCCAGGTGGAACTGGGCGGAG |
| | GACCTGGCGCTGGATCTCTGCAGCCTCTGGCTCTGGAAGGCAGCAGACAGAAAAGG |
| | GGCATCGTGGAACAGTGCTGCACCAGCATCTGCTCCCTGTACCAGCTGGAAAACTA |
| | CTGCAACTGA |
| ORF: 153 | ATGCTGAGAGGACCTGGACCAGGACTGCTGCTGCTGGCCGTGCAGTGTCTGGGAAC |
| | AGCCGTGCCTAGCACAGGCGCCTTCGTGAACCAGCACCTGTGTGGCAGCCACCTGG |
| | TGGAAGCCCTGTACCTCGTGTGTGGCGAGAGAGGCTTCTTCTACACCCCTAGGACCA |
| | AGAGAGAGGCCGAGGATCTGCAAGTGGGCCAGGTGGAACTGGGCGGAGGACCAGG |
| | CGCTGGATCTCTGCAGCCTCTGGCTCTGGAAGGCAGCAGACAGAAAAGGGGCATCG |
| | TGGAACAGTGCTGCACCAGCATCTGCTCCCTGTACCAGCTGGAAAACTACTGCAACT |
| | GA |
| ORF: 154 | ATGGCTCTGTGGATGAGACTGCTGCCCCTGCTGGCTCTGCTGGCACTGTGGGGACCT |
| | GATCCTGCCGCCGCTTTCGTGAACCAGCACCTGTGTGGCAGCCACCTGGTGGAAGCC |
| | CTGTACCTCGTGTGTGGCGAGAGAGGCTTCTTCTACACCCCTAGGACCAAGAGAGA |
| | GGCCGAGGATCTGCAAGTGGGCCAGGTGGAACTGGGCGGAGGACCTGGCGCTGGAT |
| | CTCTGCAGCCTCTGGCTCTGGAAGGCAGCAGACAGAAAAGGGGCATCGTGGAACAG |
| | TGCTGCACCAGCATCTGCTCCCTGTACCAGCTGGAAAACTACTGCAACTGA |
| ORF: 155 | ATGGCTCTGTGGATGAGACTGCTGCCCCTGCTGGCTCTGCTGGCACTGTGGGGACCT |
| | GATCCTGCCGCCGCTTTCGTGAACCAGCACCTGTGTGGCAGCCACCTGGTGGAAGCC |
| | CTGTACCTCGTGTGTGGCGAGAGAGGCTTCTTCTACACCCCTAGGACCAAGAGAGA |
| | GGCCGAGGATCTGCAAGTGGGCCAGGTGGAACTGGGCGGAGGACCTGGCGCTGGAT |
| | CTCTGCAGCCTCTGGCTCTGGAAGGCAGCAGACAGAAAAGGGGCATCGTGGAACAG |
| | TGCTGCACCAGCATCTGCTCCCTGTACCAGCTGGAAAACTACTGCAACTGA |
| ORF: 156 | ATGAACAGCTTCAGCACAAGCGCTTTCGGCCCCGTGGCTTTTTCTCTGGGACTGCTG |
| | CTGGTTCTGCCTGCCGCTTTTCCTGCTCCTTTCGTGAACCAGCACCTGTGCGGAAGCG |
| | TGCTGGTGGAAGCTCTGTATCTCGTGTGTGGCGAGAGAGGCTTCTTCTACACCCCTA |
| | GGACCAAGAGAGAGGCCGAGGATCTGCAAGTCGGCCAGGTTGAACTTGGCGGAGG |
| | ACCTGGTGCTGGATCTCTGCAGCCTCTTGCTCTGGAAGGCAGCAGACAGAAGAGGG |
| | GCATCGTCGAGCAGTGCTGCACCAGCATCTGTAGCCTGTACCAGCTGGAAAACTACT |
| | GCAACTGA |
| ORF: 157 | ATGGCACTTTGGATGAGACTGCTGCCACTGCTGGCCCTGCTTGCTCTTTGGGGACCT |
| | GATCCTGCTGCCGCCTTTGTGAACCAGCACCTGTGTGGAAGCCATCTGGTGGAAGCC |
| | CTGTACCTCGTGTGTGGCGAGAGAGGCTTCTTCTACACCGACAGGACCAAGAGAGA |
| | GGCCGAGGATCTGCAAGTCGGCCAGGTTGAACTTGGCGGAGGACCTGGTGCTGGAT |
| | CTCTGCAGCCTCTTGCTCTGGAAGGCAGCAGACAGAAGAGGGGCATCGTCGAGCAG |
| | TGCTGCACCAGCATCTGTAGCCTGTACCAGCTGGAAAACTACTGCAACTGA |
| ORF: 158 | ATGGCCTTGTGGATGCGGCTTTTGCCGCTGCTCGCGCTCTTGGCCCTCTGGGGGCCC |
| | GACCCGGCTGCCGCTTTCGTCAATCAGCACCTCTGCGGCAGCCACCTCGTGGAAGCG |
| | CTTTACTTGGTCTGCGGCGAGAGGGGCTTCTTTTATACTCCACGGACTAAGCGGGAG |
| | GCCGAAGACTTGCAAGTCGGGCAAGTGGAACTGGGAGGCGGGCCGGGTGCGGGGT |
| | CTCTGCAACCTCTTGCCCTTGAGGGGTCACGACAAAAGAGAGGGATAGTTGAGCAG |
| | TGTTGTACGTCAATTTGTTCCCTGTATCAACTTGAAAATTATTGCAATTAG |
| ORF: 159 | ATGGCTCTGTGGATGAGACTTCTTCCTCTTTTGGCCCTGCTGGCCCTTTGGGGCCCTG |
| | ATCCTGCCGCCGCCTTCGTTAACCAACATCTGTGCGGTTCACACCTGGTTGAAGCTC |
| | TTTACCTCGTTTGTGGAGAAAGAGGCTTTTTTTACACACCACGGACCAAAAGGGAGG |
| | CAGAGGACCTGCAGGTGGGACAGGTTGAGCTGGGCGGGGGCCCTGGGGCAGGCTC |
| | ACTTCAGCCACTCGCCCTGGAGGGCTCTCGCCAGAAGAGAGGTATAGTGGAGCAAT |
| | GCTGTACCAGCATTTGTTCTCTCTATCAGCTGGAGAATTACTGTAACTAA |

### Example 7: In Vitro Evaluation of Preproinsulin Variants

To evaluate insulin production and secretion levels mediated by SEQ ID NOs: 150-159 in vitro, each insulin variant was first cloned in AAV plasmids (pAAV) under the control of the miniCMV promoter (pAAV-miniCMV-InsX, where X indicates the specific ORF, i.e., SEQ ID NO: 150-159). The plasmid name and corresponding ORF sequence are listed in Table 14.

TABLE 14
Insulin Plasmid Names

| Plasmid Name | ORF SEQ ID NO |
|---|---|
| pAAV-miniCMV-Ins1 | 150 |
| pAAV-miniCMV-Ins2 | 151 |
| pAAV-miniCMV-Ins3 | 152 |
| pAAV-miniCMV-Ins4 | 153 |
| pAAV-miniCMV-Ins5 | 154 |
| pAAV-miniCMV-Ins6 | 155 |
| pAAV-miniCMV-Ins7 | 156 |
| pAAV-miniCMV-Ins8 | 157 |
| pAAV-miniCMV-Ins9 | 158 |
| pAAV-miniCMV-Ins10 | 159 |

AAV expression cassettes were obtained by cloning, between the ITRs of AAV2, the human preproinsulin variants under the control of the miniCMV promoter (pAAV-miniCMV-InsX, where X indicates the specific insulin variant).

Single-stranded AAV vectors of serotype 1 encoding preproinsulin variants under the control of the miniCMV promoter and human glucokinase under the control of the RSV promoter (AAV1-InsX, where X indicates the specific proinsulin variant) were produced by triple transfection of HEK293 cells according to standard methods (Ayuso, E. et al., 2010. Curr Gene Ther. 10 (6): 423-36). Cells were cultured in 10 roller bottles (850 cm², flat; Corning™, Sigma-Aldrich Co., Saint Louis, MO, US) in DMEM 10% FBS to 80% confluence and co-transfected by calcium phosphate method with a plasmid carrying the expression cassette flanked by the AAV2 ITRs, a helper plasmid carrying the AAV2 rep gene and the AAV of serotypes 1 cap gene, and a plasmid carrying the adenovirus helper functions. Noncoding plasmids were used to produce null vectors (pAAV-Null). AAV were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso, E. et al., 2010. Curr Gene Ther. 10 (6): 423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M, et al., Hum. Gene Ther. 2010; 21:1273-1285). The vectors were constructed according to molecular biology techniques well known in the art.

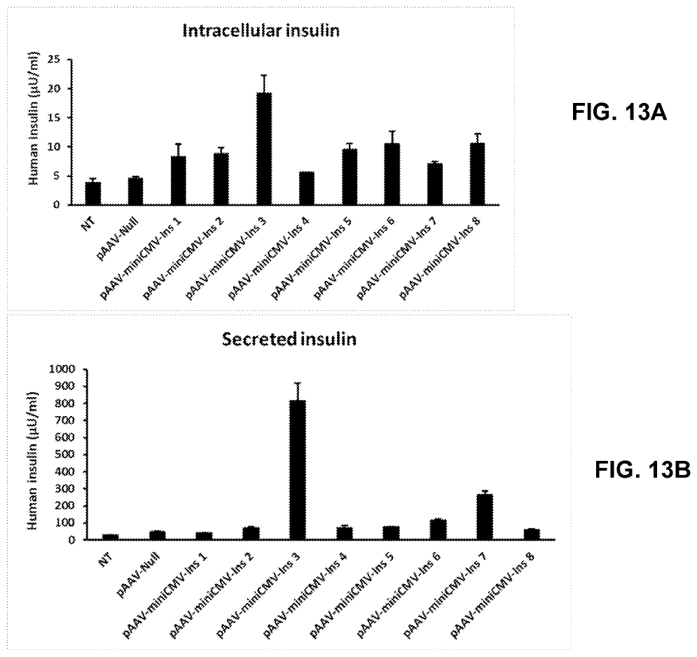

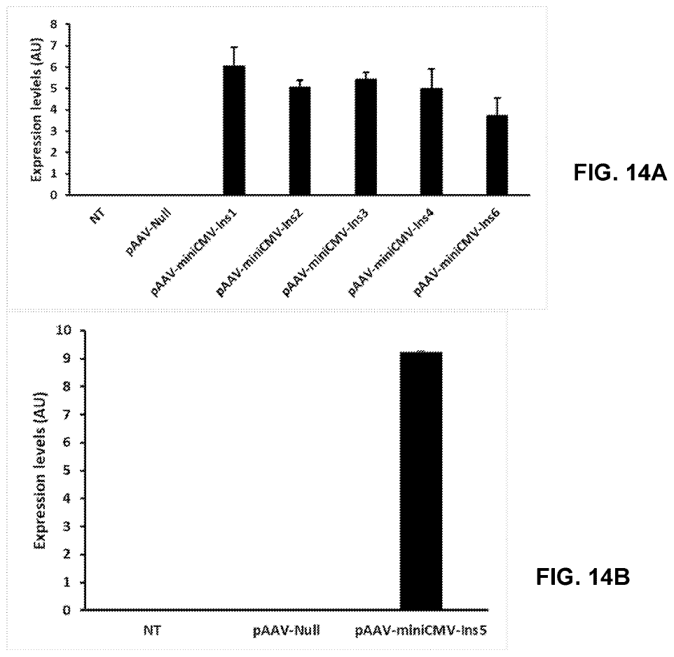

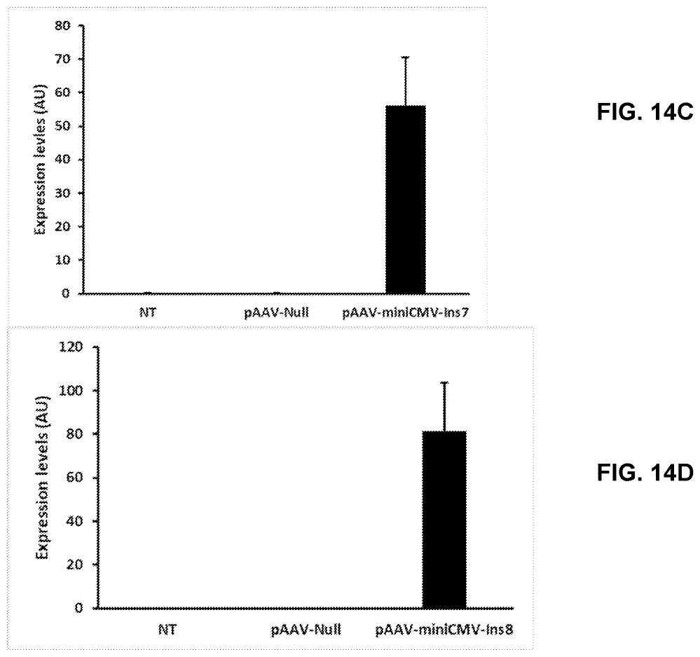

First, HEK293 cells were transfected with equimolar amounts of pAAV-miniCMV-Ins1 to 8 plasmids. HEK293 cells were cultured in a 24-well plate and transfected with 0.8 μg of DNA per well using Lipofectamine 2000 following the manufacturer's instructions (Thermo Fisher Scientific). Non-transfected HEK293 cells and HEK293 cells transfected with an AAV plasmid comprising no transgene (pAAV-Null) served as controls. The pAAV-miniCMV-Ins3 plasmid mediated both the highest intracellular insulin content and secretion of insulin into the culture media (FIGS. 13A-13B). These results were not due to improved insulin expression levels in HEK293 cells transfected with the pAAV-miniCMV-Ins3 plasmid in comparison with the rest of variants (FIGS. 14A-14D).

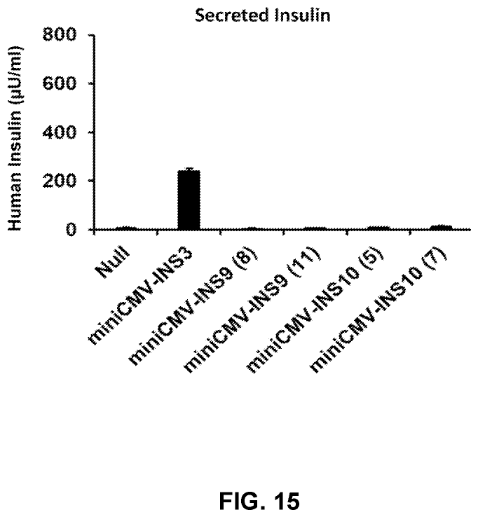

Next, HEK293 cells were transfected with pAAV-miniCMV-Ins3, pAAV-miniCMV-Ins9 or pAAV-miniCMV-Ins10. HK293 cells transfected with the pAAV-Null plasmid were used as control. Similar to the previous observations, cells transfected with pAAV-miniCMV-Ins3 outperformed those transduced with pAAV-miniCMV-Ins9 or pAAV-miniCMV-Ins9 (FIG. 15).

### Example 8: In Vivo Evaluation of Biological Activity of Preproinsulin Variants

To evaluate biological activity, AAV1 vectors encoding the preproinsulin variants AAV1-Ins1, AAV1-Ins3, AAV1-Ins4, AAV1-Ins5 or AAV1-Ins6 (See Table 13) were generated and their efficacy to enhance glucose disposal in vivo was assessed in healthy mice. To this end, CD1 mice were treated with 3×10¹¹ viral genomes (vg) of AAV-Ins or AAV1-Null vectors. Mice were anesthetized with an intraperitoneal injection of ketamine (100 mg/kg) and xylazine (10 mg/kg). Hind limbs were shaved and vectors were administered by intramuscular injection in a total volume of 180 μl divided into six injection sites distributed in the quadriceps, gastrocnemius, and tibialis cranealis of each hind limb.

Three weeks post-AAV administration, a glucose tolerance test was performed. To perform the glucose tolerance test, Awake mice were fasted overnight (16 h) and administered with an intraperitoneal injection of glucose (2 g/kg body weight). Glycemia was measured in tail vein blood samples at the indicated time points.

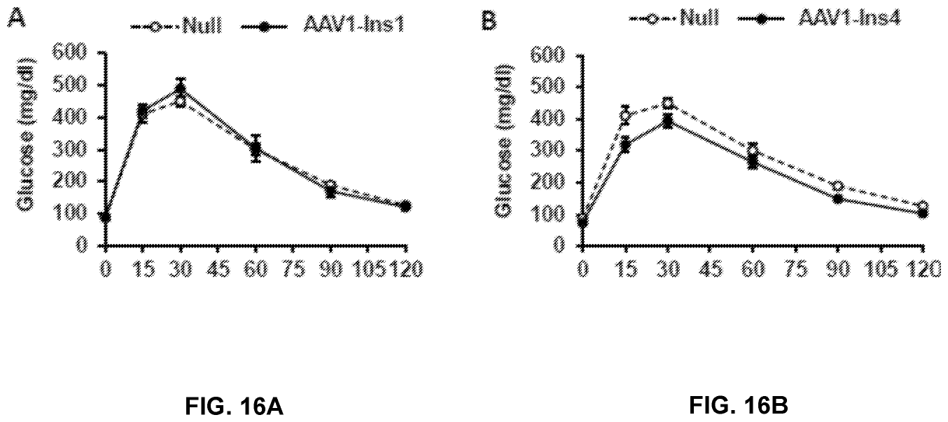

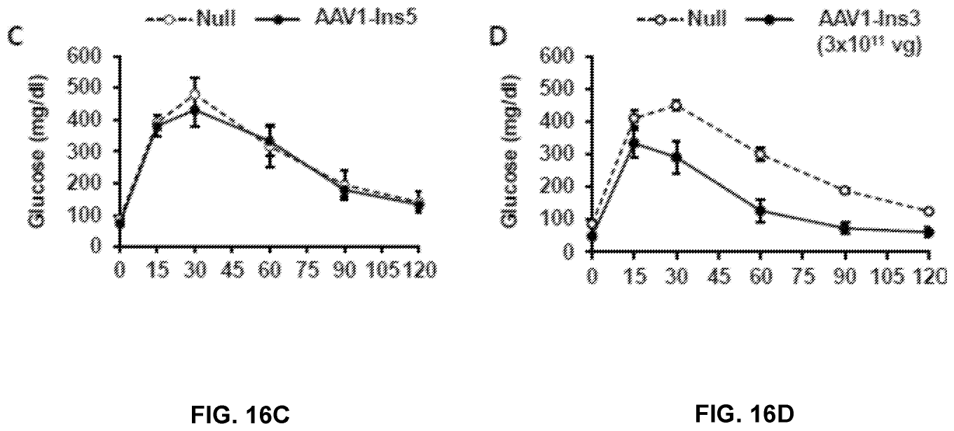

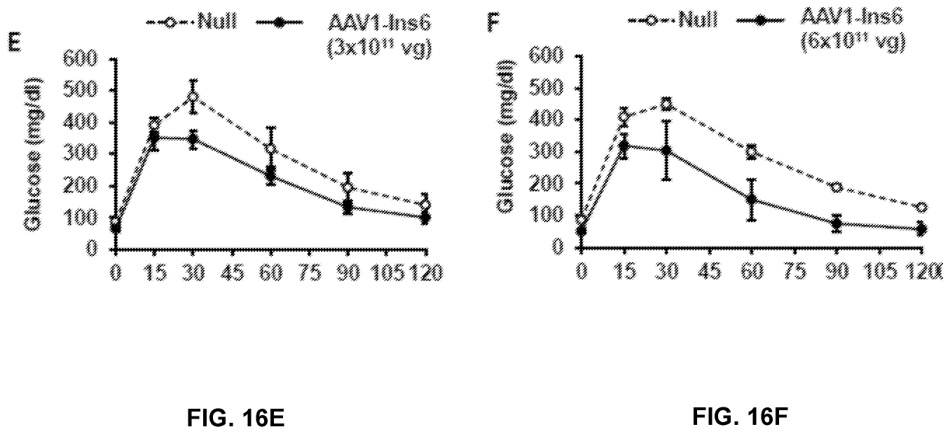

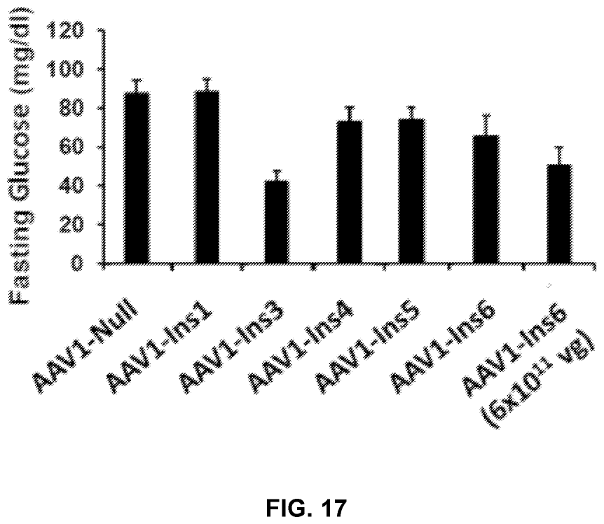

No differences in blood glucose levels following an intraperitoneal glucose tolerance test were observed among cohorts of control mice treated with AAV1 vectors encoding WT preproinsulin, preproinsulin variant 1 or 4 (FIGS. 16A-16C and FIG. 17). In contrast, mice receiving AAV1-Ins3 vectors showed improved glucose tolerance in comparison with healthy mice (FIG. 16D and FIG. 17). Treatment with AAV1-Ins6 partially enhanced glucose disposal (FIG. 16E and FIG. 17). Results for mice treated with 6×10¹¹ vg of AAV-Ins6 vectors and control mice with 3×10¹¹ vg of AAV1-Ins3 vectors are shown in FIG. 16F and FIG. 17.

### Example 9: In Vitro Evaluation of AAV-Ins or AAV-Gck Stimulation of TLR9

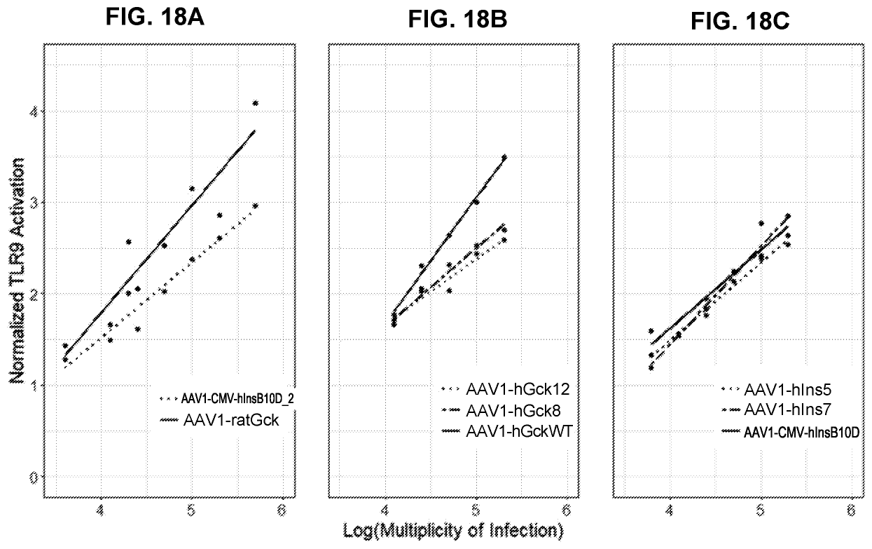

Differences in TLR9 stimulation were detected in HEK-Dual™ hTLR9 cells transduced with AAV1-ratGck or AAV1-hInsB10D_2 (FIG. 18A). TLR9 stimulation was reduced in cells transduced with modified AAV1-GCK constructs including (1) a CMV promoter and modified hGck8 coding sequence (AAV1-hGck8) and (2) a CMV promoter and modified hGck12 coding sequence (AAV1-hGck12) compared to wildtype AAV1-GCK control construct including a CMV promoter and hGckWT coding sequence (AAV1-hGckWT) (FIG. 18B). TLR9 stimulation was similar among cells transduced with control AAV1-CMV-hInsB10D and modified AAV1-Ins constructs including (1) a CMV promoter and modified hIns5 coding sequence (AAV1-hIns5) and (2) a CMV promoter and modified hIns7 coding sequence (AAV1-hIns7) (FIG. 18C). Slopes of TLR9 stimulation are provided in Table 15. The results show reduced stimulation of TLR9 with constructs including modified Gck coding sequences and suggest reduced immune activation with the modified constructs.

TABLE 15
Slope of TLR9 Stimulation after Administration of AAV-Ins and AAV-
Gck vectors.

| Vector 1 | Vector 2 | Slope 1 | Slope 2 | p |
|---|---|---|---|---|
| AAV1-ratGck | AAV1-hInsB10D_2 | 1.3 | 0.8 | 0.02 |
| AAV1-hGckWT | AAV1-hGck8 | 1.5 | 0.9 | 0.004 |
| AAV1-hGckWT | AAV1-hGck12 | 1.5 | 0.7 | 0.001 |
| AAV1-hGck8 | AAV1-hGck12 | 0.9 | 0.7 | 0.21 |
| AAV1-CMV-hInsB10D | AAV1-hIns5 | 0.7 | 0.8 | 0.54 |
| AAV1-CMV-hInsB10D | AAV1-hIns7 | 0.7 | 0.7 | 0.55 |
| AAV1-hIns5 | AAV1-hIns7 | 0.8 | 0.7 | 0.98 |
| AAV1-ratGck | AAV1-hGckWT | 1.3 | 1.5 | 0.59 |
| AAV1-hGckWT | AAV1-hInsB10D_2 | 1.5 | 0.8 | 0.001 |
| AAV1-hGckWT | AAV1-CMV-hInsB10D | 1.5 | 0.7 | 0.001 |
| AAV1-hInsB10D_2 | AAV1-CMV-hInsB10D | 0.8 | 0.7 | 0.58 |

### Example 10: In Vivo Evaluation of AAV-Ins and AAV-Gck Vectors in Diabetic Mice

The efficacy of AAV1 vector constructs expressing rat glucokinase and human insulin was evaluated in a streptozotocin-induced model of diabetic C57BL/6J mice following administration of six intramuscular injections. Two cohorts of mice (8-9 weeks of age at initiation of dosing) were obtained. One cohort was administered 5 daily i.p. doses of streptozotocin (50 mg/kg; STZ) to induce diabetes. The second cohort was administered 5 daily i.p. doses of Na-Citrate buffer to serve as a non-diabetic controls.

Following induction, animals were allowed ad lib access to food and municipal tap water treated by reverse osmosis and chlorinated to maintain 1-6 ppm of chlorine via an automatic watering system. Animals were acclimated to the vivarium for 4 days prior to the determination of baseline body weight, non-fasting blood glucose, and circulating mouse insulin levels. The animals were then blocked and assigned to treatment groups such that there were no significant differences within or between groups with regard to any of these parameters.

Prior to treatment, animals were anesthetized with isoflurane in oxygen, and a 30 microliter (μL) dose was administered by direct injection with an insulin syringe into the quadriceps, gastrocnemius, and tibialis anterior muscles of each hindlimb. The construct AAV1-926+AAV1-927 (a 1:1 mixture of rat GcK and human Insulin, respectively, which had been previously described in a published study from the Bosch laboratory (Mas et al 2006) and U.S. Pat. No. 9,309,534 which is hereby incorporated by reference), was administered as a control to for comparison. Vectors were administered at either high, middle (mid), or low doses. High doses were 3 times greater than the low dose and 1.5 times greater than the mid dose. Dosing was consistent among all experiments. Each injection (a total of six per mouse) deposited the entire vector suspension (30 uL) per injection into the selected muscle bundles (Tibialis anterior, quadricep, and gastrocnemius) bilaterally. Treatment groups are outlined in Table 16.

TABLE 16
Diabetic Mouse Model Treatment Groups

| Groups | AAV doses | N | Treatment |
|---|---|---|---|
| Healthy Control | None | 10 | None |
| Diabetic Control | None | 10 | STZ 40 mg/kg |
| AAV1hINSB10 (AAV927) + | High dose | 10 | |
| AAV1rGcK (AAV926) | | | |
| AAV1mWThINS (B10H | High dose | 10 | |
| with Insulin signal peptide + | | | |
| AAV1rGcK & AAV926 | | | |
| (B10H AAV1-GCK) | | | |
| AAV1hINSB10 (Ins-2; | Low dose | 10 | |
| B10D) + AAV1rGcK & | | | |
| AAV926 | | | |
| (hINS B10D AAV1-GCK) | | | |
| AAV1hINSB10 (Ins-2; | Medium | 10 | |
| B10D) + AAV1rGcK | dose | | |
| (AAV926) | | | |
| AAV1mWThINS (Ins-17; | Low dose | 10 | |
| B10H with IL-6 signal | | | |
| peptide) + AAV1rGcK (Ins- | | | |
| 17) (AAV926) | | | |
| (Ins-B10H + IL6 AAV1- | | | |
| GCK) | | | |
| AAV1mWThINS (INS-17; | High dose | 10 | |
| B10H with IL-6 signal | | | |
| peptide) + AAV1rGcK | | | |
| (AAV926) | | | |
| (Ins-B10H + IL6 AAV1- | | | |
| GCK) | | | |

Body weight and non-fasted blood glucose were determined weekly. For consistency, every measurement was made at the same time of day (between 1-3 PM). A fasted blood glucose was determined at week 4 and an oral glucose tolerance test (OGTT) performed at week 8. At the termination of the study, blood samples were collected from all animals to measure blood glucose, mouse and human circulating insulin, HbA1c and other metabolic parameters. Tissue samples of muscles, liver, and pancreas were collected, weighed, and preserved for evaluation of mRNA, protein levels and protein activity. Baseline values for all mice treated with either STZ or Na Citrate buffer are shown in Table 17.

TABLE 17
Baseline Values for Mice Treated with STZ or Na Citrate Buffer

| | Body | Non-fasted Blood | Circulating Mouse |
|---|---|---|---|
| Treatment | weight (g) | Glucose (mg/dl) | insulin (ng/mL) |
| Na Citrate buffer | 25.6 ± 0.4 | 182.7 ± 8.8 | 0.35 ± 0.09 |
| STZ | 21.9 ± 0.2 | 508.8 ± 15 | BLOQ |

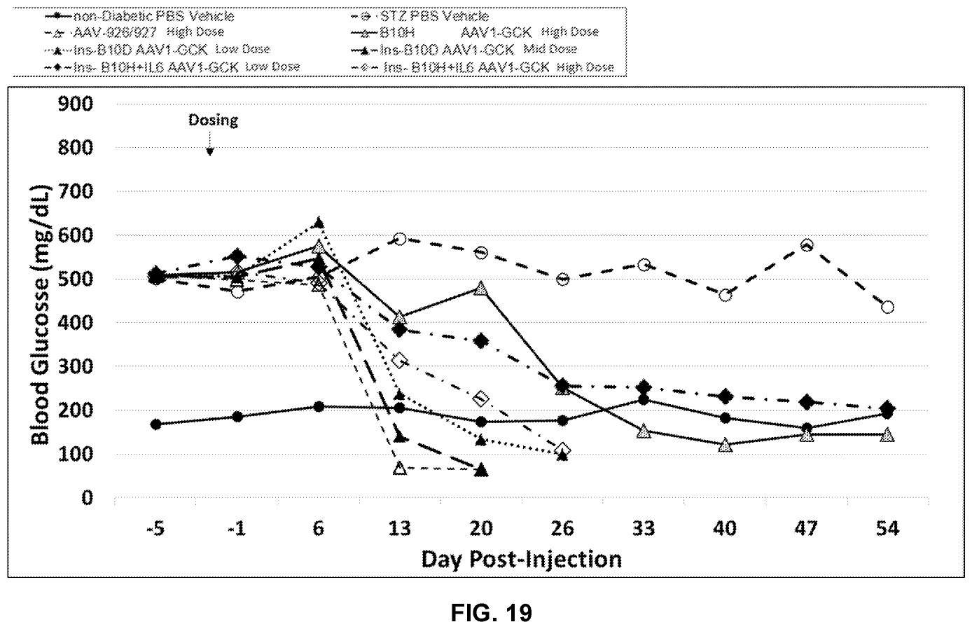

The test articles were effective in significantly lowering blood glucose to the level of non-diabetic controls by day 33 (FIG. 19). Once normoglycemia was achieved, the effect was maintained throughout the course of the study. Treatment appeared to protect against weight loss often associated with untreated type 1 diabetes and restored a number of metabolic parameters to levels associated with normoglycemia. Optimal profiles for kinetics and glucose lowering effects were demonstrated with the B10H construct containing the native insulin signal peptide (High Dose) and the B10H construct containing the IL-6 signal peptide (Low Dose).

These data further showed that insulin and glucokinase can be expressed in skeletal muscle using AAV1 vectors. Treatment with these vectors in a mouse model of Type 1 Diabetes rapidly restored and maintained normoglycemia in fed and fasted conditions for 8 weeks.

10.1 Circulating Insulin Levels in Diabetic Mice after Administration of AAV-Ins and AAV-Gck Vectors

Administration of STZ eliminated all detectable circulating mouse insulin. To assess the efficacy of the AAV1 vector constructs including coding sequences for either hInsB10D or hInsB10H with native or IL-6 signal sequences, circulating human insulin was measured in STZ mice following a 6-hr fast 4 weeks after i.m. injections of AAV1 vector constructs. All samples were analyzed via a validated plate-based ELISA method.

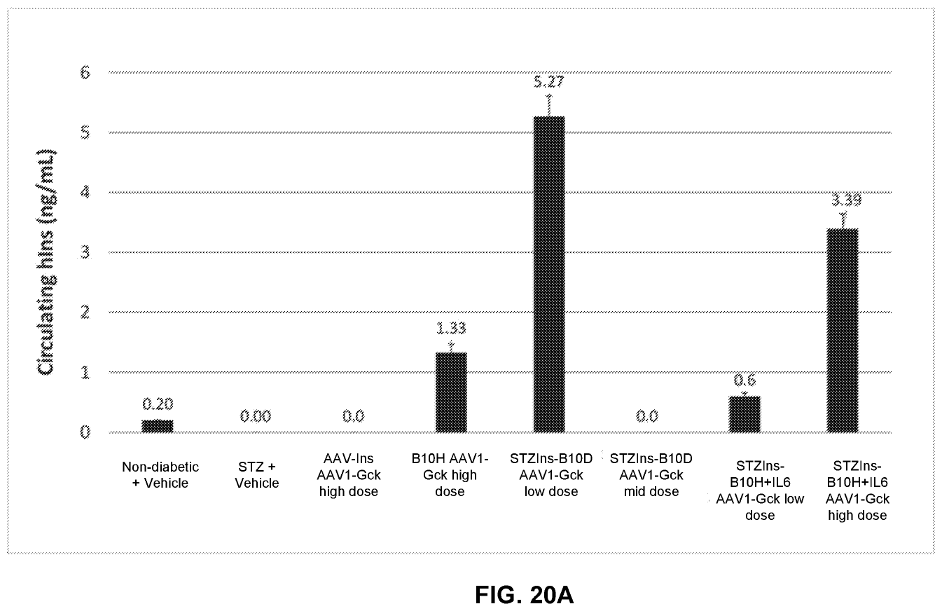

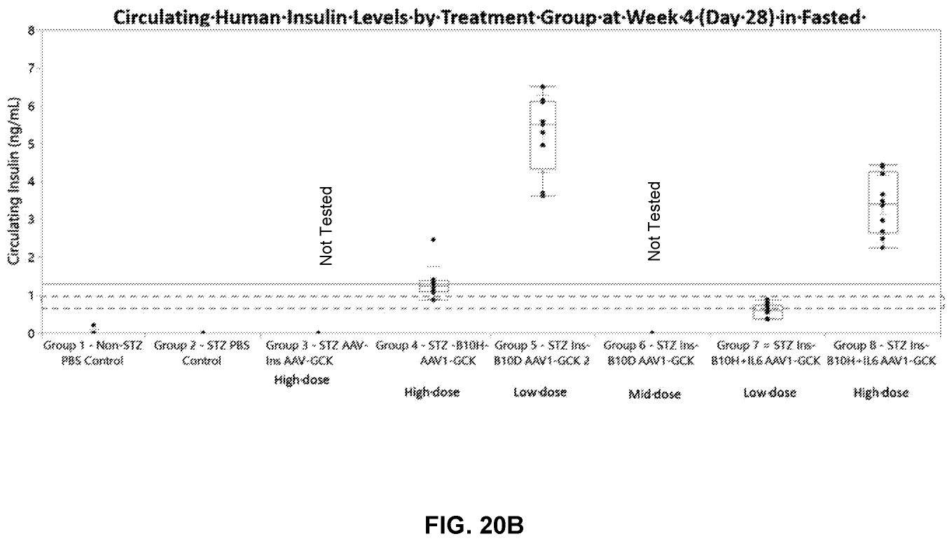

Fasting circulating insulin levels in C57Bl/6 mice reported in the literature typically range from 0.75 to 1.0 ng/mL. By week 4, mice injected with AAV1-926+AAV1-927 (high dose) and hINSB10D (included coding sequence of SEQ ID NO: 110)+AAV1-GCK (AAV926) (mid dose) had been euthanized due to persistent hypoglycemia. At the termination endpoint, mean circulating human insulin levels for those groups were 10.1 ng/ml and 4.5 ng/ml, respectively. At week 4, circulating human insulin of mice injected with hInsB10D (included coding sequence of SEQ ID NO: 110)+AAV1-GCK (AAV926) (low dose) and hInsB10H+IL6 (included coding sequence of SEQ ID NO: 120)+AAV1-GCK (AAV926) (high dose) had reached levels of 5.27±0.34 and 3.39±0.25 ng/ml respectively (FIG. 20A). Ultimately, these two groups were also euthanized due to hypoglycemia.

As shown in FIG. 19, the animals administered hInsB10H (included coding sequence of SEQ ID NO: 111)+AAV1-GCK (AAV926) (high dose) and hInsB10H+IL6 (included coding sequence of SEQ ID NO: 120)+AAV1-GCK (AAV926) (low dose) both reduced and maintained blood glucose at or near the level of non-Diabetic control mice for the duration of the study. Circulating human insulin values for these mice were 1.33±0.14 and 0.6±0.1 ng/mL respectively (FIGS. 20A and 20B). These data show that the coadministration of hINS and hGCK to skeletal muscle of STZ-treated mice can effectively control blood glucose when circulating insulin levels are within the range of normal fasting (i.e., basal) levels.

10.2 Oral Glucose Tolerance in Diabetic Mice after Administration of AAV-Ins and AAV-Gick Vectors

Following the final fed-glucose measurement on day 54, and prior to entering into the 12 hour dark period, animals were placed in clean cages. Food was removed, but access to water was provided throughout the procedure. Following a 4-6 hour fast, animals were weighed and administered a glucose solution (0.2 mg/mL glucose at 10 mL/kg) by oral gavage at a dose of 2 g/kg glucose. Blood glucose was then determined via tail snip using a hand-held glucometer with the 2nd drop (5-10 μL) of blood from the tail. Measurements were made at the following times relative to glucose dose: T=0 (just prior to glucose dose), 15, 30, 60, 90, and 120 min. Following the final blood glucose measurement, food was returned to the cages.

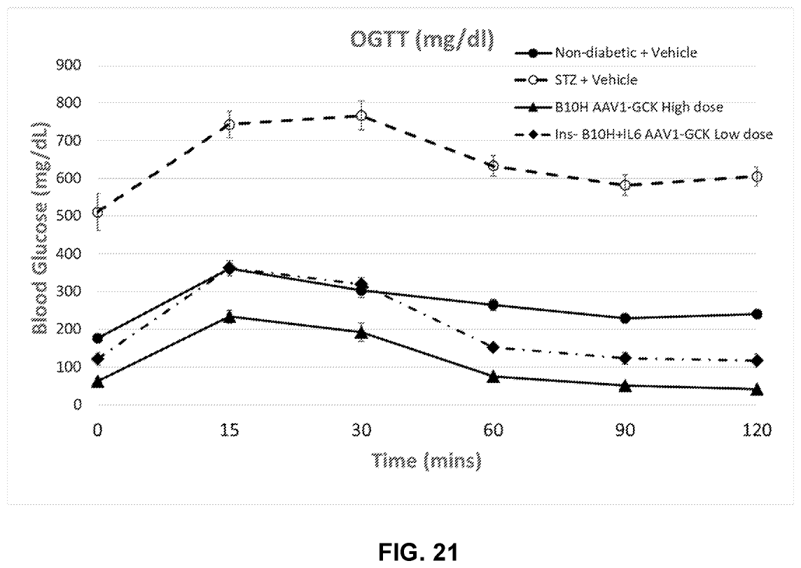

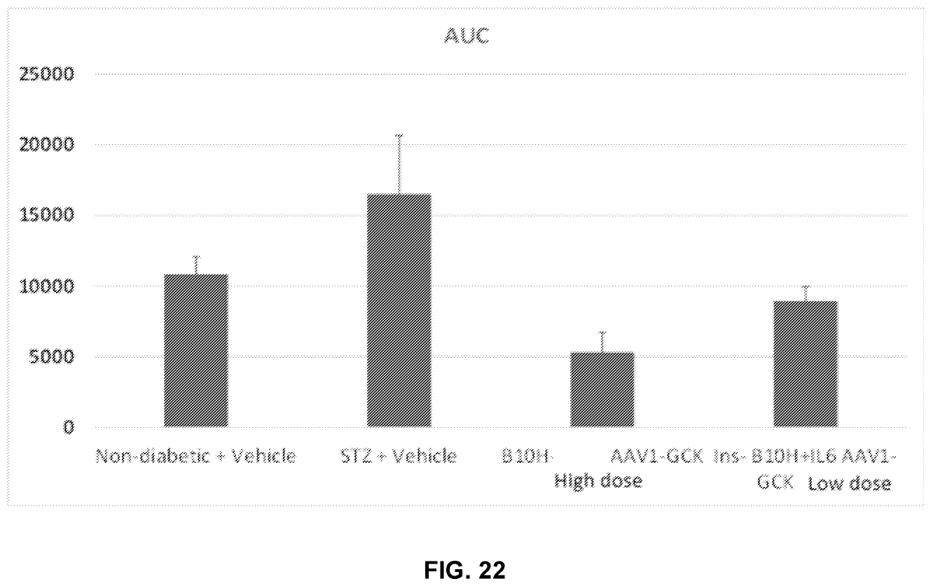

The fasting blood glucose (T=0) of non-diabetic control mice was significantly less than that of the STZ-treated animals (FIG. 21). Treatment with the AAV1 vector constructs significantly reduced fasting glucose compared to STZ control mice and to the level of the non-diabetic controls. Following oral gavage with glucose (2 g/kg) the blood glucose of non-STZ controls increased 189±17 mg/dL with a peak at 15 min and return to near-control levels in 90 mins. In contrast, STZ treated mice increased 264±37 mg/dL with a peak occurring nearer to 30 min and fail to return to control levels even at 120 mins. This data demonstrated that STZ treated mice were unable to regulate glucose disposal normally following a post-prandial excursion. Similar to non-diabetic controls, IM Injections with vector constructs hInsB10H (included coding sequence of SEQ ID NO: 111)+AAV1-GCK (AAV926) (high dose) and hInsB10H+IL6 (included coding sequence of SEQ ID NO: 120)+AAV1-GCK (AAV926) (low dose) produced peak glucose excursions of 154±16 and 218±22 mg/dL respectively at 15 minutes post challenge. Both treatments returned blood glucose to T-0 levels within 60 minutes. Results from ANCOVA analysis utilizing AUCs supported these interpretations (FIG. 22). Overall, these data suggest that IM injection of these constructs can not only control fasting glucose levels, but can also blunt the large post-prandial glucose excursions typically observed in diabetic patients.

A goal for Type 1 diabetes is to normalize glycemic control with no change in body weight while preventing diabetic ketoacidosis and medically consequential hypoglycemia. Since Type 1 diabetics have very little or no circulating insulin, they must take insulin every day to stay alive. Further, it has been reported that the hyperglycemia produced by streptozotocin (STZ)-induced diabetes, leads to progressive insulin resistance of the peripheral tissues (Ordóñez P, Moreno M, Alonso A, Fernández R, Diaz F and González C. Insulin sensitivity in streptozotocin-induced diabetic rats treated with different doses of 17beta-oestradiol or progesterone. Exp Physiol 92:241-9, 2007. doi: 10.1113/expphysiol.2006.035006. Epub 2006 Oct. 26.) The results shown here support that being able to provide an alternative supply of insulin to muscle in addition to improving insulin sensitivity of peripheral tissues provides multiple means to restore glycemic control.

10.3 HbA1c Levels in Diabetic Mice after Administration of AAV-Ins and AAV-Gck Vectors

A blood sample was obtained during the final non-fasted blood collection, 8-wk post injection. Glycated blood hemoglobin (HbA1c) was determined from the 2nd drop of blood (5-10 μL) from a tail snip using a hand-held HbA1c meter. This method has been validated with comparison testing against a National Glycohemoglobin Standardization Program (NGSP) certified method as a gold standard reference. HbA1c was not determined in animals that expired or were euthanized due to poor health/hypoglycemia (AAV1-Ins+AAV1 GCK high dose).

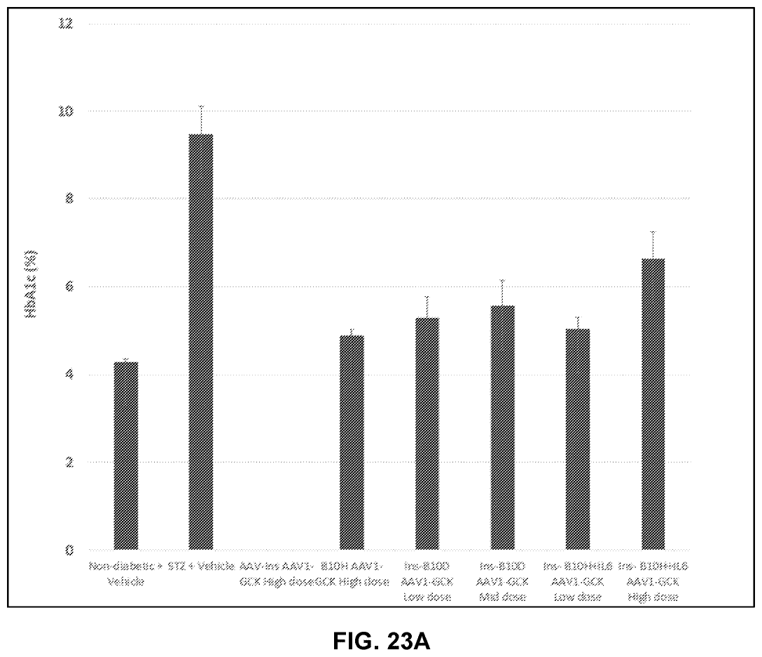

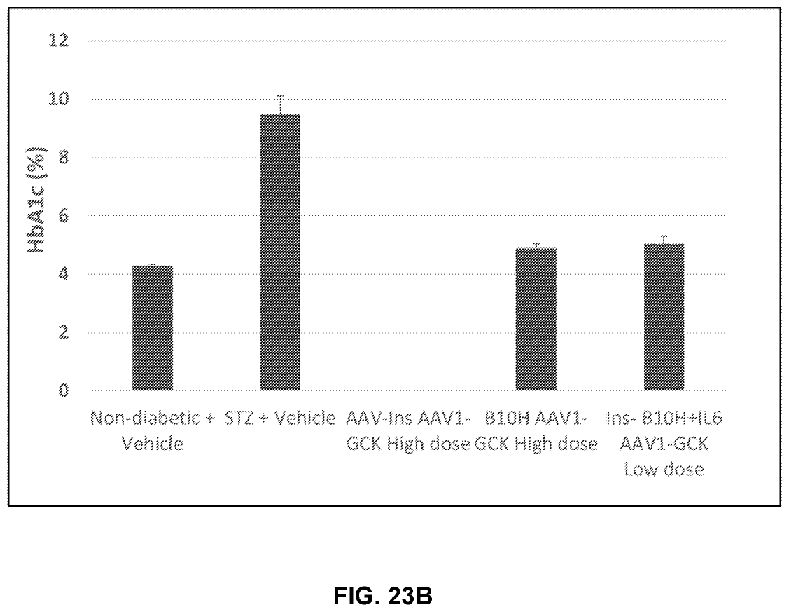

The normal range of HbA1c in non-diabetic mice is 4%-5.6% with diabetes defined as an HbA1c>6.5%. Data from this study are shown in FIG. 23. The HbA1c value for non-STZ control mice was 4.3±0.05%. Administration of STZ produced a significant increase in HbA1c (9.48±0.64%; p<0.001) compared to non-diabetic controls. Treatment with vector constructs hInsB10H+AAV1-GCK (included coding sequence of SEQ ID NO: 111) (AAV926) high dose (4.89±0.15) and hInsB10H+IL6 (included coding sequence of SEQ ID NO: 120)+AAV1-GCK (AAV926) low dose (5.03±0.27%) significantly reduced blood glucose and HbA1c to the level of non-diabetic controls.

HbA1c is an integrated signal reflecting average glycemia over a period of time; in the case of a mouse, the red blood cell t_{1/2} is ˜14 days. Clinically, this test is the major tool for assessing glycemic control and has strong predictive value for diabetes and comorbidities. A goal of diabetes therapy is to maintain HbA1c in the normal range (<6.5%), and most marketed products lower HBA1C between 0.5 and 1.25%. Here, chemical induction of type 1 diabetes in mice with STZ increased HbA1c from 4.3 to 9.48%. IM Injections with AAV1 vectors containing hINS and rGCK essentially normalized HbA1c to those of non-diabetic controls within 8 wks. Further, these vectors produced a >4% reduction in HbA1c compared to STZ controls. All the groups lowered HbA1c, however only hInsB10H (included coding sequence of SEQ ID NO: 111)+AAV1-Gck (high dose) and hIns-B10H-IL6 (included coding sequence of SEQ ID NO: 120)+AAV1-Gck (low dose) survived to the 8 week blood draw meaning that the HbA1c levels are the average over the 8 weeks.

HbA1c along with the temporal measurements of weekly blood glucose provided a quantitative measure of both an improved post-prandial glucose exposure over a period of time and a reduced degree of glycemic variability suggesting that both factors can be normalized with this treatment. The results support that hIns and Gck AAV constructs co-administered IM to a Type 1 Diabetic patient could represent a single dose reversal of this chronic and debilitating disease.

10.4 Serum Triglycerides and Ketone Body Levels in Diabetic Mice after Administration of AAV-Ins and AAV-Gck Vectors

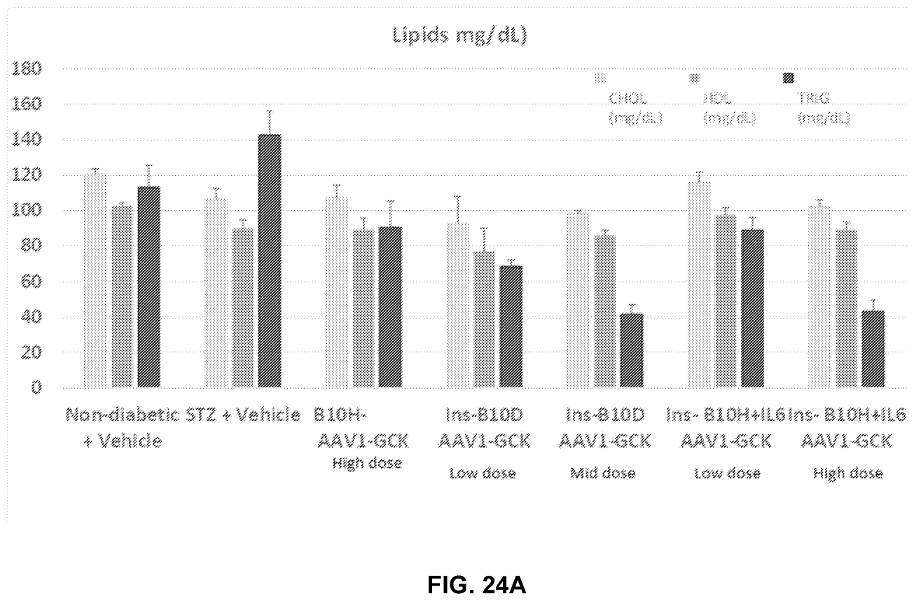

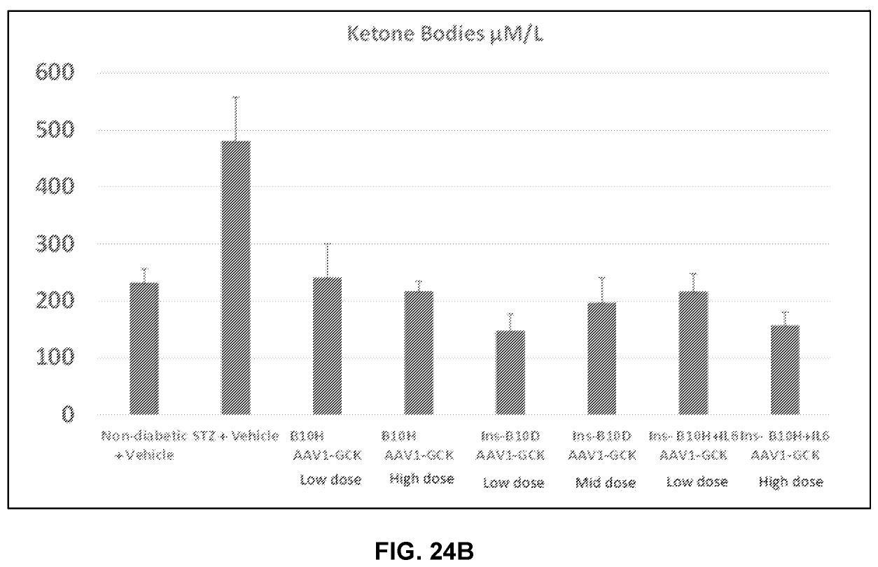

Mouse models of STZ-induced diabetes have both elevated blood triglyceride and ketone levels compared to non-diabetic controls and have shifted to a predominantly lipid-based energy source. IM injection of vector constructs hInsB10H (included coding sequence of SEQ ID NO: 111)+AAV1-GCK (AAV926) high dose and hInsB10H+IL6 (included coding sequence of SEQ ID NO: 120)+AAV1-GCK (AAV926) low dose reduced circulating triglyceride (FIG. 24A) and ketone (FIG. 24B) levels to the level of non-diabetic controls or below. This data showed that multiple metabolic endpoints were normalized after administration of AAV-hInsB10H (with native or IL-6 signal sequence) and AAV-Gck vectors.

10.5 Liver hINS mRNA Levels in Diabetic Mice after Administration of AAV-Ins and AAV-Gck Vectors

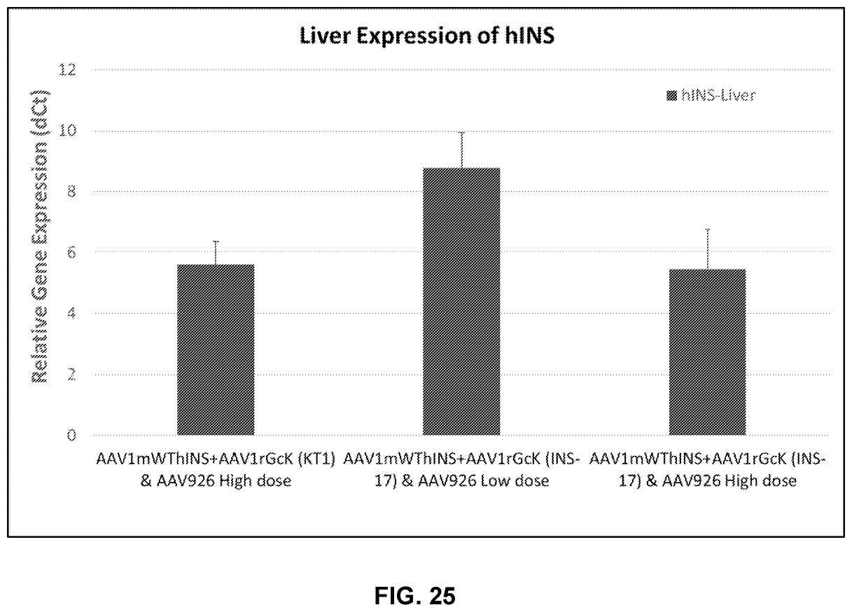

qPCR analysis was used to assess mRNA expression in STZ-induced diabetic mice after administration of AAV-Ins and AAV-Gck constructs. hINS mRNA levels in the liver was assessed to determine whether the AAV-1 vectors escaped the muscle, entered the circulation and transduced the liver, subsequently becoming transcribed at a detectible level. Samples were not measured in non-Diabetic Control or STZ-Control mice since no vectors were injected in those groups. Animals who experienced unexpected deaths or showed distress were not analyzed. The results are shown in Table 18 and FIG. 25.

TABLE 18
Liver Expression of hINS

| Vector Construct | Δ Ct hINS |
|---|---|
| AAV1mWThINS + AAV1rGcK & AAV926 | 5.61 ± .77 |
| (B10H AAV1-GCK) | |
| High dose | |
| AAV1mWThINS + AAV1rGcK (INS-17) & | 8.78 ± 1.15 |
| AAV926 | |
| (Ins-B10H + IL6 AAV1-GCK) | |
| Low dose | |
| AAV1mWThINS + AAV1rGcK (INS-17) & | 5.46 ± 1.30 |
| AAV926 | |
| (Ins-B10H + IL6 AAV1-GCK) | |
| High dose | |

The results of this assay show a A Ct of >5 cycles suggesting very low or no hINS mRNA present in the liver of mice administered the three constructs tested (AAV1-mWTIns+AAV1-rGck (AAV926) high dose; AAV1-mWTIns (Ins17)+AAV1-rGck (AAV926) low dose; and AAV1-mWTIns (Ins17)+AAV1-rGck (AAV926) high dose) and therefore the IM injected AAVs remain mostly in the target muscles. These results show that the IM delivered AAV1-Ins constructs resulted in observed normalization of blood glucose, HbA1c, ketones, and triglycerides due to the transduction of the AAV-Ins and AAV-Gck vectors and protein expression in the muscle.

1-35. (canceled)

36. A polynucleotide comprising a nucleic acid encoding a human glucokinase (Gck) protein, wherein the nucleic acid comprises
a sequence at least 85%, 90%, 95%, at least 96%, at least 97%, at least 98%, at least 99%, or 100% identical to any one of SEQ ID NOs: 61-80, or any combination thereof.

37. The polynucleotide of claim 36, wherein the human Gck protein comprises the sequence of SEQ ID NO: 82.

38. The polynucleotide of claim 36, which further comprises a 5′ UTR comprising a sequence at least 85%, at least 90%, at least 95%, at least 99%, or 100% identical to nucleic acids 5-329 of SEQ ID NO: 42.

39. The polynucleotide of claim 36, which further comprises a 5′ UTR comprising a sequence at least 85%, at least 90%, at least 95%, at least 99%, or 100% identical to any one of SEQ ID NO: 42, SEQ ID NO: 83, SEQ ID NO: 146, or SEQ ID NO: 148.

40. The polynucleotide of claim 26, which further comprises a 3′ UTR comprising a nucleotide-sequence at least 85%, at least 90%, at least 95%, at least 99%, or 100% identical to SEQ ID NO: 60, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, or SEQ ID NO: 149.

41. (canceled)

42. The polynucleotide of claim 36, which comprises
a sequence at least 85%, at least 90%, at least 95%, at least 96%, at least 97%, at least 98%, at least 99%, or 100% identical to any one of SEQ ID NOs: 20-39 and 89-96.

43-44. (canceled)

45. An expression cassette comprising the polynucleotide of claim 36 and a promoter operably linked to the nucleic acid sequence.

46. (canceled)

47. The expression cassette of claim 45, wherein the promoter is a constitutive promoter.

48. The expression cassette of claim 45, wherein the promoter is a CMV promoter.

49. The expression cassette of claim 36, wherein the nucleic acid is operably linked to a polyadenylation (polyA) element.

50. A vector comprising the polynucleotide of claim 36.

51. The vector of claim 50, wherein the vector is a viral vector or a non-viral vector.

52. The vector of claim 51, wherein the vector is an adeno-associated virus (AAV) vector.

53. A recombinant AAV (rAAV) particle, comprising an AAV capsid and a vector genome comprising a polynucleotide, wherein the polynucleotide comprises a nucleic acid encoding a human glucokinase (Gck) protein, wherein the nucleic acid encoding the human Gck protein comprises a sequence at least 95%, at least 96%, at least 97%, at least 98%, at least 99%, or 100% identical to any one of SEQ ID NOs: 61-80.

54. The rAAV particle of claim 53, wherein the AAV capsid serotype is selected from the group consisting of AAV1, AAV2, AAV3, AAV4, AAV5, AAV6, AAV7, AAV8, AAVRH8, AAVrh9, AAV9, AAVrh10, and AAV10, AAV11, AAV12.

55. A host cell comprising the polynucleotide of claim 36.

56. The host cell of claim 55, wherein the host cell is a mammalian cell.

57. A method of producing human Gck protein in a cell, comprising contacting and/or transforming the cell with the polynucleotide of claim 36, thereby producing the human Gck protein in the cell.

58. A method of producing human Gck protein in a subject, comprising administering to the subject the polynucleotide of claim 36, thereby producing the human Gck protein in the subject.

59. A method of treating or ameliorating the symptoms associated with diabetes in a subject in need thereof, comprising delivering to the subject a therapeutically effective amount of a polynucleotide comprising a nucleic acid encoding a human glucokinase (Gck) protein, wherein the nucleic acid encoding the human Gck protein comprises a sequence at least 95%, at least 96%, at least 97%, at least 98%, at least 99%, or 100% identical to any one of SEQ ID NOs: 61-80, thereby treating diabetes in the subject.

60. The method of claim 59, wherein the diabetes is diabetes mellitus type 1 (T1DM) or diabetes mellitus type 2 (T2DM).

61-64. (canceled)

65. The method of claim 59, wherein the delivery and/or administration is intramuscular.

66. The method of claim 59, wherein (i) glycated blood hemoglobin (HbA1c) levels are reduced and/or regulated in the subject; (ii) circulating ketones are reduced in the subject, (iii) triglycerides are reduced in the subject, or (iv) any combination thereof.

67. The polynucleotide of claim 36, wherein the nucleic acid encoding the human Gck protein comprises a sequence at least 95%, at least 96%, at least 97%, at least 98%, at least 99%, or 100% identical to SEQ ID NO: 68.

68. The polynucleotide of claim 42, which comprises a sequence at least 95%, at least 96%, at least 97%, at least 98%, at least 99%, or 100% identical to SEQ ID NO: 27.

69. The method of claim 59, wherein the method further comprises separate administration of a recombinant insulin to the subject.

70. The method of claim 69, wherein the polynucleotide is a single administration and the recombinant insulin is administered via regular injections.

71. A recombinant AAV1 (rAAV1) vector, comprising an AAV1 serotype capsid and a vector genome comprising a nucleic acid encoding a human Gck protein, wherein the nucleic acid encoding the human Gck protein comprises a sequence at least 99% or 100% identical to SEQ ID NO: 68, wherein the nucleic acid encoding the human Gck is operably linked to a CMV promoter.

72. A method of treating or ameliorating the symptoms associated with diabetes in a subject comprising administering a combination therapy to the subject, wherein the combination therapy comprises a composition comprising the rAAV1 vector of claim 71 and a separate composition comprising a recombinant insulin.

73. The method of claim 72, wherein the rAAV1 vector is a single administration and the recombinant insulin is administered via regular injections.
